SEQUENCE LISTING (254 sequences)

SEQ ID NO: 1 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTVITKDDAFDIWGQGTMVTVSS

SEQ ID NO: 2 - protein, 113 aa, synthetic construct
  REGION 1..113: note = Recombinant Antibody Sequence
DIVMTQSPDSLAVSLGERATINCKSSQNILYSSNNKNYLAWYQQKPGQPPKLLFYWASTRESGVPDRFSGSGSGTDFTLTISSLQAEDVAVYYCHQYYSSPLTFGGGTKVEIK

SEQ ID NO: 3 - protein, 121 aa, synthetic construct
  REGION 1..121: note = Recombinant Antibody Sequence
QVQLVQSGAEVKKPGASVKVSCKASGYTFTRYAIHWVRQAPGQRLEWMGWIKAGNGNTRYSQKFQDRVTITRDTSASTAYMELSSLRSEDTAVYYCALLTVITPDDAFDIWGQGTMVTVSS

SEQ ID NO: 4 - protein, 113 aa, synthetic construct
  REGION 1..113: note = Recombinant Antibody Sequence
DIVMTQSPDSLAVSLGERATINCKSSQNIFFSSKNKNYLAWYQQIPGQPPKLLIYWASTRESGVPDRFSGSGSGTDFTLTISSLQAEDVAVYYCHQYYSSPLTFGGGTKVEIQ

SEQ ID NO: 5 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVIRQDDTFDIWGQGTMVTVSS

SEQ ID NO: 6 - protein, 113 aa, synthetic construct
  REGION 1..113: note = Recombinant Antibody Sequence
DIVMTQSPDSLAVSLGERATINCKSSQNILYSSNNKNYLAWYQQKPGQAPKLLIYWASTRESGVPDRFSGSGSGTDFTLTISSLQAEDVAVYYCHQYYSSPLTFGGGTKVEIK

SEQ ID NO: 7 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTVITPDDRFDIWGQGTMVTVSS

SEQ ID NO: 8 - protein, 113 aa, synthetic construct; identical to SEQ ID NO: 2
  REGION 1..113: note = Recombinant Antibody Sequence

SEQ ID NO: 9 - protein, 121 aa, synthetic construct
  REGION 1..121: note = Recombinant Antibody Sequence
QVQLVQSGAEVKKPGASVKVSCKTSGYTFTNYALHWVRQAPGQRLEWMGWIKTGNGDTRYSQKFQDRVTITRDTSASTAYMELGSLRSEDTAVYYCALLTVITPDDAFDIWGQGTMVTVSS

SEQ ID NO: 10 - protein, 113 aa, synthetic construct
  REGION 1..113: note = Recombinant Antibody Sequence
DIVMTQSPDSLAVSLGERATINCKSSQSVLYRTNNKNYLAWYQQKPGQPPKLLIYWASTRESGVPDRFSGSGSGTDFTLTISSLQAEDVAVYYCHQYYSSPLTFGGGTKVEIK

SEQ ID NO: 11 - protein, 121 aa, synthetic construct
  REGION 1..121: note = Recombinant Antibody Sequence
QVHLVQSGAEVKKPGASVKVSCKASGYTFTRYAIHWVRQAPGQRLEWMGWIKGGNGNTRYSQKFQDRVTITRDTSASTAYMELSSLRSEDTAVYYCALLTVITPDDAFDIWGQGTMVTVSS

SEQ ID NO: 12 - protein, 113 aa, synthetic construct
  REGION 1..113: note = Recombinant Antibody Sequence
DIVMTQSPDSLAVSLGERATINCKSSQNIFFSSNNKNYLAWYQQKPGQPPKLLFYWASTRESGVPDRFSGSGSGTDFTLTISSLQAEDVAVYYCHQYYSSPLTFGGGTKVEIK

SEQ ID NO: 13 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLMVITPDDAFDIWGQGTMVTVSS

SEQ ID NO: 14 - protein, 113 aa, synthetic construct; identical to SEQ ID NO: 2
  REGION 1..113: note = Recombinant Antibody Sequence

SEQ ID NO: 15 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQGRVTITRDTSASTAYMELSSLRSEDTAGYYCALLTVITPDDAFDIWGQGTMVTVSS

SEQ ID NO: 16 - protein, 113 aa, synthetic construct
  REGION 1..113: note = Recombinant Antibody Sequence
DIVMTQSPDSLAVSLGERASINCKSSQNILFSSNNKNYLAWYQQKPGQPPKLLIYWASTRESGVPDRFSGSGSGTDFTLTISSLQAEDVAVYICHQYYSSPLTFGGGTKVEIK

SEQ ID NO: 17 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVITPDDTFDIWGQGTMVTVSS

SEQ ID NO: 18 - protein, 113 aa, synthetic construct; identical to SEQ ID NO: 6
  REGION 1..113: note = Recombinant Antibody Sequence

SEQ ID NO: 19 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTVITPDDAFDIWGQGTMVTVSS

SEQ ID NO: 20 - protein, 113 aa, synthetic construct; identical to SEQ ID NO: 2
  REGION 1..113: note = Recombinant Antibody Sequence

SEQ ID NO: 21 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTKYSQKFQGRVTITRDTSASTAYMELSSLRSEDTAVYYCALLTVITPDDAFDIWGQGTMVTVSS

SEQ ID NO: 22 - protein, 113 aa, synthetic construct
  REGION 1..113: note = Recombinant Antibody Sequence
DIVMTQSPDSLAVSLGERATINCKSSQNILYSSNNKNYLAWYQQKPGQPPKLLIYWASTRESGVPDRFSGSGSGTDFTLTISSLQAEDVAVYYCHQYYSSPLTFGGGTKVEIK

SEQ ID NO: 23 - protein, 5 aa, synthetic construct
  REGION 1..5: note = Antibody CDR Sequence
SYAIH

SEQ ID NO: 24 - protein, 17 aa, synthetic construct
  REGION 1..17: note = Antibody CDR Sequence
WIKGGNGNTRYSQKFQD

SEQ ID NO: 25 - protein, 12 aa, synthetic construct
  REGION 1..12: note = Antibody CDR Sequence
LTVITKDDAFDI

SEQ ID NO: 26 - protein, 17 aa, synthetic construct
  REGION 1..17: note = Antibody CDR Sequence
KSSQNILYSSNNKNYLA

SEQ ID NO: 27 - protein, 7 aa, synthetic construct
  REGION 1..7: note = Antibody CDR Sequence
WASTRES

SEQ ID NO: 28 - protein, 9 aa, synthetic construct
  REGION 1..9: note = Antibody CDR Sequence
HQYYSSPLT

SEQ ID NO: 29 - protein, 5 aa, synthetic construct
  REGION 1..5: note = Antibody CDR Sequence
RYAIH

SEQ ID NO: 30 - protein, 17 aa, synthetic construct
  REGION 1..17: note = Antibody CDR Sequence
WIKAGNGNTRYSQKFQD

SEQ ID NO: 31 - protein, 12 aa, synthetic construct
  REGION 1..12: note = Antibody CDR Sequence
LTVITPDDAFDI

SEQ ID NO: 32 - protein, 17 aa, synthetic construct
  REGION 1..17: note = Antibody CDR Sequence
KSSQNIFFSSKNKNYLA

SEQ ID NO: 33 - protein, 12 aa, synthetic construct
  REGION 1..12: note = Antibody CDR Sequence
LTVIRQDDTFDI

SEQ ID NO: 34 - protein, 12 aa, synthetic construct
  REGION 1..12: note = Antibody CDR Sequence
LTVITPDDRFDI

SEQ ID NO: 35 - protein, 5 aa, synthetic construct
  REGION 1..5: note = Antibody CDR Sequence
NYALH

SEQ ID NO: 36 - protein, 17 aa, synthetic construct
  REGION 1..17: note = Antibody CDR Sequence
WIKTGNGDTRYSQKFQD

SEQ ID NO: 37 - protein, 17 aa, synthetic construct
  REGION 1..17: note = Antibody CDR Sequence
KSSQSVLYRTNNKNYLA

SEQ ID NO: 38 - protein, 17 aa, synthetic construct
  REGION 1..17: note = Antibody CDR Sequence
KSSQNIFFSSNNKNYLA

SEQ ID NO: 39 - protein, 12 aa, synthetic construct
  REGION 1..12: note = Antibody CDR Sequence
LMVITPDDAFDI

SEQ ID NO: 40 - protein, 17 aa, synthetic construct
  REGION 1..17: note = Antibody CDR Sequence
WIKAGNGNTRYSQKFQG

SEQ ID NO: 41 - protein, 17 aa, synthetic construct
  REGION 1..17: note = Antibody CDR Sequence
KSSQNILFSSNNKNYLA

SEQ ID NO: 42 - protein, 12 aa, synthetic construct
  REGION 1..12: note = Antibody CDR Sequence
LTVITPDDTFDI

SEQ ID NO: 43 - protein, 17 aa, synthetic construct
  REGION 1..17: note = Antibody CDR Sequence
WIKAGNGNTKYSQKFQG

SEQ ID NO: 44 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSTTTAYMELSSLRSEDTAVYYCALLTVLTPDDAFDIWGQGTMVTVSS

SEQ ID NO: 45 - protein, 113 aa, synthetic construct
  REGION 1..113: note = Recombinant Antibody Sequence
DIVMTQSPDSLAVSLGERATINCKSSQSVLYSSNNKNYLAWYQQKPGQPPNLLIYWASTRQSGVPDRFSGSGSGTDFTLTISSLQAEDVAVYYCHQYYSSPLTFGGGTKVEIK

SEQ ID NO: 46 - protein, 120 aa, synthetic construct
  REGION 1..120: note = Recombinant Antibody Sequence
QVKLVESGGGVVQPGRSLRLSCEASGFIFSTYGMHWVRQAPGKGLEWVAVIWFDGSNIYYADSVKGRFTISRDNSKNTVFMQMDSLRAEDTAVYYCHRNFYDGSGPFDYWGQGTLVTVSS

SEQ ID NO: 47 - protein, 107 aa, synthetic construct
  REGION 1..107: note = Recombinant Antibody Sequence
DIQMTQSPSTLSASVGDRVTITCRASQFISRWLAWYQQKPGKAPKLLIYKASSLESGVPSRFSGSGSETHFTLTISSLQPDDVATYYCQEYTSYGRTFGQGTKVEIK

SEQ ID NO: 48 - protein, 397 aa, Plasmodium falciparum
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 49 - protein, 451 aa, synthetic construct
  REGION 1..451: note = Recombinant Antibody Sequence
QVQLVQSGAEVKKPGASVKVSCKASGYTFTSYAIHWVRQAPGQRLEWMGWIKAGNGNTRYSQKFQDRVTITRDTSTTTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 220 aa, synthetic construct
  REGION 1..220: note = Recombinant Antibody Sequence
DIVMTQSPDSLAVSLGERATINCKSSQSVLYSSNNKNYLAWYQQKPGQPPNLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 15 aa, synthetic construct
  REGION 1..15: note = Recombinant Antibody Sequence
PADGNPDPNANPNVD

SEQ ID NO: 52 - protein, 15 aa, synthetic construct
  REGION 1..15: note = Recombinant Antibody Sequence
NPDPNANPNVDPNAN

SEQ ID NO: 53 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALMTVITPDDAFDIWGQGTMVTVSS

SEQ ID NO: 54 - protein, 113 aa, synthetic construct; identical to SEQ ID NO: 12
  REGION 1..113: note = Recombinant Antibody Sequence

SEQ ID NO: 55 - protein, 121 aa, synthetic construct; identical to SEQ ID NO: 53
  REGION 1..121: note = Recombinant Antibody Sequence

SEQ ID NO: 56 - protein, 113 aa, synthetic construct
  REGION 1..113: note = Recombinant Antibody Sequence
DIVMTQSPDSLAVSLGERATINCKSSQNIFYSSNNKNYLAWYQQKPGQPPKLLFYWASTRESGVPDRFSGSGSGTDFTLTISSLQAEDVAVYYCHQYYSSPLTFGGGTKVEIK

SEQ ID NO: 57 - protein, 121 aa, synthetic construct; identical to SEQ ID NO: 11
  REGION 1..121: note = Recombinant Antibody Sequence

SEQ ID NO: 58 - protein, 113 aa, synthetic construct
  REGION 1..113: note = Recombinant Antibody Sequence
DIVMTQSPDSLAVSLGERATINCKSSQNIFFSSNNKNYLAWYQQIPGQPPKLLFYWASTRESGVPDRFSGSGSGTDFTLTISSLQAEDVAVYYCHQYYSSPLTFGGGTKVEIQ

SEQ ID NO: 59 - protein, 121 aa, synthetic construct; identical to SEQ ID NO: 3
  REGION 1..121: note = Recombinant Antibody Sequence

SEQ ID NO: 60 - protein, 113 aa, synthetic construct
  REGION 1..113: note = Recombinant Antibody Sequence
DIVMTQSPDSLAVSLGERATINCKSSQNIFFSSNNKNYLAWYQQIPGQPPKLLIYWASTRESGVPDRFSGSGSGTDFTLTISSLQAEDVAVYYCHQYYSSPLTFGGGTKVEIQ

SEQ ID NO: 61 - protein, 121 aa, synthetic construct; identical to SEQ ID NO: 3
  REGION 1..121: note = Recombinant Antibody Sequence

SEQ ID NO: 62 - protein, 113 aa, synthetic construct; identical to SEQ ID NO: 4
  REGION 1..113: note = Recombinant Antibody Sequence

SEQ ID NO: 63 - protein, 121 aa, synthetic construct
  REGION 1..121: note = Recombinant Antibody Sequence
QVQLVQSGAEVKKPGASVKVSCKASGYTFTTYAIHWVRQAPGQRLEWMGWIKVGDGNTRYSPKFQDRVTITRDTSASTAYMELSSLRSEDTAVYFCALLTVITPDDAFDIWGQGTMVTVSS

SEQ ID NO: 64 - protein, 113 aa, synthetic construct
  REGION 1..113: note = Recombinant Antibody Sequence
DIVMTQSPDSLAVSLGERATINCKSSQNIFFSSKNKNYLAWYQQIPGQSPKLLIYWASTRESGVPDRFSGSGSGTDFTLTISSLQAEDVAVYYCHQYYSSPLTFGGGTKVEIQ

SEQ ID NO: 65 - protein, 121 aa, synthetic construct
  REGION 1..121: note = Recombinant Antibody Sequence
QVQLVQSGAEVKKPGASVKVSCKASGYTFTRYAIHWVRQAPGQRLEWMGWIKAGNGNTRYSQKFQDRVTITRDTSASTAYMELSSLRSEDTAVYYCALLTVITPDDTFDIWGQGTMVTVSS

SEQ ID NO: 66 - protein, 113 aa, synthetic construct
  REGION 1..113: note = Recombinant Antibody Sequence
DIVMTQSPDSLAVSLGERATINCKSSQNIFFSSNNKNYLAWYQQIPGQAPKLLIYWASTRESGVPDRFSGSGSGTDFTLTISSLQAEDVAVYYCHQYYSSPLTFGGGTKVEIQ

SEQ ID NO: 67 - protein, 121 aa, synthetic construct
  REGION 1..121: note = Recombinant Antibody Sequence
QVQLVQSGAELKKPGASVKVSCKASGYTFTRYAIHWVRQAPGQRLEWMGWIKAGNGNTRYSQKFQDRVTITRDTSASTAYMELNSLRSEDTAVYYCALLTVITPDDAFDIWGQGTMVTVSS

SEQ ID NO: 68 - protein, 113 aa, synthetic construct
  REGION 1..113: note = Recombinant Antibody Sequence
DIVMTQSPDSLAVSLGERATINCKSSQNIFFSSNNKNYLAWYQQIPGQPPQLLIYWASTRESGVPDRFSGSGSGTDFTLTISSLQAEDVAVYYCHQYYSSPLTFGGGTKVEIQ

SEQ ID NO: 69 - protein, 121 aa, synthetic construct
  REGION 1..121: note = Recombinant Antibody Sequence
QVHLVQSGAEVKKPGASVKVSCKASGYTFTSYWIHWVRQAPGQRLEWMGWIKGGNGNTRYSQKFQDRVTITRDTSASTAYMELSSLRSEDTAVYYCALLTVITPDDAFDIWGQGTMVTVSS

SEQ ID NO: 70 - protein, 113 aa, synthetic construct; identical to SEQ ID NO: 2
  REGION 1..113: note = Recombinant Antibody Sequence

SEQ ID NO: 71 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTWITPDDAFDIWGQGTMVTVSS

SEQ ID NO: 72 - protein, 113 aa, synthetic construct; identical to SEQ ID NO: 2
  REGION 1..113: note = Recombinant Antibody Sequence

SEQ ID NO: 73 - protein, 121 aa, synthetic construct
  REGION 1..121: note = Recombinant Antibody Sequence
QVHLVQSGAEVKKPGASVKVSCKASGYTFTSYWIHWVRQAPGQRLEWMGWIDGGNGNTRYSQKFQDRVTITRDTSASTAYMELSSLRSEDTAVYYCALLTVITPDDAFDIWGQGTMVTVSS

SEQ ID NO: 74 - protein, 113 aa, synthetic construct; identical to SEQ ID NO: 2
  REGION 1..113: note = Recombinant Antibody Sequence

SEQ ID NO: 75 - protein, 121 aa, synthetic construct
  REGION 1..121: note = Recombinant Antibody Sequence
QVHLVQSGAEVKKPGASVKVSCKASGYTFTSYWIHWVRQAPGQRLEWMGWIDGGKGNTRYSQKFQDRVTITRDTSASTAYMELSSLRSEDTAVYYCALLTVITPDDAFDIWGQGTMVTVSS

SEQ ID NO: 76 - protein, 113 aa, synthetic construct; identical to SEQ ID NO: 2
  REGION 1..113: note = Recombinant Antibody Sequence

SEQ ID NO: 77 - protein, 121 aa, synthetic construct
  REGION 1..121: note = Recombinant Antibody Sequence
QVHLVQSGAEVKKPGASVKVSCKASGYTFTSYWIHWVRQAPGQRLEWMGWIKGGNGNTRYSQKFQDRVTITRDTSASTAYMELSSLRSEDTAVYYCALLTWITPDDAFDIWGQGTMVTVSS

SEQ ID NO: 78 - protein, 113 aa, synthetic construct
  REGION 1..113: note = Recombinant Antibody Sequence
DIVMTQSPDSLAVSLGERATINCKSSQNILYSSYNKNYLAWYQQKPGQPPKLLFYWASTRESGVPDRFSGSGSGTDFTLTISSLQAEDVAVYYCHQYYSSPLTFGGGTKVEIK

SEQ ID NO: 79 - protein, 121 aa, synthetic construct; identical to SEQ ID NO: 44
  REGION 1..121: note = Recombinant Antibody Sequence

SEQ ID NO: 80 - protein, 113 aa, synthetic construct
  REGION 1..113: note = Recombinant Antibody Sequence
DIVMTQSPDSLAVSLGERATINCKSSQSVLYSSNNKNYLAWYQQKPGQPPNLLIYWASTRQSGVPDRFSGSGSGTDFTLTISSLQAEDVAVYYCHQYYRSPLTFGGGTKVEIK

SEQ ID NO: 81 - protein, 121 aa, synthetic construct
  REGION 1..121: note = Recombinant Antibody Sequence
QVQLVQSGAEVKKPGASVKVSCKASGYTFTSYWIHWVRQAPGQRLEWMGWIKAGNGNTRYSQKFQDRVTITRDTSTTTAYMELSSLRSEDTAVYYCALLTVLTPDDAFDIWGQGTMVTVSS

SEQ ID NO: 82 - protein, 113 aa, synthetic construct; identical to SEQ ID NO: 45
  REGION 1..113: note = Recombinant Antibody Sequence

SEQ ID NO: 83 - protein, 113 aa, synthetic construct
  REGION 1..113: note = Recombinant Antibody Sequence
DIVMTQSPDSLAVSLGERATINCKSSQSVLWSSNNKNYLAWYQQKPGQPPNLLIYWASTRQSGVPDRFSGSGSGTDFTLTISSLQAEDVAVYYCHQYYSSPLTFGGGTKVEIK

SEQ ID NO: 84 - protein, 121 aa, synthetic construct
  REGION 1..121: note = Recombinant Antibody Sequence
QVQLVQSGAEVKKPGASVKVSCKASGYTFTNYWIHWVRQAPGQRLEWMGWINAVIGNTRYSQKFQGRVTITRDTSASTAYMELSSLRSEDTAVYYCALLTVITPDDAFDVWGQGTMVTVSS

SEQ ID NO: 85 - protein, 113 aa, synthetic construct
  REGION 1..113: note = Recombinant Antibody Sequence
DIVMTQSPDSLAVSLGERATINCKSSQSILYTSNNKKYLAWYQQKPGQPPKLLIYWASTRISGVPDRFSGSGSGTDFTLTISSLQAEDVAVYFCHQYYSSPLTFGGGTKVEIK

SEQ ID NO: 86 - protein, 121 aa, synthetic construct
  REGION 1..121: note = Recombinant Antibody Sequence
QVQLVQSGAEVKKPGASVKVSCKASGYTFTNYAIHWVRQAPGQRLEWMGWINAVIGNTRYSQKFQGRVTITRDTSASTAYMELSSLRSEDTAVYYCALLTVIRPDDAFDVWGQGTMVTVSS

SEQ ID NO: 87 - protein, 113 aa, synthetic construct; identical to SEQ ID NO: 85
  REGION 1..113: note = Recombinant Antibody Sequence

SEQ ID NO: 88 - protein, 121 aa, synthetic construct
  REGION 1..121: note = Recombinant Antibody Sequence
QVQLVQSGAEVKKPGASVKVSCKASGYTFTNYWIHWVRQAPGQRLEWMGWINAVIGNTRYSQKFQGRVTITRDTSASTAYMELSSLRSEDTAVYYCALLTVIRPDDAFDVWGQGTMVTVSS

SEQ ID NO: 89 - protein, 113 aa, synthetic construct; identical to SEQ ID NO: 85
  REGION 1..113: note = Recombinant Antibody Sequence

SEQ ID NO: 90 - protein, 113 aa, synthetic construct
  REGION 1..113: note = Recombinant Antibody Sequence
DIVMTQSPDSLAVSLGERATINCKSSQSVLYSSNNKNYLAWYQQKPGQPPNLLIYWASTRQSGVPDRFSGSGSGTDFTLTISSLQAEDVAVYYCHQYYNSPLTFGGGTKVEIK

SEQ ID NO: 91 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSTTTAYMELSSLRSEDTAVYYCALFTVLTPDDAFDIWGQGTMVTVSS

SEQ ID NO: 92 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVIRPDDTFDIWGQGTMVTVSS

SEQ ID NO: 93 - protein, 113 aa, synthetic construct; identical to SEQ ID NO: 6
  REGION 1..113: note = Recombinant Antibody Sequence

SEQ ID NO: 94 - protein, 121 aa, synthetic construct
  REGION 1..121: note = Recombinant Antibody Sequence
QVQLVQSGAEVKKPGASVKVSCKASGYTFTSYWIHWVRQAPGQRLEWMGWIKAGNGNTRYSQKFQDRVTITRDTSASTAYMELSSLRSEDTAVYYCALLTVITPDDTFDIWGQGTMVTVSS

SEQ ID NO: 95 - protein, 113 aa, synthetic construct; identical to SEQ ID NO: 6
  REGION 1..113: note = Recombinant Antibody Sequence

SEQ ID NO: 96 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVIKPDDTFDIWGQGTMVTVSS

SEQ ID NO: 97 - protein, 113 aa, synthetic construct; identical to SEQ ID NO: 6
  REGION 1..113: note = Recombinant Antibody Sequence

SEQ ID NO: 98 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTRITPDDTFDIWGQGTMVTVSS

SEQ ID NO: 99 - protein, 113 aa, synthetic construct; identical to SEQ ID NO: 6
  REGION 1..113: note = Recombinant Antibody Sequence

SEQ ID NO: 100 - protein, 121 aa, synthetic construct; identical to SEQ ID NO: 17
  REGION 1..121: note = Recombinant Antibody Sequence

SEQ ID NO: 101 - protein, 113 aa, synthetic construct
  REGION 1..113: note = Recombinant Antibody Sequence
DIVMTQSPDSLAVSLGERATINCKSSQNILYSSWNKNYLAWYQQKPGQAPKLLIYWASTRESGVPDRFSGSGSGTDFTLTISSLQAEDVAVYYCHQYYSSPLTFGGGTKVEIK

SEQ ID NO: 102 - protein, 121 aa, synthetic construct; identical to SEQ ID NO: 17
  REGION 1..121: note = Recombinant Antibody Sequence

SEQ ID NO: 103 - protein, 113 aa, synthetic construct
  REGION 1..113: note = Recombinant Antibody Sequence
DIVMTQSPDSLAVSLGERATINCKSSQNILYSSNNKNYLAWYQQKPGQAPKLLIYWASTRESGVPDRFSGSGSGTDFTLTISSLQAEDVAVYYCHQYYHSPLTFGGGTKVEIK

SEQ ID NO: 104 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLMVITPDDTFDIWGQGTMVTVSS

SEQ ID NO: 105 - protein, 113 aa, synthetic construct; identical to SEQ ID NO: 6
  REGION 1..113: note = Recombinant Antibody Sequence

SEQ ID NO: 106 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTKITPDDTFDIWGQGTMVTVSS

SEQ ID NO: 107 - protein, 113 aa, synthetic construct; identical to SEQ ID NO: 6
  REGION 1..113: note = Recombinant Antibody Sequence

SEQ ID NO: 108 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVITMDDTFDIWGQGTMVTVSS

SEQ ID NO: 109 - protein, 113 aa, synthetic construct; identical to SEQ ID NO: 6
  REGION 1..113: note = Recombinant Antibody Sequence

SEQ ID NO: 110 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVITQDDTFDIWGQGTMVTVSS

SEQ ID NO: 111 - protein, 113 aa, synthetic construct; identical to SEQ ID NO: 6
  REGION 1..113: note = Recombinant Antibody Sequence

SEQ ID NO: 112 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTWITPDDTFDIWGQGTMVTVSS

SEQ ID NO: 113 - protein, 113 aa, synthetic construct; identical to SEQ ID NO: 6
  REGION 1..113: note = Recombinant Antibody Sequence

SEQ ID NO: 114 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCELLTVITPDDTFDIWGQGTMVTVSS

SEQ ID NO: 115 - protein, 113 aa, synthetic construct; identical to SEQ ID NO: 6
  REGION 1..113: note = Recombinant Antibody Sequence

SEQ ID NO: 116 - protein, 121 aa, synthetic construct; identical to SEQ ID NO: 17
  REGION 1..121: note = Recombinant Antibody Sequence

SEQ ID NO: 117 - protein, 113 aa, synthetic construct
  REGION 1..113: note = Recombinant Antibody Sequence
DIVMTQSPDSLAVSLGERATINCKSSQNILYSSRNKNYLAWYQQKPGQAPKLLIYWASTRESGVPDRFSGSGSGTDFTLTISSLQAEDVAVYYCHQYYSSPLTFGGGTKVEIK

SEQ ID NO: 118 - protein, 121 aa, synthetic construct; identical to SEQ ID NO: 17
  REGION 1..121: note = Recombinant Antibody Sequence

SEQ ID NO: 119 - protein, 113 aa, synthetic construct
  REGION 1..113: note = Recombinant Antibody Sequence
DIVMTQSPDSLAVSLGERATINCKSSQNILYSWNNKNYLAWYQQKPGQAPKLLIYWASTRESGVPDRFSGSGSGTDFTLTISSLQAEDVAVYYCHQYYSSPLTFGGGTKVEIK

SEQ ID NO: 120 - protein, 121 aa, synthetic construct; identical to SEQ ID NO: 17
  REGION 1..121: note = Recombinant Antibody Sequence

SEQ ID NO: 121 - protein, 113 aa, synthetic construct
  REGION 1..113: note = Recombinant Antibody Sequence
DIVMTQSPDSLAVSLGERATINCKSSQNILYSSNNKNYLAWYQQKPGQAPKLLIYWASTRESGVPDRFSGSGSGTDFTLTISSLQAEDVAVYYCHQYYFSPLTFGGGTKVEIK

SEQ ID NO: 122 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLCVITPDDTFDIWGQGTMVTVSS

SEQ ID NO: 123 - protein, 113 aa, synthetic construct; identical to SEQ ID NO: 6
  REGION 1..113: note = Recombinant Antibody Sequence

SEQ ID NO: 124 - protein, 121 aa, synthetic construct
  REGION 1..121: note = Recombinant Antibody Sequence
QVQLVQSGAEVKKPGASVKVSCKASGYTFTSKAIHWVRQAPGQRLEWMGWIKAGNGNTRYSQKFQDRVTITRDTSASTAYMELSSLRSEDTAVYYCALLTVITPDDTFDIWGQGTMVTVSS

SEQ ID NO: 125 - protein, 113 aa, synthetic construct; identical to SEQ ID NO: 6
  REGION 1..113: note = Recombinant Antibody Sequence

SEQ ID NO: 126 - protein, 121 aa, synthetic construct; identical to SEQ ID NO: 17
  REGION 1..121: note = Recombinant Antibody Sequence

SEQ ID NO: 127 - protein, 113 aa, synthetic construct
  REGION 1..113: note = Recombinant Antibody Sequence
DIVMTQSPDSLAVSLGERATINCKSSQNILYSSNNKNWLAWYQQKPGQAPKLLIYWASTRESGVPDRFSGSGSGTDFTLTISSLQAEDVAVYYCHQYYSSPLTFGGGTKVEIK

SEQ ID NO: 128 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKAGNGNTRYSQKFQDRVTITRDTSASTAYMELSSLRSEDTAVYYCALLTVIRRDDTFDIWGQGTMVTVSS

SEQ ID NO: 129 - protein, 113 aa, synthetic construct; identical to SEQ ID NO: 6
  REGION 1..113: note = Recombinant Antibody Sequence

SEQ ID NO: 130 - protein, 121 aa, synthetic construct; identical to SEQ ID NO: 92
  REGION 1..121: note = Recombinant Antibody Sequence

SEQ ID NO: 131 - protein, 121 aa, synthetic construct; identical to SEQ ID NO: 5
  REGION 1..121: note = Recombinant Antibody Sequence

SEQ ID NO: 132 - protein, 113 aa, synthetic construct; identical to SEQ ID NO: 6
  REGION 1..113: note = Recombinant Antibody Sequence

SEQ ID NO: 133 - protein, 121 aa, synthetic construct; identical to SEQ ID NO: 92
  REGION 1..121: note = Recombinant Antibody Sequence

SEQ ID NO: 134 - protein, 113 aa, synthetic construct; identical to SEQ ID NO: 119
  REGION 1..113: note = Recombinant Antibody Sequence

SEQ ID NO: 135 - protein, 121 aa, synthetic construct
  REGION 1..121: note = Recombinant Antibody Sequence
QVQLVQSGAEVKKPGASVKVSCKASGYTFTSYWIHWVRQAPGQRLEWMGWIKAGNGNTRYSQKFQDRVTITRDTSASTAYMELSSLRSEDTAVYYCALLTVIRPDDTFDIWGQGTMVTVSS

SEQ ID NO: 136 - protein, 113 aa, synthetic construct
  REGION 1..113: note = Recombinant Antibody Sequence
DIVMTQSPDSLAVSLGERATINCKSSQNHLYSSWNKNYLAWYQQKPGQAPKLLIYWASTRESGVPDRFSGSGSGTDFTLTISSLQAEDVAVYYCHQYYFSPLTFGGGTKVEIK

SEQ ID NO: 137 - protein, 121 aa, synthetic construct
  REGION 1..121: note = Recombinant Antibody Sequence
QVQLVQSGAEVKKPGASVKVSCKASGYTFTSYWIHWVRQAPGQRLEWMGWIKAGNGNTRYSQKFQDRVTITRDTSASTAYMELSSLRSEDTAVYYCALLTVIRKDDTFDIWGQGTMVTVSS

SEQ ID NO: 138 - protein, 113 aa, synthetic construct
  REGION 1..113: note = Recombinant Antibody Sequence
DIVMTQSPDSLAVSLGERATINCKSSQNHLYSSNNKNYLAWYQQKPGQAPKLLIYWASTRESGVPDRFSGSGSGTDFTLTISSLQAEDVAVYYCHQYYYSPLTFGGGTKVEIK

SEQ ID NO: 139 - protein, 121 aa, synthetic construct
  REGION 1..121: note = Recombinant Antibody Sequence
QVQLVQSGAEVKKPGASVKVSCKASGYTFTSYWIHWVRQAPGQRLEWMGWIKAGNGNTRYSQKFQDRVTITRDTSASTAYMELSSLRSEDTAVYYCALLTVIRMDDTFDIWGQGTMVTVSS

SEQ ID NO: 140 - protein, 113 aa, synthetic construct
  REGION 1..113: note = Recombinant Antibody Sequence
DIVMTQSPDSLAVSLGERATINCKSSQNILYSSWNKNYLAWYQQKPGQAPKLLIYWASTRESGVPDRFSGSGSGTDFTLTISSLQAEDVAVYYCHQYYSSMLTFGGGTKVEIK

SEQ ID NO: 141 - protein, 121 aa, synthetic construct
  REGION 1..121: note = Recombinant Antibody Sequence
QVQLVQSGAEVKKPGASVKVSCKASGYTFTSRAIEWVRQAPGQRLEWMGWIKRGNGNTRYSQKFQDRVTITRDTSASTAYMELSSLRSEDTAVYYCALLTVITRDDTFDIWGQGTMVTVSS

SEQ ID NO: 142 - protein, 113 aa, synthetic construct
  REGION 1..113: note = Recombinant Antibody Sequence
DIVMTQSPDSLAVSLGERATINCKSSQNILYSSNNKNWLAWYQQKPGQAPKLLIYWASTRESGVPDRFSGSGSGTDFTLTISSLQAEDVAVYYCHQYYFSPLTFGGGTKVEIK

SEQ ID NO: 143 - protein, 121 aa, synthetic construct
  REGION 1..121: note = Recombinant Antibody Sequence
QVQLVQSGAEVKKPGASVKVSCKASGYTFTRYAIHWVRQAPGQRLEWMGWIKAGNGNTRYSQKFQDRVTITRDTSASTAYMELSSLRSEDTAVYYCALLTVIRRDDTFDIWGQGTMVTVSS

SEQ ID NO: 144 - protein, 113 aa, synthetic construct
  REGION 1..113: note = Recombinant Antibody Sequence
DIVMTQSPDSLAVSLGERATINCKSSQNILYSINNKNYLAWYQQKPGQAPKLLIYWASTRESGVPDRFSGSGSGTDFTLTISSLQAEDVAVYYCHQYYSSPLTFGGGTKVEIK

SEQ ID NO: 145 - protein, 121 aa, synthetic construct
  REGION 1..121: note = Recombinant Antibody Sequence
QVQLVQSGAEVKKPGASVKVSCKASGYTFTRYWIHWVRQAPGQRLEWMGWIKAGNGNTRYSQKFQDRVTITRDTSASTAYMELSSLRSEDTAVYYCALLTQIRPDDTFDIWGQGTMVTVSS

SEQ ID NO: 146 - protein, 113 aa, synthetic construct
  REGION 1..113: note = Recombinant Antibody Sequence
DIVMTQSPDSLAVSLGERATINCKSSQNILYSSWNKNYLAWYQQKPGQAPKLLIYWASTRESGVPDRFSGSGSGTDFTLTISSLQAEDVAVYYCHQYYFSPLTFGGGTKVEIK

SEQ ID NO: 147 - protein, 121 aa, synthetic construct
  REGION 1..121: note = Recombinant Antibody Sequence
QVQLVQSGAEVKKPGASVKVSCKASGYTFTSRAIHWVRQAPGQRLEWMGWIKRGNGNTRYSQKFQDRVTITRDTSASTAYMELSSLRSEDTAVYYCALLTVITMDDTFDIWGQGTMVTVSS

SEQ ID NO: 148 - protein, 113 aa, synthetic construct
  REGION 1..113: note = Recombinant Antibody Sequence
DIVMTQSPDSLAVSLGERATINCKSSQNHLYSSNNKNWLAWYQQKPGQAPKLLIYWASTRESGVPDRFSGSGSGTDFTLTISSLQAEDVAVYYCHQYYFSPLTFGGGTKVEIK

SEQ ID NO: 149 - protein, 121 aa, synthetic construct
  REGION 1..121: note = Recombinant Antibody Sequence
QVQLVQSGAEVKKPGASVKVSCKASGYTFTSYAIHWVRQAPGQRLEWMGWIKRGNGNTRYSQKFQDRVTITRDTSASTAYMELSSLRSEDTAVYYCALLTVITMDDTFDIWGQGTMVTVSS

SEQ ID NO: 150 - protein, 113 aa, synthetic construct
  REGION 1..113: note = Recombinant Antibody Sequence
DIVMTQSPDSLAVSLGERATINCKSSQNILYSSWNKNWLAWYQQKPGQAPKLLIYWASTRESGVPDRFSGSGSGTDFTLTISSLQAEDVAVYYCHQYYHSPLTFGGGTKVEIK

SEQ ID NO: 151 - protein, 121 aa, synthetic construct
  REGION 1..121: note = Recombinant Antibody Sequence
QVQLVQSGAEVKKPGASVKVSCKASGYTFTSRAIHWVRQAPGQRLEWMGWIKRGNGNTRYSQKFQDRVTITRDTSASTAYMELSSLRSEDTAVYYCALLTVITRDDTFDIWGQGTMVTVSS

SEQ ID NO: 152 - protein, 113 aa, synthetic construct
  REGION 1..113: note = Recombinant Antibody Sequence
DIVMTQSPDSLAVSLGERATINCKSSQNILYSSWNKNYLAWYQQKPGQAPKLLIYWASTRESGVPDRFSGSGSGTDFTLTISSLQAEDVAVYYCHQYYHSPLTFGGGTKVEIK

SEQ ID NO: 153 - protein, 121 aa, synthetic construct
  REGION 1..121: note = Recombinant Antibody Sequence
QVQLVQSGAEVKKPGASVKVSCKASGYTFTSRAIHWVRQAPGQRLEWMGWIKRGNGNTRYSQKFQDRVTITRDTSASTAYMELSSLRSEDTAVYYCALLTVIKRDDTFDIWGQGTMVTVSS

SEQ ID NO: 154 - protein, 113 aa, synthetic construct
  REGION 1..113: note = Recombinant Antibody Sequence
DIVMTQSPDSLAVSLGERATINCKSSQNILYTMNNKNYLAWYQQKPGQAPKLLIYWASYRESGVPDRFSGSGSGTDFTLTISSLQAEDVAVYYCHQYYSSPLTFGGGTKVEIK

SEQ ID NO: 155 - protein, 121 aa, synthetic construct
  REGION 1..121: note = Recombinant Antibody Sequence
QVQLVQSGAEVKKPGASVKVSCKASGYTFTSRAIHWVRQAPGQRLEWMGWIKRGNGNTRYSQKFQDRVTITRDTSASTAYMELSSLRSEDTAVYYCALLTVIRPDDTFDIWGQGTMVTVSS

SEQ ID NO: 156 - protein, 113 aa, synthetic construct
  REGION 1..113: note = Recombinant Antibody Sequence
DIVMTQSPDSLAVSLGERATINCKSSQNILYSINNKNYLAWYQQKPGQAPKLLIYWASTRESGVPDRFSGSGSGTDFTLTISSLQAEDVAVYYCHQYYSSMLTFGGGTKVEIK

SEQ ID NO: 157 - protein, 121 aa, synthetic construct
  REGION 1..121: note = Recombinant Antibody Sequence
QVQLVQSGAEVKKPGASVKVSCKASGYTFTSRAIHWVRQAPGQRLEWMGWIKAGNGNTRYSQKFQDRVTITRDTSASTAYMELSSLRSEDTAVYYCALLTVITRDDVFDIWGQGTMVTVSS

SEQ ID NO: 158 - protein, 113 aa, synthetic construct
  REGION 1..113: note = Recombinant Antibody Sequence
DIVMTQSPDSLAVSLGERATINCKSSQNHLYSSNNKNWLAWYQQKPGQAPKLLIYWASHRESGVPDRFSGSGSGTDFTLTISSLQAEDVAVYYCHQYYFSPLTFGGGTKVEIK

SEQ ID NO: 159 - protein, 121 aa, synthetic construct
  REGION 1..121: note = Recombinant Antibody Sequence
QVQLVQSGAEVKKPGASVKVSCKASGYTFTRYAIHWVRQAPGQRLEWMGWIKAGNGNTRYSQKFQDRVTITRDTSASTAYMELSSLRSEDTAVYYCALLTVIRMDDTFDIWGQGTMVTVSS

SEQ ID NO: 160 - protein, 113 aa, synthetic construct; identical to SEQ ID NO: 142
  REGION 1..113: note = Recombinant Antibody Sequence

SEQ ID NO: 161 - protein, 121 aa, synthetic construct; identical to SEQ ID NO: 128
  REGION 1..121: note = Recombinant Antibody Sequence

SEQ ID NO: 162 - protein, 113 aa, synthetic construct
  REGION 1..113: note = Recombinant Antibody Sequence
DIVMTQSPDSLAVSLGERATINCKSSQNILYTSNNKNYLAWYQQKPGQAPKLLIYWASTRESGVPDRFSGSGSGTDFTLTISSLQAEDVAVYYCHQWYSSPLTFGGGTKVEIK

SEQ ID NO: 163 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTRITPDDAFDIWGQGTMVTVSS

SEQ ID NO: 164 - protein, 113 aa, synthetic construct; identical to SEQ ID NO: 2
  REGION 1..113: note = Recombinant Antibody Sequence

SEQ ID NO: 165 - protein, 121 aa, synthetic construct; identical to SEQ ID NO: 71
  REGION 1..121: note = Recombinant Antibody Sequence

SEQ ID NO: 166 - protein, 113 aa, synthetic construct; identical to SEQ ID NO: 2
  REGION 1..113: note = Recombinant Antibody Sequence

SEQ ID NO: 167 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLIVITPDDAFDIWGQGTMVTVSS

SEQ ID NO: 168 - protein, 113 aa, synthetic construct; identical to SEQ ID NO: 2
  REGION 1..113: note = Recombinant Antibody Sequence

SEQ ID NO: 169 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTVINPDDAFDIWGQGTMVTVSS

SEQ ID NO: 170 - protein, 113 aa, synthetic construct; identical to SEQ ID NO: 2
  REGION 1..113: note = Recombinant Antibody Sequence

SEQ ID NO: 171 - protein, 121 aa, synthetic construct; identical to SEQ ID NO: 19
  REGION 1..121: note = Recombinant Antibody Sequence

SEQ ID NO: 172 - protein, 113 aa, synthetic construct
  REGION 1..113: note = Recombinant Antibody Sequence
DIVMTQSPDSLAVSLGERATINCKSSQNILYSSNNKNRLAWYQQKPGQPPKLLFYWASTRESGVPDRFSGSGSGTDFTLTISSLQAEDVAVYYCHQYYSSPLTFGGGTKVEIK

SEQ ID NO: 173 - protein, 121 aa, synthetic construct; identical to SEQ ID NO: 7
  REGION 1..121: note = Recombinant Antibody Sequence

SEQ ID NO: 174 - protein, 113 aa, synthetic construct; identical to SEQ ID NO: 2
  REGION 1..113: note = Recombinant Antibody Sequence

SEQ ID NO: 175 - protein, 121 aa, synthetic construct; identical to SEQ ID NO: 1
  REGION 1..121: note = Recombinant Antibody Sequence

SEQ ID NO: 176 - protein, 113 aa, synthetic construct; identical to SEQ ID NO: 2
  REGION 1..113: note = Recombinant Antibody Sequence

SEQ ID NO: 177 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTVITRDDAFDIWGQGTMVTVSS

SEQ ID NO: 178 - protein, 113 aa, synthetic construct; identical to SEQ ID NO: 2
  REGION 1..113: note = Recombinant Antibody Sequence

SEQ ID NO: 179 - protein, 121 aa, synthetic construct; identical to SEQ ID NO: 13
  REGION 1..121: note = Recombinant Antibody Sequence

SEQ ID NO: 180 - protein, 113 aa, synthetic construct; identical to SEQ ID NO: 2
  REGION 1..113: note = Recombinant Antibody Sequence

SEQ ID NO: 181 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLQVITPDDAFDIWGQGTMVTVSS

SEQ ID NO: 182 - protein, 113 aa, synthetic construct; identical to SEQ ID NO: 2
  REGION 1..113: note = Recombinant Antibody Sequence

SEQ ID NO: 183 - protein, 121 aa, synthetic construct
  REGION 1..121: note = Recombinant Antibody Sequence
QVHLVQSGAEVKKPGASVKVSCKASGYTFTSRAIHWVRQAPGQRLEWMGWIKGGNGNTRYSQKFQDRVTITRDTSASTAYMELSSLRSEDTAVYYCALLTVITPDDAFDIWGQGTMVTVSS

SEQ ID NO: 184 - protein, 113 aa, synthetic construct; identical to SEQ ID NO: 2
  REGION 1..113: note = Recombinant Antibody Sequence

SEQ ID NO: 185 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTRITRDDAFDIWGQGTMVTVSS

SEQ ID NO: 186 - protein, 113 aa, synthetic construct; identical to SEQ ID NO: 2
  REGION 1..113: note = Recombinant Antibody Sequence

SEQ ID NO: 187 - protein, 121 aa, synthetic construct
  REGION 1..121: note = Recombinant Antibody Sequence
QVHLVQSGAEVKKPGASVKVSCKASGYTFTSRAIHWVRQAPGQRLEWMGWIKGGNGNTRYSQKFQDRVTITRDTSASTAYMELSSLRSEDTAVYYCALLIVITPDDAFDIWGQGTMVTVSS

SEQ ID NO: 188 - protein, 113 aa, synthetic construct; identical to SEQ ID NO: 2
  REGION 1..113: note = Recombinant Antibody Sequence

SEQ ID NO: 189 - protein, 121 aa, synthetic construct
  REGION 1..121: note = Recombinant Antibody Sequence
QVHLVQSGAEVKKPGASVKVSCKASGYTFTSYWIHWVRQAPGQRLEWMGWIKGGNGNTRYSQKFQDRVTITRDTSASTAYMELSSLRSEDTAVYYCALLTRITPDDAFDIWGQGTMVTVSS

SEQ ID NO: 190 - protein, 113 aa, synthetic construct
  REGION 1..113: note = Recombinant Antibody Sequence
DIVMTQSPDSLAVSLGERATINCKSSQNILYSSNNKNYLAWYQQKPGQPPKLLFYWASTRESGVPDRFSGSGSGTDFTLTISSLQAEDVAVYYCHQYYESPLTFGGGTKVEIK

SEQ ID NO: 191 - protein, 121 aa, synthetic construct; identical to SEQ ID NO: 189
  REGION 1..121: note = Recombinant Antibody Sequence

SEQ ID NO: 192 - protein, 113 aa, synthetic construct; identical to SEQ ID NO: 2
  REGION 1..113: note = Recombinant Antibody Sequence

SEQ ID NO: 193 - protein, 121 aa, synthetic construct; identical to SEQ ID NO: 163
  REGION 1..121: note = Recombinant Antibody Sequence

SEQ ID NO: 194 - protein, 113 aa, synthetic construct
  REGION 1..113: note = Recombinant Antibody Sequence
DIVMTQSPDSLAVSLGERATINCKSSQNILYSSMNKNYLAWYQQKPGQPPKLLFYWASTRESGVPDRFSGSGSGTDFTLTISSLQAEDVAVYYCHQYYSSPQTFGGGTKVEIK

SEQ ID NO: 195 - protein, 121 aa, synthetic construct
  REGION 1..121: note = Recombinant Antibody Sequence
QVHLVQSGAEVKKPGASVKVSCKASGYTFTSYWIHWVRQAPGQRLEWMGWIKGGNGNTRYSQKFQDRVTITRDTSASTAYMELSSLRSEDTAVYYCALLTVIQPDDAFDIWGQGTMVTVSS

SEQ ID NO: 196 - protein, 113 aa, synthetic construct
  REGION 1..113: note = Recombinant Antibody Sequence
DIVMTQSPDSLAVSLGERATINCKSSQNILYFSNNKNRLAWYQQKPGQPPKLLFYWASTRESGVPDRFSGSGSGTDFTLTISSLQAEDVAVYYCHQYYSSPLTFGGGTKVEIK

SEQ ID NO: 197 - protein, 121 aa, synthetic construct; identical to SEQ ID NO: 163
  REGION 1..121: note = Recombinant Antibody Sequence

SEQ ID NO: 198 - protein, 113 aa, synthetic construct
  REGION 1..113: note = Recombinant Antibody Sequence
DIVMTQSPDSLAVSLGERATINCKSSQNILYSSMNKNYLAWYQQKPGQPPKLLFYWASTRESGVPDRFSGSGSGTDFTLTISSLQAEDVAVYYCHQYYSSPLTFGGGTKVEIK

SEQ ID NO: 199 - protein, 121 aa, synthetic construct
  REGION 1..121: note = Recombinant Antibody Sequence
QVHLVQSGAEVKKPGASVKVSCKRSGYTFTSYAIHWVRQAPGQRLEWMGWIKGGNGNTRYSQKFQDRVTITRDTSASTAYMELSSLRSEDTAVYYCALLTKITRDDAFDIWGQGTMVTVSS

SEQ ID NO: 200 - protein, 113 aa, synthetic construct
  REGION 1..113: note = Recombinant Antibody Sequence
DIVMTQSPDSLAVSLGERATINCKSSQNILYSSNNKNYLAWYQQKPGQPPKYLFYWASTRESGVPDRFSGSGSGTDFTLTISSLQAEDVAVYYCHQYYSSPLTFGGGTKVEIK

SEQ ID NO: 201 - protein, 121 aa, synthetic construct
  REGION 1..121: note = Recombinant Antibody Sequence
QVHLVQSGAEVKKPGASVKVSCKASGYTFTSYWIHWVRQAPGQRLEWMGWIKGGNGNTRYSQKFQDRVTITRDTSASTAYMELSSLRSEDTAVYYCALFTVITPDDAFDIWGQGTMVTVSS

SEQ ID NO: 202 - protein, 113 aa, synthetic construct; identical to SEQ ID NO: 2
  REGION 1..113: note = Recombinant Antibody Sequence

SEQ ID NO: 203 - protein, 121 aa, synthetic construct
  REGION 1..121: note = Recombinant Antibody Sequence
QVHLVQSGAEVKKPGASVKVSCKASGYTFTTRAIHWVRQAPGQRLEWMGWIKGGNGNTRYSQKFQDRVTITRDTSASTAYMELSSLRSEDTAVYYCALLTVITRDDAFDIWGQGTMVTVSS

SEQ ID NO: 204 - protein, 113 aa, synthetic construct; identical to SEQ ID NO: 2
  REGION 1..113: note = Recombinant Antibody Sequence

SEQ ID NO: 205 - protein, 121 aa, synthetic construct
  REGION 1..121: note = Recombinant Antibody Sequence
QVHLVQSGAEVKKPGASVKVSCKASGYTFTSYAWHWVRQAPGQRLEWMGWIKGGNGNTRYSQKFQDRVTITRDTSASTAYMELSSLRSEDTAVYYCALLTRITHDDAFDIWGQGTMVTVSS

SEQ ID NO: 206 - protein, 113 aa, synthetic construct
  REGION 1..113: note = Recombinant Antibody Sequence
DIVMTQSPDSLAVSLGERATINCKSSQNILYDSNNKNYLAWYQQKPGQPPKLLFYWASTRESGVPDRFSGSGSGTDFTLTISSLQAEDVAVYYCHQYYSSPLTFGGGTKVEIK

SEQ ID NO: 207 - protein, 121 aa, synthetic construct
  REGION 1..121: note = Recombinant Antibody Sequence
QVHLVQSGAEVKKPGASVKVSCKASGYTFTRYAIHWVRQAPGQRLEWMGWIKGGNGNTRYSQKFQDRVTITRDTSASTAYMELSSLRSEDTAVYYCALLTRITPDDAFDIWGQGTMVTVSS

SEQ ID NO: 208 - protein, 113 aa, synthetic construct
  REGION 1..113: note = Recombinant Antibody Sequence
DIVMTQSPDSLAVSLGERATINCKSSQNILYSSNNKNYLAWYQQKPGQPPKLLFYWASTRESGVPDRFSGSGSGTDFTLTISSLQAEDVAVYYCHQYYSSYLTFGGGTKVEIK

SEQ ID NO: 209 - protein, 121 aa, synthetic construct; identical to SEQ ID NO: 167
  REGION 1..121: note = Recombinant Antibody Sequence

SEQ ID NO: 210 - protein, 113 aa, synthetic construct
  REGION 1..113: note = Recombinant Antibody Sequence
DIVMTQSPDSLAVSLGERATINCKSSQNILYSSNNKNYLAWYQQKPGQPPKLLFYWASTRESGVPDRFSGSGSGTDFTLTISSLQAEDVAVYYCHQYYHSPLTFGGGTKVEIK

SEQ ID NO: 211 - protein, 121 aa, synthetic construct
  REGION 1..121: note = Recombinant Antibody Sequence
QVHLVQSGAEVKKPGASVKVSCKASGYTFTSRAIHWVRQAPGQRLEWMGWIKGGNGNTRYSQKFQDRVTITRDTSASTAYMELSSLRSEDTAVYYCALLMVITPDDAFDIWGQGTMVTVSS

SEQ ID NO: 212 - protein, 113 aa, synthetic construct; identical to SEQ ID NO: 2
  REGION 1..113: note = Recombinant Antibody Sequence

SEQ ID NO: 213 - protein, 121 aa, synthetic construct
  REGION 1..121: note = Recombinant Antibody Sequence
QVHLVQSGAEVKKPGASVKVSCKASGYTFTSRAIHWVRQAPGQRLEWMGWIKGGNGNTRYSQKFQDRVTITRDTSASTAYMELSSLRSEDTAVYYCALLTRITPDDAFDIWGQGTMVTVSS

SEQ ID NO: 214 - protein, 113 aa, synthetic construct; identical to SEQ ID NO: 2
  REGION 1..113: note = Recombinant Antibody Sequence

SEQ ID NO: 215 - protein, 451 aa, synthetic construct
  REGION 1..451: note = Recombinant Antibody Sequence
QVQLVQSGAEVKKPGASVEVSCEASGYTFTSYAIHWVRQAPGQELEWMGWIKAGNGNTRYSQKFQDRVTITRDTSTTTAYMELSSLRSEDTAVYYCALLTVLTPDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 216 - protein, 220 aa, synthetic construct
  REGION 1..220: note = Recombinant Antibody Sequence
DIVMTQSPDSLAVSLGEEATINCKSSQSVLYSSNNKNYLAWYQQKPGQPPNLLIYWASTRQSGVPDRFSGSGSGTDFTLTISSLQAEDVAVYYCHQYYSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 121 aa, synthetic construct
  REGION 1..121: note = Recombinant Antibody Sequence
QVHLVQSGAEVKKPGASVEVSCEASGYTFTRYAIHWVRQAPGQELEWMGWIKGGNGNTRYSQKFQDRVTITRDTSASTAYMELSSLRSEDTAVYYCALLTVITPDDAFDIWGQGTMVTVSS

SEQ ID NO: 218 - protein, 113 aa, synthetic construct
  REGION 1..113: note = Recombinant Antibody Sequence
DIVMTQSPDSLAVSLGEEATINCKSSQNIFFSSNNKNYLAWYQQKPGQPPKLLFYWASTRESGVPDRFSGSGSGTDFTLTISSLQAEDVAVYYCHQYYSSPLTFGGGTKVEIK

SEQ ID NO: 219 - protein, 121 aa, synthetic construct
  REGION 1..121: note = Recombinant Antibody Sequence
QVQLVQSGAEVKKPGASVQVSCQASGYTFTRYAIHWVRQAPGQQLEWMGWIKAGNGNTRYSQKFQDRVTITRDTSASTAYMELSSLRSEDTAVYYCALLTVITPDDAFDIWGQGTMVTVSS

SEQ ID NO: 220 - protein, 113 aa, synthetic construct
  REGION 1..113: note = Recombinant Antibody Sequence
DIVMTQSPDSLAVSLGEEATINCKSSQNIFFSSKNKNYLAWYQQIPGQPPKLLIYWASTRESGVPDRFSGSGSGTDFTLTISSLQAEDVAVYYCHQYYSSPLTFGGGTKVEIQ

SEQ ID NO: 221 - protein, 121 aa, synthetic construct
  REGION 1..121: note = Recombinant Antibody Sequence
QVHLVQSGAEVKKPGASVEVSCEASGYTFTSYAIHWVRQAPGQELEWMGWIKGGNGNTRYSQKFQDRVTITRDTSASTAYMELSSLRSEDTAVYYCALLTVITKDDAFDIWGQGTMVTVSS

SEQ ID NO: 222 - protein, 113 aa, synthetic construct
  REGION 1..113: note = Recombinant Antibody Sequence
DIVMTQSPDSLAVSLGEEATINCKSSQNILYSSNNKNYLAWYQQKPGQPPKLLFYWASTRESGVPDRFSGSGSGTDFTLTISSLQAEDVAVYYCHQYYSSPLTFGGGTKVEIK

SEQ ID NO: 223 - DNA, 22 bp, synthetic construct
  misc_feature 1..22: note = DNA primer
  source 1..22: mol_type = other DNA
gctcagggaartagcccttgac

SEQ ID NO: 224 - DNA, 19 bp, synthetic construct
  misc_feature 1..19: note = DNA primer
  source 1..19: mol_type = other DNA
tgggaagatggatacagtt

SEQ ID NO: 225 - protein, 121 aa, synthetic construct
  REGION 1..121: note = Recombinant Antibody Sequence
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCALLTVVTPDDAFDIWGQGTMVTVSS

SEQ ID NO: 226 - protein, 113 aa, synthetic construct
  REGION 1..113: note = Recombinant Antibody Sequence
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIK

SEQ ID NO: 227 - protein, 15 aa, synthetic construct
  REGION 1..15: note = Peptide sequence
KLKQPADGNPDPNAN

SEQ ID NO: 228 - protein, 15 aa, synthetic construct
  REGION 1..15: note = Peptide sequence
NANPNVDPNANPNVD

SEQ ID NO: 229 - protein, 15 aa, synthetic construct
  REGION 1..15: note = Peptide sequence
NVDPNANPNVDPNAN

SEQ ID NO: 230 - protein, 15 aa, synthetic construct
  REGION 1..15: note = Peptide sequence
NVDPNANPNANPNAN

SEQ ID NO: 231 - protein, 15 aa, synthetic construct
  REGION 1..15: note = Peptide sequence
NANPNANPNANPNAN

SEQ ID NO: 232 - protein, 15 aa, synthetic construct
  REGION 1..15: note = Peptide sequence
NANPNANPNANPNVD

SEQ ID NO: 233 - protein, 15 aa, synthetic construct
  REGION 1..15: note = Peptide sequence
NANPNANPNVDPNAN

SEQ ID NO: 234 - protein, 15 aa, synthetic construct
  REGION 1..15: note = Peptide sequence
NANPNVDPNANPNAN

SEQ ID NO: 235 - protein, 15 aa, synthetic construct
  REGION 1..15: note = Peptide sequence
EDNEKLRKPKHKKLR

SEQ ID NO: 236 - protein, 15 aa, synthetic construct
  REGION 1..15: note = Peptide sequence
KLRKPKHKKLKQPAD

SEQ ID NO: 237 - protein, 15 aa, synthetic construct
  REGION 1..15: note = Peptide sequence
KLKQPADGNPDPNAN

SEQ ID NO: 238 - protein, 15 aa, synthetic construct
  REGION 1..15: note = Peptide sequence
NANPNANPNANPNKN

SEQ ID NO: 239 - protein, 121 aa, synthetic construct; identical to SEQ ID NO: 11
  REGION 1..121: note = Recombinant Antibody Sequence

SEQ ID NO: 240 - protein, 113 aa, synthetic construct
  REGION 1..113: note = Recombinant Antibody Sequence
DIVMTQSPDSLAVSLGERATINCKSSQNIFFSSNNKNYLAWYQQKPGQPPKLLFYWASTRESGVPDRFSGSGSGTDFTLTISSLQAEDVAVYYCHQYFSSPLTFGGGTKVEIK

SEQ ID NO: 241 - protein, 121 aa, synthetic construct
  REGION 1..121: note = Recombinant Antibody Sequence
QVHLVQSGAEVKKPGASVKVSCKASGYTFTRYAIHWVRQAPGQRLEWMGWIKGGNGNTRYSQKFQDRVTITRDTSASTAYMELSSLRSEDTAVYYCALMTVITPDDAFDIWGQGTMVTVSS

SEQ ID NO: 242 - protein, 113 aa, synthetic construct; identical to SEQ ID NO: 240
  REGION 1..113: note = Recombinant Antibody Sequence

SEQ ID NO: 243 - protein, 121 aa, synthetic construct; identical to SEQ ID NO: 241
  REGION 1..121: note = Recombinant Antibody Sequence

SEQ ID NO: 244 - protein, 113 aa, synthetic construct
  REGION 1..113: note = Recombinant Antibody Sequence
DIVMTQSPDSLAVSLGERATINCKSSQNIFYSSNNKNYLAWYQQKPGQPPKLLFYWASTRESGVPDRFSGSGSGTDFTLTISSLQAEDVAVYYCHQYFSSPLTFGGGTKVEIK

SEQ ID NO: 245 - protein, 121 aa, synthetic construct; identical to SEQ ID NO: 11
  REGION 1..121: note = Recombinant Antibody Sequence

SEQ ID NO: 246 - protein, 113 aa, synthetic construct; identical to SEQ ID NO: 58
  REGION 1..113: note = Recombinant Antibody Sequence

SEQ ID NO: 247 - protein, 121 aa, synthetic construct
  REGION 1..121: note = Recombinant Antibody Sequence
QVHLVQSGAEVKKPGASVKVSCKASGYTFTTYAIHWVRQAPGQRLEWMGWIKVGDGNTRYSPKFQDRVTITRDTSASTAYMELSSLRSEDTAVYFCALLTVITPDDAFDIWGQGTMVTVSS

SEQ ID NO: 248 - protein, 113 aa, synthetic construct
  REGION 1..113: note = Recombinant Antibody Sequence
DIVMTQSPDSLAVSLGERATINCKSSQNILYSSKNKNYLAWYQQKPGQSPKLLFYWASTRESGVPDRFSGSGSGTDFTLTISSLQAEDVAVYYCHQYYSSPLTFGGGTKVEIK

SEQ ID NO: 249 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAVYYCALLTVITPDDTFDIWGQGTMVTVSS

SEQ ID NO: 250 - protein, 113 aa, synthetic construct
  REGION 1..113: note = Recombinant Antibody Sequence
DIVMTQSPDSLAVSLGERATINCKSSQNILYSSNNKNYLAWYQQKPGQAPKLLFYWASTRESGVPDRFSGSGSGTDFTLTISSLQAEDVAVYYCHQYYSSPLTFGGGTKVEIK

SEQ ID NO: 251 - protein, 121 aa, synthetic construct
  REGION 1..121: note = Recombinant Antibody Sequence
QVHLVQSGAELKKPGASVKVSCKASGYTFTSYAIHWVRQAPGQRLEWMGWIKGGNGNTRYSQKFQDRVTITRDTSASTAYMELNSLRSEDTAVYYCALLTVITPDDAFDIWGQGTMVTVSS

SEQ ID NO: 252 - protein, 113 aa, synthetic construct
  REGION 1..113: note = Recombinant Antibody Sequence
DIVMTQSPDSLAVSLGERATINCKSSQNILYSSNNKNYLAWYQQKPGQPPQLLFYWASTRESGVPDRFSGSGSGTDFTLTISSLQAEDVAVYYCHQYYSSPLTFGGGTKVEIK

SEQ ID NO: 253 - protein, 121 aa, synthetic construct
  REGION 1..121: note = Recombinant Antibody Sequence
QVHLVQSGAEVKKPGASVKVSCKASGYTFTSYAIHWVRQAPGQRLEWMGWIKGGNGNTRYSQKFQDRVTITRDTSASTAYMELSSLRSEDTAGYYCALLTVITPDDAFDIWGQGTMVTVSS

SEQ ID NO: 254 - protein, 113 aa, synthetic construct
  REGION 1..113: note = Recombinant Antibody Sequence
DIVMTQSPDSLAVSLGERASINCKSSQNILFSSNNKNYLAWYQQKPGQPPKLLFYWASTRESGVPDRFSGSGSGTDFTLTISSLQAEDVAVYICHQYYSSPLTFGGGTKVEIK

(19) United States
(12) Patent Application Publication
(10) Pub. No.: US 2025/0011406 A1
(43) Pub. Date: Jan. 9, 2025

(54) NEUTRALIZING ANTIBODIES TO PLASMODIUM FALCIPARUM CIRCUMSPOROZOITE PROTEIN AND THEIR USE

(71) Applicants: THE UNITED STATES OF AMERICA, AS REPRESENTED BY THE SECRETARY, DEPARTMENT OF HEALTH AND HUMAN SERVIC, Bethesda, MD (US); The General Hospital Corporation, Boston, MA (US)

(72) Inventors: Young Do Kwon, Kensington, MD (US); Amarendra Pegu, Rockville, MD (US); Eun Sung Yang, Bethesda, MD (US); Peter Kwong, Washington, DC (US); Robert Seder, Chevy Chase, MD (US); Facundo Batista, Cambridge, MA (US); Sven Kratochvil, Mainz (DE); Chen-Hsiang Shen, Atlanta, GA (US); Reda Rawi, North Bethesda, MD (US); Mateo Reveiz, Potomac, MD (US); Prabhanshu Tripathi, Rockville, MD (US)

(73) Assignees: The USA, as represented by the Secretary, Department of Health and Human Services, Bethesda, MD (US); The General Hospital Corporation, Boston, MA (US)

(21) Appl. No.: 18/707,305

(22) PCT Filed: Nov. 4, 2022
(86) PCT No.: PCT/US2022/079340

§ 371 Date: May 3, 2024

Related U.S. Application Data
(60) Provisional application No. 63/275,936, filed on Nov. 4, 2021.

(51) Int. Cl. C07K 16/20; A61K 39/00; A61P 33/02

(52) U.S. Cl. CPC C07K 16/205; A61P 33/02; A61K 2039/505; C07K 2317/24; C07K 2317/55; C07K 2317/76; C07K 2317/92; C07K 2317/94

(57) ABSTRACT

Antibodies and antigen binding fragments that specifically bind to P. falciparum circumsporozoite protein are disclosed. Nucleic acids encoding these antibodies, vectors and host cells are also provided. The disclosed antibodies, antigen binding fragments, nucleic acids and vectors can be used, for example, to inhibit a P. falciparum infection.

### CROSS REFERENCE TO RELATED APPLICATIONS

This application claims priority to U.S. Provisional Application No. 63/275,936, filed Nov. 4, 2021, which is incorporated by reference in its entirety.

### FIELD

This relates to monoclonal antibodies and antigen binding fragments that specifically bind to Plasmodium falciparum (P. falciparum or Pf) circumsporozoite protein (CSP) and their use, for example, in methods of inhibiting P. falciparum infection in a subject.

### BACKGROUND

Malaria ranks as one of the world's deadliest infectious diseases, with approximately 300 million cases per year. Malaria in humans is caused by five species of the Plasmodium parasite: P. falciparum, P. vivax, P. ovale, P. knowlesi and P. malariae. P. falciparum causes the most severe form of malaria disease, leading to the death of about ˜600,000 people annually, most of whom are young children.

Each of the Plasmodium species that infect humans is transmitted through the bite of an infected female Anopheles mosquito, which introduces Plasmodium sporozoites into the bloodstream of the human host. The major protein on the surface of the infecting P. falciparum sporozoites is the circumsporozoite protein (PfCSP) and provides a major target for antibodies and vaccines. The sporozoites rapidly reach the liver where they are sequestered by hepatocytes and undergo asexual expansion. One week later, the infected hepatocytes rupture and release mature parasites, the merozoites. These then begin the erythrocytic phase of malaria by attaching to and invading red blood cells, or erythrocytes. The invasion of the erythrocytes by the malarial parasites leads to malarial pathogenesis and clinical infection.

While there is no FDA approved vaccine for malaria, the World Health Organization (WHO) recently approved the RTS,S vaccine, which has modest efficacy against malaria. Moreover, malarial parasites are increasingly becoming resistant to antimalarial drugs used to treat the disease. Therefore, preventive interventions to inhibit malaria infection are urgently needed for limiting morbidity, mortality, and ultimately eliminating malaria.

### SUMMARY

This disclosure provides monoclonal antibodies and antigen binding fragments directed against PfCSP. In an example, data shows that passive transfer of the disclosed antibodies confers sterile protection in an animal model of malaria infection containing PfCSP, and also that the disclosed antibodies are more potent for inhibiting malaria infection that prior PfCSP monoclonal antibodies.

In some aspects, a monoclonal antibody or antigen binding fragment is provided that comprises a heavy chain variable region (V_{H}) and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as SEQ ID NOs: 1 and 2, respectively (P3-43), SEQ ID NOs: 3 and 4, respectively (D13), SEQ ID NOs: 5 and 6, respectively (P3-21), SEQ ID NOs: 7 and 8, respectively (P3-42), SEQ ID NOs: 9 and 10, respectively (P4-39), SEQ ID NOs: 11 and 12, respectively (D3), SEQ ID NOs: 13 and 14, respectively (P3-45), SEQ ID NOs: 15 and 16, respectively (m43.160), SEQ ID NOs: 17 and 18, respectively (m42.127), SEQ ID NOs: 19 and 20, respectively (m43.151), or SEQ ID NOs: 21 and 22, respectively (Core8_H-K58R). Optionally, the V_{H} and the V_{L} further comprise glutamate or glutamine substitutions at one or more of K13, K19, K23, or R44 in the V_{H} and R18 in the V_{L} (such as K19E, K23E, and R44E substitutions in the V_{H}, and a R18E substitution in the V_{L}). The monoclonal antibody or antigen binding fragment specifically binds to PfCSP and neutralizes P. falciparum.

In some aspects, the V_{H} and the V_{L} comprise amino acid sequences set forth as SEQ ID NOs: 1 and 2, respectively, SEQ ID NOs: 3 and 4, respectively, SEQ ID NOs: 5 and 6, respectively, SEQ ID NOs: 7 and 8, respectively, SEQ ID NOs: 9 and 10, respectively, SEQ ID NOs: 11 and 12, respectively, SEQ ID NOs: 13 and 14, respectively, SEQ ID NOs: 15 and 16, respectively, SEQ ID NOs: 17 and 18, respectively, SEQ ID NOs: 19 and 20, respectively, or SEQ ID NOs: 21 and 22, respectively, SEQ ID NOs: 217 and 218, respectively, SEQ ID NOs: 219 and 220, respectively, or SEQ ID NOs: 221 and 222, respectively. The monoclonal antibody or antigen binding fragment specifically binds to PfCSP and neutralizes P. falciparum.

Also disclosed are compositions including the antibodies and antigen binding fragments, nucleic acids encoding the antibodies and antigen binding fragments, expression vectors comprising the nucleic acids, and isolated host cells that comprise the nucleic acids. In several aspects, the nucleic acid molecule encoding a disclosed antibody or antigen binding fragment can be a cDNA or RNA molecule that encodes the antibody or antigen binding fragment. In additional aspects, the nucleic acid molecule can be a bicistronic expression construct encoding the V_{H} and V_{L} of the antibody or antigen binding fragment.

The disclosed antibodies and antigen binding fragments potently neutralize PfCSP expressed on infectious sporozoites in vivo. Accordingly, a method is disclosed for inhibiting (including preventing) P. falciparum infection in a subject. The method comprises administering an effective amount (that is, an amount effective to inhibit P. falciparum infection in a subject) of one or more of the disclosed antibodies, antigen binding fragments, nucleic acid molecules, vectors, or compositions, to the subject, such as a subject at risk of or having a P. falciparum infection.

The antibodies, antigen binding fragments, nucleic acid molecules, vectors, and compositions disclosed herein can be used for a variety of additional purposes, such as for diagnosing P. falciparum infection in a subject, or detecting P. falciparum in a sample.

The foregoing and other features and advantages of this disclosure will become more apparent from the following detailed description of several aspects, which proceeds with reference to the accompanying figures.

### BRIEF DESCRIPTION OF THE FIGURES

FIGS. 1A-G. Activation of H^{GL-CIS43}κ^{iGL-CIS43} B cells by PfCSP-immunization is dampened by host competitor B cells. (1A) (Left) PfCSP-binding of peripheral B cells in naïve iGL-CIS43 heavy and light antibody chain KI mouse (H^{iGL-CIS43}κ^{iGL-CIS43}) models. Events were pre-gated on lymphocytes/singlets/CD4-CD8⁻F4/80⁻Grl^{-/}B220⁺ B cells and C57BL/6J mice were used as negative controls. (Right) Quantification of PfCSP-binding blood peripheral B cells from H^{iGL-CIS43}κ^{iGL-CIS43} KI model compared to C57BL/6J mice. (1B) Nested pie charts show human iGL-CIS43 heavy chain, human iGL-CIS43 light and murine light chain sequences amplified from single-cell sorted (a) antigen-agnostic (left) or PfCSP-specific (right) naive B cells. The number in the center indicates sequence pairs amplified. (1C) Titration of cell transfer model to generate precise H^{iGL-CIS43}κ^{iGL-CIS43} precursor frequencies at time of immunization (Day 0). Spleen of non-immunized CD45.1 recipient mice were stained for flow cytometric analysis and absolute precursor quantification via bead assay. See also FIGS. 10C-10E for metrics of total B cells counted and recovered H^{iGL-CIS43}κ^{iGL-CIS43} B cells. (1D) Calculation of precursor frequencies corresponding to number of B cells transferred (left) as indicated on x-axis and analysis of linearity of CD45.2 H^{iGL-CIS43}κ^{iGL-CIS43} B cells recovered 24 h post transfer (right). (1E) Schematic of the H^{iGL-CIS43}κ^{iGL-CIS43} B cell transfer/immunization system used for (1F-1H). (1F) Representative flow cytometry graphs at 7 days post immunization showing that PfCSP can induce the formation of small GCs and prime CD45.2 H^{iGL-CIS43}κ^{iGL-CIS43} B cells at precursor frequencies of ˜1:10⁴ (n=5) to 1:10⁵ (n=5). (1G) Quantification of B cell subsets (in order of left to right: total GCs, ratios of GC CD45.1⁺/CD45.2⁺ B cells, PfCSP-reactive CD45.2⁺ B cells and PfCSP-reactive CD45.1⁺ competitor B cells) responsive to PfCSP-immunization at 7 days post immunization. P values were calculated by Mann-Whitney test (FIGS. 1A and 1H). ***P<0.001; ns, statistically non-significant differences.

FIGS. 2A-2E. Immunization with NPDP19-KLH leads to specific and strong activation of H^{iGL-CIS43}κ^{iGL-CIS43} B cells. (2A) (I) Schematic representation of PfCSP (NF54), with region 1 (R1), NANP repeats, NPDP/NVDP repeats, and region 2 (R2) thrombospondin repeats (TSR) indicated. Junctional epitope region NPDP19 included in the (II) KLH-based prototype malaria vaccine candidate and malaria peptide21 are indicated. Numbering (95-115) corresponds to the PfCSP numbering. (2B) ELISA to assess if NPDP19-peptide can be recognized by both iGL-CIS43 and mature CIS43 antibodies in the context of KLH. The HIV-1 bNAb VRCO1 was used as a negative control. (2C) Schematic of the H^{iGL-CIS43}κ^{iGL-CIS43} B cell transfer system used for (2D). (2D) Comparison of representative flow cytometry graphs for recipient mice adoptively transferred to achieve precursor frequencies of 1:10⁴, 1:10⁵ and 1:10⁶, immunized with PfCSP [50 μg/mouse]/Alhydrogel (n=5 per cell dilution) or NPDP19-KLH [50 μg/mouse]/Alhydrogel (n=5 per cell dilution). (Left to right) Graphs indicate total GC-responses, H^{iGL-CIS43}κ^{iGL-CIS43} CD45.2 B cell responses, and PfCSP-binding of CD45.2 B cells. Percentages are based on parent populations. (2E) Quantifications from (2D). Data was normalized to B220+ B cells. P values were calculated by 2-way ANOVA for multiple comparisons (FIG. 2E). *P<0.05; **P<0.01***; P<0.001; ****P<0.0001; ns, statistically non-significant differences.

FIGS. 3A-3E. B cell kinetics of H^{iGL-CIS43}κ^{iGL-CIS43} B cells following immunization with NPDP19-KLH. (3A) Schematic of the H^{iGL-CIS43}κ^{iGL-CIS43} B cell transfer system used for (3B-3D). (3B) Gating strategy to identify PfCSP-specific H^{iGL-CIS43}κ^{iGL-CIS43} B cells. Representative flow cytometry plots are shown for two time points, 13 and 28 DPI. For 13 DPI representative graphs for the two control groups (Sham-recipient mice were injected ip with 200 μl PBS; Alhydrogel only controls were injected ip with 200 μl of 1:1 PBS-Alhydrogel formulation) are shown. (3C) Quantifications of immune response by B cell subsets from (B). (+/−) on x-axis indicate whether Alhydrogel (Alum) and/or NPDP19-KLH were used for immunizations. (3D) Profiles of IgG-binding determined in ELISA to PfCSP and to malaria peptides NPDP19 and (NANP)5 at 13 and 28 days post immunization. mAb 2A10 with mouse variable and mouse IgG1 constant region was used as a standard to determine concentrations of antigen-specific IgG. (3E) Left, binding of mature mAb CIS43 and polyclonal mouse sera from 13 and 28 days post NPDP19-KLH immunization (middle) to rPfCSP in the presence of varying concentrations of peptides. Right, specified amino acid sequences numbered 19-29 are shown. P values were calculated by Kruskal-Wallis test with Dunn's correction.*P<0.05; **P<0.01. The sequences shown in FIG. 3E are KLKQPADGNPDPNAN (SEQ ID NO: 227), PADGNPDPNANPNVD (SEQ ID NO: 51), NPDPNANPNVDPNAN (SEQ ID NO: 52), NANPNVDPNANPNVD (SEQ ID NO: 228), NVDPNANPNVDPNAN (SEQ ID NO: 229), NVDPNANPNANPNAN (SEQ ID NO: 230), NANPNANPNANPNAN (SEQ ID NO: 231), NANPNANPNANPNVD (SEQ ID NO: 232), NANPNANPNVDPNAN (SEQ ID NO: 233), and NANPNVDPNANPNAN (SEQ ID NO: 234).

FIGS. 4A-4G. A single priming immunization with NPDP19-KLH induces antibodies with key CIS43-like mutations N52K and K59R (IGHV) and H89Q and T94S (IGKV). Antigen-specific splenic CD95⁺CD38^{low} CD45.2⁺ H^{iGL-CIS43}κ^{iGL-CIS43} B cells were sorted at day 13 and 28 post immunization (DPI) for single-cell BCR sequence analysis (see FIG. 10E). (4A) Clonal lineage trees were generated from bioinformatically assembled heavy-light chain sequence pairs. The branch length is representative of sequence distance. (4B) Number of total nucleotide (nt) and amino acid (AA) mutations acquired in iGL-CIS43 heavy chains at 13 and 28 DPI. The horizontal line indicates the median number of mutations. (4C) Number of total nucleotide (NT) and amino acid (AA) mutations acquired in the iGL-CIS43 light chain at 13 and 28 DPI. The red line indicates the median number of mutations. Accumulation of CIS43-like aa mutations shown for both the human heavy (left) and light chains (right) isolated at 13 and 28 DPI. The stair step indicate calculated antigen-agnostic mutations. The numbers shown inside each square indicates the number of sequences that have the total AA mutations shown on the x-axis and the CIS43-like AA mutations shown on the y-axis. (3D) Hotspot analysis shows frequency of observed heavy chain mutations per residue at day 13 and day 28 post-immunization. HCDRs are highlighted in gray. Letters in gray (only present in mature CIS43 heavy chain) indicate key amino acid residues for the recognition of the junctional malaria epitope. AA positions 52 and 58 were analyzed in (4F). Kabat numbering was followed. (4E) Distribution of select iGL-CIS43 B cell heavy chain aa mutations in positions 52 and 58 over time. (4F) Hotspot analysis shows frequency of observed light chain mutations per residue at day 13 and day 28 post-immunization. LCDRs are highlighted in gray. Letters in gray (only present in mature CIS43 light chain) indicate key amino acid residues for the recognition of the junctional malaria epitope. AA positions 89 and 94 were analyzed in (H). Kabat numbering was followed. (3G) Distribution of select iGL-CIS43 B cell light chain aa mutations in positions 89 and 94 over time. P values were calculated by unpaired Mann-Whitney test (panels B and C). ****P<0.0001.

FIGS. 5A-5D. Informatics-based analyses identifies affinity- and sequences-based correlates of improved protection. (5A) Sequence-based sieving of genetic features of 161 iGL-CIS43 B cells with heavy and light sequences, identified the top 10 sequences for 5 features, comprising 37 unique antibody sequences of which 34 expressed. (5B) Mice were passively infused with either 200 μg antibody (left, experiments A, B and C) or 50 ug antibody (right, experiment D and E) before being challenged with transgenic P. berghei sporozoites expressing PfCSP and a green fluorescent protein/luciferase fusion protein. Bioluminescent quantification of liver burden is shown 42 h post challenge, with each group of 11 antibodies assessed with controls: naïve (non-infected), max burden (no passively infected antibody), and both iGL and mature forms of CIS43. In experiments D and E, the L9 antibody was added as a control. Relative to mature CIS43, only 3 of the tested iGL-CIS43 B cell-derived antibodies were statistically superior in experiment D, all were superior in experiment E; relative to L9, only antibody m43.151 was statistically superior and then only in experiment D. (5C) BLI affinity for 34 expressed antibodies was measured against PfCSP, junctional peptide and NANP₅-repeat antigens (X-axis) and correlated to normalized liver burden (Y-axis), as assessed at 200 μg/ml. (5D) Correlations between five genetic features chosen for sequence sieving and normalize protection, as assessed at 200 μg/ml.

FIGS. 6A-6C. Thermodynamic and structural basis of improved CIS43 antibodies from iGL-CIS43 mice. (6A) Isothermal calorimetry titrations of PfCSPm. Isothermal titration calorimetry of PfCSPm with various iGL-CIS43-derived antibodies at pH 7.4 and 25° C. The affinities and stoichiometry are shown for both K_{D}1 and K_{D}2. (6B) Crystal structures of junctional peptide (peptide 21) in complex with the most potent iGL-CIS43 derived antibodies from m42 and m43, highlighting similarity in SHM (left) and variation in bound epitope—especially in relation to isoleucine or leucine at heavy chain position 98 (right). Numbering on peptide 21 corresponds to the PfCSP numbering. (6C) Sequences of iGL-CIS43-derived antibodies statistically superior to mature CIS43 are shown with that of mature and iGL-CIS43 along with the heavy (top) and light (bottom) V-gene mutational profiles.

FIGS. 7A-7D.: Design based on iGL-CIS43 mice information yields superior antibody iGL-CIS43.D3. (7A) m43.151 variants designed using (i) expanded amino acid contact mutations (D1-D6) and (ii) mutations on top antibodies (D7-D11). (7B) ALPHALISA®-measured apparent affinity to NPDP19 correlates strongly with protection (R=0.873) for 34 genetically identified antibodies analyzed in FIG. 5 (leftmost panel), with two, iGL-CIS43.D1 and iGL-CIS43.D3 showing especially high ALPHALISA® counts (2^{nd} panel from left). BLI and ITC affinities for these two antibodies are shown for NPDP19 and PfCSPm, respectively (right panels). (7C) Mice were passively infused with either 50 μg (filed circle) or 25 μg (empty circle) antibody before being challenged with transgenic P. berghei sporozoites expressing PfCSPm and a green fluorescent protein/luciferase fusion protein. Bioluminescent quantification was done for each mouse on day 2 and day 6. Relative to mature L9, iGL-CIS43.D3 was statistically superior in both 50 μg and 25 μg group. (7D) Structure-function analysis. Initial core 8 mutation were transplanted onto iGL-CIS43 backbone sequence (iGL_Core8) and structure-function analysis was performed, which included the reversion of single mutations and addition of mutations from iGL-CIS43.D3 onto iGL_Core8 construct. ALPHALISA®-measured apparent affinities to NPDP19 were determined (D3 has ALPHALISA® value 5.53×10⁶), and liver protection was measured (D3 has normalized liver burden 0.03) (left panels). These measurements correlated (middle panel), and the location of each alteration is depicted on the iGL-CIS43.D3 structure (right panel).

FIG. 8A-8F. Characterization of inferred germline CIS43 heavy antibody chain KI mouse, related to FIG. 1. (8A) Representative flow cytometry plots showing binding to PfCSP fluorescent probes (top panel), IgD/IgM B cell subsets (middle panel) and lambda-kappa B cell subsets (bottom panel) for iGL-CIS43 KI mouse models. C57BL/6 mice were used as controls. (8B) Quantifications B cells from (8A). (8C) Representative flow cytometric plots of peripheral B cells binding to PfCSP fluorescent probe in heterozygous mice (H^{iGL-CIS43/WT}κ^{iGL-CIS43/WT}) and homozygous mice (H^{iGL-CIS43/iGL-CIS43}κ^{iGL-CIS43/iGL-CIS43}) (8D) An example of the single B cell sorting schematic for sequencing analysis. (8E) Frequency of H^{iGL-CIS43}κ^{iGL-CIS43} B cells recovered 24 hours after adoptive transfer. Spleen of unimmunized CD45.1 recipients were stained and analyzed. Gated as B220⁺, CD4⁻, CD8⁻, F4/80⁻, GR1⁻, CD45.2+. (8F) Gate for selection of CountBright beads. (8G) Metrics of B cells and/or beads counted and H^{iGL-CIS43}κ^{iGL-CIS43} B cells recovered as in C/D. P values were calculated by multiple comparisons Kruskal-Wallis test (panel B). **P<0.01; ns, statistically non-significant differences.

FIG. 9A-9H. Immunization with NPDP19-KLH is more specific and leads to strong activation of H^{iGL-CIS43} B cells, related to FIGS. 1 and 2. (9A) Comparison of representative flow cytometry graphs for recipient mice adoptively transferred to achieve precursor frequencies of 1:10⁶, 1:105⁵ and 1:10⁴ (top to bottom), immunized with PfCSP or NPDP19-KLH(n=5 per cell dilution). Graphs indicate total PfCSP-binding of CD45.1⁺ host B cells. Percentages are based on parent populations. Quantifications from (9D). Data was normalized to B220+ B cells. (9B) Representative flow cytometric plots of murine CD45.1⁺ host B cells binding to PfCSP fluorescent probe (top panel) and NPDP19 fluorescent probe (bottom panel) on Day 7 following immunization with NPDP19-KLH (blue) or PfSP (red). Quantifications of B cells from are shown (right panel). (9C) Schematic of the H^{iGL-CIS43} B cell transfer system used for (9B-9D). (9D) Comparison of representative flow cytometry graphs for recipient mice adoptively transferred 0.5×10⁶ donor B cells, immunized with PfCSP/Alhydrogel (n=3, top panel) or NPDP19-KLH/Alhydrogel (n=3, bottom panel). (Left to right) Graphs indicate total GC-responses, H^{iGL-CIS43} CD45.2. B cell responses and PfCSP-binding of H^{iGL-CIS43} CD45.2⁺ B cells. (9E) Nested pie charts show human heavy chain and murine light chain sequences amplified from single-cell sorted PfCSP⁺ IgG1⁺ GC⁺ CD45.2⁺ H^{iGL-CIS43} B cells. The number in the center of the nested pie charts indicates sequence pairs amplified. (9F) Number of nucleotide (nt) and amino acid mutations (aa) in the human IGHV and murine IGKV of single-sorted PfCSP⁺ IgG1⁺GC⁺ CD45.2⁺ H^{iGL-CIS43} B cells. (9G) Quantifications of H^{iGL-CIS43} B cells from (E). (9H) Bioinformatic approach to identify mouse germline V gene (extracted from IMGT) most similar to human VK4-1. Murine VK8-30 had the highest similarity to human Vk4-1. P values were calculated by 2-way ANOVA for multiple comparisons (panel 9A) and Wilcoxon matched pairs signed rank test (panel 9G). *P<0.05; ****P<0.0001; ns, statistically non-significant differences.

FIGS. 10A-10G. Quantification of C57BL/6J control groups, validation of PfCSP fluorescent probe and/or NPDP19-fluorescent probe staining and silent/non-silent mutation analysis, related to FIGS. 3 and 4. (10A) Comparison of binding to PfCSP fluorescent probe (left), NPDP19 fluorescent probe (right) in CD45.2 H^{iGL-CIS43}κ^{iGL-CIS43} B cells at 13 and 28 days following NPDP19-KLH immunization. (10B) Quantifications of B cells from (10D). (10C) Representative flow cytometric plots showing that NPDP19-KLH immunization does not activate adoptively transferred CD45.2 C57BL/6J B cells. (10D) Quantifications of B cells from (10C). (10E) An example of the single B cell sorting schematic for sequencing analysis. The shown flow plot is from an NPDP19-KLH immunized mouse on day 13. (10F) Ratios of silent and non-silent mutations for human IGHV1-3 and IGKV4-1 from paired sequences isolated at 13 (top) and 28 days(bottom) following NPDP19-KLH immunization were. determined using the IMGT-alignment software (imgt.org) (10G) P values were calculated by Mann-Whitney test (panel B. **P<0.01. The sequences shown in FIG. 10H are EDNEKLRKPKHKKLR (SEQ ID NO: 235), KLRKPKHKKLKQPAD (SEQ ID NO: 236), KLKQPADGNPDPNAN (SEQ ID NO: 237), KLKQPADGNPDPNAN (SEQ ID NO: 227), PADGNPDPNANPNVD (SEQ ID NO: 51), NPDPNANPNVDPNAN (SEQ ID NO: 52), NANPNVDPNANPNVD (SEQ ID NO: 228), NVDPNANPNVDPNAN (SEQ ID NO: 229), NVDPNANPNANPNAN (SEQ ID NO: 230), NANPNANPNANPNAN (SEQ ID NO: 231), NANPNANPNANPNVD (SEQ ID NO: 232), NANPNANPNVDPNAN (SEQ ID NO: 233), NANPNVDPNANPNAN (SEQ ID NO: 234), and NANPNANPNANPNKN (SEQ ID NO: 238).

FIGS. 11A-11C. Informatic analyses of elicited heavy-light sequenced antibodies, related to FIG. 5. (11A) Schematic showing 5 properties used to select antibodies for affinity and functional analyses. (11B) Bar plots represent the value of each property for each antibody. (11C) Venn diagrams and a matrix showing overlaps of antibodies ranked top 10 in each of the properties. (Left) Color keys for the Venn diagrams and table. (Center) Two Venn diagrams show the relationship of selected top 10 antibodies between 13 DPI and 28 DPI antibodies. (Right) Table summarizing the relationship between the properties. (11D) Presence of restricted set mutations versus protective efficacy as assessed on top 10-ranked antibodies. (11E) Binding of iGL-CIS43 B cell mAbs to malaria sporozoites. P. berghei sporozoites expressing PfCSP (Pb-PfCSP-SPZ) were harvested from mosquito salivary glands and incubated with varying concentrations (2-2×10-7 μg/mL) of CIS43, iGL-CIS43_UCA, and iGL-CIS43 B cell mAbs, followed by secondary antibody detection, and binding was quantitated by flow cytometry. Graph indicates median fluorescence intensity (MFI) of the population of mAb-bound SPZ.

FIGS. 12A-12F. Isothermal titration calorimetry measurements and structure of variant CIS43 recognition of PfCSP, related to FIG. 6. (12A) Thermodynamic parameters of PfCSPm recognition by iGL-CIS43 variant antibodies. (12B) Correlation between ITC-measured CIS43 variant KD1 (related to recognition of the junction epitope) and protection. (12C) ITC-measured CIS43 variant KD2 (related to recognition of repeat region) does not correlate with protection. (12D) ITC assessment of mutants L98I (mature CIS43) and I98L (m43.151) interactions with PfCSP. (12E) Alternation in binding affinity from I98L for CIS43 and L98I for m43.151 based on in silico calculation, respectively. (12F) Structure of peptide 21 in complex of CIS43-like antibodies. SHMs were shown in stick with eight core mutations in red and seven additional mutations in purple.

FIGS. 13A-13D. Analyses of ALPHALISA® readout, ITC and Core8 variants mediated liver burden protection, related to FIG. 7. (13A) Correlation of BLI measured K_{D}s and ALPHALISA® signal. (13B) Titrations of PfCSPm with iGL-CIS43.D1 and iGL-CIS43.D3. (13C) Bioluminescent quantification of liver burden is shown 42 h post challenge, with each group of 16 antibodies assessed with controls: naïve (non-infected), and max burden (no passively infected antibody). (13D) Titrations of PfCSPm with the different Core8 mutant mAbs. In addition to the two-site binding model used to analyze the data, the binding curves for iGL-Core8_K52N_{H}, iGL-Core8_I98V_{H}, and iGL-Core8_S94T_{L} were also analyzed using a single site binding model, as the two site behavior was less apparent with these variants. Comparison of calculated biding parameters between one- and two-site models, suggested these three antibodies to have two sites.

FIG. 14. A mouse model to improve repeat-antigen-targeting antibodies, related to FIG. 7. Schematic outlining steps employed to improve the repeat-antigen-targeting antibodies like CIS43. Steps 1-6 are delineated in the current manuscript and led to the development of D3, with ˜15-fold improved affinity to PfCSP and 5-8-fold improved malaria-protective capacity. Step 7 would involve repeating steps 1-6, with D3 as the informatics template and with improved vaccination, perhaps the use of VLPs encoding junctional peptide (Francica et al., 2021) or of another immunization regime that yields superior SHM as assessed in step 3. Step 3 would also be altered to focus on contact and total AA mutations, which we observed to correlate with increase protection.

FIGS. 15, 16, 17A-17B, 18, and 19A-19B show results of ALPHALISA® assessment of variant CIS43 antibodies.

FIG. 20. Assessment of variant CIS43 antibodies using the P. berghei liver invasion mouse model.

FIG. 21. Comparison of liver burden data for variant CIS43 antibodies assessed using the P. berghei liver invasion mouse model.

FIG. 22. Comparison of liver burden (day 2 post infection) and parasitemia (day 6 post infection) for variant CIS43 antibodies assessed using the P. berghei liver invasion mouse model.

FIGS. 23A and 23B. ALPHALISA® assessment of variant CIS43 antibodies, and heparin chromatography assay.

FIG. 24. Assessment of CIS43 antibody, and CIS43.C20, CIS43.C21, and CIS43.C22 charge variant antibodies using the P. berghei liver invasion mouse model.

FIG. 25. Pharmokinetic assessment of variant CIS43 antibodies with the “C21” set of mutations and with or without the “LS” mutation were assessed in a human FcRn knock-in mouse model.

FIGS. 26A-26C. Assessment of variant CIS43 antibodies using the P. berghei liver invasion mouse model.

FIG. 27 shows outline linear regression model for half-life estimation using data from antibodies containing the “LS” substitution.

FIG. 28 illustrates the population pharmacokinetic analysis using a two-compartment model with first-order absorption for CIS43LS half-life estimation.

FIG. 29 illustrates modeling of CIS43LS and CIS43LS.C21 concentration over time based on mean serum concentrations in hFcRn transgenic mice.

FIG. 30 illustrates modeling of the mean serum concentration of CIS43LS and CIS43LS.c21 over time in humans.

### SEQUENCES

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “4239_106253_02_Sequence_Listing” (348,160 bytes), which was created on Nov. 4, 2022, which is incorporated by reference herein.

SEQ ID NO: 1 is the amino acid sequence of the m43_HH28K_17_PH104K (also called P3-43) V_{H}.
QVHLVQSGAEVKKPGASVKVSCKASGYTFTSYAIHWVRQAPGQRLEWMGWIKGGNGNTRYSQKFQDRVTITRDTSASTAY
MELSSLRSEDTAVYYCALLTVITKDDAFDIWGQGTMVTVSS
SEQ ID NO: 2 is the amino acid sequence of the m43_HH28K_17_PH104K (also called P3-43) V_{L}.
DIVMTQSPDSLAVSLGERATINCKSSQNILYSSNNKNYLAWYQQKPGQPPKLLFYWASTRESGVPDRFSGSGSGTDFTLT
ISSLQAEDVAVYYCHQYYSSPLTFGGGTKVEIK
SEQ ID NO: 3 is the amino acid sequence of the D13 (also called iGL-CIS43.D13) V_{H}.
QVQLVQSGAEVKKPGASVKVSCKASGYTFTRYAIHWVRQAPGQRLEWMGWIKAGNGNTRYSQKFQDRVTITRDTSASTAY
MELSSLRSEDTAVYYCALLTVITPDDAFDIWGQGTMVTVSS
SEQ ID NO: 4 is the amino acid sequence of the D13 (also called iGL-CIS43.D13) V_{L}.
DIVMTQSPDSLAVSLGERATINCKSSQNIFFSSKNKNYLAWYQQIPGQPPKLLIYWASTRESGVPDRFSGSGSGTDFTL
TISSLQAEDVAVYYCHQYYSSPLTFGGGTKVEIQ
SEQ ID NO: 5 is the amino acid sequence of the m42_HH28K_13_TH103R_PH104Q (also called P3-
21) V_{H}.
QVQLVQSGAEVKKPGASVKVSCKASGYTFTSYAIHWVRQAPGQRLEWMGWIKAGNGNTRYSQKFQDRVTITRDTSASTAY
MELSSLRSEDTAVYYCALLTVIRQDDTFDIWGQGTMVTVSS
SEQ ID NO: 6 is the amino acid sequence of the m42_HH28K_13_TH103R_PH104Q (also called P3-
21) V_{L}.
DIVMTQSPDSLAVSLGERATINCKSSQNILYSSNNKNYLAWYQQKPGQAPKLLIYWASTRESGVPDRFSGSGSGTDFTLT
ISSLQAEDVAVYYCHQYYSSPLTFGGGTKVEIK
SEQ ID NO: 7 is the amino acid sequence of the m43_HH28K_17_AH107R (also called P3-42) V_{H}.
QVHLVQSGAEVKKPGASVKVSCKASGYTFTSYAIHWVRQAPGQRLEWMGWIKGGNGNTRYSQKFQDRVTITRDTSASTAY
MELSSLRSEDTAVYYCALLTVITPDDRFDIWGQGTMVTVSS
SEQ ID NO: 8 is the amino acid sequence of the m43_HH28K_17_AH107R (also called P3-42) V_{L}.
DIVMTQSPDSLAVSLGERATINCKSSQNILYSSNNKNYLAWYQQKPGQPPKLLFYWASTRESGVPDRFSGSGSGTDFTLT
ISSLQAEDVAVYYCHQYYSSPLTFGGGTKVEIK
SEQ ID NO: 9 is the amino acid sequence of the iGL-CIS43-KLH-D42.39 (P4-39) V_{H}.
QVQLVQSGAEVKKPGASVKVSCKTSGYTFTNYALHWVRQAPGQRLEWMGWIKTGNGDTRYSQKFQDRVTITRDTSASTAY
MELGSLRSEDTAVYYCALLTVITPDDAFDIWGQGTMVTVSS
SEQ ID NO: 10 is the amino acid sequence of the iGL-CIS43-KLH-D42.39 (P4-39) V_{L}.
DIVMTQSPDSLAVSLGERATINCKSSQSVLYRTNNKNYLAWYQQKPGQPPKLLIYWASTRESGVPDRFSGSGSGTDFTLT
ISSLQAEDVAVYYCHQYYSSPLTFGGGTKVEIK
SEQ ID NO: 11 is the amino acid sequence of the D3 (also called iGL-CIS43.D3) V_{H}.
QVHLVQSGAEVKKPGASVKVSCKASGYTFTrYAIHWVRQAPGQRLEWMGWIKGGNGNTRYSQKFQDRVTITRDTSASTAY
MELSSLRSEDTAVYYCALLTVITPDDAFDIWGQGTMVTVSS
SEQ ID NO: 12 is the amino acid sequence of the D3 (also called iGL-CIS43.D3) V_{L}.
DIVMTQSPDSLAVSLGERATINCKSSQNIffSSNNKNYLAWYQQKPGQPPKLLFYWASTRESGVPDRFSGSGSGTDFTLT
ISSLQAEDVAVYYCHQYYSSPLTFGGGTKVEIK
SEQ ID NO: 13 is the amino acid sequence of the m43_HH28K_17_TH100M (also called P3-45) V_{H}.
QVHLVQSGAEVKKPGASVKVSCKASGYTFTSYAIHWVRQAPGQRLEWMGWIKGGNGNTRYSQKFQDRVTITRDTSASTAY
MELSSLRSEDTAVYYCALLMVITPDDAFDIWGQGTMVTVSS
SEQ ID NO: 14 is the amino acid sequence of the m43_HH28K_17_TH100M (also called P3-45) V_{L}.
DIVMTQSPDSLAVSLGERATINCKSSQNILYSSNNKNYLAWYQQKPGQPPKLLFYWASTRESGVPDRFSGSGSGTDFTLT
ISSLQAEDVAVYYCHQYYSSPLTFGGGTKVEIK
SEQ ID NO: 15 is the amino acid sequence of the m43.160 (also called m43_HH28K.26) V_{H}.
QVQLVQSGAEVKKPGASVKVSCKASGYTFTSYAIHWVRQAPGQRLEWMGWIKAGNGNTRYSQKFQGRVTITRDTSASTAY
MELSSLRSEDTAGYYCALLTVITPDDAFDIWGQGTMVTVSS
SEQ ID NO: 16 is the amino acid sequence of the m43.160 (also called m43_HH28K.26) V_{L}.
DIVMTQSPDSLAVSLGERASINCKSSQNILF SSNNKNYLAWYQQKPGQPPKLLIYWASTRE SGVPDRFSGSGSGTDFTLT
ISSLQAEDVAVYICHQYYSSPLTFGGGTKVEIK
SEQ ID NO: 17 is the amino acid sequence of the m42.127 (also called m42_HH28K.13) V_{H}.
QVQLVQSGAEVKKPGASVKVSCKASGYTFTSYAIHWVRQAPGQRLEWMGWI KAGNGNTRYSQKFQDRVTITRDTSASTAY
MELSSLRSEDTAVYYCALLTVITPDDTFDIWGQGTMVTVSS
SEQ ID NO: 18 is the amino acid sequence of the m42.127 (also called m42_HH28K.13) V_{L}.
DIVMTQSPDSLAVSLGERATINCKSSQNILYSSNNKNYLAWYQQKPGQAPKLLIYWASTRESGVPDRFSGSGSGTDFTLT
ISSLQAEDVAVYYCHQYYSSPLTFGGGTKVEIK
SEQ ID NO: 19 is the amino acid sequence of the m43.151 (also called m43_HH28K.17) V_{H}.
QVHLVQSGAEVKKPGASVKVSCKASGYTFTSYAIHWVRQAPGQRLEWMGWIKGGNGNTRYSQKFQDRVTITRDTSASTAY
MELSSLRSEDTAVYYCALLTVITPDDAFDIWGQGTMVTVSS
SEQ ID NO: 20 is the amino acid sequence of the m43.151 (also called m43_HH28K.17) V_{L}.
DIVMTQSPDSLAVSLGERATINCKSSQNILYSSNNKNYLAWYQQKPGQPPKLLFYWASTRESGVPDRFSGSGSGTDFTLT
ISSLQAEDVAVYYCHQYYSSPLTFGGGTKVEIK
SEQ ID NO: 21 is the amino acid sequence of Core8_H-K58R (also called Core8-H:K58R) V_{H}.
QVQLVQSGAEVKKPGASVKVSCKASGYTFTSYAIHWVRQAPGQRLEWMGWI KAGNGNTKYSQKFQGRVTI TRDTSASTAY
MELSSLRSEDTAVYYCALLTVITPDDAFDIWGQGTMVTVSS
SEQ ID NO: 22 is the amino acid sequence of the Core8_H-K58R (also called Core8-H:K58R) V_{L}.
DIVMTQSPDSLAVSLGERATINCKSSQNILYSSNNKNYLAWYQQKPGQPPKLLIYWASTRESGVPDRFSGSGSGTDFTLT
ISSLQAEDVAVYYCHQYYSSPLTFGGGTKVEIK
SEQ ID NOs: 23-43 are CDR sequences.
SEQ ID NO: 44 is the amino acid sequence of the CIS43 V_{H}.
QVQLVQSGAEVKKPGASVKVSCKASGYTFTSYAIHWVRQAPGQRLEWMGWI KAGNGNTRYSQKFQDRVTITRDTSTTTA
YMELSSLRSEDTAVYYCALLTVLTPDDAFDIWGQGTMVTVSS
SEQ ID NO: 45 is the amino acid sequence of the CIS43 V_{L}.
DIVMTQSPDSLAVSLGERATINCKSSQSVLYSSNNKNYLAWYQQKPGQPPNLLIYWASTRQSGVPDRFSGSGSGTDFTL
TISSLQAEDVAVYYCHQYYSSPLTFGGGTKVEIK
SEQ ID NO: 46 is the amino acid sequence of the L9 V_{H}.
QVKLVESGGGVVQPGRSLRLSCEASGFIFSTYGMHWVRQAPGKGLEWVAVIWFDGSNIYYADSVKGRFTISRDNSKNTVF
MQMDSLRAEDTAVYYCHRNFYDGSGPFDYWGQGTLVTVSS
SEQ ID NO: 47 is the amino acid sequence of the L9 V_{L}.
DIQMTQSPSTLSASVGDRVTITCRASQFISRWLAWYQQKPGKAPKLLIYKASSLESGVPSRFSGSGSETHFTLTI SSLQP
DDVATYYCQEYTSYGRTFGQGTKVEIK
SEQ ID NO: 48 is an exemplary amino acid sequence for PfCSP (GenBank Acc. No. CAB38998.2,
incorporated by reference herein)
MMRKLAILSVSSELFVEALFQEYQCYGSSSNTRVLNELNYDNAGINLYNELEMNYYGKQENWYSLKKNSRSLGENDDGN
NEDNEKLRKPKHKKLKQPADGNPDPNANPNVDPNANPNVDPNANPNVDPNANPNANPNANPNANPNANPNANPNANPNA
NPNANPNANPNANPNANPNANPNANPNANPNANPNANPNVDPNANPNANPNANPNANPNANPNANPNANPNANPNANPN
ANPNANPNANPNANPNANPNANPNANPNANPNANPNKNNQGNGQGHNMPNDPNRNVDENANANSAVKNNNNEEPSDKHI
KEYLNKIQNSLSTEWSPCSVTCGNGIQVRIKPGSANKPKDELDYANDIEKKICKMEKCSSVENVVNSSIGLIMVLSFLF
LN
SEQ ID NO: 49 is the amino acid sequence of an IgG1 heavy chain including the CIS43 V_{H}.
QVQLVQSGAEVKKPGASVKVSCKASGYTFTSYAIHWVRQAPGQRLEWMGWIKAGNGNTRYSQKFQDRVTITRDTSTTTA
YMELSSLRSEDTAVYYCALLTVLTPDDAFDIWGQGTMVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKENWYVDGVEVHNAKTKPREEQYNSTYRVVSVLIVLHQD
WLNGKEYKCKVSNKALPAPIEKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK
SEQ ID NO: 50 is the amino acid sequence of an IgG1 light chain including the CIS43 V_{L}.
DIVMTQSPDSLAVSLGERATINCKSSQSVLYSSNNKNYLAWYQQKPGQPPNLLIYWASTRQSGVPDRFSGSGSGTDFTL
TISSLQAEDVAVYYCHQYYSSPLTFGGGTKVEIKRTVAAPSVE IFPPSDEQLKSGTASVVCLLNNFYPREAKVQWKVDN
ALQSGNSQESVTEQDSKDSTYSLSSTLTLSKADYEKHKVYACEVTHQGLSSPVTKSENRGEC

SEQ ID NOs: 51 and 52 are peptide sequences.
SEQ ID NOs: 53-214 are heavy and light chain variable region sequences for variant CIS43 antibodies.
SEQ ID NOs: 215 and 216 are the heavy and light chain sequences of the CAIS43LS.C21 antibody.
SEQ ID NOs: 217-222 are heavy and light chain variable region sequences for variant CIS43 antibodies.
SEQ ID NOs: 223 and 224 are primer sequences.
SEQ ID NOs: 225 and 226 are CIS43 VH and VL iGL sequences.
SEQ ID NOs: 227-238 are peptide sequences.
SEQ ID NOs: 239-254 are heavy and light chain variable region sequences for variant CIS43 antibodies.

### DETAILED DESCRIPTION

Malaria is a mosquito-borne parasitic disease causing high morbidity and mortality, primarily in infants and young children in sub-Saharan Africa. Development of a highly effective vaccine or antibodies that can prevent and ultimately eliminate malaria is urgently needed. This disclosure provides monoclonal antibodies and antigen binding fragments directed against PfCSP. Data in the examples show that passive transfer of the disclosed antibodies confers high-level, sterile protection in an animal model, and that the disclosed antibodies are more potent for inhibiting malaria infection than prior antibodies against PfCSP. Thus, the PfCSP-specific antibodies and antigen binding fragments provided herein are effective for passive prevention of malaria for use in suitable subjects, such as travelers, military personnel, and subjects in elimination campaigns.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

About: Unless context indicated otherwise, “about” refers to plus or minus 5% of a reference value. For example, “about” 100 refers to 95 to 105.

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Antibody and Antigen Binding Fragment: An immunoglobulin, antigen-binding fragment, or derivative thereof, that specifically binds and recognizes an analyte (antigen) such as PfCSP. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Diibel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some aspects, a disclosed antibody includes a heterologous constant domain. For example, the antibody includes a constant domain that is different from a native constant domain, such as a constant domain including one or more modifications (such as the “LS” mutations) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one aspect, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some aspects, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008).

Antibody or antigen binding fragment that neutralizes P. falciparum: An antibody or antigen binding fragment that specifically binds to a P. falciparum antigen (such as PfCSP) in such a way as to inhibit a biological function associated with P. falciparum that inhibits P. falciparum infection. The antibody can neutralize the activity of P. falciparum at various points during the lifecycle of the pathogen. For example, an antibody or antigen binding fragment that neutralizes P. falciparum may interfere with the pathogen by binding it in the skin and limiting entry into the blood or entry into the hepatocytes in the liver by interfering with the interaction of the pathogen and one or more cell surface receptors. Alternately, an antibody may interfere with one or more post-attachment interactions of the pathogen with its receptors, for example, by interfering with pathogen internalization by receptor-mediated endocytosis.

In some aspects, an antibody or antigen binding fragment that specifically binds to PfCSP and neutralizes P. falciparum inhibits sporozoite invasion of hepatocytes, for example, by at least 50% (such as at least 60%, at least 70%, at least 80%, at least 90%, or more) compared to a control antibody or antigen binding fragment. In some aspects, an antibody or antigen binding fragment that specifically binds to PfCSP and neutralizes P. falciparum inhibits infection of a human subject by P. falciparum, for example, by at least 50% compared to a control antibody or antigen binding fragment.

Biological sample: A sample obtained from a subject. Biological samples include all clinical samples useful for detection of disease or infection (for example, P. falciparum infection) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having a P. falciparum infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

Circumsporozoite protein (CSP): The circumsporozoite protein (CSP) is a major malaria parasite surface protein during the sporogonic cycle. PfCSP covers the surface of P. falciparum sporozoites, which are transmitted from the mosquito salivary gland to host hepatocytes. An exemplary PfCSP amino acid sequence is provided as SEQ ID NO: 84.

CIS43 Antibody: A monoclonal antibody that specifically binds to an epitope on PfCSP and neutralizes malaria infection. The CIS43 antibody and methods for its production are described, for example, in PCT Pub. No. WO 2018/148660, which is incorporated by reference herein in its entirety. The amino acid sequences of the heavy and light variable regions of the CIS43 antibody are provided herein as SEQ ID NOs: 80 and 81.

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one aspect, an antibody is linked to an effector molecule; for example, an antibody that specifically binds to CSP from P. falciparum covalently linked to an effector molecule. The linkage can be by chemical or recombinant means. In one aspect, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a PfCSP-specific antibody can include up to 1, 2, 3, 4, 5, 6, 7, 8, 9, or up to 10 conservative substitutions compared to a reference antibody sequence and retain specific binding activity for CSP, and/or P. falciparum neutralization activity. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some aspects less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the PfCSP specific antibody, such as the ability to specifically bind to PfCSP or neutralize P. falciparum. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some aspects, the control is a negative control, such as sample obtained from a healthy patient not infected with P. falciparum. In other aspects, the control is a positive control, such as a tissue sample obtained from a patient diagnosed with P. falciparum infection. In still other aspects, the control is a historical control or standard reference value or range of values (such as a previously tested control sample, such as a group of P. falciparum patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

Effective amount: A quantity of a specific substance sufficient to achieve a desired effect in a subject to whom the substance is administered. For instance, this can be the amount necessary to inhibit a P. falciparum infection, such as the amount necessary to inhibit or prevent P. falciparum sporozoites from invading the liver in the subject or to measurably alter outward symptoms of the P. falciparum infection.

In some aspects, administration of an effective amount of a disclosed antibody or antigen binding fragment that binds to PfCSP can reduce or inhibit a P. falciparum infection (for example, as measured by infection of cells, or by number or percentage of subjects infected by the P. falciparum, or by an increase in the survival time of infected subjects, or reduction in symptoms associated with P. falciparum infection) by a desired amount, for example by at least 10%, at least 20%, at least 50%, at least 60%, at least 70%, at least 80%, at least 90%, at least 95%, at least 98%, or even at least 100% (elimination or prevention of detectable P. falciparum infection), as compared to a suitable control.

The effective amount of an antibody or antigen binding fragment that specifically binds PfCSP that is administered to a subject to inhibit P. falciparum infection will vary depending upon a number of factors associated with that subject, for example the overall health and/or weight of the subject. An effective amount can be determined by varying the dosage and measuring the resulting response, such as, for example, a reduction in pathogen titer. Effective amounts also can be determined through various in vitro, in vivo or in situ immunoassays.

An effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid) or syringe having sterile components.

Effector molecule: A molecule intended to have or produce a desired effect; for example, a desired effect on a cell to which the effector molecule is targeted. Effector molecules can include, for example, polypeptides and small molecules. In one non-limiting example, the effector molecule is a toxin. Some effector molecules may have or produce more than one desired effect.

Epitope: An antigenic determinant. These are particular chemical groups or peptide sequences on a molecule that are antigenic, i.e. that elicit a specific immune response. An antibody specifically binds a particular antigenic epitope on a polypeptide. In some examples a disclosed antibody specifically binds to an epitope on CSP from P. falciparum.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cy2 and Cy3 and optionally the lower part of the hinge between Cγ1 and Cγ2. Although the boundaries of the Fc region may vary, the human IgG heavy chain Fc region is usually defined to include residues following C226 or P230 to the Fc carboxyl-terminus, wherein the numbering is according to Kabat. For IgA, the Fc region includes immunoglobulin domains Cα2 and Cα3 and optionally the lower part of the hinge between Cα1 and Cα2.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgA: A polypeptide belonging to the class of antibodies that are substantially encoded by a recognized immunoglobulin alpha gene. In humans, this class or isotype comprises IgA₁ and IgA₂. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

IgG: A polypeptide belonging to the class or isotype of antibodies that are substantially encoded by a recognized immunoglobulin gamma gene. In humans, this class comprises IgG₁, IgG₂, IgG₃, and IgG₄.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Inhibiting a disease or condition: Reducing the full development of a disease or condition in a subject, for example, reducing the full development of a P. falciparum infection in a subject who is at risk of a P. falciparum infection. This includes neutralizing, antagonizing, prohibiting, preventing, restraining, slowing, disrupting, stopping, or reversing progression or severity of the disease or condition.

Inhibiting a disease or condition refers to a prophylactic intervention administered before the disease or condition has begun to develop (for example a treatment initiated in a subject at risk of P. falciparum infection, but not infected by P. falciparum) that reduces subsequent development of the disease or condition and/or ameliorates a sign or symptom of the disease or condition following development. The term “ameliorating,” with reference to inhibiting a disease or condition refers to any observable beneficial effect of the prophylactic intervention intended to inhibit the disease or condition. The beneficial effect can be evidenced, for example, by a delayed onset of clinical symptoms of the disease or condition in a susceptible subject, a reduction in severity of some or all clinical symptoms of the disease or condition, a slower progression of the disease or condition, an improvement in the overall health or well-being of the subject, a reduction in infection, or by other parameters that are specific to the particular disease or condition.

In some aspects, the disclosed PfCSP-specific antibodies and antigen binding fragments inhibit the invasion of Plasmodium falciparum: sporozoites into human liver cells (hepatocytes). As mentioned above, the invasion of liver cells is a key event in the infection of a subject with the malaria parasite. Inhibition of the invasion of human liver cells can be measured by one or more of several standard assays (see, for example, Example 1). For example, the disclosed PfCSP-specific antibodies and antigen binding fragments can inhibit the invasion of Plasmodium falciparum: sporozoites into human liver cells by at least 25%, such as at least 50%, at least 75%, at least 90%, at least 95%, or 100% compared to a suitable control.

In some aspects, the disclosed PfCSP-specific antibodies and antigen binding fragments inhibit the growth of Plasmodium falciparum: in a subject, for example, the antibodies and antigen binding fragments inhibit the multiplication of Plasmodium falciparum: in the subject, resulting in a reduction in pathogen load in the subject compared to a relevant control. For example, the disclosed PfCSP-specific antibodies and antigen binding fragments can inhibit the growth of Plasmodium falciparum: in a subject by at least 25%, such as at least 50%, at least 75%, at least 90%, at least 95%, or 100% compared to a suitable control.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Kabat position: A position of a residue in an amino acid sequence that follows the numbering convention delineated by Kabat et al. (Sequences of Proteins of Immunological Interest, 5^{th} Edition, Department of Health and Human Services, Public Health Service, National Institutes of Health, Bethesda, NIH Publication No. 91-3242, 1991).

L9 Antibody: A monoclonal antibody that specifically binds to an epitope on PfCSP and neutralizes malaria infection. The L9 antibody and methods for its production are described, for example, in PCT Pub. No. WO 2020/227228, which is incorporated by reference herein in its entirety. The amino acid sequences of the heavy and light variable regions of the L9 antibody are provided herein as SEQ ID NOs: 82 and 83.

Linker: A bi-functional molecule that can be used to link two molecules into one contiguous molecule, for example, to link an effector molecule to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Malaria: Malaria is a parasitic infection of humans by the Plasmodium species P. falciparum, P. vivax, P. ovale, P. malariae, and P. knowlesi. Humans become infected following the bite of an infected mosquito, the host of the malarial parasite. Malaria rarely occurs in humans following a blood transfusion or subsequent to needle-sharing. Clinical manifestations of malarial infection which may occur include blackwater fever, cerebral malaria, respiratory failure, hepatic necrosis, occlusion of myocardial capillaries and death.

Infection begins when malaria sporozoites gain access to or are directly injected into the bloodstream of a host by a mosquito. After injection, they migrate to the liver and multiply in hepatocytes for one week. The sporozoites substantially expand in the liver and differentiate to merozoites which are released from the liver into the blood stream, where they infect erythrocytes. When the merozoite matures in the red blood cell, it is known as a trophozoite and, when fully developed, as a schizont. A schizont is the stage when nuclear division occurs to form individual merozoites which are released to invade other red cells. Malaria clinical symptoms appear during the blood-stage. After several schizogonic cycles, some parasites, instead of becoming schizonts through asexual reproduction, develop into large uninucleate parasites, known as gametocytes. These gametocytes are the sexual blood cell stage forms of the parasite.

Sexual development of the malaria parasites involves the female macrogametocyte and the male microgametocyte. If a mosquito feeds on the blood of an infected host, it can ingest gametocytes within the blood. Fertilization and sexual recombination of the parasite occurs in the mosquito's gut. The fertilized parasite, which is known as a zygote, then develops into an ookinete. The ookinete penetrates the midgut wall of the mosquito and develops into an oocyst, within which many small sporozoites form. When the oocyst ruptures, the sporozoites migrate to the salivary gland of the mosquito via the hemolymph. Once in the saliva of the mosquito, the parasite can be injected into a host, repeating the life cycle.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some aspects, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some aspects, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one aspect, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several aspects, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The identity between two or more nucleic acid sequences, or two or more amino acid sequences, is expressed in terms of the identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Any suitable method may be used to align sequences for comparison. Non-limiting examples of programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics, 5(2):151-3, 1989; Corpet, Nucleic Acids Res. 16(22):10881-10890, 1988; Huang et al. Bioinformatics, 8(2):155-165, 1992; and Pearson, Methods Mol. Biol. 24:307-331, 1994. Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example PfCSP) and does not bind in a significant amount to other proteins present in the sample or subject. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

An antibody that specifically binds to an epitope on PfCSP is an antibody that binds substantially to PfCSP, including cells or tissue expressing PfCSP, substrate to which the PfCSP is attached, or PfCSP in a biological specimen. It is, of course, recognized that a certain degree of non-specific interaction may occur between an antibody and a non-target (such as a cell that does not express PfCSP). Typically, specific binding results in a much stronger association between the antibody and protein or cells bearing the antigen than between the antibody and protein or cells lacking the antigen. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Subject: Living multi-cellular vertebrate organisms, a category that includes human and non-human mammals. In an example, a subject is a human. In an additional example, a subject is selected that is in need of inhibiting a P. falciparum infection. For example, the subject is uninfected and at risk of P. falciparum infection.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some aspects, a viral vector comprises a nucleic acid molecule encoding a disclosed antibody or antigen binding fragment that specifically binds to PfCSP and neutralizes P. falciparum. In some aspects, the viral vector can be an adeno-associated virus (AAV) vector.

### II. Description of Several Aspects

### A. Neutralizing Monoclonal Antibodies to PfCSP and Antigen Binding Fragments Thereof

Isolated monoclonal antibodies and antigen binding fragments that specifically bind an epitope on PfCSP are provided. The antibodies and antigen binding fragments can be fully human. The antibodies and antigen binding fragments can neutralize P. falciparum, for example the disclosed antibodies can inhibit P. falciparum sporozoite infection of hepatocytes in vitro and P. falciparum sporozoite invasion of liver in vivo. Also disclosed herein are compositions comprising the antibodies and antigen binding fragments and a pharmaceutically acceptable carrier. Nucleic acids encoding the antibodies or antigen binding fragments, expression vectors (such as DNA and RNA vectors for expression and delivery, as well as adeno-associated virus (AAV) viral vectors) comprising these nucleic acids are also provided. The antibodies, antigen binding fragments, nucleic acid molecules, host cells, and compositions can be used for research, diagnostic and prophylactic purposes. For example, the disclosed antibodies and antigen binding fragments can be used to diagnose a subject with a P. falciparum infection, or can be administered prophylactically to inhibit P. falciparum infection in a subject.

### 1. Exemplary Monoclonal Antibodies and Antigen Binding Fragments

The discussion of monoclonal antibodies below refers to isolated monoclonal antibodies that include heavy and/or light chain variable domains (or antigen binding fragments thereof) comprising a CDR1, CDR2, and/or CDR3 with reference to the kabat numbering scheme (unless the context indicates otherwise). Various CDR numbering schemes (such as the Kabat, Chothia or IMGT numbering schemes) can be used to determine CDR positions. The amino acid sequence and the CDR positions (according to the IMGT numbering scheme) of the heavy and light chains of exemplary monoclonal antibodies that bind to PfCSP and neutralize P. falciparum are shown in Table 1.

TABLE 1
Kabat CDR sequences of PfCSP specific antibodies
m43_HH28K_17_PH104K V_{H} (P3-43)

| | SEQ ID NO: 1 | | CDR |
|---|---|---|---|
| V_{H} | residues | A.A. Sequence | SEQ ID NO |
| HCDR1 | 31-35 | SYAIH | 23 |
| HCDR2 | 50-66 | WIKGGNGNTRYSQKFQD | 24 |
| HCDR3 | 99-110 | LTVITkDDAFDI | 25 |

m43_HH28K_17_PH104K V_{L} (P3-43)

| | SEQ ID NO: 2 | | CDR |
|---|---|---|---|
| V_{L} | residues | A.A. Sequence | SEQ ID NO |
| LCDR1 | 24-40 | KSSQNILYSSNNKNYLA | 26 |
| LCDR2 | 56-62 | WASTRES | 27 |
| LCDR3 | 95-103 | HQYYSSPLT | 28 |

D13 V_{H}

| | SEQ ID NO: 3 | | CDR |
|---|---|---|---|
| V_{H} | residues | A.A. Sequence | SEQ ID NO |
| HCDR1 | 31-35 | RYAIH | 29 |
| HCDR2 | 50-66 | WIKAGNGNTRYSQKFQD | 30 |
| HCDR3 | 99-110 | LTVITPDDAFDI | 31 |

D13 V_{L}

| | SEQ ID NO: 4 | | CDR |
|---|---|---|---|
| V_{L} | residues | A.A. Sequence | SEQ ID NO |
| LCDR1 | 24-40 | KSSQNIFFSSkNKNYLA | 32 |
| LCDR2 | 56-62 | WASTRES | 27 |
| LCDR3 | 95-103 | HQYYSSPLT | 28 |

m42_HH28K_13_TH103R_PH104Q V_{H} (P3-21)

| | SEQ ID NO: 5 | | CDR |
|---|---|---|---|
| V_{H} | residues | A.A. Sequence | SEQ ID NO |
| HCDR1 | 31-35 | SYAIH | 23 |
| HCDR2 | 50-66 | WIKAGNGNTRYSQKFQD | 30 |
| HCDR3 | 99-110 | LTVIRQDDTFDI | 33 |

m42_HH28K_13_TH103R_PH104Q V_{L} (P3-21)

| | SEQ ID NO: 6 | | CDR |
|---|---|---|---|
| V_{L} | residues | A.A. Sequence | SEQ ID NO |
| LCDR1 | 24-40 | KSSQNILYSSNNKNYLA | 26 |
| LCDR2 | 56-62 | WASTRES | 27 |
| LCDR3 | 95-103 | HQYYSSPLT | 28 |

m43_HH28K_17_AH107R V_{H} (P3-42)

| | SEQ ID NO: 7 | | CDR |
|---|---|---|---|
| V_{H} | residues | A.A. Sequence | SEQ ID NO |
| HCDR1 | 31-35 | SYAIH | 23 |
| HCDR2 | 50-66 | WIKGGNGNTRYSQKFQD | 24 |
| HCDR3 | 99-110 | LTVITPDDRFDI | 34 |

m43_HH28K_17_AH107R V_{L} (P3-42)

| | SEQ ID NO: 8 | | CDR |
|---|---|---|---|
| V_{L} | residues | A.A. Sequence | SEQ ID NO |
| LCDR1 | 24-40 | KSSQNILYSSNNKNYLA | 26 |
| LCDR2 | 56-62 | WASTRES | 27 |
| LCDR3 | 95-103 | HQYYSSPLT | 28 |

iGL-CIS43-KLH-D42.39 (P4-39) V_{H}

| | SEQ ID NO: 9 | | CDR |
|---|---|---|---|
| V_{H} | residues | A.A. Sequence | SEQ ID NO |
| HCDR1 | 31-35 | NYALH | 35 |
| HCDR2 | 50-66 | WIKTGNGDTRYSQKFQD | 36 |
| HCDR3 | 99-110 | LTVITPDDAFDI | 31 |

iGL-CIS43-KLH-D42.39 (P4-39) V_{L}

| | SEQ ID NO: 10 | | CDR |
|---|---|---|---|
| V_{L} | residues | A.A. Sequence | SEQ ID NO |
| LCDR1 | 24-40 | KSSQSVLYRTNNKNYLA | 37 |
| LCDR2 | 56-62 | WASTRES | 27 |
| LCDR3 | 95-103 | HQYYSSPLT | 28 |

D3 V_{H}

| | SEQ ID NO: 11 | | CDR |
|---|---|---|---|
| V_{H} | residues | A.A. Sequence | SEQ ID NO |
| HCDR1 | 31-35 | rYAIH | 29 |
| HCDR2 | 50-66 | WIKGGNGNTRYSQKFQD | 24 |
| HCDR3 | 99-110 | LTVITPDDAFDI | 31 |

D3 V_{L}

| | SEQ ID NO: 12 | | CDR |
|---|---|---|---|
| V_{L} | residues | A.A. Sequence | SEQ ID NO |
| LCDR1 | 24-40 | KSSQNIffSSNNKNYLA | 38 |
| LCDR2 | 56-62 | WASTRES | 27 |
| LCDR3 | 95-103 | HQYYSSPLT | 28 |

m43_HH28K_17_TH100M V_{H} (P3-45)

| | SEQ ID NO: 13 | | CDR |
|---|---|---|---|
| V_{H} | residues | A.A. Sequence | SEQ ID NO |
| HCDR1 | 31-35 | SYAIH | 23 |
| HCDR2 | 50-66 | WIKGGNGNTRYSQKFQD | 24 |
| HCDR3 | 100-111 | LMVITPDDAFDI | 39 |

m43_HH28K_17_TH100M V_{L} (P3-45)

| | SEQ ID NO: 14 | | CDR |
|---|---|---|---|
| V_{L} | residues | A.A. Sequence | SEQ ID NO |
| LCDR1 | 24-40 | KSSQNILYSSNNKNYLA | 26 |
| LCDR2 | 56-62 | WASTRES | 27 |
| LCDR3 | 95-103 | HQYYSSPLT | 28 |

m43.160 V_{H}

| V_{H} | SEQ ID NO: 15 | | CDR |
|---|---|---|---|
| | residues | A.A. Sequence | SEQ ID NO |
| HCDR1 | 31-35 | SYAIH | 23 |
| HCDR2 | 50-66 | WIKAGNGNTRYSQKFQG | 40 |
| HCDR3 | 99-110 | LTVITPDDAFDI | 31 |

m43.160 V_{L}

| | SEQ ID NO: 16 | | CDR |
|---|---|---|---|
| V_{L} | residues | A.A. Sequence | SEQ ID NO |
| LCDR1 | 24-40 | KSSQNILFSSNNKNYLA | 41 |
| LCDR2 | 56-62 | WASTRES | 27 |
| LCDR3 | 95-103 | HQYYSSPLT | 28 |

m42.127 V_{H}

| | SEQ ID NO: 17 | | CDR |
|---|---|---|---|
| V_{H} | residues | A.A. Sequence | SEQ ID NO |
| HCDR1 | 31-35 | SYAIH | 23 |
| HCDR2 | 50-66 | WIKAGNGNTRYSQKFQD | 30 |
| HCDR3 | 99-110 | LTVITPDDTFDI | 42 |

m42.127 V_{L}

| | SEQ ID NO: 18 | | CDR |
|---|---|---|---|
| V_{L} | residues | A.A. Sequence | SEQ ID NO |
| LCDR1 | 24-40 | KSSQNILYSSNNKNYLA | 26 |
| LCDR2 | 56-62 | WASTRES | 27 |
| LCDR3 | 95-103 | HQYYSSPLT | 28 |

m43.151 V_{H}

| | SEQ ID NO: 19 | | CDR |
|---|---|---|---|
| V_{H} | residues | A.A. Sequence | SEQ ID NO |
| HCDR1 | 31-35 | SYAIH | 23 |
| HCDR2 | 50-66 | WIKGGNGNTRYSQKFQD | 24 |
| HCDR3 | 99-110 | LTVITPDDAFDI | 31 |

m43.151 V_{L}

| | SEQ ID NO: 20 | | CDR |
|---|---|---|---|
| V_{L} | residues | A.A. Sequence | SEQ ID NO |
| LCDR1 | 24-40 | KSSQNILYSSNNKNYLA | 26 |
| LCDR2 | 56-62 | WASTRES | 27 |
| LCDR3 | 95-103 | HQYYSSPLT | 28 |

Core8_H-K58R V_{H}

| | SEQ ID NO: 21 | | CDR |
|---|---|---|---|
| V_{H} | residues | A.A. Sequence | SEQ ID NO |
| HCDR1 | 31-35 | SYAIH | 23 |
| HCDR2 | 50-66 | WIKAGNGNTKYSQKFQG | 43 |
| HCDR3 | 100-111 | LTVITPDDAFDI | 31 |

Core8_H-K58R V_{L}

| | SEQ ID NO: 22 | | CDR |
|---|---|---|---|
| V_{L} | residues | A.A. Sequence | SEQ ID NO |
| LCDR1 | 24-40 | KSSQNILYSSNNKNYLA | 26 |
| LCDR2 | 56-62 | WASTRES | 27 |
| LCDR3 | 95-103 | HQYYSSPLT | 28 |

a. m43_HH28K_17_PH104K (P3-43)

In some aspects, the antibody or antigen binding fragment is based on or derived from the m43_HH28K_17_PH104K antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the m43_HH28K_17_PH104K antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 0, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 2, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 25, respectively, and a V_{L} comprising a LCDR1, a LCDR2, and a LCDR3 as set forth as SEQ ID NOs: 26, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 25, respectively, a V_{L} comprising a LCDR1, a LCDR2, and a LCDR3 as set forth as SEQ ID NOs: 26, 27, and 28, respectively, wherein the V_{H} comprises an amino acid sequence at least 90% identical to SEQ ID NO: 1 (such as 95%, 96%, 97%, 98% or 99% identical to SEQ ID NO: 1), the V_{L} comprises an amino acid sequence at least 90% identical to SEQ ID NO: 2 (such as 95%, 96%, 97%, 98% or 99% identical to SEQ ID NO: 2 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 25, respectively, a V_{L} comprising a LCDR1, a LCDR2, and a LCDR3 as set forth as SEQ ID NOs: 26, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 2, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 1, and specifically binds to PfCSP and neutralizes P. falciparum. In more aspects, the antibody or antigen binding fragment comprises a V_{L} comprising the amino acid sequence set forth as SEQ ID NO: 2, and specifically binds to PfCSP and neutralizes P. falciparum. In some aspects, the antibody or antigen binding fragment comprises a V_{H} and a V_{L} comprising the amino acid sequences set forth as SEQ ID NOs: 1 and 2, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}. In one such aspect, the V_{H} and the V_{L} of the antibody or antigen binding fragment comprises amino acid sequences set forth as SEQ ID NOs: 221 and 222, respectively.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

b. D13

In some aspects, the antibody or antigen binding fragment is based on or derived from the D13 antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the D13 antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO:3, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 4, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 3 and 4, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 29, 30, and 31, respectively, and a V_{L} comprising a LCDR1, a LCDR2, and a LCDR3 as set forth as SEQ ID NOs: 32, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 29, 30, and 31, respectively, a V_{L} comprising a LCDR1, a LCDR2, and a LCDR3 as set forth as SEQ ID NOs: 32, 27, and 28, respectively, wherein the V_{H} comprises an amino acid sequence at least 90% identical to SEQ ID NO: 3 (such as 95%, 96%, 97%, 98% or 99% identical to SEQ ID NO: 3), the V_{L} comprises an amino acid sequence at least 90% identical to SEQ ID NO: 4 (such as 95%, 96%, 97%, 98% or 99% identical to SEQ ID NO: 4 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 29, 30, and 31, respectively, a V_{L} comprising a LCDR1, a LCDR2, and a LCDR3 as set forth as SEQ ID NOs: 32, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 3, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 4, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 3, and specifically binds to PfCSP and neutralizes P. falciparum. In more aspects, the antibody or antigen binding fragment comprises a V_{L} comprising the amino acid sequence set forth as SEQ ID NO: 4, and specifically binds to PfCSP and neutralizes P. falciparum. In some aspects, the antibody or antigen binding fragment comprises a V_{H} and a V_{L} comprising the amino acid sequences set forth as SEQ ID NOs: 3 and 4, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}. In one such aspect, the V_{H} and the V_{L} of the antibody or antigen binding fragment comprises amino acid sequences set forth as SEQ ID NOs: 219 and 220, respectively.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

c. m42_HH28K_13_TH103R_PH104Q (P3-21)

In some aspects, the antibody or antigen binding fragment is based on or derived from the m42_HH28K_13_TH103R_PH104Q antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the m42_HH28K_13_TH103R_PH104Q antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 5, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 6, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 5 and 6, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 30, and 33, respectively, and a V_{L} comprising a LCDR1, a LCDR2, and a LCDR3 as set forth as SEQ ID NOs: 26, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 30, and 33, respectively, a V_{L} comprising a LCDR1, a LCDR2, and a LCDR3 as set forth as SEQ ID NOs: 26, 27, and 28, respectively, wherein the V_{H} comprises an amino acid sequence at least 90% identical to SEQ ID NO: 5 (such as 95%, 96%, 97%, 98% or 99% identical to SEQ ID NO: 5), the V_{L} comprises an amino acid sequence at least 90% identical to SEQ ID NO: 6 (such as 95%, 96%, 97%, 98% or 99% identical to SEQ ID NO: 6 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 30, and 33, respectively, a V_{L} comprising a LCDR1, a LCDR2, and a LCDR3 as set forth as SEQ ID NOs: 26, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 6, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 5, and specifically binds to PfCSP and neutralizes P. falciparum. In more aspects, the antibody or antigen binding fragment comprises a V_{L} comprising the amino acid sequence set forth as SEQ ID NO: 6, and specifically binds to PfCSP and neutralizes P. falciparum. In some aspects, the antibody or antigen binding fragment comprises a V_{H} and a V_{L} comprising the amino acid sequences set forth as SEQ ID NOs: 5 and 6, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

d. m43_HH28K_17_AH107R (P3-42)

In some aspects, the antibody or antigen binding fragment is based on or derived from the m43_HH28K_17_AH107R antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the m43_HH28K_17_AH107R antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 7, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 8, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 7 and 8, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 34, respectively, and a V_{L} comprising a LCDR1, a LCDR2, and a LCDR3 as set forth as SEQ ID NOs: 26, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 34, respectively, a V_{L} comprising a LCDR1, a LCDR2, and a LCDR3 as set forth as SEQ ID NOs: 26, 27, and 28, respectively, wherein the V_{H} comprises an amino acid sequence at least 90% identical to SEQ ID NO: 7 (such as 95%, 96%, 97%, 98% or 99% identical to SEQ ID NO: 7), the V_{L} comprises an amino acid sequence at least 90% identical to SEQ ID NO: 8 (such as 95%, 96%, 97%, 98% or 99% identical to SEQ ID NO: 8 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 34, respectively, a V_{L} comprising a LCDR1, a LCDR2, and a LCDR3 as set forth as SEQ ID NOs: 26, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 7, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 8, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 7, and specifically binds to PfCSP and neutralizes P. falciparum. In more aspects, the antibody or antigen binding fragment comprises a V_{L} comprising the amino acid sequence set forth as SEQ ID NO: 8, and specifically binds to PfCSP and neutralizes P. falciparum. In some aspects, the antibody or antigen binding fragment comprises a V_{H} and a V_{L} comprising the amino acid sequences set forth as SEQ ID NOs: 7 and 8, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

e. iGL-CIS43-KLH-D42.39 (P4-39)

In some aspects, the antibody or antigen binding fragment is based on or derived from the iGL-CIS43-KLH-D42.39 (P4-39) antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the iGL-CIS43-KLH-D42.39 (P4-39) antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 9, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 10, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 9 and 10, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 35, 36, and 31, respectively, and a V_{L} comprising a LCDR1, a LCDR2, and a LCDR3 as set forth as SEQ ID NOs: 37, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 35, 36, and 31, respectively, a V_{L} comprising a LCDR1, a LCDR2, and a LCDR3 as set forth as SEQ ID NOs: 37, 27, and 28, respectively, wherein the V_{H} comprises an amino acid sequence at least 90% identical to SEQ ID NO: 9 (such as 95%, 96%, 97%, 98% or 99% identical to SEQ ID NO: 9), the V_{L} comprises an amino acid sequence at least 90% identical to SEQ ID NO: 10 (such as 95%, 96%, 97%, 98% or 99% identical to SEQ ID NO: 10 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 35, 36, and 31, respectively, a V_{L} comprising a LCDR1, a LCDR2, and a LCDR3 as set forth as SEQ ID NOs: 37, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 9, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 9, and specifically binds to PfCSP and neutralizes P. falciparum. In more aspects, the antibody or antigen binding fragment comprises a V_{L} comprising the amino acid sequence set forth as SEQ ID NO: 10, and specifically binds to PfCSP and neutralizes P. falciparum. In some aspects, the antibody or antigen binding fragment comprises a V_{H} and a V_{L} comprising the amino acid sequences set forth as SEQ ID NOs: 9 and 10, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

### f. D3

In some aspects, the antibody or antigen binding fragment is based on or derived from the D3 antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the D3 antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 11, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 12, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 11 and 12 respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 29, 24, and 31, respectively, and a V_{L} comprising a LCDR1, a LCDR2, and a LCDR3 as set forth as SEQ ID NOs: 38, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 29, 24, and 31, respectively, a V_{L} comprising a LCDR1, a LCDR2, and a LCDR3 as set forth as SEQ ID NOs: 38, 27, and 28, respectively, wherein the V_{H} comprises an amino acid sequence at least 90% identical to SEQ ID NO: 11 (such as 95%, 96%, 97%, 98% or 99% identical to SEQ ID NO: 11), the V_{L} comprises an amino acid sequence at least 90% identical to SEQ ID NO: 12 (such as 95%, 96%, 97%, 98% or 99% identical to SEQ ID NO: 12 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 29, 24, and 31, respectively, a V_{L} comprising a LCDR1, a LCDR2, and a LCDR3 as set forth as SEQ ID NOs: 38, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 11, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 12, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 11, and specifically binds to PfCSP and neutralizes P. falciparum. In more aspects, the antibody or antigen binding fragment comprises a V_{L} comprising the amino acid sequence set forth as SEQ ID NO: 12, and specifically binds to PfCSP and neutralizes P. falciparum. In some aspects, the antibody or antigen binding fragment comprises a V_{H} and a V_{L} comprising the amino acid sequences set forth as SEQ ID NOs: 11 and 12, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}. In one such aspect, the V_{H} and the V_{L} of the antibody or antigen binding fragment comprises amino acid sequences set forth as SEQ ID NOs: 217 and 218, respectively.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

g. m43_HH28K_17_TH100M (P3-45)

In some aspects, the antibody or antigen binding fragment is based on or derived from the m43_HH28K_17_TH100M antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the m43_HH28K_17_TH100M antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 13, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 14, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 13 and 14, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 39, respectively, and a V_{L} comprising a LCDR1, a LCDR2, and a LCDR3 as set forth as SEQ ID NOs: 26, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 39, respectively, a V_{L} comprising a LCDR1, a LCDR2, and a LCDR3 as set forth as SEQ ID NOs: 26, 27, and 28, respectively, wherein the V_{H} comprises an amino acid sequence at least 90% identical to SEQ ID NO: 13 (such as 95%, 96%, 97%, 98% or 99% identical to SEQ ID NO: 13), the V_{L} comprises an amino acid sequence at least 90% identical to SEQ ID NO: 14 (such as 95%, 96%, 97%, 98% or 99% identical to SEQ ID NO: 14 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 39, respectively, a V_{L} comprising a LCDR1, a LCDR2, and a LCDR3 as set forth as SEQ ID NOs: 26, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 13, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 14, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 13, and specifically binds to PfCSP and neutralizes P. falciparum. In more aspects, the antibody or antigen binding fragment comprises a V_{L} comprising the amino acid sequence set forth as SEQ ID NO: 14, and specifically binds to PfCSP and neutralizes P. falciparum. In some aspects, the antibody or antigen binding fragment comprises a V_{H} and a V_{L} comprising the amino acid sequences set forth as SEQ ID NOs: 13 and 14, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

h. m43.160

In some aspects, the antibody or antigen binding fragment is based on or derived from the m43.160 antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the m43.160 antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 15, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 16, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 15 and 16, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 40, and 31, respectively, and a V_{L} comprising a LCDR1, a LCDR2, and a LCDR3 as set forth as SEQ ID NOs: 41, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 40, and 31, respectively, a V_{L} comprising a LCDR1, a LCDR2, and a LCDR3 as set forth as SEQ ID NOs: 41, 27, and 28, respectively, wherein the V_{H} comprises an amino acid sequence at least 90% identical to SEQ ID NO: 15 (such as 95%, 96%, 97%, 98% or 99% identical to SEQ ID NO: 15), the V_{L} comprises an amino acid sequence at least 90% identical to SEQ ID NO: 16 (such as 95%, 96%, 97%, 98% or 99% identical to SEQ ID NO: 16 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 40, and 31, respectively, a V_{L} comprising a LCDR1, a LCDR2, and a LCDR3 as set forth as SEQ ID NOs: 41, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 15, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 16, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 15, and specifically binds to PfCSP and neutralizes P. falciparum. In more aspects, the antibody or antigen binding fragment comprises a V_{L} comprising the amino acid sequence set forth as SEQ ID NO: 16, and specifically binds to PfCSP and neutralizes P. falciparum. In some aspects, the antibody or antigen binding fragment comprises a V_{H} and a V_{L} comprising the amino acid sequences set forth as SEQ ID NOs: 15 and 16, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

i. m42.127

In some aspects, the antibody or antigen binding fragment is based on or derived from the m42.127 antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the m42.127 antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 17, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 18, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 17 and 18, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 30, and 42, respectively, and a V_{L} comprising a LCDR1, a LCDR2, and a LCDR3 as set forth as SEQ ID NOs: 26, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 30, and 42, respectively, a V_{L} comprising a LCDR1, a LCDR2, and a LCDR3 as set forth as SEQ ID NOs: 26, 27, and 28, respectively, wherein the V_{H} comprises an amino acid sequence at least 90% identical to SEQ ID NO: 17 (such as 95%, 96%, 97%, 98% or 99% identical to SEQ ID NO: 17), the V_{L} comprises an amino acid sequence at least 90% identical to SEQ ID NO: 18 (such as 95%, 96%, 97%, 98% or 99% identical to SEQ ID NO: 18 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 30, and 42, respectively, a V_{L} comprising a LCDR1, a LCDR2, and a LCDR3 as set forth as SEQ ID NOs: 26, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 18, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 17, and specifically binds to PfCSP and neutralizes P. falciparum. In more aspects, the antibody or antigen binding fragment comprises a V_{L} comprising the amino acid sequence set forth as SEQ ID NO: 18, and specifically binds to PfCSP and neutralizes P. falciparum. In some aspects, the antibody or antigen binding fragment comprises a V_{H} and a V_{L} comprising the amino acid sequences set forth as SEQ ID NOs: 17 and 18, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

j. m43.151

In some aspects, the antibody or antigen binding fragment is based on or derived from the m43.151 antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the m43.151 antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 19, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 19 and 20, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 31, respectively, and a V_{L} comprising a LCDR1, a LCDR2, and a LCDR3 as set forth as SEQ ID NOs: 26, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 31, respectively, a V_{L} comprising a LCDR1, a LCDR2, and a LCDR3 as set forth as SEQ ID NOs: 26, 27, and 28, respectively, wherein the V_{H} comprises an amino acid sequence at least 90% identical to SEQ ID NO: 19 (such as 95%, 96%, 97%, 98% or 99% identical to SEQ ID NO: 19), the V_{L} comprises an amino acid sequence at least 90% identical to SEQ ID NO: 20 (such as 95%, 96%, 97%, 98% or 99% identical to SEQ ID NO: 20 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 24, and 31, respectively, a V_{L} comprising a LCDR1, a LCDR2, and a LCDR3 as set forth as SEQ ID NOs: 26, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 19, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 19, and specifically binds to PfCSP and neutralizes P. falciparum. In more aspects, the antibody or antigen binding fragment comprises a V_{L} comprising the amino acid sequence set forth as SEQ ID NO: 20, and specifically binds to PfCSP and neutralizes P. falciparum. In some aspects, the antibody or antigen binding fragment comprises a V_{H} and a V_{L} comprising the amino acid sequences set forth as SEQ ID NOs: 19 and 20, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

k. Core8_H-K58R

In some aspects, the antibody or antigen binding fragment is based on or derived from the Core8_H-K58R antibody, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of the Core8_H-K58R antibody, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 21, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 22, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 21 and 22, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 43, and 31, respectively, and a V_{L} comprising a LCDR1, a LCDR2, and a LCDR3 as set forth as SEQ ID NOs: 26, 27, and 28, respectively, and specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 43, and 31, respectively, a V_{L} comprising a LCDR1, a LCDR2, and a LCDR3 as set forth as SEQ ID NOs: 26, 27, and 28, respectively, wherein the V_{H} comprises an amino acid sequence at least 90% identical to SEQ ID NO: 21 (such as 95%, 96%, 97%, 98% or 99% identical to SEQ ID NO: 21), the V_{L} comprises an amino acid sequence at least 90% identical to SEQ ID NO: 22 (such as 95%, 96%, 97%, 98% or 99% identical to SEQ ID NO: 22 and the antibody or antigen binding fragment specifically binds to PfCSP and neutralizes P. falciparum.

In some aspects, the antibody or antigen binding fragment comprises a V_{H} comprising a HCDR1, a HCDR2, and a HCDR3 as set forth as SEQ ID NOs: 23, 43, and 31, respectively, a V_{L} comprising a LCDR1, a LCDR2, and a LCDR3 as set forth as SEQ ID NOs: 26, 27, and 28, respectively, wherein the framework regions of the V_{H} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 21, and the framework regions of the V_{L} comprise up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 22, and the antibody or antigen binding fragment specifically binds to PfCSP and neutralizes P. falciparum.

In additional aspects, the antibody or antigen binding fragment comprises a V_{H} comprising the amino acid sequence set forth as SEQ ID NO: 21, and specifically binds to PfCSP and neutralizes P. falciparum. In more aspects, the antibody or antigen binding fragment comprises a V_{L} comprising the amino acid sequence set forth as SEQ ID NO: 22, and specifically binds to PfCSP and neutralizes P. falciparum. In some aspects, the antibody or antigen binding fragment comprises a V_{H} and a V_{L} comprising the amino acid sequences set forth as SEQ ID NOs: 21 and 22, respectively, and specifically binds to PfCSP and neutralizes P. falciparum.

In some aspects, the antibody of antigen binding fragment further comprises glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

1. Additional Variant CIS43 Antibodies that Bind to the CIS43 Epitope on PfCSP

In some aspects, the antibody or antigen binding fragment is based on or derived from any one of the antibodies provided in Appendices A-F of U.S. Provisional Application No. 63/275,936, filed Nov. 4, 2021, and specifically binds to PfCSP and neutralizes P. falciparum. For example, the antibody or antigen binding fragment comprises a V_{H} and a V_{L} comprising the HCDR1, the HCDR2, and the HCDR3, and the LCDR1, the LCDR2, and the LCDR3, respectively (for example, according to IMGT, Kabat, or Chothia), of any one of the antibodies provided in Appendices A-F or U.S. Provisional Application No. 63/275,936, filed Nov. 4, 2021, and specifically binds to PfCSP and neutralizes P. falciparum.

In some aspects, the antibody or antigen binding fragment comprises a V_{H} comprising an amino acid sequence at least 90% (such as at least 95%, at least 96%, at least 97%, at least 98%, or at least 99%) identical to the amino acid sequence set forth as the V_{H} of any one of the antibodies provided in Appendices A-F of U.S. Provisional Application No. 63/275,936, filed Nov. 4, 2021, and specifically binds to PfCSP and neutralizes P. falciparum. In more aspects, the antibody or antigen binding fragment comprises a V_{L} comprising an amino acid sequence at least 90% (such as at least 95%, at least 96%, at least 97%, at least 98%, or at least 99%) identical to the amino acid sequence set forth as the VL of any one of the antibodies provided in Appendices A-F of U.S. Provisional Application No. 63/275,936, filed Nov. 4, 2021, and specifically binds to PfCSP and neutralizes P. falciparum. In additional aspects, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as the V_{H} and the V_{L} of any one of the antibodies provided in Appendices A-F of U.S. Provisional Application No. 63/275,936, filed Nov. 4, 2021, and specifically binds to PfCSP and neutralizes P. falciparum.

In some aspects, the antibody or antigen binding fragment comprises a V_{H} comprising a HCDR1, a HCDR2, a HCDR3, and a V_{L} comprising a LCDR1, a LCDR2, and a LCDR3, as set forth as the HCDR1, the HCDR2, the LCDR1, the LCDR2, and the LCDR3 of the V_{H} and V_{L} of any one of the antibodies provided in Appendices A-F of U.S. Provisional Application No. 63/275,936, filed Nov. 4, 2021 (According to Kabat), respectively, and specifically binds to PfCSP and neutralizes P. falciparum.

In some aspects, the antibody or antigen binding fragment comprises a V_{H} comprising a HCDR1, a HCDR2, a HCDR3, and a V_{L} comprising a LCDR1, a LCDR2, and a LCDR3, as set forth as the HCDR1, the HCDR2, the LCDR1, the LCDR2, and the LCDR3 of the V_{H} and V_{L} of any one of the antibodies provided in Appendices A-F of U.S. Provisional Application No. 63/275,936, filed Nov. 4, 2021 (according to Kabat), respectively, wherein the V_{H} comprises an amino acid sequence at least 90% (such as 95%, 96%, 97%, 98% or 99%) identical to the V_{H} of the antibody of the Appendix and the V_{L} comprises an amino acid sequence at least 90% (such as 95%, 96%, 97%, 98% or 99%) identical to the V_{L} of the antibody of the appendix, and the antibody or antigen binding fragment specifically binds to PfCSP and neutralizes P. falciparum.

In additional aspects, the antibody or antigen binding fragment comprises a V_{H} comprising the amino acid sequence of the V_{H} of any one of the antibodies provided in Appendices A-F of U.S. Provisional Application No. 63/275,936, filed Nov. 4, 2021. In additional aspects, the antibody or antigen binding fragment comprises a V_{L} comprising the amino acid sequence of the V_{L} of any one of the antibodies provided in Appendices A-F of U.S. Provisional Application No. 63/275,936, filed Nov. 4, 2021. In additional aspects, the antibody or antigen binding fragment comprises a V_{H} and a V_{L} comprising amino acid sequences of the V_{H} and the V_{L} of any one of the antibodies provided in Appendices A-F provided herein.

In some aspects, the antibody of antigen binding fragment comprises further glutamate or glutamine substitutions at one or more of K13, K19, K23, or R44 in the V_{H} and R18 in the V_{L}. In some aspects, the V_{H} and the V_{L} of the antibody or antigen binding fragment further comprise K19E, K23E, and R44E substitutions in the V_{H}, and a R18E substitution in the V_{L}. In some aspects, the V_{H} and the V_{L} of the antibody or antigen binding fragment further comprise K19Q, K23Q, and R44Q substitutions in the V_{H}, and a R18Q substitution in the V_{L}.

In some aspects, the disclosed antibodies and antigen binding fragments inhibit the invasion of Plasmodium falciparum: sporozoites into human liver cells, and/or reduce pathogen load Plasmodium falciparum in a subject, compared to a control.

### 2. Additional Description of Antibodies and Antigen Binding Fragments

The antibody or antigen binding fragment can be a human antibody or fragment thereof. Chimeric antibodies are also provided. The antibody or antigen binding fragment can include any suitable framework region, such as (but not limited to) a human framework region. Alternatively, a heterologous framework region, such as, but not limited to a mouse or monkey framework region, can be included in the heavy or light chain of the antibodies.

The antibody can be of any isotype. The antibody can be, for example, an IgM or an IgG antibody, such as IgG₁, IgG₂, IgG₃, or IgG₄. The class of an antibody that specifically binds PfCSP can be switched with another. In one aspect, a nucleic acid molecule encoding V_{L} or V_{H} is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved, for example, using a vector or nucleic acid molecule that comprises a C_{L} or C_{H} chain. For example, an antibody that specifically binds PfCSP, that was originally IgG may be class switched to an IgM. Class switching can be used to convert one IgG subclass to another, such as from IgG₁ to IgG₂, IgG₃, or IgG₄.

In some examples, the disclosed antibodies are oligomers of antibodies, such as dimers, trimers, tetramers, pentamers, hexamers, septamers, octomers and so on.

The antibody or antigen binding fragment can be derivatized or linked to another molecule (such as another peptide or protein). In general, the antibody or antigen binding fragment is derivatized such that the binding to P. falciparum is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

a. Binding Affinity

In several aspects, the antibody or antigen binding fragment specifically binds PfCSP with an affinity (e.g., measured by K_{D}) of no more than 1.0×10⁻⁸ M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹⁰ M, or no more than 1.0×10⁻¹¹ M. K_{D} can be measured, for example, by a radiolabeled antigen binding assay (RIA) performed with the Fab version of an antibody of interest and its antigen. In one assay, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293(4):865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc™ Catalog #269620), 100 μM or 26 pM [¹²⁵I]-antigen are mixed with serial dilutions of a Fab of interest (e.g., consistent with assessment of the anti-VEGF antibody, Fab-12, in Presta et al., Cancer Res. 57(20):4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MicroScint™_20; PerkinEmler) is added, and the plates are counted on a TOPCOUNT™ gamma counter (PerkinEmler) for ten minutes. Concentrations of each Fab that give less than or equal to 20% of maximal binding are chosen for use in competitive binding assays.

In another assay, K_{D} can be measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

b. Multispecific Antibodies

In some aspects, a multi-specific antibody, such as a bi-specific antibody, is provided that comprises an antibody or antigen binding fragment as provided herein, such as any one of the P3-43, D13, P3-21, P3-42, P4-39, D3, P3-45, m43.160, m42.127, m43.151, or Core8_H-K58R antibodies, or an antigen binding fragment thereof. Any suitable method can be used to design and produce the multi-specific antibody, such as crosslinking two or more antibodies, antigen binding fragments (such as scFvs) of the same type or of different types. Exemplary methods of making multispecific antibodies include those described in PCT Pub. No. WO2013/163427, which is incorporated by reference herein in its entirety. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate).

The multi-specific antibody may have any suitable format that allows for antigen binding by the antibody or antigen binding fragment as provided herein, such as any one of the P3-43, D13, P3-21, P3-42, P4-39, D3, P3-45, m43.160, m42.127, m43.151, or Core8_H-K58R antibodies, or an antigen binding fragment thereof. Bispecific single chain antibodies can be encoded by a single nucleic acid molecule.

Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158(8):3965-3970, 1997; Mack et al., Proc. Natl. Acad. Sci. U.S.A., 92(15):7021-7025, 1995; Kufer et al., Cancer Immunol. Immunother., 45(3-4):193-197, 1997; Löffler et al., Blood, 95(6):2098-2103, 2000; and Brühl et al., J. Immunol., 166(4):2420-2426, 2001. Production of bispecific Fab-scFv (“bibody”) molecules are described, for example, in Schoonjans et al. (J. Immunol., 165(12):7050-7057, 2000) and Willems et al. (J. Chromatogr. B Analyt. Technol. Biomed Life Sci. 786(1-2):161-176, 2003). For bibodies, a scFv molecule can be fused to one of the VL-CL (L) or VH-CH1 chains, e.g., to produce a bibody one scFv is fused to the C-term of a Fab chain.

c. Fragments

Antigen binding fragments are encompassed by the present disclosure, such as Fab, F(ab′)₂, and Fv which include a heavy chain and V_{L} and specifically bind PfCSP. These antibody fragments retain the ability to selectively bind with the antigen and are “antigen-binding” fragments. Non-limiting examples of such fragments include:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, a genetically engineered fragment containing the V_{L} and V_{L} expressed as two chains; and

(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.

(6) A dimer of a single chain antibody (scFV₂), defined as a dimer of a scFV. This has also been termed a “miniantibody.”

Any suitable method of producing the above-discussed antinge binding fragments may be used. Non-limiting examples are provided in Harlow and Lane, Antibodies: A Laboratory Manual, 2^{nd}, Cold Spring Harbor Laboratory, New York, 2013.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

d. Variants

In some aspects, amino acid sequence variants of the antibodies provided herein (such as any one of the P3-43, D13, P3-21, P3-42, P4-39, D3, P3-45, m43.160, m42.127, m43.151, or Core8_H-K58R antibodies) are provided. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some aspects, antibody variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions to increase yield.

In some aspects, the antibody or antigen binding fragment can include up to 10 (such as up to 1, up to 2, up to 3, up to 4, up to 5, up to 6, up to 7, up to 8, or up to 9) amino acid substitutions (such as conservative amino acid substitutions) in the framework regions of the heavy chain of the antibody, or the light chain of the antibody, or the heavy and light chains of the antibody, compared to known framework regions, or compared to the framework regions of an antibody as provided herein (such as any one of the P3-43, D13, P3-21, P3-42, P4-39, D3, P3-45, m43.160, m42.127, m43.151, or Core8_H-K58R antibodies), and maintain the specific binding activity for PfCSP.

In some aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some aspects of the variant V_{H} and V_{L} sequences provided above, each CDR either is unaltered, or contains no more than one, two or three amino acid substitutions.

To increase binding affinity of the antibody, the V_{L} and V_{H} segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for PfCSP.

In some aspects, an antibody (such as any one of the P3-43, D13, P3-21, P3-42, P4-39, D3, P3-45, m43.160, m42.127, m43.151, or Core8_H-K58R antibodies) or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody (such as any one of the P3-43, D13, P3-21, P3-42, P4-39, D3, P3-45, m43.160, m42.127, m43.151, or Core8_H-K58R antibodies) comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody may be made in order to create antibody variants with certain improved properties.

In one aspect, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336(5):1239-1249, 2004; Yamane-Ohnuki et al., Biotechnol. Bioeng. 87(5):614-622, 2004. Examples of cell lines capable of producing defucosylated antibodies include Lee 13 CHO cells deficient in protein fucosylation (Ripka et al., Arch. Biochem. Biophys. 249(2):533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87(5): 614-622, 2004; Kanda et al., Biotechnol. Bioeng., 94(4):680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several aspects, the constant region of the antibody (such as any one of P3-43, D13, P3-21, P3-42, P4-39, D3, P3-45, m43.160, m42.127, m43.151, or Core8_H-K58R antibodies) comprises one or more amino acid substitutions to optimize in vivo half-life of the antibody. The serum half-life of IgG Abs is regulated by the neonatal Fc receptor (FcRn). Thus, in several aspects, the antibody comprises an amino acid substitution that increases binding to the FcRn. Non-limiting examples of such substitutions include substitutions at IgG constant regions T250Q and M428L (see, e.g., Hinton et al., J Immunol., 176(1):346-356, 2006); M428L and N434S (the “LS” mutation, see, e.g., Zalevsky, et al., Nature Biotechnol., 28(2):157-159, 2010); N434A (see, e.g., Petkova et al., Int. Immunol., 18(12):1759-1769, 2006); T307A, E380A, and N434A (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies (such as any one of the P3-43, D13, P3-21, P3-42, P4-39, D3, P3-45, m43.160, m42.127, m43.151, or Core8_H-K58R antibodies) and antigen binding fragments can be linked to or comprise an Fc polypeptide including any of the substitutions listed above, for example, the Fc polypeptide can include the M428L and N434S substitutions.

In some aspects, the constant region of the antibody comprises one or more amino acid substitutions to optimize ADCC. ADCC is mediated primarily through a set of closely related Fcγ receptors. In some aspects, the antibody comprises one or more amino acid substitutions that increase binding to FcγRIIIa. Non-limiting examples of such substitutions include substitutions at IgG constant regions S239D and 1332E (see, e.g., Lazar et al., Proc. Natl., Acad. Sci. U.S.A., 103(11):4005-4010, 2006); and S239D, A330L, and 1332E (see, e.g., Lazar et al., Proc. Natl., Acad. Sci. U.S.A., 103(11):4005-4010, 2006).

Combinations of the above substitutions are also included, to generate an IgG constant region with increased binding to FcRn and FcγRIIIa. The combinations increase antibody half-life and ADCC. For example, such combinations include antibodies with the following amino acid substitutions in the Fc region: (1) S239D/I332E and T250Q/M428L; (2) S239D/I332E and M428L/N434S; (3) S239D/I332E and N434A; (4) S239D/I332E and T307A/E380A/N434A; (5) S239D/I332E and M252Y/S254T/T256E; (6) S239D/A330L/I332E and 250Q/M428L; (7) S239D/A330L/I332E and M428L/N434S; (8) S239D/A330L/I332E and N434A; (9) S239D/A330L/I332E and T307A/E380A/N434A; or (10) S239D/A330L/I332E and M252Y/S254T/T256E. In some examples, the antibodies, or an antigen binding fragment thereof is modified such that it is directly cytotoxic to infected cells, or uses natural defenses such as complement, ADCC, or phagocytosis by macrophages.

In some aspects, an antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### B. Conjugates

The antibodies and antigen binding fragments that specifically bind to PfCSP (such as any one of the P3-43, D13, P3-21, P3-42, P4-39, D3, P3-45, m43.160, m42.127, m43.151, or Core8_H-K58R antibodies) can be conjugated to an agent, such as an effector molecule or detectable marker. Both covalent and noncovalent attachment means may be used. Various effector molecules and detectable markers can be used, including (but not limited to) toxins and radioactive agents such as ¹²⁵I, ³²P, ¹⁴C, ³H and ³⁵S and other labels, target moieties and ligands, etc. The choice of a particular effector molecule or detectable marker depends on the particular target molecule or cell, and the desired biological effect.

The procedure for attaching an effector molecule or detectable marker to an antibody or antigen binding fragment varies according to the chemical structure of the effector. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody or antigen binding fragment is derivatized to expose or attach additional reactive functional groups. The derivatization may involve attachment of any suitable linker molecule. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or other polypeptide can be determined.

The antibody or antigen binding fragment can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody or antigen binding fragment can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody or antigen binding fragment may also be conjugated with biotin, and detected through indirect measurement of avidin or streptavidin binding. It should be noted that the avidin itself can be conjugated with an enzyme or a fluorescent label.

The antibody or antigen binding fragment can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitope recognized by a secondary reporter (such as leucine zipper pair sequences, binding sites for secondary antibodies, metal binding domains, epitope tags).

The antibody or antigen binding fragment can also be conjugated with a radiolabeled amino acid, for example, for diagnostic purposes. For instance, the radiolabel may be used to detect PfCSP expressing cells by radiography, emission spectra, or other diagnostic techniques. Examples of labels for polypeptides include, but are not limited to, the following radioisotopes: ³H, ¹⁴C, ³⁵S, ⁹⁰Y ^{99m}Tc, ¹¹¹In, ¹²⁵I, ¹³¹. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of effector molecule or detectable marker moieties per antibody or antigen binding fragment in a conjugate can range, for example, from 1 to 20 moieties per antibody or antigen binding fragment. In some aspects, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acid molecules (for example, cDNA or RNA molecules) encoding the amino acid sequences of antibodies, antigen binding fragments, and conjugates that specifically bind to PfCSP (such as any one of the P3-43, D13, P3-21, P3-42, P4-39, D3, P3-45, m43.160, m42.127, m43.151, or Core8_H-K58R antibodies) are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several aspects, a nucleic acid molecules can encode the V_{H}, the V_{L}, or both the V_{H} and V_{L} (for example in a bicistronic expression vector) of a disclosed antibody or antigen binding fragment. In several aspects, the nucleic acid molecules can be expressed in a host cell (such as a mammalian cell) to produce a disclosed antibody or antigen binding fragment.

The genetic code can be used to construct a variety of functionally equivalent nucleic acid sequences, such as nucleic acids which differ in sequence but which encode the same antibody sequence, or encode a conjugate or fusion protein including the V_{L} and/or V_{H} nucleic acid sequence.

Nucleic acid molecules encoding the antibodies, antigen binding fragments, and conjugates that specifically bind to PfCSP can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The antibodies, antigen binding fragments, and conjugates can be expressed as individual proteins including the V_{H} and/or V_{L} (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly₄-Ser)₃, such that the V_{H} and V_{L} sequences can be expressed as a contiguous single-chain protein, with the V_{L} and V_{H} domains joined by the flexible linker (see, e.g., Bird et al., Science, 242(4877):423-426, 1988; Huston et al., Proc. Natl. Acad. Sci. U.S.A., 85(16):5879-5883, 1988; McCafferty et al., Nature, 348:552-554, 1990; Kontermann and Dilbel (Eds.), Antibody Engineering, Vols. 1-2, 2^{nd} ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to PfCSP and another antigen. The encoded V_{H} and V_{L} optionally can include a furin cleavage site between the V_{H} and V_{L} domains.

One or more DNA sequences encoding the antibodies, antigen binding fragments, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed antibodies and antigen binding fragments. Methods of stable transfer, meaning that the foreign DNA is continuously maintained in the host may be used. Hybridomas expressing the antibodies of interest are also encompassed by this disclosure.

The expression of nucleic acids encoding the antibodies and antigen binding fragments described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, antigen binding fragments, and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The antibodies, antigen binding fragment, and conjugates need not be 100% pure. Once purified, partially or to homogeneity as desired, if to be used prophylatically, the polypeptides should be substantially free of endotoxin.

Methods for expression of antibodies, antigen binding fragments, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### D. Methods and Compositions

1. Inhibiting P. falciparum Infection

Methods are disclosed herein for the inhibition of a P. falciparum infection in a subject. The methods include administering to the subject an effective amount (that is, an amount effective to inhibit P. falciparum infection in the subject) of a disclosed antibody (such as any one of the P3-43, D13, P3-21, P3-42, P4-39, D3, P3-45, m43.160, m42.127, m43.151, or Core8_H-K58R antibodies), antigen binding fragment, conjugate, or a nucleic acid encoding such an antibody, antigen binding fragment, or conjugate, to a subject at risk of a P. falciparum infection. The methods can be used pre-exposure or post-exposure.

P. falciparum infection does not need to be completely eliminated or inhibited for the method to be effective. For example, the method can decrease P. falciparum infection by a desired amount, for example by at least 10%, at least 20%, at least 50%, at least 60%, at least 70%, at least 80%, at least 90%, at least 95%, at least 98%, or even at least 100% (elimination or prevention of detectable P. falciparum infection) as compared to P. falciparum infection in the absence of the treatment. In some aspects, the subject can also be treated with an effective amount of an additional agent, such as anti-malaria agent.

In some aspects, administration of an effective amount of a disclosed antibody, antigen binding fragment, conjugate, or nucleic acid molecule, inhibits the establishment of P. falciparum infection and/or subsequent P. falciparum disease progression in a subject, which can encompass any statistically significant reduction in P. falciparum activity (for example, growth or invasion) or symptoms of P. falciparum infection in the subject.

Antibodies and antigen binding fragments thereof are typically administered by intravenous infusion. Doses of the antibody or antigen binding fragment vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some aspects, the dose of the antibody or antigen binding fragment can be from about 0.5 mg/kg to about 5 mg/kg, such as a dose of about 1 mg/kg, about 2 mg/kg, about 3 mg/kg, about 4 mg/kg or about 5 mg/kg. The antibody or antigen binding fragment is administered according to a dosing schedule determined by a medical practitioner. In some examples, the antibody or antigen binding fragment is administered weekly, every two weeks, every three weeks or every four weeks.

In some aspects, the method of inhibiting P. falciparum infection in a subject further comprises administration of one or more additional agents to the subject. Additional agents of interest include, but are not limited to, anti-malaria agents.

In some aspects, the method of inhibiting P. falciparum infection in a subject comprises administration of a first antibody that specifically binds to PfCSP as disclosed herein (such as any one of the P3-43, D13, P3-21, P3-42, P4-39, D3, P3-45, m43.160, m42.127, m43.151, or Core8_H-K58R antibodies) and a second antibody that that specifically binds to PfCSP (such as L9 disclosed in PCT Pub. No. WO 2020/227228, which is incorporated by reference herein).

In some aspects, a subject is administered DNA or RNA encoding a disclosed antibody to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, or antigen binding fragments thereof, can be placed under the control of a promoter to increase expression. The methods include liposomal delivery of the nucleic acids. Such methods can be applied to the production of an antibody, or antigen binding fragments thereof. In some aspects, a disclosed antibody or antigen binding fragment is expressed in a subject using the pVRC8400 vector (described in Barouch et al., J. Virol., 79(14), 8828-8834, 2005, which is incorporated by reference herein).

In several aspects, a subject (such as a human subject at risk of P. falciparum infection) can be administered an effective amount of an AAV viral vector that comprises one or more nucleic acid molecules encoding a disclosed antibody or antigen binding fragment. The AAV viral vector is designed for expression of the nucleic acid molecules encoding a disclosed antibody or antigen binding fragment, and administration of the effective amount of the AAV viral vector to the subject leads to expression of an effective amount of the antibody or antigen binding fragment in the subject. Non-limiting examples of AAV viral vectors that can be used to express a disclosed antibody or antigen binding fragment in a subject include those provided in Johnson et al., Nat. Med., 15(8):901-906, 2009 and Gardner et al., Nature, 519(7541):87-91, 2015, each of which is incorporated by reference herein in its entirety.

In one aspect, a nucleic acid encoding a disclosed antibody, or antigen binding fragment thereof, is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed PfCSP-specific antibody, antigen binding fragment, conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit P. falciparum infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The PfCSP-specific antibody, antigen binding fragment, conjugate, or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an aspect, the antibody, antigen binding fragment, conjugate, or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site.

### 2. Compositions

Compositions are provided that include one or more of the PfCSP-specific antibody, antigen binding fragment, conjugate, or nucleic acid molecule encoding such molecules, that are disclosed herein in a pharmaceutically acceptable carrier. In some aspects, the composition comprises an antibody as provided herein (such as any one of P3-43, D13, P3-21, P3-42, P4-39, D3, P3-45, m43.160, m42.127, m43.151, or Core8_H-K58R antibodies). In some aspects, the composition comprises an antibody as provided herein (such as any one of the P3-43, D13, P3-21, P3-42, P4-39, D3, P3-45, m43.160, m42.127, m43.151, or Core8_H-K58R antibodies) and one or more additional PfCSP-specific antibody, such as L9. The compositions are useful, for example, for example, for the inhibition or detection of a P. falciparum infection. The compositions can be prepared in unit dosage forms for administration to a subject. The amount and timing of administration are at the discretion of the administering physician to achieve the desired purposes. The PfCSP-specific antibody, antigen binding fragment, conjugate, or nucleic acid molecule encoding such molecules can be formulated for systemic or local administration. In one example, the PfCSP-specific antibody, antigen binding fragment, conjugate, or nucleic acid molecule encoding such molecules, is formulated for parenteral administration, such as intravenous administration.

In some aspects, the antibody, antigen binding fragment, or conjugate thereof, in the composition is at least 70% (such as at least 75%, at least 80%, at least 85%, at least 90%, at least 95%, at least 96%, at least 97%, at least 98% or at least 99%) pure. In some aspects, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the PfCSP-specific antibody, antigen binding fragment, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration comprises about 0.01 to about 30 mg/kg of antibody or antigen binding fragment or conjugate per subject per day (or the corresponding dose of a conjugate including the antibody or antigen binding fragment). Any suitable method may be used for preparing administrable compositions; non-limiting examples are provided in such publications as Remington: The Science and Practice of Pharmacy, 22^{nd} ed., London, UK: Pharmaceutical Press, 2013. In some aspects, the composition can be a liquid formulation including one or more antibodies, antigen binding fragments (such as an antibody or antigen binding fragment that specifically binds to PfCSP), in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Antibodies, or an antigen binding fragment thereof or a conjugate or a nucleic acid encoding such molecules, can be provided in lyophilized form and rehydrated with sterile water before administration, although they are also provided in sterile solutions of known concentration. The antibody solution, or an antigen binding fragment or a nucleic acid encoding such antibodies or antigen binding fragments, can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 m are generally referred to as nanoparticles, nanospheres, and nanocapsules, respectively. Capillaries have a diameter of approximately 5 m so that only nanoparticles are administered intravenously. Microparticles are typically around 100 μm in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the antibody compositions disclosed herein. Any suitable polymer may be used, such as a degradable or nondegradable polymeric matrix designed for use in controlled drug delivery. Alternatively, hydroxyapatite has been used as a microcarrier for controlled release of proteins. In yet another aspect, liposomes are used for controlled release as well as drug targeting of the lipid-capsulated drug.

### 3. Methods of Detection and Diagnosis

Methods are also provided for the detection of the presence of PfCSP in vitro or in vivo. In one example, the presence of PfCSP is detected in a biological sample from a subject, and can be used to identify a subject with P. falciparum infection. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with an antibody or antigen binding fragment that specifically binds to PfCSP, or conjugate thereof (e.g., a conjugate including a detectable marker) under conditions sufficient to form an immune complex, and detecting the immune complex (e.g., by detecting a detectable marker conjugated to the antibody or antigen binding fragment.

In one aspect, the antibody or antigen binding fragment is directly labeled with a detectable marker. In another aspect, the antibody that binds P. falciparum (the primary antibody) is unlabeled and a secondary antibody or other molecule that can bind the primary antibody is utilized for detection. The secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the antibody, antigen binding fragment or secondary antibody are known and described above, and include various enzymes, prosthetic groups, fluorescent materials, luminescent materials, magnetic agents and radioactive materials.

In some aspects, the disclosed antibodies or antigen binding fragments thereof are used to test vaccines. For example to test if a vaccine composition including a PfCSP or fragment thereof assumes a conformation including the epitope of a disclosed antibody. Thus provided herein is a method for testing a vaccine, wherein the method comprises contacting a sample containing the vaccine, such as a PfCSP immunogen, with a disclosed antibody or antigen binding fragment under conditions sufficient for formation of an immune complex, and detecting the immune complex, to detect the vaccine with an PfCSP immunogen including the epitope in the sample. In one example, the detection of the immune complex in the sample indicates that vaccine component, such as a PfCSP immunogen assumes a conformation capable of binding the antibody or antigen binding fragment.

### III. EXAMPLES

The following example is provided to illustrate particular features of certain aspects, but the scope of the claims should not be limited to those features exemplified.

### Example 1

### Elicitation of Highly Protective Anti-Malarial Antibodies in a Humanized Mouse Model Summary

Repeat antigens, such as the Plasmodium falciparum: circumsporozoite protein (PfCSP), use both sequence degeneracy and structural diversity to evade the immune response. A few PfCSP-directed antibodies have been identified that are effective at preventing malaria infection, including antibody CIS43, but how these repeat-targeting antibodies might be improved has been unclear. Here, we engineer a humanized mouse model in which B cells express inferred human germline CIS43 (iGL-CIS43) B cell receptors and use both vaccination and informatics to obtain variant CIS43 antibodies with improved protective capacity. Notably, we obtained an improved antibody, iGL-CIS43.D3, which was significantly more potent than the current best-in-class PfCSP-directed antibody. Moreover, we observed vaccination with a junctional epitope peptide to be more effective than full-length PfCSP at recruiting iGL-CIS43 B cells to germinal centers. This new mouse model can thus be used to understand vaccine immunogens and to develop highly potent anti-malaria antibodies with improved therapeutic potential.

### Introduction

Plasmodium falciparum: (Pf) is the etiological agent of malaria, a vector-borne infectious disease that poses a significant challenge to global public health with an estimated disease burden of 229 million cases and 409,000 deaths in 2019 alone (Global Malaria Programme, 2020). While both medical and non-medical interventions, such as artemisinin-based combination therapies (ACTs) and insecticide-treated nets (ITNs), have proven to be highly effective in controlling malaria parasite infections and its vector, the female Anopheles mosquito, respectively, these measures are offset by the appearance of drug-resistant parasite strains and insecticide-resistant mosquitos. Thus, to limit morbidity and mortality and ultimately eradicate malaria, there is an urgent need for interventions that can prevent malaria.

Malaria infection is initiated following a mosquito bite when sporozoites (SPZ), the infectious form of Plasmodium, are injected into the skin and blood. Shortly thereafter, SPZ travel to the liver to infect hepatocytes and initiate the infection. The Pf circumsporozoite protein (PfCSP) is the most abundant protein covering the SPZ surface and is required for motility and hepatocyte invasion. PfCSP is comprised of an N-terminus, a central repeating tetrapeptide region, and a C-terminus.

Repeat antigens such as PfCSP can assume divergent structures, which confound both recognition and maturation of the humoral immune response. Nevertheless, PfCSP is currently the best target for neutralizing antibodies, and the most advanced malaria vaccine-RTS,S-presents a truncated portion of the PfCSP repeat containing only NANP and C-terminal regions (Partnership, 2012; RTS, 2015). Phase II and III trials of RTS,S/AS01 have found that vaccine efficacy was approximately 36% among 5-17-month-olds after 4 years and wanes over time with decreasing antibody titers (RTS, 2015; White et al., 2014), as protection appears to require extraordinarily high titer of PfCSP-directed antibody, suggesting most elicited PfCSP-directed antibodies to be poorly protective. An alternative immune approach that could potentially enhance the protective efficacy over a defined period is with long-lasting, highly potent monoclonal antibodies (mAbs). mAbs offer an advantage in that their protection may be independent of any host-parasite factors that can limit the effectiveness of vaccines.

The majority of mouse and human neutralizing mAbs demonstrating protection in vivo bind the central repeat region, containing an immunodominant NPNA sequence (Imkeller et al., 2018; Oyen et al., 2017; Zavala et al., 1983). The isolation of a highly potent human mAb that preferentially binds a unique tetrapeptide, NPDP, at the junction of the N-terminus and repeat region, identifying this subdominant “junctional epitope” as a site of vulnerability was recently reported (Kisalu et al., 2018). CIS43 was altered to express an LS mutation in its Fc region to increase its half-life (Kisalu et al., 2020), and is currently undergoing clinical trials.

To investigate fundamental questions in malaria vaccinology and CIS43 ontogeny, a pre-clinical knock-in (KI) mouse model was established. Recently, a one-step CRISPR/Cas9-induced homology-directed repair (HDR) approach to accelerate the generation of knock-in mice was developed (Lin et al., 2018a; Wang et al., 2020b), and applied that technique to insert inferred human germline CIS43 (iGL-CIS43) IgH and IgK chains at their respective native mouse loci (Lin et al., 2018b; Wang et al., 2020b). Using this KI model, we demonstrate herein the utility of an immunofocusing strategy: there were notable improvements in iGL-CIS43 B cell recruitment to germinal centers in animals immunized with the junctional peptide rather than full-length PfCSP. The epitope-focused immunization strategy induced substantial diversification via somatic hypermutation and facilitated the generation of an antibody library comprising over a hundred CIS43 variant antibodies, a subset of which was bioinformatically selected to assess biophysical and structural properties to characterize epitope-specificities and to measure their ability to protect against malaria parasite challenge in vivo. Correlation of antibody properties with in vivo protection enabled the engineering of CIS43-variant antibodies that are more protective against malaria than mature CIS43 and current best-in-class mAbs.

### Results

### Generation of Knock-In Mice Expressing Inferred Germline Sequences of CIS43

To generate a new knock-in (KI) mouse model for studying malaria vaccine strategies, we used inferred germline-antibody sequences of the anti-malarial antibody CIS43 (Kisalu et al., 2018). CIS43 uses heavy chain variable genes V_{H}1-3*03, D_{H}4-23*01 and J_{H}3*02 and the cognate light variable genes Vκ4-1*01 and Jκ4*01; it was originally isolated from a malaria-naive participant exposed to radiation-attenuated live sporozoites (PfSPZ vaccine) in a malaria clinical trial (Seder et al., 2013).

By using CRISPR/Cas9 protocols that were previously established (Lin et al., 2018b; Wang et al., 2020b), the iGL-CIS43 heavy and variable light chain regions were inserted into their respective native loci. Briefly, fertilized mouse oocytes were microinjected with (a) two donor plasmids, each containing pre-rearranged inferred germline CIS43 heavy and light chain sequences driven by the mouse V_{H}J558 or Vκ4-53 promoters, respectively; (b) four single-guided RNAs (sgRNAs)—with two sgRNAs targeting each H or K locus; and (c) AltR-Cas9 (Wang et al., 2020b). After implantation of the injected fertilized zygotes into pseudopregnant C57BL/6J females, the resulting F0 and F1 pups were genotyped to ascertain the presence of iGL-CIS43 heavy and/or light chains: four (14.8%) were double-positive, expressing both inferred germline heavy and light chains; the resulting heterozygous mouse lines are referred to as H^{iGL-CIS43}κ^{iGL-CIS43}.

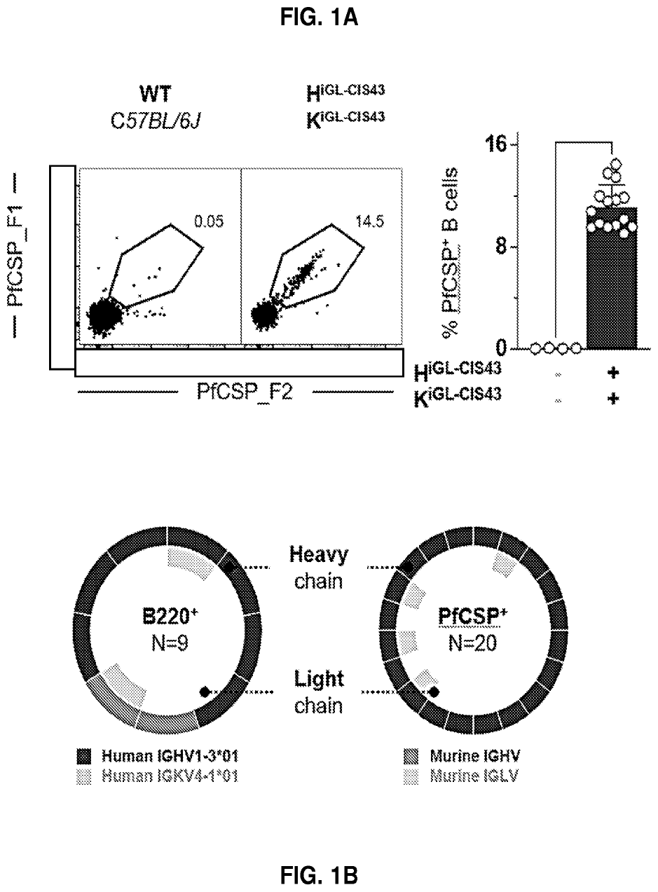

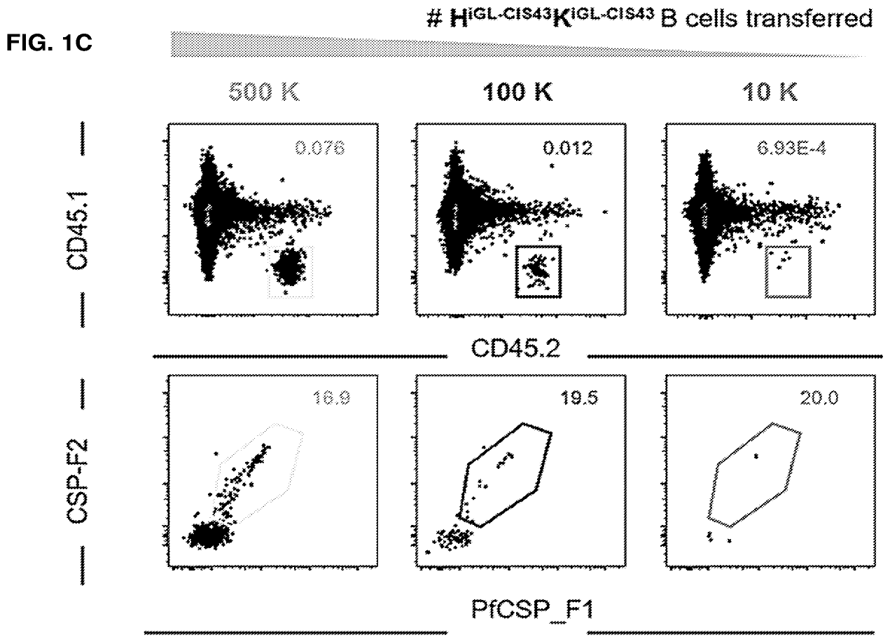

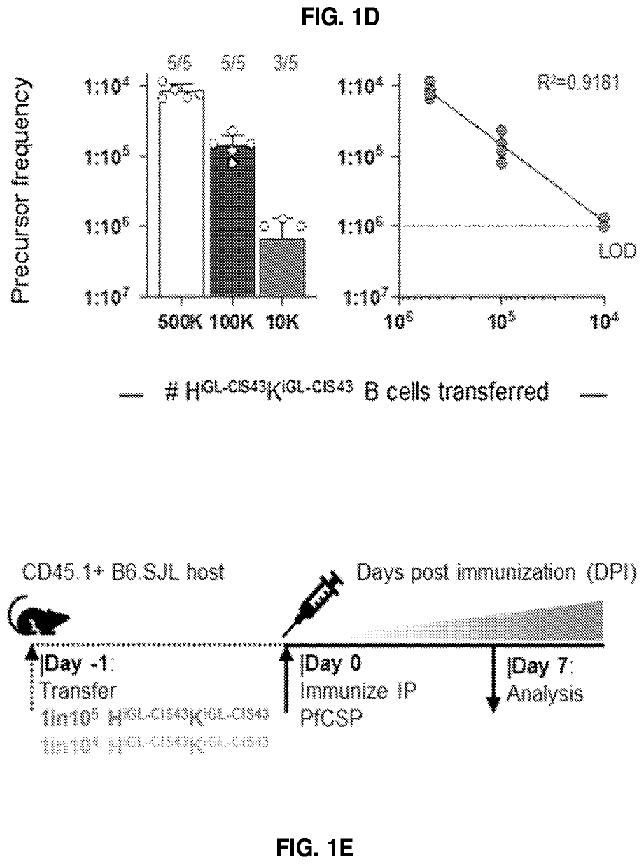

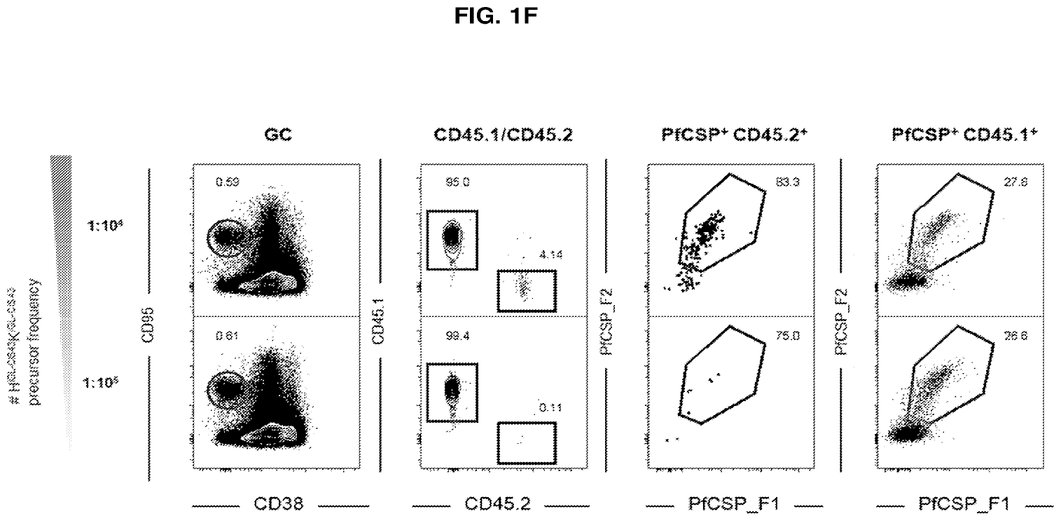

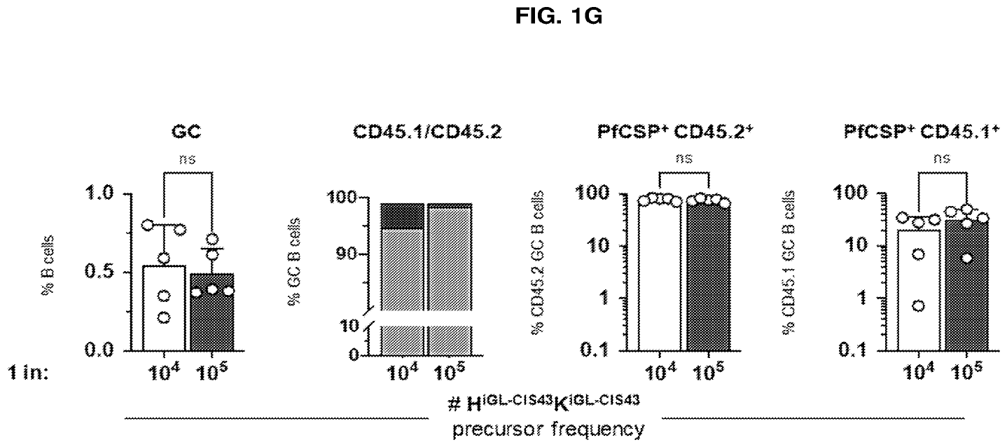

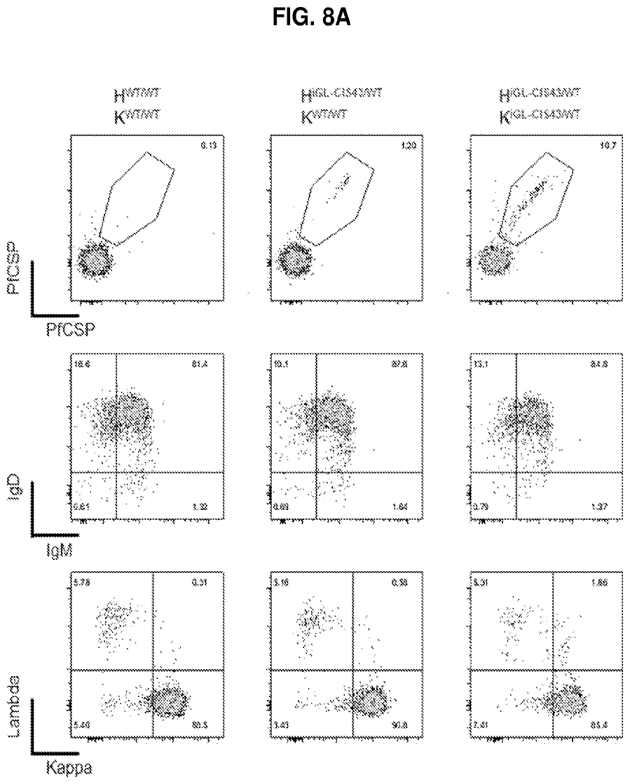

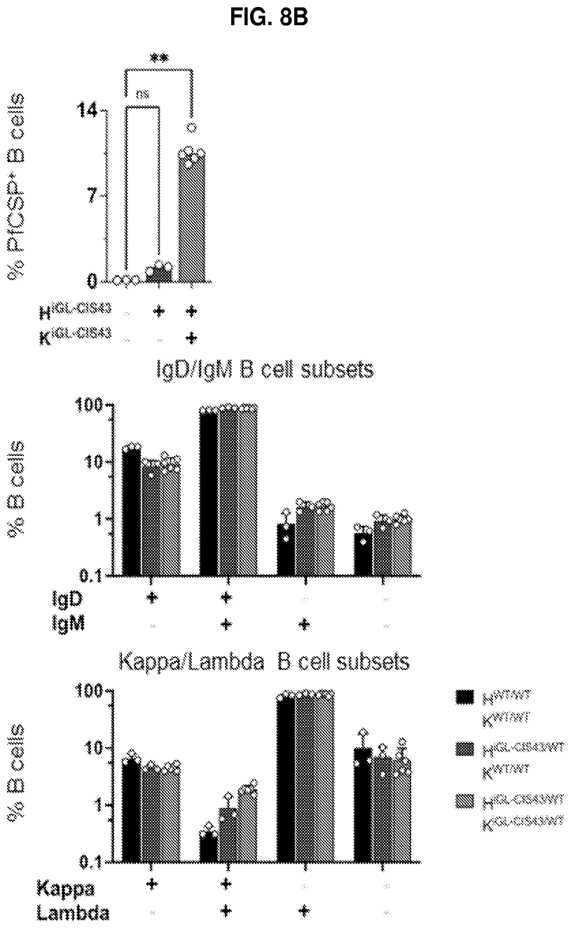

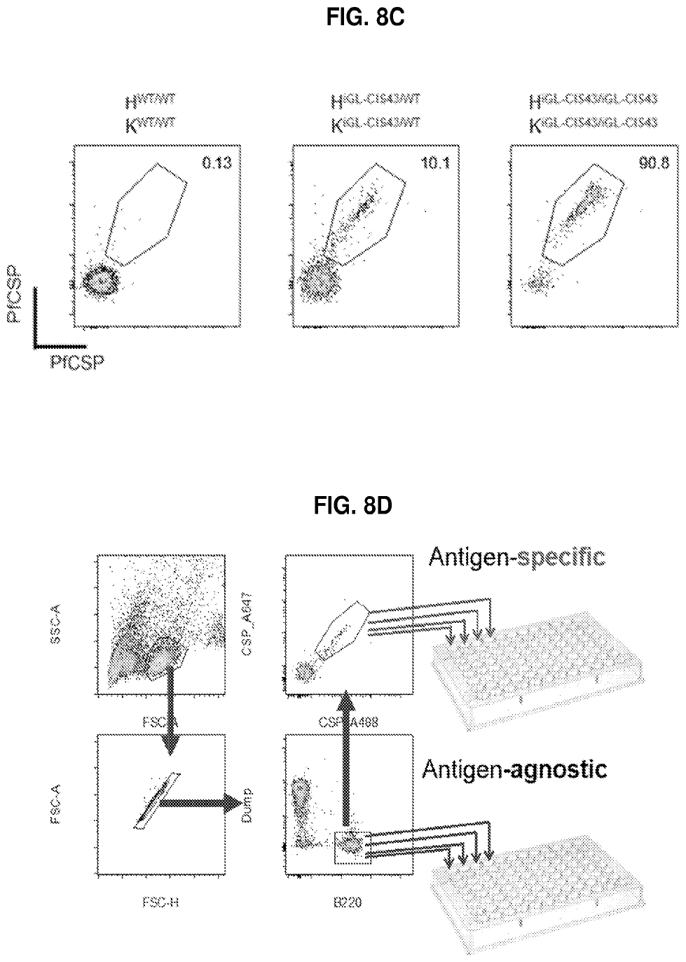

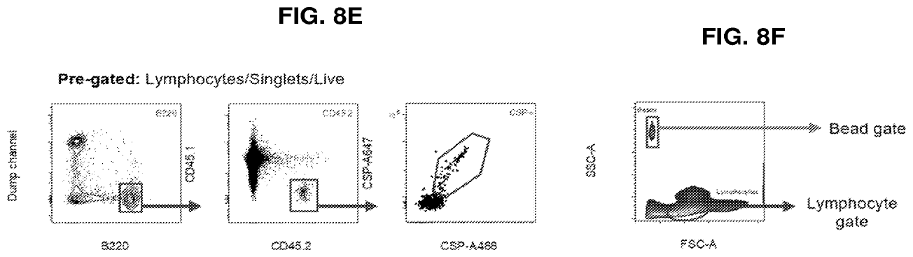

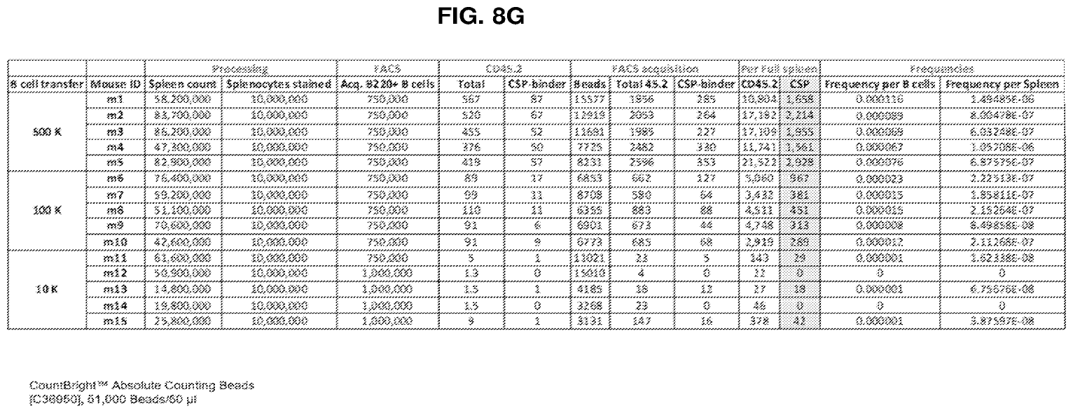

To determine whether iGL-CIS43 heavy and/or light chains were expressed as part of functional BCRs, we performed flow cytometric analysis using a PfCSP-probe. Heterozygous H^{iGL-CIS43}κ^{iGL-CIS43} KI mice showed significantly higher median PfCSP-binding of peripheral B cells (10.80%) than either wild-type C57BL/6J mice (0.07%) (FIG. 1A) or the heavy-chain only H^{iGL-CIS43} KI model (1.20%) (FIGS. 8A and 8B). In homozygous H^{iGL-CIS43}κ^{iGL-CIS43} mice, ˜90% of naïve B cells bind to PfCSP (FIG. 8C). Based on the flow cytometric analysis of several surface markers, peripheral B cell populations of iGL-CIS43 mice appeared comparable to C57BL/6 control mice (FIGS. 8A and 8B). To confirm that BCRs from the H^{iGL-CIS43}κ^{iGL-CIS43} line predominantly consist of both human iGL-CIS43 heavy and light chains, we performed both antigen-agnostic (B220⁺) and antigen-specific (B220⁺PfCSP⁺) single-cell sorting and sequencing (FIGS. 1B, 8D). While ˜67% (6/9) of B220⁺-sorted B cells were positive for the iGL-CIS43 heavy and light chains, B220⁺PfCSP⁺-sorted B cells were predominantly positive for both (80%, 16/20) (FIG. 1B). Having confirmed that the KI produced functional PfCSP-binders at high frequency and did not disrupt B cell differentiation, we used heterozygous iGL-CIS43 mice were used in all subsequent experiments.

### KI B Cells are Outcompeted After PfCSP Immunization

The vaccine-mediated activation of specific B cell clones is dependent on their frequency in the total B cell repertoire (Abbott et al., 2018; Dosenovic et al., 2018). Given the high precursor numbers in the H^{iGL-CIS43}κ^{iGL-CIS43} KI mice (FIGS. 1A and 1B), we lowered the precursor frequency by adoptive transfer of CD45.2⁺ H^{iGL-CIS43}κ^{iGL-CIS43} B cells into CD45.1⁺ C57BL/6 recipient mice at three different concentrations (500,000, 100,000 and 10,000 naive B cells). To establish the absolute number of PfCSP-reactive H^{iGL-CIS43}κ^{iGL-CIS43} precursor B cells in the spleen, recipient mice were sacrificed at day 0 (24 h post transfer) and flow cytometric analysis was performed (FIGS. 1C, 8E-8G). Numbers of PfCSP-reactive CD45.2 B cells are linearly dependent on the total number of transferred B cells and were calculated to be present at approximately 1 in 10⁴, 1 in 10⁵ and 1 in 10⁶ at the time of immunization (FIGS. 1C and 1D).

Mice with rare precursor frequencies (1 in 10⁴ and 1 in 10⁵) were used for the vaccine studies based on prior analysis in other models (Abbott et al., 2018; Havenar-Daughton et al., 2018). Cohorts of recipient mice were immunized intraperitoneally with PfCSP (10 μg/mouse) in Alhydrogel 24 hours after adoptive transfer, then sacrificed at day 7 following immunization and their spleens harvested to measure GC formation (CD38^{low}CD95⁺) (FIG. 1E). At this timepoint, ˜0.6% of the B cells were CD38^{low} CD95⁺, demonstrating the induction of GC formation. Next, we used CD45.2 as a marker to interrogate the percentage of the GC B cells represented by our adoptively transferred H^{iGL-CIS43}κ^{iGL-CIS43} B cells. H^{iGL-CIS43}κ^{iGL-CIS43} B cells accounted for ˜4.3% and 0.6% in animals with precursor frequencies of 1:10⁴ to 1:10⁵ respectively of the total GC responses. These data show that the transferred B cells were specifically recruited to the GC, which confirms that H^{iGL-CIS43}κ^{iGL-CIS43} B cells are competent to respond to antigen-challenge in vivo (FIG. 1F). Notably, these immune responses were low in magnitude and few H^{iGL-CIS43}κ^{iGL-CIS43} B cells entered the GC when present at the low frequency of 1:10⁵, though total GC responses were equivalent at both frequencies (FIGS. 1F and 1G). Further, we observed significant activation of host CD45.1 B cells with high specificity for PfCSP (FIG. 1G, rightmost panel). This outgrowth among GC B cells by host CD45.1 B cells may be attributable to the immunodominant malaria NANP-repeat epitopes expressed in PfCSP, which could cause B cell diversion (Foquet et al., 2014; Kisalu et al., 2018; McNamara et al., 2020; Oyen et al., 2017; Triller et al., 2017; Zavala et al., 1983). These data suggest that alternative immunogens may be preferable in activating the iGL-CIS43 B cells.

### Immunofocusing Enhances Immune Responses by CIS43-Precursor B Cells

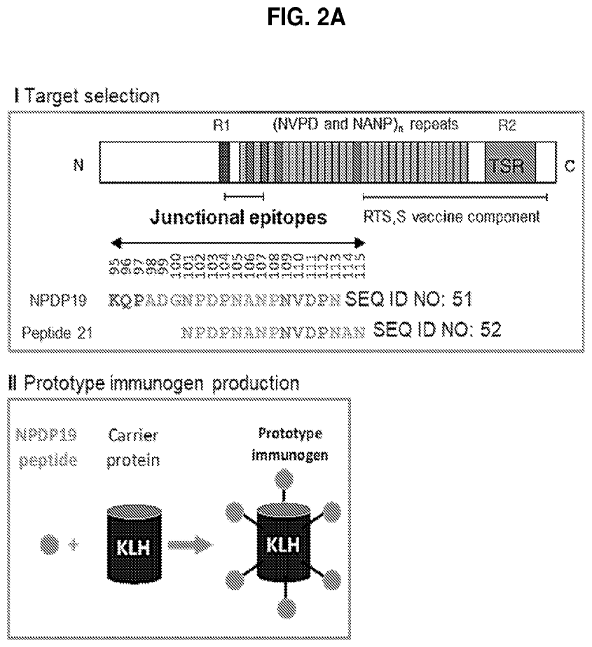

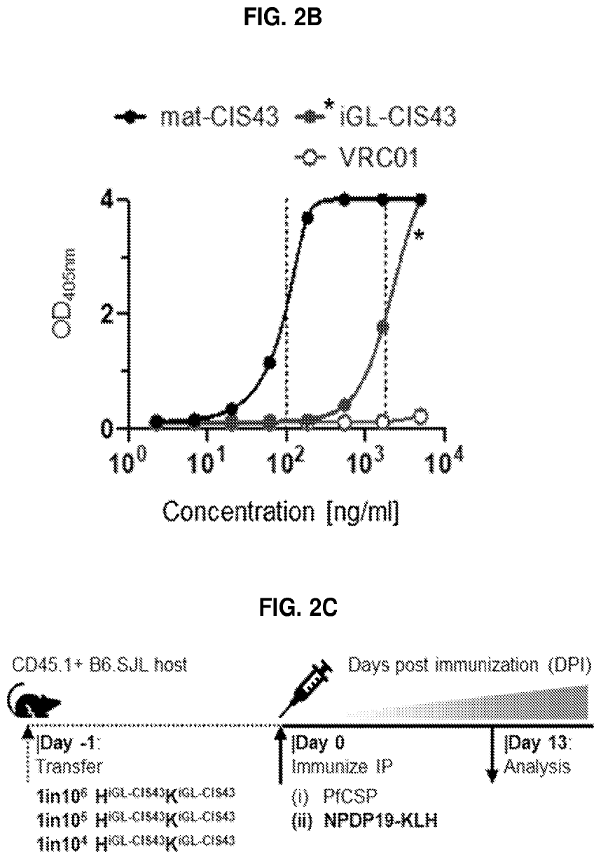

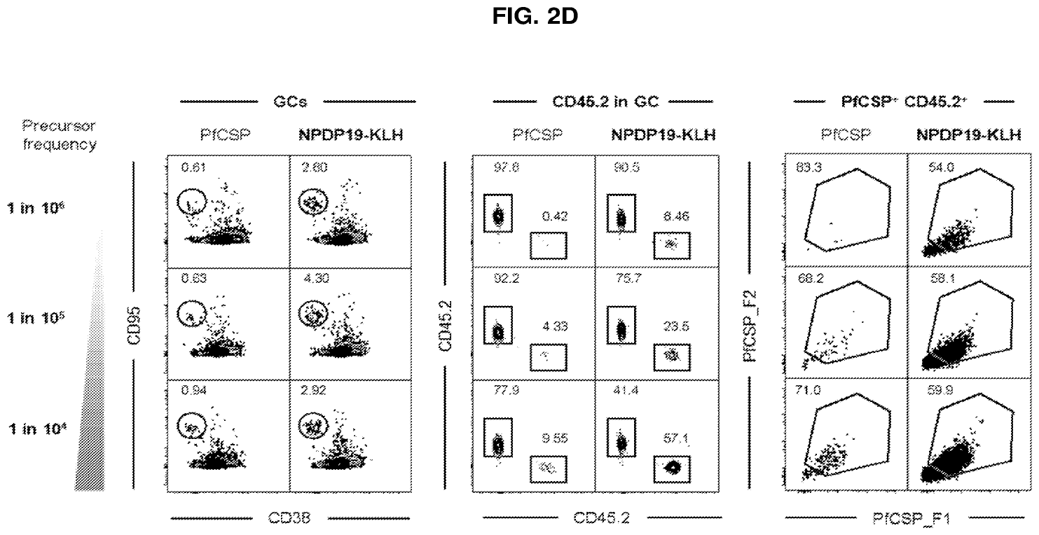

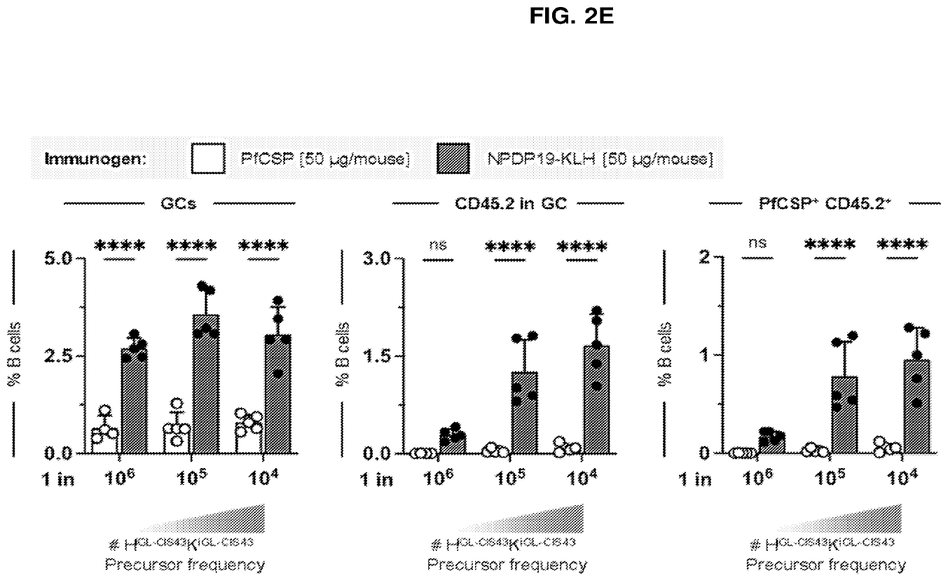

Since PfCSP is a multi-epitope immunogen (Cockburn and Seder, 2018b; Wardemann and Murugan, 2018), we hypothesized that an immunofocusing vaccine strategy could enhance the activation of inferred germline CIS43 (iGL-CIS43) B cells and lead to improved germinal center recruitment. The PfCSP-junctional region, which links the N-terminal and NANP-repeat region of PfCSP, is contained within 19 amino acids (KQPADGNPDPNANPNVDPN (SEQ ID NO: 51); corresponding to residues 95-115 of PfCSP). Indeed, the CIS43 mAb preferentially targets peptide 21 (NPDPNANPNVDPNAN (SEQ ID NO: 52), (Kisalu et al., 2018)), while the MGG4 mAb targets NPDP19 (KQPADGNPDPNANPNVDPN (SEQ ID NO: 51), (Tan et al., 2018)) (FIG. 2A, Target selection). Since the 19-mer NPDP19 contained all tetrapeptide epitopes of interest and had previously induced anti-PfCSP responses in BALB/c mice (Tan et al., 2018) following immunization 50 μg/mouse, this was used for our immunofocusing vaccine strategy.

To enhance the immunogenicity of the peptide, NPDP19 was covalently linked to KLH (FIG. 2A, Prototype immunogen production), and confirmed by ELISA that the NPDP19 peptide is recognized by the iGL-CIS43 mAb in the context of the large carrier protein KLH (˜390 kDa) (FIG. 2B).

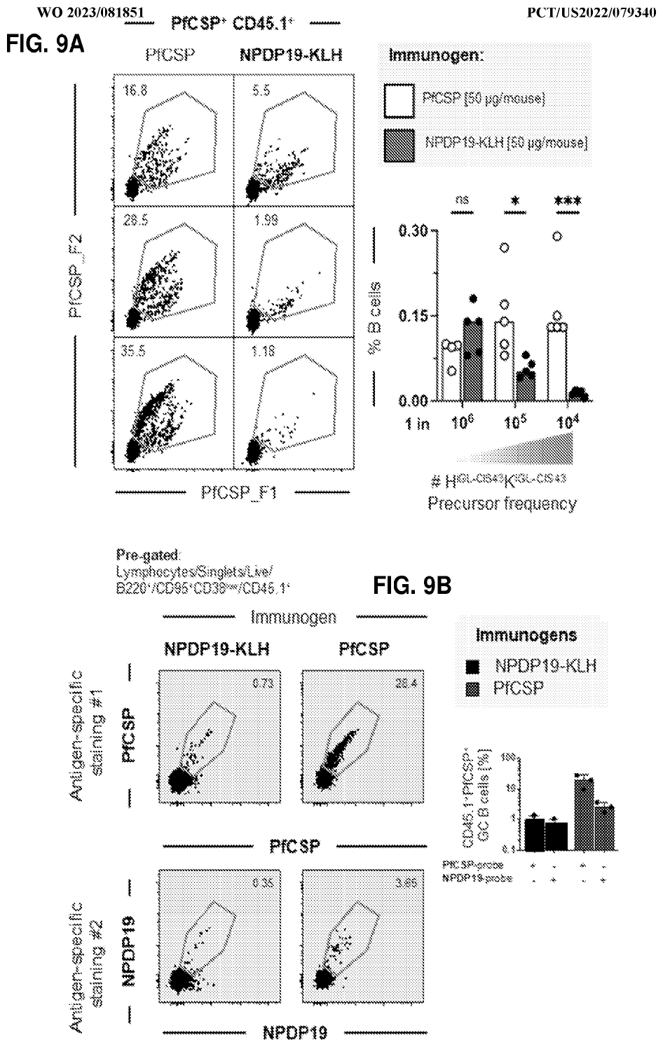

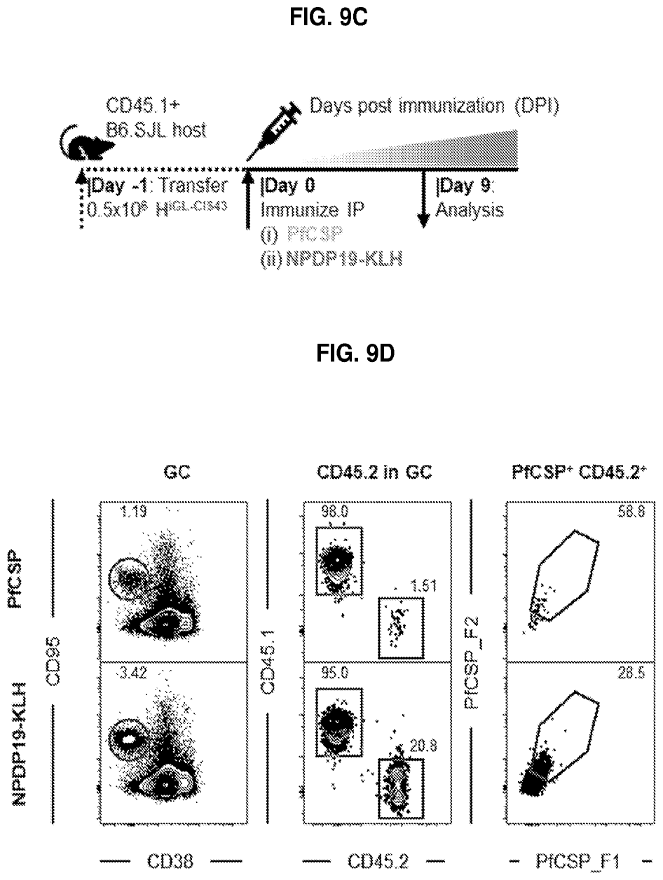

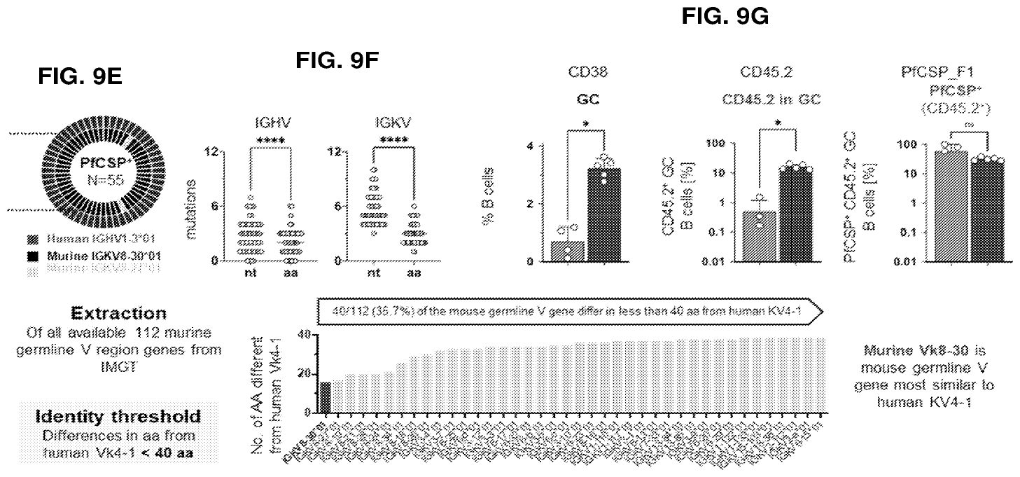

H^{iGL-CIS43}κ^{iGL-CIS43} B cells were adoptively transferred into congenic mice to achieve precursor frequencies of 1:10⁴, 1:10⁵ and 1:10⁶ for immunizations with either PfCSP/Alhydrogel or immunizations with NPDP19-KLH/Alhydrogel at 50 μg/mouse. Splenocytes were analyzed by flow cytometry at 13 days following immunization (FIG. 2C). While GC B cells (CD38^{low}CD95⁺) were detected in both immunization conditions, the number of GC B cells induced by PfCSP (at 1:10⁶: N=0.003±0.004%; 1:10⁵: N=0.043±0.014%; at 1:10⁴: N=0.086±0.060%) were significantly lower than GCs induced by NPDP19-KLH (at 1:10⁶: N=0.29±0.088%; 1:10⁵: N=1.26±0.493%; at 1:10⁴: N=1.67±0.47%). Significantly more transferred CD45.2 PfCSP-reactive B cells were recruited to GCs following NPDP19-KLH immunization in comparison to PfCSP-immunization in animals with higher precursor frequencies (FIGS. 2D and 2E). Of note, NPDP19-KLH immunization increased junctional epitope-specific activation while reducing the recruitment of PfCSP-specific competitor CD45.1 GC B cells into the GCs (FIG. 9A). Indeed, staining NPDP19-specific B cells revealed a significantly lower number of the host CD45.1 GC B cells bound the junctional epitope (FIG. 9B). These data suggest that other regions of the full-length PfCSP protein may divert or sterically hinder B cell responses from the junctional site of vulnerability (Kisalu et al., 2018; Zavala et al., 1983).

Since most of the residues characterized as critical to CIS43 binding are on the heavy chain (Kisalu et al., 2018), we also tested our PfCSP and NPDP19-KLH immunization strategies in the H^{iGL-CIS43} KI model (in which the human H^{iGL-CIS43} heavy chain remains free to pair with endogenous murine light chains). Interestingly, NPDP19-KLH immunization also led increased and more specific activation of H^{iGL-CIS43} B cells (FIGS. 9C-9H), further confirming our immunofocusing strategy. Notably, corresponding murine light chains were highly enriched for murine IGVK8-30*01 (96.3% (53/55)), indicating a preferential usage of this particular murine light chain in combination with human IGHV1-3*01 in BCRs capable of binding to PfCSP (FIG. 9E); strikingly, murine IGKV8-30*01 exhibits a high degree of sequence homology with its human counterpart (FIG. 9H): of 112 murine germline V-genes sequenced from H^{iGL-CIS43} PfCSP-binders, IGKV8-30*01 had the greatest similarity to human KV4-1*01, with a difference of only 16 amino acids.

Overall, these results indicate that using the minimal junctional epitope instead of full-length PfCSP significantly enhanced H^{iGL-CIS43}κ^{iGL-CIS43} B cell responses while reducing the responses of the undesired host B cells.

### NPDP19-KLH Immunization Recruits CIS43-Precursors to Long-Lasting Germinal Centers

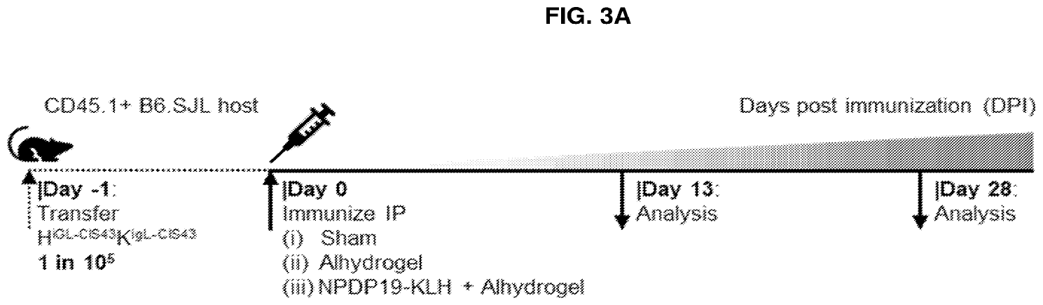

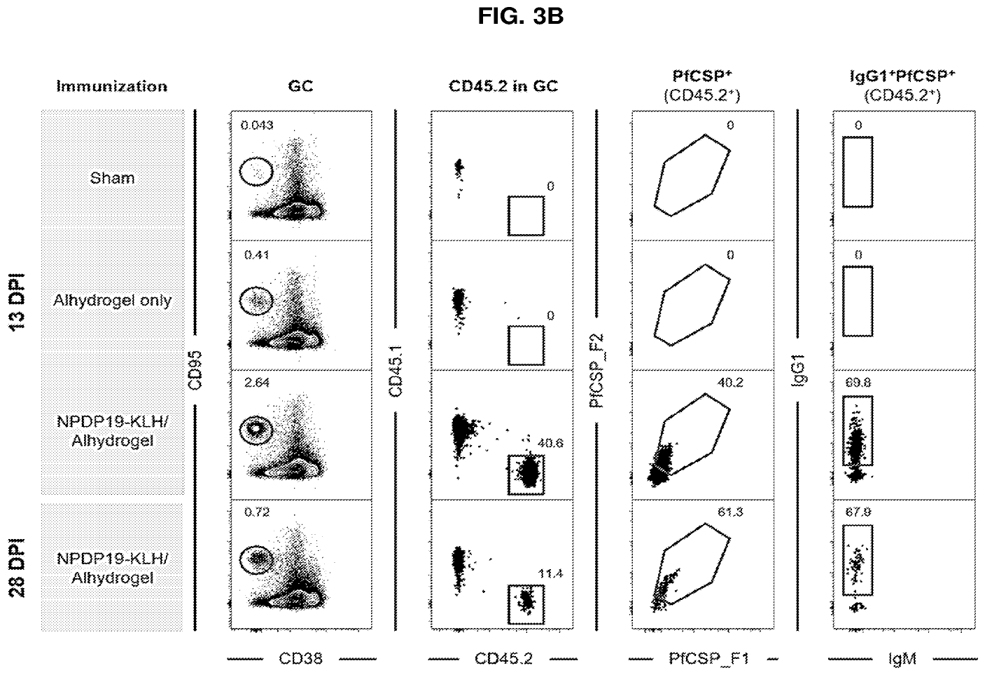

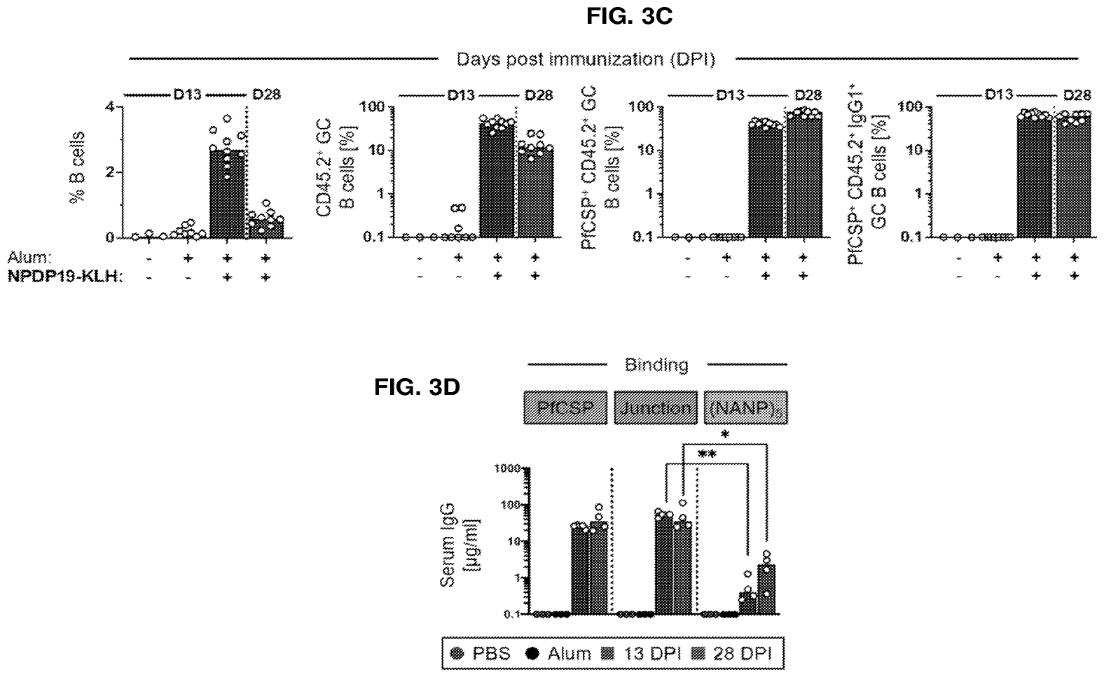

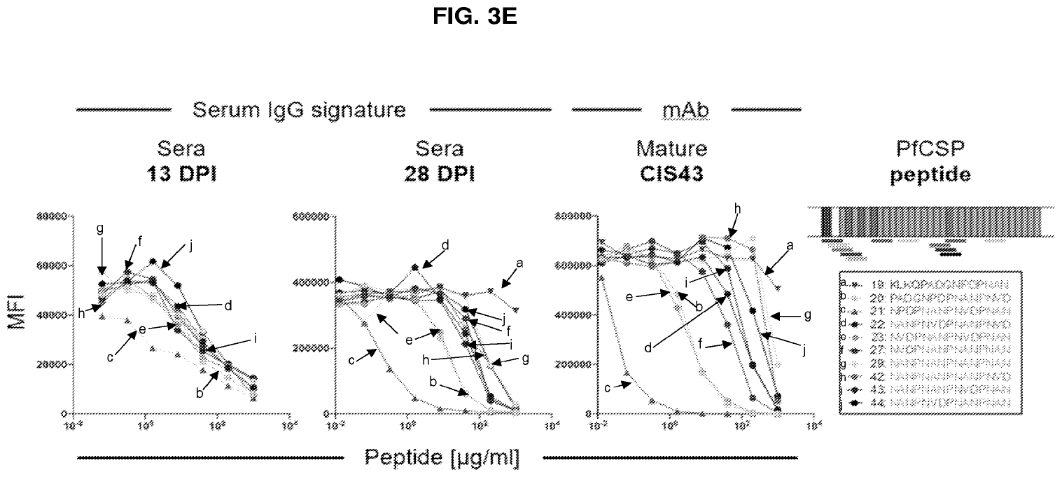

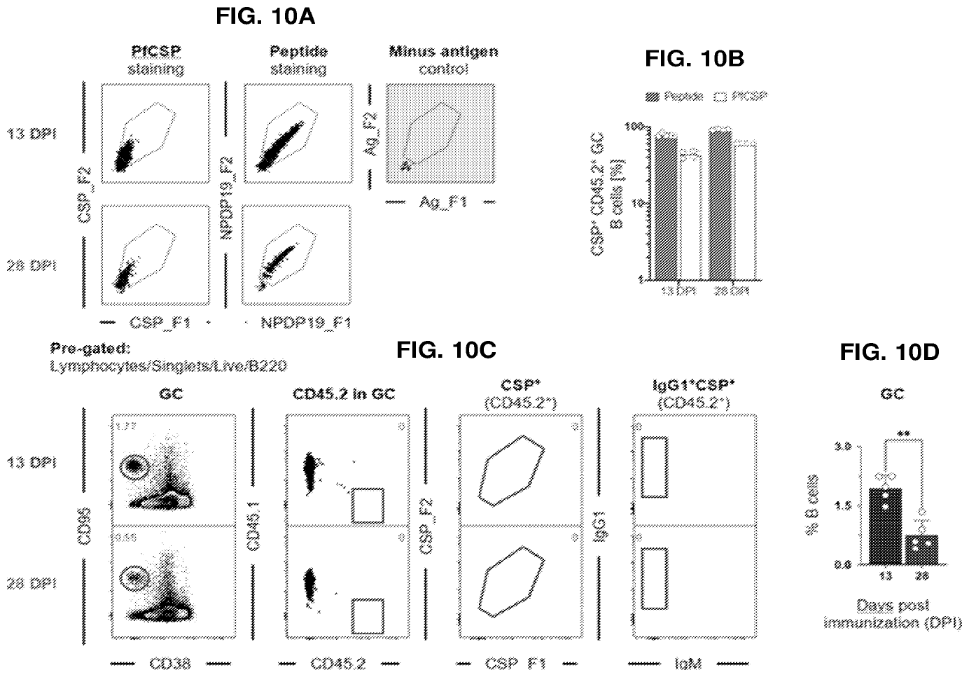

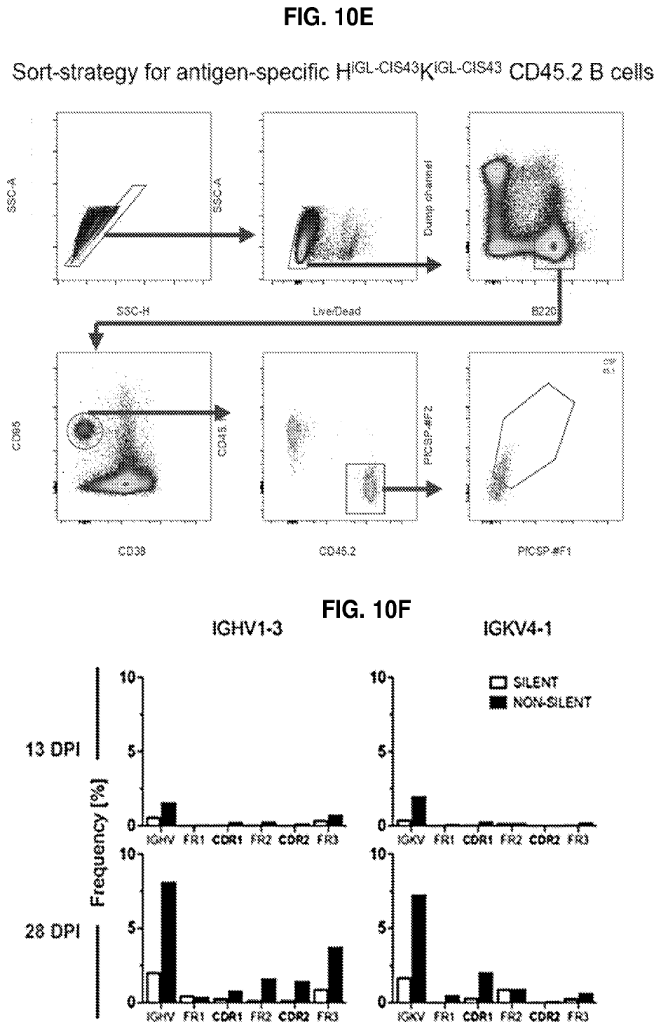

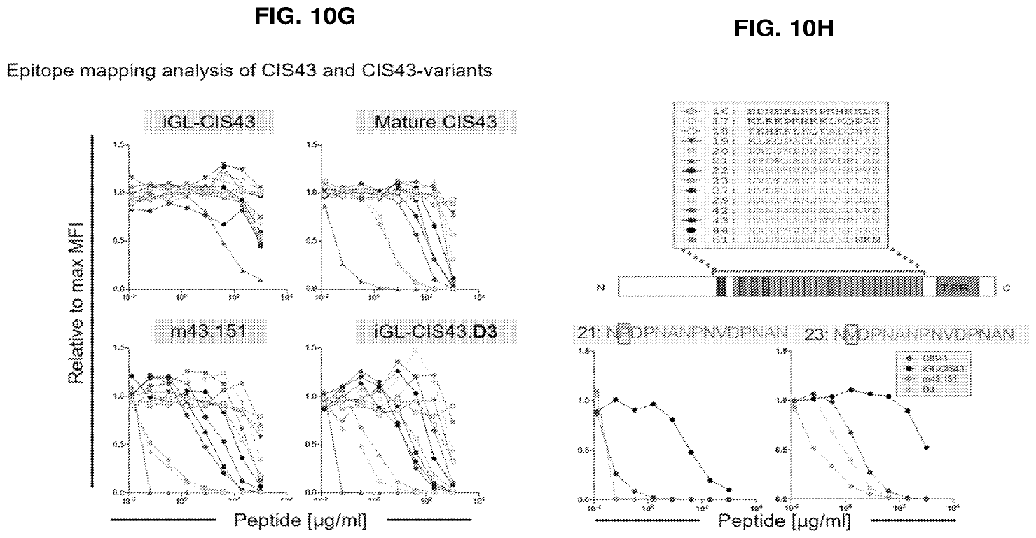

To determine whether NPDP19-KLH/Alhydrogel, can induce durable responses and mature-CIS43-like antibodies (Kisalu et al., 2018), GC responses were assessed at 13 and 28 days post immunization (DPI), starting from a precursor frequency of 1:10′; for 13 DPI we included sham and adjuvant-only control groups to assess if the resulting B cell responses were directly linked to NPDP19-KLH immunization (FIG. 3A). As expected, in the sham and adjuvant-only control groups CD45.2 H^{iGL-CIS43}κ^{iGL-CIS43} B cells could not be recruited to GCs (FIG. 3B), while CD45.2 H^{iGL-CIS43}κ^{iGL-CIS43} B cells reached a mean peak frequency of ˜42.3% at 13 days and ˜13.8% at 28 days following immunization with NPDP19-KLH/Alhydrogel (FIG. 3C). A substantial proportion of these CD45.2 H^{iGL-CIS43}κ^{iGL-CIS43} GC B cells bound to PfCSP (13 DPI: N=41.0±4.9%; 28 DPI: N=71.5±10.2%) and was predominantly class-switched to IgG1 (13 DPI: N=65.7±8.2%; 28 DPI: N=57.6±12.2%), confirming normal GC functionality. Parallel staining using PfCSP or NPDP19 as probes for assessment of antigen-specific B cell responses revealed a high level of epitope-specificity in responses by CD45.2 GC B cells with high levels of antigen-binding at 13 DPI (PfCSP: N=44.0±4.0%; NPDP19: N=77.9±3.4%) and 28 DPI (PfCSP: N=92.5±1.5%; NPDP19: N=61.1±0.39%) (FIGS. 10A and 10B). Differences in PfCSP- and NPDP19-recognition were not significant (13 DPI: p=0.06, 28 DPI: p=0.13) and are likely attributable to the more complex nature of PfCSP, in which steric clashes with immunodominant NANP-repeats can restrict epitope-accessibility (Zavala et al., 1983).

We also included wildtype (WT) controls for both timepoints, for which equivalent numbers of CD45.2 B cells from C57BL/6J mice (WT CD45.2) were transferred into CD45.1 recipient mice and immunized with NPDP19-KLH/Alhydrogel. In contrast to CD45.2 H^{iGL-CIS43}κ^{iGL-CIS43} B cells, which responded strongly to NPDP19-KLH immunization, WT CD45.2 cells transferred at rare frequencies could not be recruited to GCs (FIGS. 10C and 10D).

To quantitatively assess the amount of antibody produced in response to NPDP19-KLH immunization, IgG binding analysis was performed against PfCSP-, Junction- and (NANP)₅. No antigen-specific serum IgG was detected in either the sham or the adjuvant only control, while high PfCSP- and Junction-specific IgG-levels were detected at both 13 DPI (PfCSP: N=25.8±3.2 μg/ml; Junction: N=53.8±8 μg/ml) and 28 DPI (PfCSP: N=44.7±30.5 μg/ml; Junction: N=42.8±42.0 μg/ml) in mice immunized with NPDP19-KLH/Alhydrogel, while binding to the (NANP)₅ repeat region was minimal (13 DPI: N=0.59±0.5 μg/ml; 28 DPI: N=2.4±1.8 μg/ml) (FIG. 3D). These findings were further substantiated by epitope mapping of serum responses, showing that CIS43-like peptide-binding signatures were detectable at 28 DPI (FIGS. 3E, 10G and 10H). Collectively, these data reveal that immunofocusing with the junctional epitope led to sustained, junctional specific antibody responses from CD45.2 H^{iGL-CIS43}κ^{iGL-CIS43} B cells.

### NPDP19-KLH Immunization Recapitulates Mature CIS43-Like SHM

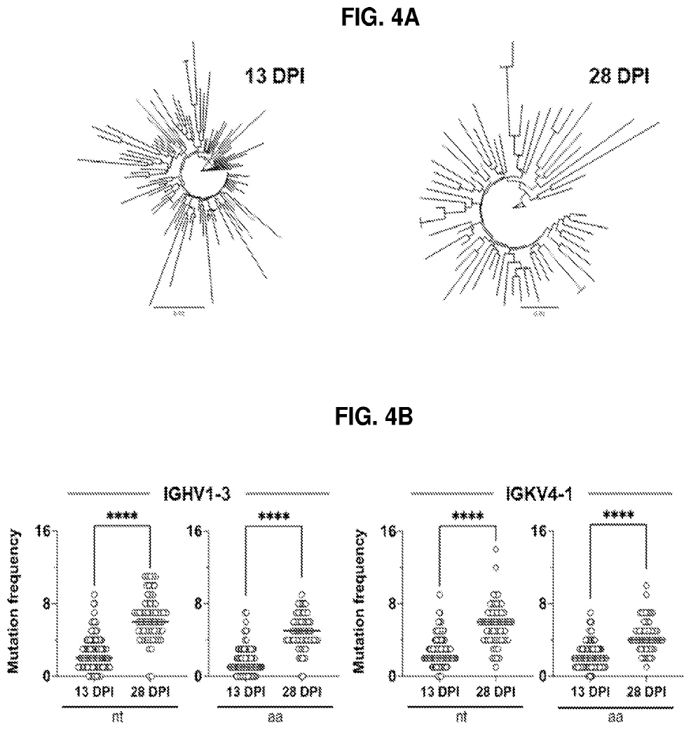

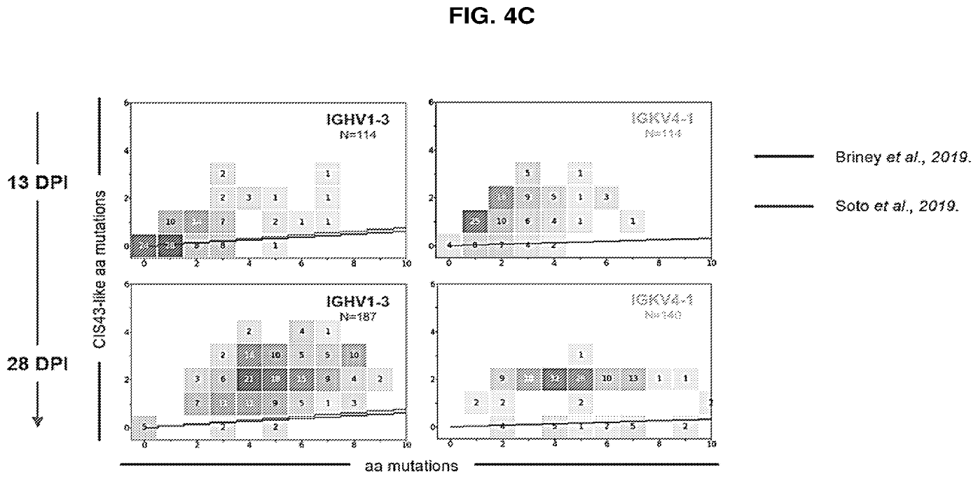

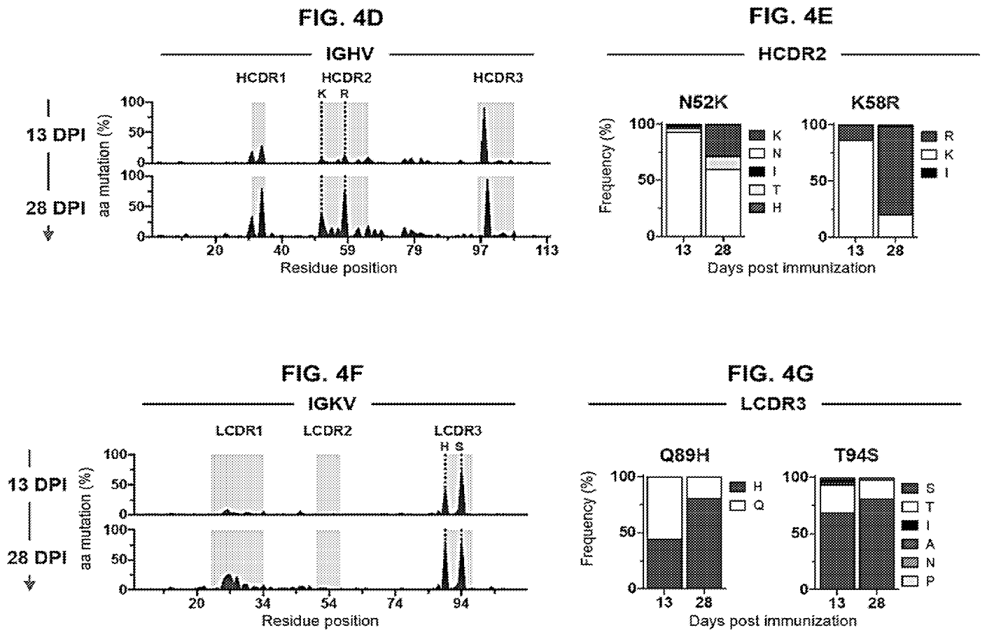

A notable feature of the mature CIS43 mAb is the low level of SHM (˜3-4%). To assess if iGL-CIS43 BCRs can undergo SHM and accumulate key mutations reported for mature CIS43 following immunization with NPDP19-KLH (Kisalu et al., 2018), single-cell paired sequence analysis was performed from antigen-specific H^{iGL-CIS43}κ^{iGL-CIS43} B cells at two different time points (13 DPI and 28 DPI) (FIG. 4 and FIG. 10E). Phylogenetic trees generated from bioinformatically assembled heavy-light chain sequence pairs revealed a high degree of sequence diversity in isolated CIS43-variant sequences (FIG. 4A). Substantial levels of SHM in iGL-CIS43 heavy chains were detected within 13 days of immunization, with a maximum of 7 amino acid (AA) mutations and a median of 1 aa mutation. SHM was significantly (p<0.0001) increased 28 days after immunization to a maximum of 9 aa mutations and a median of 5 aa mutations. Corresponding iGL-CIS43 light chains also accumulated SHM that significantly increased (p<0.0001) from a median of 2 aa mutations at 13 days to a median of 4 mutations at 28 days after immunization. Further, we observed that the mean level of nucleotide mutations in iGL-CIS43 heavy chains (13 DPI: N=2.5±1.8; 28 DPI; N=6.4±2.5) and light chains (13 DPI: N=2.8±1.6; 28 DPI; N=5.7±2.3) were strikingly similar to levels of aa mutations in heavy (13 DPI: N=1.7±1.6; 28 DPI; N=4.9±1.9) and light chains (13 DPI: N=2.3±1.4; 28 DPI; N=4.5±1.8) at both time points (FIGS. 4B and 4C). This was confirmed via the analysis of silent and non-silent mutations, which revealed that most of the nucleotide mutations in the IGHV-region led to observable changes in the corresponding amino acid sequences (FIG. 10F).

Next, we evaluated whether immunization with NPDP19-KLH could induce B cells toward an affinity maturation pathway with CIS43 antibody-like mutations (FIG. 4D). Thirteen days following NPDP19-KLH priming, 39% (45/114) of isolated heavy and 78.7% (89/114) light chains exhibited at least one CIS43-like mutation, and after 28 days 95.2% (178/187) of heavy chains and 86.4% (121/140) light chains had at least one CIS43-like mutation (FIG. 4D). In the iGL-CIS43 heavy chain, aa mutations occurred at reasonable frequencies in CDR-H2-proximal key residues N52K (1.8%) and K58R (13.2%) at 13 days after immunization and became more predominant (N52K: 28.6%; K58R: 77.8%) at 28 days (FIGS. 4E and 4F); these residues are important for binding to the junctional malaria epitope (Kisalu et al., 2018). Interestingly, CIS43-like mutations in corresponding light chains were largely confined to the CDR-L3 (FIG. 4G) and occurred at high frequencies in key residues Q89H (13 DPI: 44.7%; 28 DPI: 55.3%) and T94S (13 DPI: 69.3%; 28 DPI: 81.0%) (FIG. 4H), highlighting the importance of CIS43-like light chains for the recognition of the junctional epitope.

These data show that B cell bearing iGL-CIS43 BCRs can accrue significant levels of SHM and CIS43-like mutations after a single-immunization with the junctional malaria epitope NPDP19.

Informatic Sieving Yields CIS43-Variant Antibodies with Improved Protection

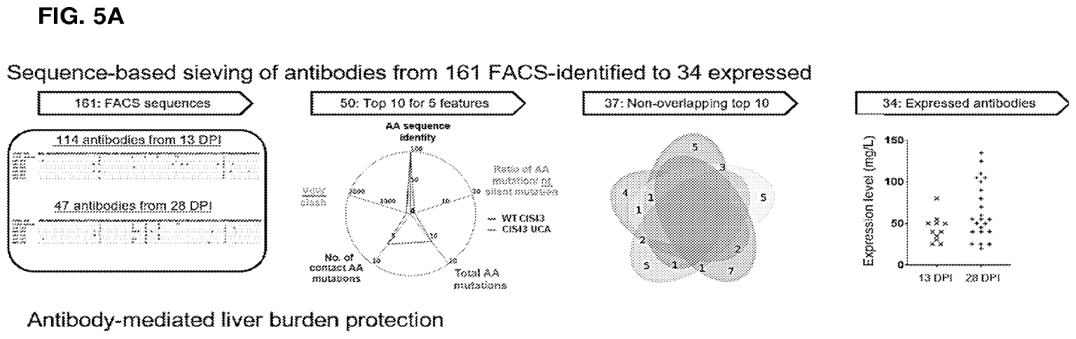

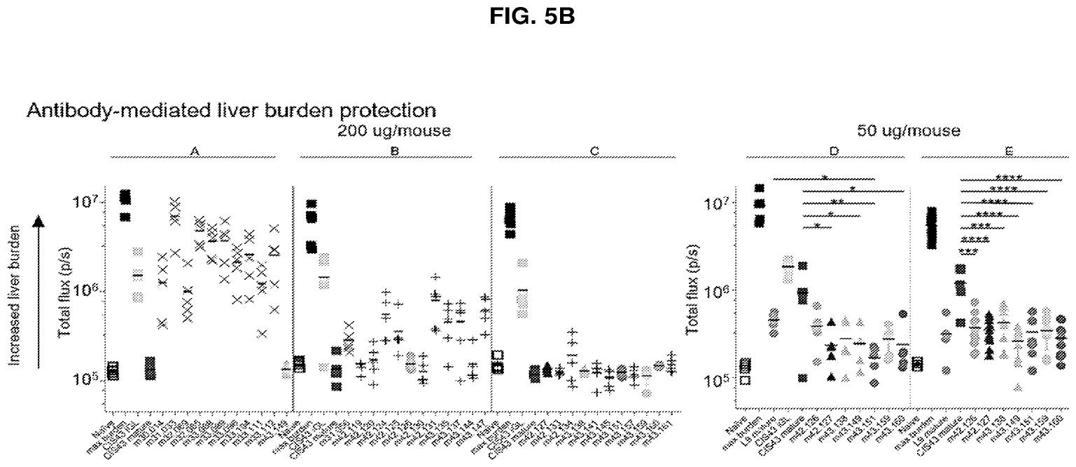

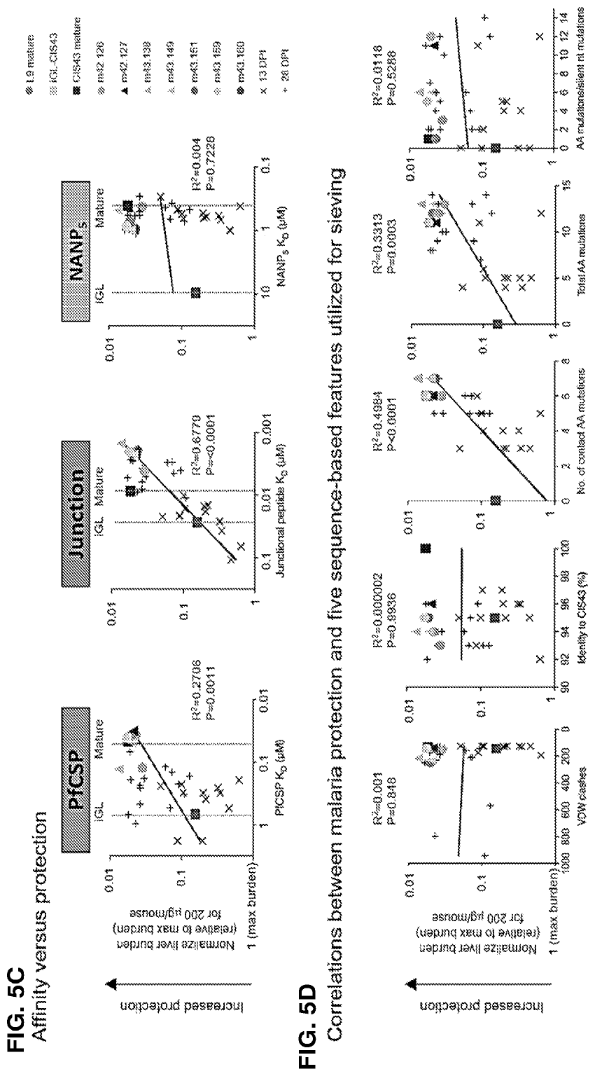

The recapitulation of many of the features of mature CIS43 sequences following adoptive transfer of H^{iGL-CIS43}K^{iGL-CIS43} B cells and vaccination with junctional peptide-KLH suggested their encoded antibodies might have protective efficacy comparable or higher than the parental CIS43 antibody. However, to identify which of the 161 paired heavy-light chain sequences obtained from 13 DPI (114 sequences) and 28 DPI (47 sequences) might have improved efficacy, we sieved sequences based on genetic features such as sequence identity to mature CIS43 or measures of somatic hypermutation (SHM) that might be expected a priori to correlate with increased malaria-protective efficacy (FIG. 5A).

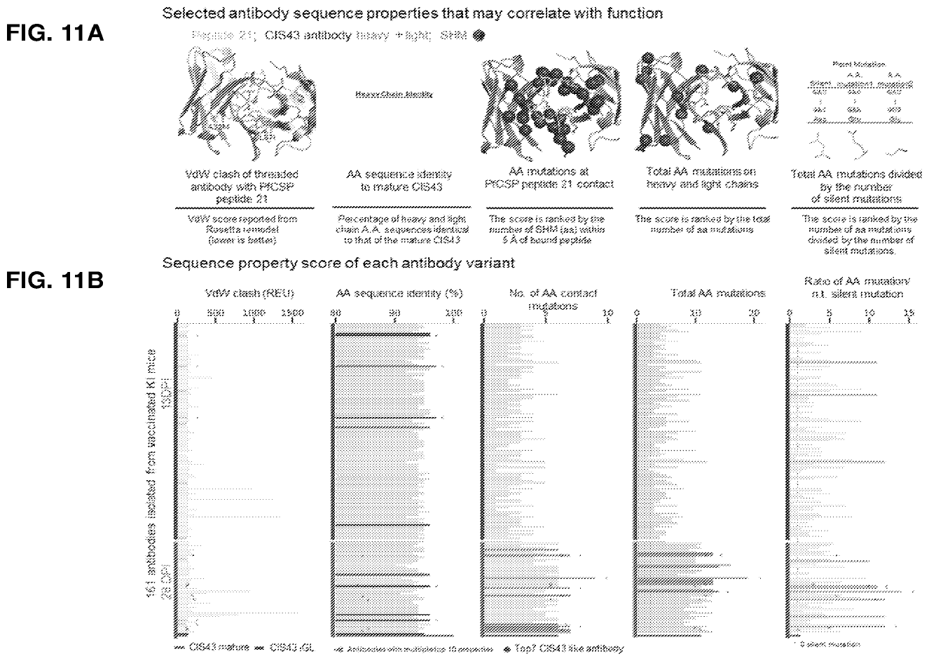

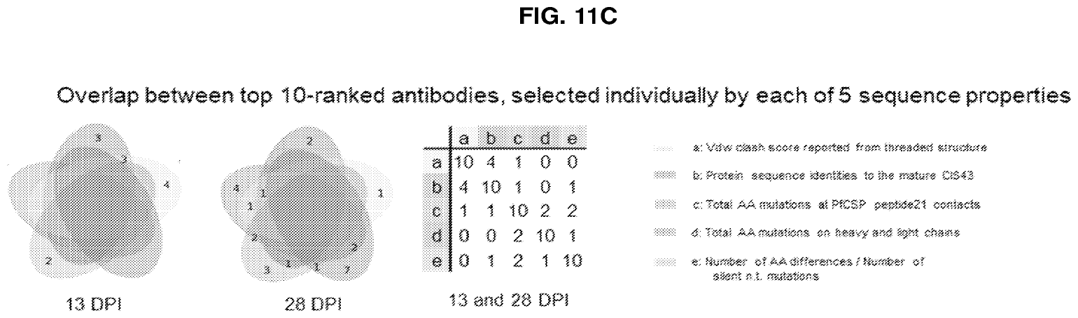

First, as antibodies with van der Waals clashes with the CIS43-recognized epitope were unlikely to have high protective efficacy, we identified sequences that when threaded on the CIS43-peptide 21 structure (PDB-ID: 6B5M) had low van der Waals clash scores; interestingly, many of the antibody variants with optimal clash scores were from 13 DPI (FIGS. 11A and 11B leftmost panels). Second, we identified elicited antibodies with high sequence identity to mature CIS43 as they would likely have protective efficacy; a majority of the 10 antibodies with highest identity were from 13 DPI (FIGS. 11A and 11B 2nd panels from left). Third, as germline-reverted CIS43 is poorly efficacious and somatic hypermutation (SHM) is required for increased efficacy, we quantified SHM in two ways: (i) the number of amino acid mutations within 5 Å of bound peptide in the threaded CIS43-peptide 21 structure, and (ii) the total number of amino acid mutations. With both of these SHM-related features, the 10 highest scoring variants were all from 28 DPI (FIGS. 11A and 11B, 3^{rd} and 4th panels from left). Lastly, as the ratio of amino acid mutations versus silent mutations can reflect the degree of selection, we analyzed this ratio. Roughly 10% of the sequences (especially those from 13 DPI) had no silent mutations, while the highest defined ratios were observed more frequently at 28 DPI (FIGS. 11A and 11B far right panels).

Multiple sequences were identified as being among the top 10 in several categories (FIG. 11C). Indeed, 37 sequences covered the top 10 sequences of all 5 features (FIG. 4A). Thirty four of these expressed at a sufficient level (Table 2) to enable assessment of in vivo protective efficacy in a mouse model of malaria infection (Raghunandan et al., 2020). The ability of these 34 antibodies to reduce parasite liver burden following passive transfer at 200 μg/mouse was determined in mice challenged intravenously with transgenic P. berghei sporozoites expressing PfCSP and a green fluorescent protein/luciferase fusion protein (Pb-PfCSP-GFP/Luc-SPZ; hereafter Pb-PfCSP-SPZ) (Flores-Garcia et al., 2019a). Bioluminescent quantification of liver burden was carried out 42 h post challenge with all 34 antibodies, benchmarked against mature and inferred germline versions of CIS43. Mature CIS43 confers roughly a 2-log reduction in liver parasite burden at this dose (Kisalu et al., 2018), while the same dose of the inferred germline version was significantly less protective. Notably, at a dose of 200 μg/mouse, multiple variant antibodies showed protective efficacy that was statistically indistinguishable from mature CIS43 (FIG. 4B, left panel). To better differentiate their potency, we assessed seven of the most protective antibodies, all from 28 DPI, at a dose of 50 μg/mouse, adding as an additional benchmark the recently identified L9 antibody (Wang et al., 2020), which has ˜3-fold higher protective efficacy than CIS43. In two independent experiments, five of the antibodies showed significantly better protection compared to mature CIS43, and similar protection to L9, suggesting at least 3-fold or higher efficacy for these antibodies than mature CIS43 (Table 3).

Delineation of CIS43 Features that Correlate with Protective Efficacy

The differential protection by the 34 CIS43 variants as well as by mature and iGL-versions of CIS43 provided an opportunity to determine how antibody properties relate to protective efficacy. We used bilayer surface interferometry to measure the affinity of these CIS43 variants to PfCSP, junctional peptide, and NANP repeats (Table 4). Binding to PfCSP was fit using a 2-component binding model, and the primary binding K_{D} correlated moderately (R=0.52) with protection (FIG. 5C, left panel). Binding to the junctional peptide fit well to a single component binding model, and junctional peptide affinity correlated more strongly (R=0.82) with protection (FIG. 5C, middle panel). However, binding to the penta-NANP repeat did not correlate with protection (FIG. 5C, right panel). In all three cases, the difference in affinity between iGL and mature CIS43 was similar, between 10- to 30-fold, with differences in correlation reflecting the distribution of the sequence-feature-selected antibodies. Interestingly, binding of these 34 antibodies to sporozoites assessed by flow cytometry did not trend with protective efficacy, though mature CIS43 did show a higher degree of interaction than iGL-CIS43 (FIG. 11E). With binding to PfCSP and NANP₅, affinity of mature CIS43 rivaled variants with highest protective potency; with junctional peptide, affinity of mature CIS43 was substantially less than variants with highest protective efficacy. Overall, these correlations reflect the expected binding properties of CIS43 (Kisalu et al., 2018), with junctional affinity being the most critical property of CIS43 in vivo protective potency.

We also analyzed the correlation of protective efficacy with the five sequence-related features that we used in our sequence-based genetic sieving. Notably, only the two properties associated with SHM, contact amino acid mutations and total amino acid mutations, correlated significantly with protection. Of these, contact mutations (R=0.71) correlated more strongly than total mutations (R=0.58) (FIG. 5D). The lack of correlation with van der Waals clash scores, with CIS43 identity, or with the ratio of amino acid versus silent mutations suggested that there were multiple ways to satisfy these features—some with limited efficacy improvement—and this was perhaps anticipated by the prevalence of top-ranked antibodies with these features in 13 DPI antibodies.

### Binding and Structural Basis of Improved CIS43 Antibodies

To gain insight into the binding and structural properties of the improved CIS43 antibody variants, we analyzed the most protective antibodies. Antibody iGL-CIS43-HL.K28.m43.151, named for transferred B cells (H^{iGL-CIS43}κ^{iGL-CIS43}), immunization with junctional peptide-coupled to KLH (K), isolation date (28 DPI), mouse (43), and a unique identifying number (151)—and referred to hereafter and in figures by ‘mouse.number’, showed the greatest overall reduction in liver parasite burden—comparable to L9 and ˜3-fold improved over mature CIS43 (FIG. 5B, Table 3). Three other antibodies, m42.127, m43.149, and m43.160, also showed statistically significant reduced liver parasite burden versus mature CIS43 with the lower dose of antibody transferred, whereas three others, m42.126, m43.138, and m43.159 were statistically superior only in the 2^{nd} experiment (FIG. 5B).

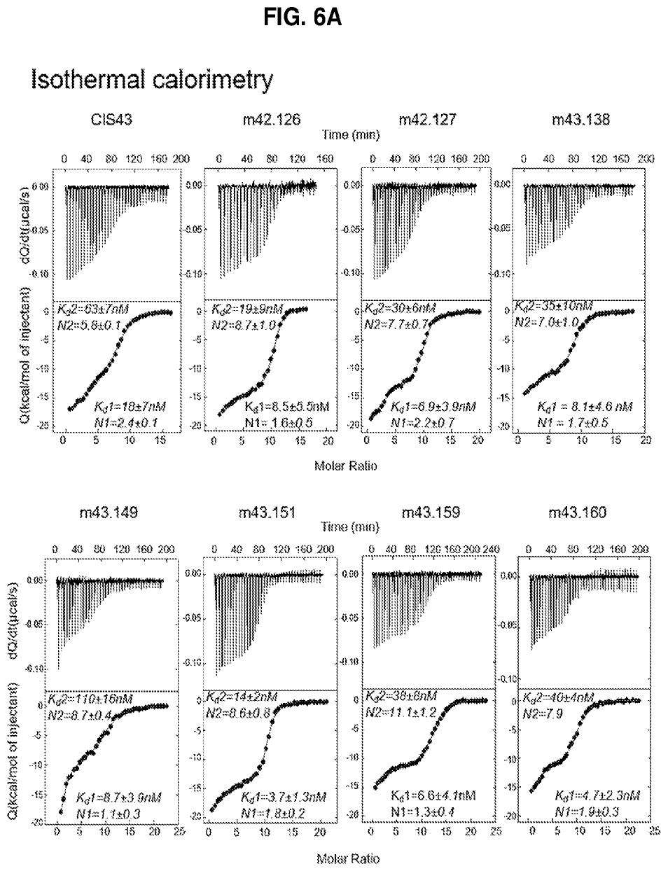

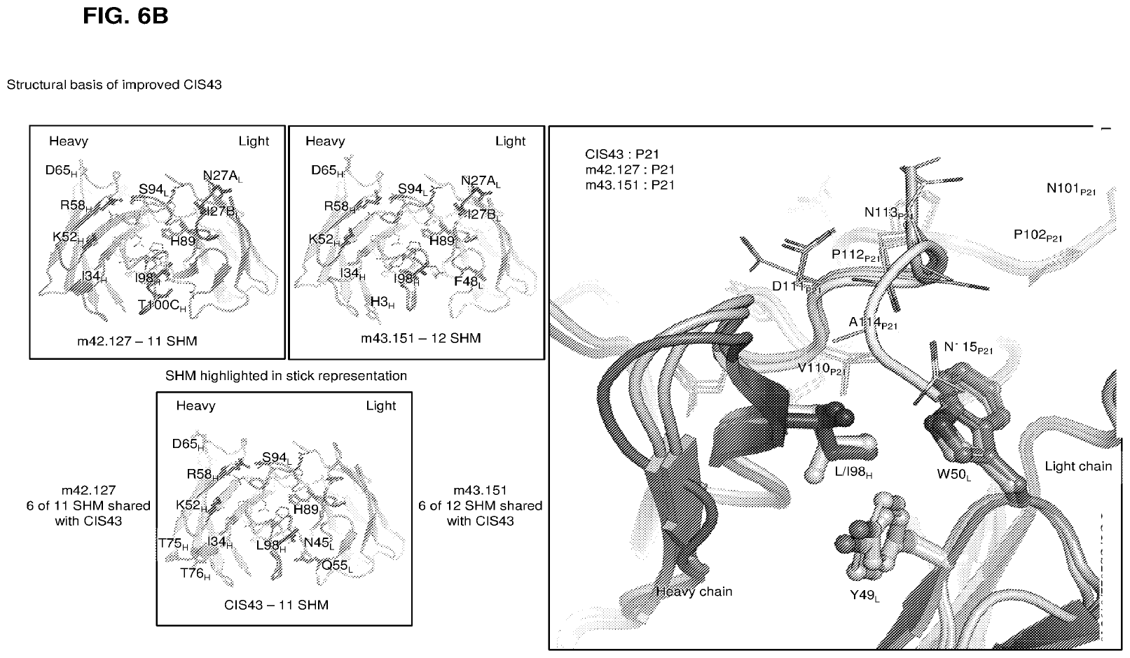

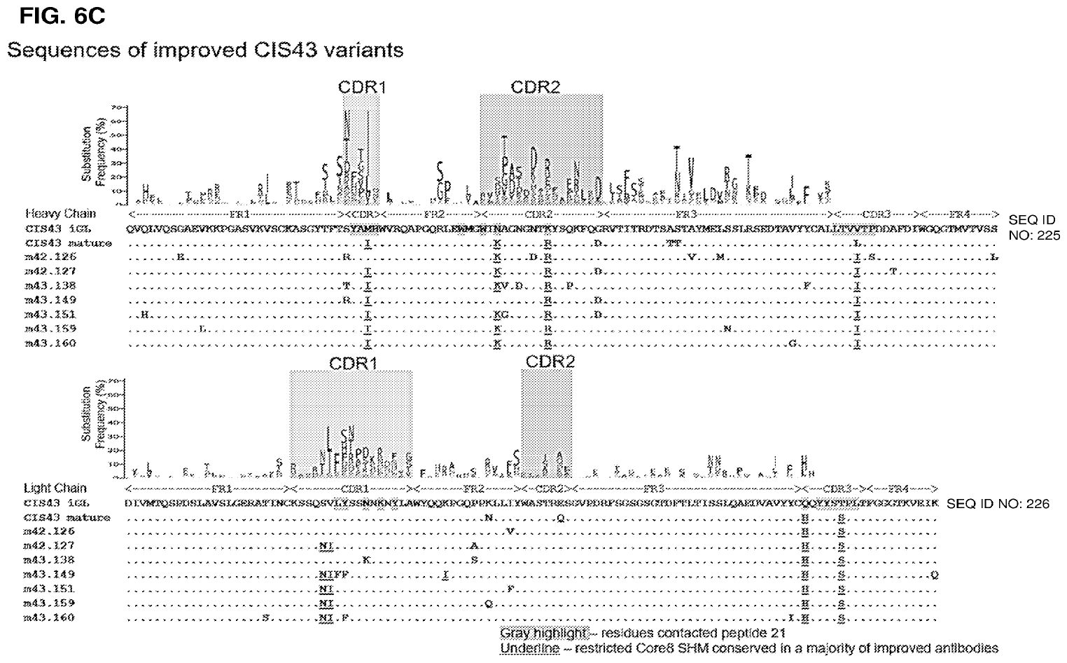

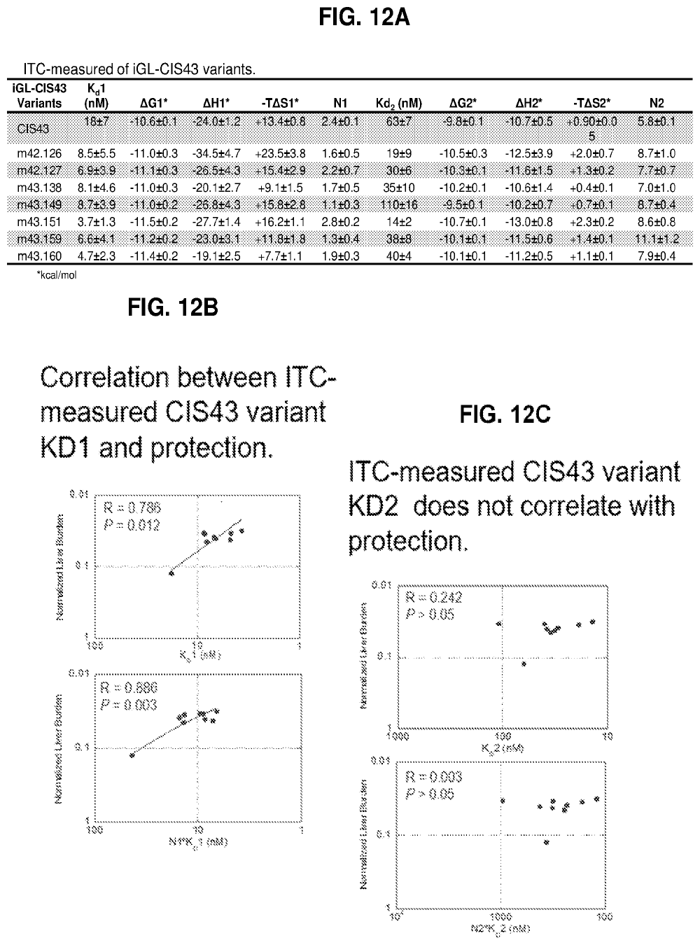

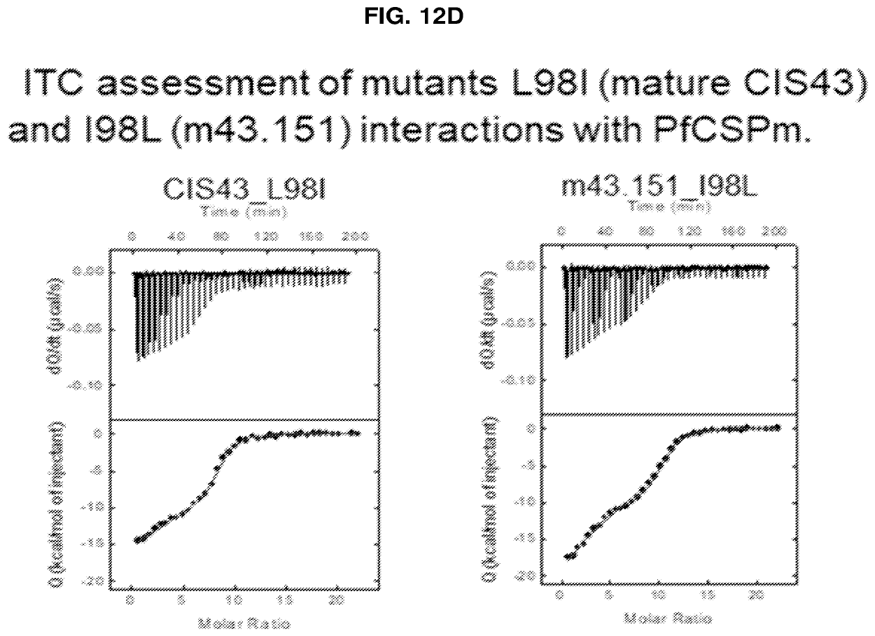

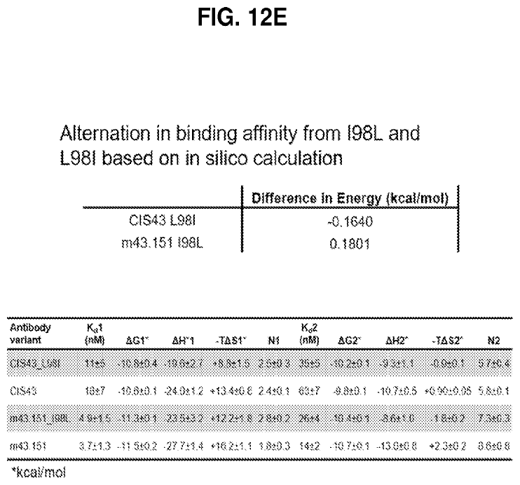

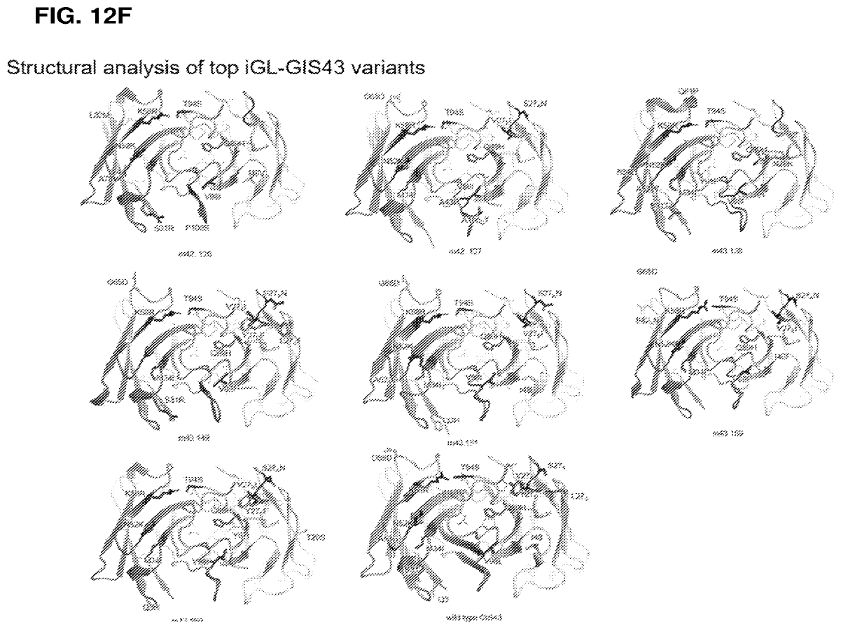

We used isothermal titration calorimetry to characterize the interactions of all seven of these antibodies with a mutant form of PfCSP with four amino acid mutations that removed processing sites and prevented dimerization upon solubilization to increase yield and to facilitate consistent analyses and referred to subsequently as ‘PfCSPm’ (Wang et al., 2020a) (FIGS. 6A and 12A). Mature CIS43 showed the characteristic two-step recognition observed previously (Kisalu et al., 2018), with binding affinities of 18 nM and 63 nM and with stoichiometries of 2.4 and 5.8 respectively. Notably, m43.151 showed substantial 4-5-fold affinity increases to both sites (3.7 and 14 nM). Other variants also showed higher affinities to both primary (junctional) and secondary (non-junctional) sites. The primary (junctional) affinity correlated with protection, with the correlation increasing if the number of sites were taken into consideration (FIGS. 12B and 12C). Interestingly, the secondary affinity did not correlate with protection; indeed, it trended negative, suggesting that increased secondary affinity might correlate with off-target responses. To provide insight into the atomic-level characteristics of binding by these variant antibodies, we determined their structures in complex with peptide 21 (residues 101-115 of PfCSP) for two CIS43 variants: the most potent variant from mouse 43, iGL-CIS43-HL.K28.m43.151, and the most potent variant from mouse 42, iGL-CIS43-HL.K28.m42.127 to resolutions of 2.0 and 1.8 Å, respectively (FIG. 6B, Table 5). Superimposition of the two structures with that of mature CIS43 revealed conformational conservation, with m43.151 and m42.127 structures differing from mature CIS43 by 0.60 Å and 0.29 Å root-mean square deviation (RMSD) (all atoms) in antibody variable regions respectively; and 0.156 Å and 0.158 Å RMSD (all atoms) in the antibody-recognized junctional peptide, respectively. A substantial proportion of SHM in these two antibodies was in common with that of mature CIS43 (FIG. 6B, left panels), with 6 of 12 SHM shared between m43.151 and CIS43 and 6 of 11 shared between m42.127 and CIS43. Thus, the maturation pathway from germline CIS43 to mature antibodies with protective efficacy appeared to require a restricted set of SHM along with a diverse set of changes.

Defining SHM observed in a majority of the CIS43-improved antibodies as the restricted set of SHM identified four aa residues on the heavy chain (M34I_{H}, N52K_{H}, K58R_{H} and V98I_{H}) and four aa residues on the light chain (S27_{A}N_{L}, V27_{B}I_{L}, Q89H_{L} and T94S_{L}) (FIG. 6C). Residue, G65D in the heavy chain, was a near miss, observed in three of the improved antibodies and also in CIS43. The presence of these restricted “Core8” set SHM as assessed on the 34 ‘top-10’ antibodies correlated highly with malaria protection (R=0.73, p<0.0001). Sequence-feature analysis indicated K52H and R58H to be associated with peptide 21 binding, only K52_{H} to be associated with PfCSP binding, and both K52_{H} and R58_{H} to be significantly associated with liver burden protection (Table 6). Many of these SHM were prevalent V-gene substitutions (Guo et al., 2019), though the mutational specificity was remarkable, with even single atom changes observed reproducibly. Notably, three mutations of the restricted Core8 set were not found in mature CIS43. These included the Asn-Ile dipeptide in the light chain CDR-L1, observed in five of the seven improved antibodies and attractively positioned near the N-terminus of peptide 21 (FIG. 6B, upper left panels) to interact with PfCSP, and well as Val98_{H} in the CDR-H3, which changed to Ile in all of the improved variants, but to Leu in mature CIS43.

We investigated the impact of a Leu to Ile change at residue 98_{H} both mutationally and computationally. For the former, we observed L98I_{H} in mature CIS43 to increase affinity to PfCSP by 1.6-fold (to 11 nM), whereas mutation of I98L in m43.151 decreased the affinity to PfCSP by 1.3-fold (to 4.9 nM); ITC analysis indicated the change in ΔG to be 0.2 kcal/mol (FIG. 12D). Computationally, we calculated a reduction in binding energy upon changing Leu to Ile to be −0.16 kcal/mol (CIS43 L98I) and changing Ile to Leu to be 0.18 kcal/mol (FIG. 12E).

Structural analysis revealed Ile98_{H} to stabilize the antigen-binding pocket by enhancing the heavy-light chain contacts through hydrophobic interactions with Y49_{L} and W50_{L}; the Ile change also enables additional contact with the C-terminus of junctional region including residue Val110 on PfCSP (FIG. 6B, right panel). Overall, these results confirmed the ability of single atom changes to have functional impact on the CIS43 antibody, potentially explaining the repeated selection of the exact same SHMs in the restricted set of mutations (FIGS. 6C and 12F). The higher prevalence of the restricted Core8 mutations in the improved antibodies provides an intriguing explanation for the increase in their protective capacity.

### Improved CIS43 Antibody “D3”

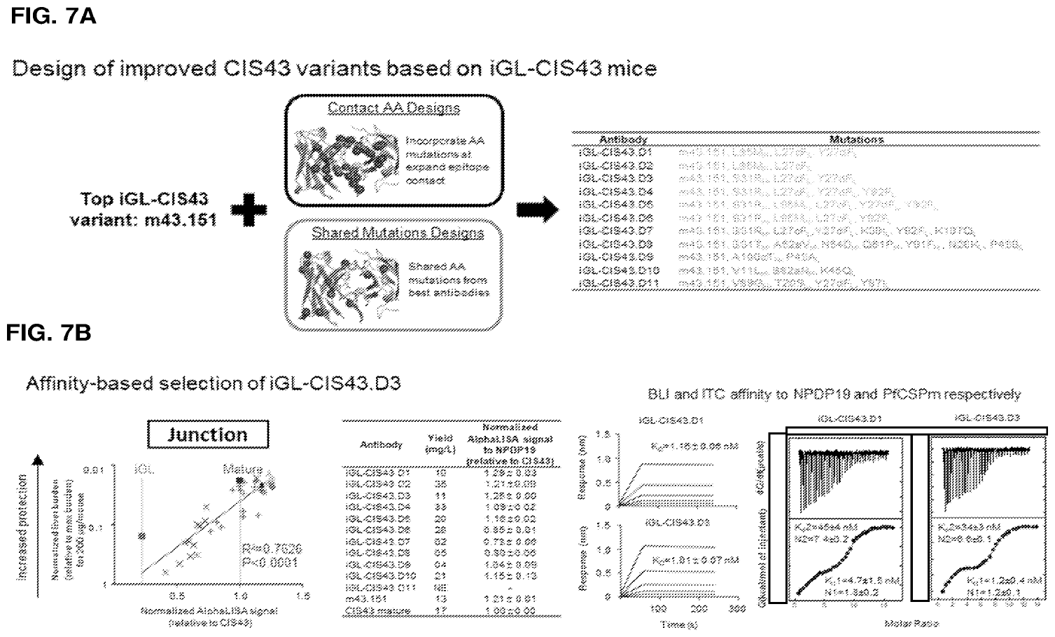

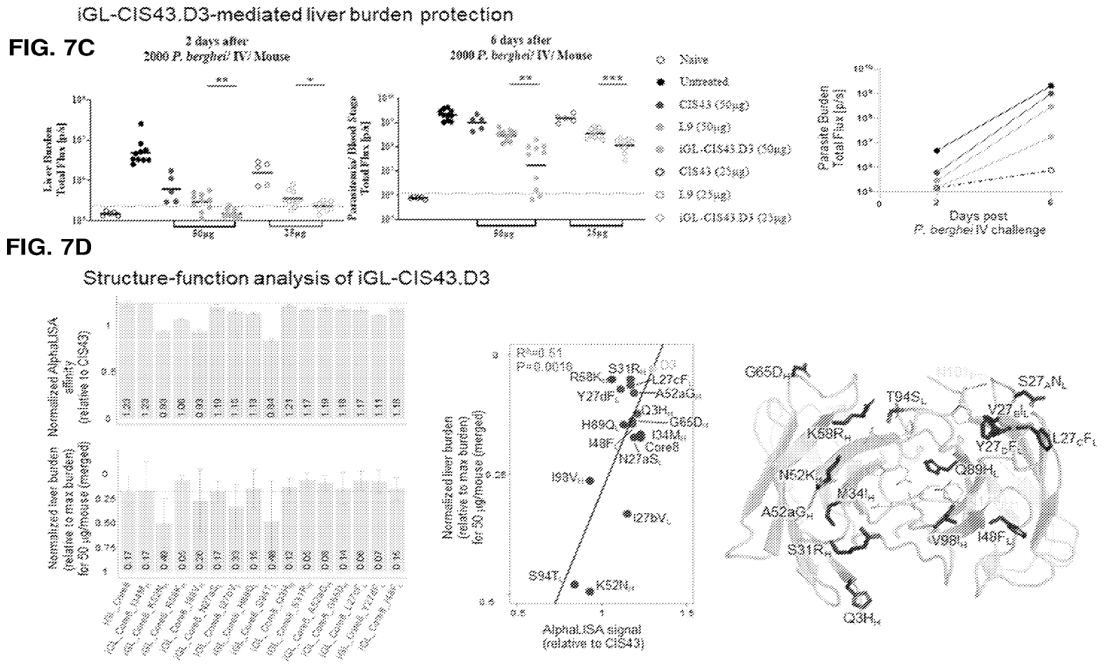

Several variant CIS43 antibodies were as potent for protection as antibody L9, a benchmark for the best-in-class malaria-protective antibody (Wang et al., 2020a). Thus, we sought to obtain a further improved CIS43 antibody, by exploiting the high correlation between protective function and both contact and total amino acids mutations. For contact mutations, we used an expanded definition of contact residues to include direct neighbors with an added requirement that these expanded contact mutations were observed in more than one mouse; we used this definition to sieve the database of CIS43-SHM obtained from adoptively transferred immunized mice to identify five potential beneficially mutations, S31R_{H} and L95M_{H} on the heavy chain, and L27cF_{L}, Y27dF_{L}, and Y92F_{L} on the light chain, and added these in various combinations to m43.151 (design D1-D6). For total mutations, we identified amino acid mutations that occurred in the top clones, identifying eight mutations on heavy chain and 11 mutations on light chain, and added these to m43.151 (design D7-D11) (FIG. 7A).

The VH and VL sequences of the D1-D11 antibodies are provided below:

```
>D1
(SEQ ID NO: 53)
VH: QVHLVQSGAEVKKPGASVKVSCKASGYTFTSYAIHWVRQAPGQRLE
        WMGWIKGGNGNTRYSQKFQDRVTITRDTSASTAYMELSSLRSEDTA
        VYYCALmTVITPDDAFDIWGQGTMVTVSS
(SEQ ID NO: 54)
VL: DIVMTQSPDSLAVSLGERATINCKSSQNIffSSNNKNYLAWYQQKP
        GQPPKLLFYWASTRESGVPDRFSGSGSGTDFTLTISSLQAEDVAVY
        YCHQYYSSPLTFGGGTKVEIK
>D2
(SEQ ID NO: 55)
VH: QVHLVQSGAEVKKPGASVKVSCKASGYTFTSYAIHWVRQAPGQRLE
        WMGWIKGGNGNTRYSQKFQDRVTITRDTSASTAYMELSSLRSEDTA
        VYYCALmTVITPDDAFDIWGQGTMVTVSS
(SEQ ID NO: 56)
VL: DIVMTQSPDSLAVSLGERATINCKSSQNIfYSSNNKNYLAWYQQKP
        GQPPKLLFYWASTRESGVPDRFSGSGSGTDFTLTISSLQAEDVAVY
        YCHQYYSSPLTFGGGTKVEIK
>D3
(SEQ ID NO: 11)
VH: QVHLVQSGAEVKKPGASVKVSCKASGYTFTrYAIHWVRQAPGQRLE
        WMGWIKGGNGNTRYSQKFQDRVTITRDTSASTAYMELSSLRSEDTA
        VYYCALLTVITPDDAFDIWGQGTMVTVSS
(SEQ ID NO: 12)
VL: DIVMTQSPDSLAVSLGERATINCKSSQNIffSSNNKNYLAWYQQKP
        GQPPKLLFYWASTRESGVPDRFSGSGSGTDFTLTISSLQAEDVAVY
        YCHQYYSSPLTFGGGTKVEIK
>D4
(SEQ ID NO: 239)
VH: QVHLVQSGAEVKKPGASVKVSCKASGYTFTrYAIHWVRQAPGQRLE
        WMGWIKGGNGNTRYSQKFQDRVTITRDTSASTAYMELSSLRSEDTA
        VYYCALLTVITPDDAFDIWGQGTMVTVSS
(SEQ ID NO: 240)
VL: DIVMTQSPDSLAVSLGERATINCKSSQNIffSSNNKNYLAWYQQKP
        GQPPKLLFYWASTRESGVPDRESGSGSGTDFTLTISSLQAEDVAVY
        YCHQYfSSPLTFGGGTKVEIK
>D5
(SEQ ID NO: 241)
VH: QVHLVQSGAEVKKPGASVKVSCKASGYTFTrYAIHWVRQAPGQRLE
        WMGWIKGGNGNTRYSQKFQDRVTITRDTSASTAYMELSSLRSEDTA
        SVYYCALmTVITPDDAFDIWGQGTMVTVS
(SEQ ID NO: 242)
VL: DIVMTQSPDSLAVSLGERATINCKSSQNIffSSNNKNYLAWYQQKP
        GQPPKLLFYWASTRESGVPDRESGSGSGTDFTLTISSLQAEDVAVY
        YCHQYfSSPLTFGGGTKVEIK
>D6
(SEQ ID NO: 243)
VH: QVHLVQSGAEVKKPGASVKVSCKASGYTFTrYAIHWVRQAPGQRLE
        WMGWIKGGNGNTRYSQKFQDRVTITRDTSASTAYMELSSLRSEDTA
        VYYCALmTVITPDDAFDIWGQGTMVTVSS
(SEQ ID NO: 244)
VL: DIVMTQSPDSLAVSLGERATINCKSSQNIfYSSNNKNYLAWYQQKP
        GQPPKLLFYWASTRESGVPDRFSGSGSGTDFTLTISSLQAEDVAVY
        YCHQYfSSPLTFGGGTKVEIK
>D7
(SEQ ID NO: 245)
VH: QVHLVQSGAEVKKPGASVKVSCKASGYTFTrYAIHWVRQAPGQRLE
        WMGWIKGGNGNTRYSQKFQDRVTITRDTSASTAYMELSSLRSEDTA
        VYYCALLTVITPDDAFDIWGQGTMVTVSS
(SEQ ID NO: 246)
VL: DIVMTQSPDSLAVSLGERATINCKSSQNIffSSNNKNYLAWYQQiP
        GQPPKLLFYWASTRESGVPDRFSGSGSGTDFTLTISSLQAEDVAVY
        YCHQYYSSPLTFGGGTKVEIq
>D8
(SEQ ID NO: 247)
VH: QVHLVQSGAEVKKPGASVKVSCKASGYTFTtYAIHWVRQAPGQRLE
        WMGWIKvGdGNTRYSpKFQDRVTITRDTSASTAYMELSSLRSEDTA
        VYfCALLTVITPDDAFDIWGQGTMVTVSS
(SEQ ID NO: 248)
VL: DIVMTQSPDSLAVSLGERATINCKSSONILYSSkNKNYLAWYQQKP
        GQsPKLLFYWASTRESGVPDRESGSGSGTDFTLTISSLQAEDVAVY
        YCHQYYSSPLTFGGGTKVEIK
>D9
(SEQ ID NO: 249)
VH: QVHLVQSGAEVKKPGASVKVSCKASGYTFTSYAIHWVRQAPGQRLE
        WMGWIKGGNGNTRYSQKFQDRVTITRDTSASTAYMELSSLRSEDTA
        VYYCALLTVITPDDtFDIWGQGTMVTVSS
(SEQ ID NO: 250)
VL: DIVMTQSPDSLAVSLGERATINCKSSQNILYSSNNKNYLAWYQQKP
        GQaPKLLFYWASTRESGVPDRESGSGSGTDFTLTISSLQAEDVAVY
        YCHQYYSSPLTFGGGTKVEIK
>D10
(SEQ ID NO: 251)
VH: QVHLVQSGAElKKPGASVKVSCKASGYTFTSYAIHWVRQAPGQRLE
        WMGWIKGGNGNTRYSQKFQDRVTITRDTSASTAYMELnSLRSEDTA
        VYYCALLTVITPDDAFDIWGQGTMVTVSS
(SEQ ID NO: 252)
VL: DIVMTQSPDSLAVSLGERATINCKSSQNILYSSNNKNYLAWYQQKP
        GQPPqLLFYWASTRESGVPDRFSGSGSGTDFTLTISSLQAEDVAVY
        YCHQYYSSPLTFGGGTKVEIK
>D11
(SEQ ID NO: 253)
VH: QVHLVQSGAEVKKPGASVKVSCKASGYTFTSYAIHWVRQAPGQRLE
        WMGWIKGGNGNTRYSQKFQDRVTITRDTSASTAYMELSSLRSEDTA
        gYYCALLTVITPDDAFDIWGQGTMVTVSS
(SEQ ID NO: 254)
VL: DIVMTQSPDSLAVSLGERAsINCKSSQNILfSSNNKNYLAWYQQKP
        GQPPKLLFYWASTRESGVPDRFSGSGSGTDFTLTISSLQAEDVAVY
        iCHQYYSSPLTFGGGTKVEIK
```

As the affinity for NPDP19 showed high correlation with function, we used this biochemical property to select which of the 11 antibodies to assess functionally. We used ALPHALISA® apparent affinity, measured in the context of full antibody, as it demonstrated higher correlation with function (R²=0.7626) than the Fab-derived BLI KD (R²=0.6779) (FIGS. 7B, left panels and 13A). Two design, iGL-CIS43.D1 and iGL-CIS43.D3 (also called D1 and D3), yielded the highest ALPHALISA® signals. The BLI-measured affinity for NPDP19 was 1.16 nM for D1 and 1.01 nM for D3, while ITC-measured affinity for PfCSPm was 4.7 nM for D1 and 1.2 nM for D3 (FIGS. 7B, right panels and 13B). Because of the virtually equivalent affinity of D1 and D3 for NPDP19 and the much higher affinity of D3 for PfCSPm, we chose to assess the ability of D3 to reduce parasite liver burden following passive transfer of 25 or of 50 μg/mouse (FIG. 7C). In both cases, D3 provided significantly reduced liver burden versus L9.

Structure-Function Assessment and Crystal Structure of iGL-CIS43.D3

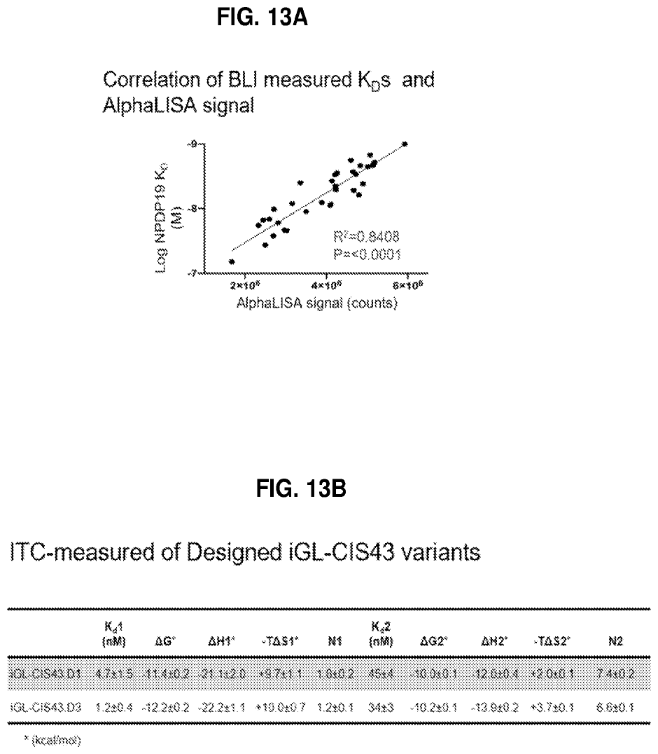

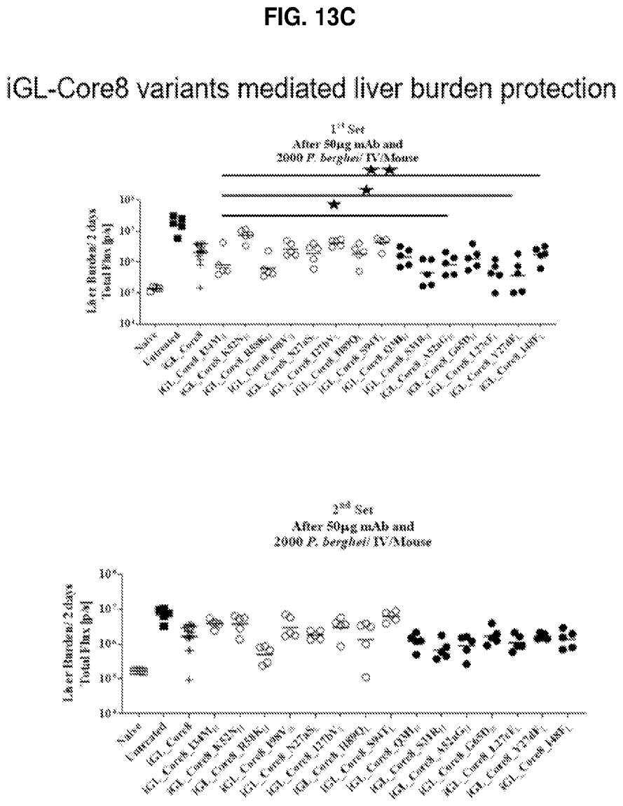

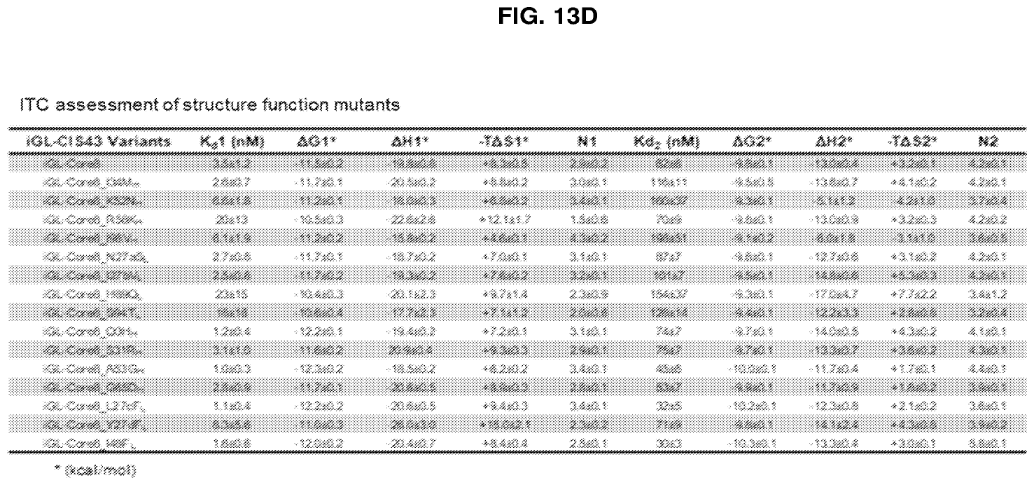

To provide insight into the specific alterations that improved iGL-CIS43.D3 function, we investigated the 15 amino acid alterations in D3 versus the inferred germline. We used Core8 mutations as a reference, either removing single changes from Core8, or adding single changes onto Core8 (FIG. 7D, left panel and FIGS. 13C-13D). Core8 showed an ALPHALISA®-measured apparent affinity that was 50 equivalent or higher than each of the mutated forms (though ˜10% lower than D3). Reversion of heavy chain I34M_{H}, R58K_{H}, or I98V_{H} or of light chain S94T_{L} significantly reduced affinity. In terms of protective function, the normalized liver burden was slightly less for Core8 versus the single added mutations from D3, and with the two most substantial reduction in protective efficacy (K52N_{H} and S94T_{L}) trending with reduced affinity. Each of the seven additional mutations showed affinities and protective function that were very similar to Core8, which were substantially below the affinity and protective function of D3, suggesting the seven additional mutations to act collectively to improve D3 affinity and function.

Overall, ALPHALISA®-measured apparent affinity correlated with reduction in liver burden (FIG. 7D, middle panel). We determined the crystal structure of D3 in complex with junctional peptide (FIG. 7D, right panel). Notably, half of the alterations found in D3 were distal from the junctional peptide, though still on the exposed portion of the Fab, suggesting the interactions between D3 and PfCSP to involve more than those observed with the junctional peptide; indeed, the three mutations, S31R_{H}, L27cF_{L}, and Y27dF_{L}, which improve D3 relative to m43.151, were each located on the face of the antibody that would be expected to contact PfCSP, but distal from bound peptide, consistent with D3 having ˜3-folder higher affinity for PfCSP, but similar junctional peptide affinity as m43.151.

### Discussion

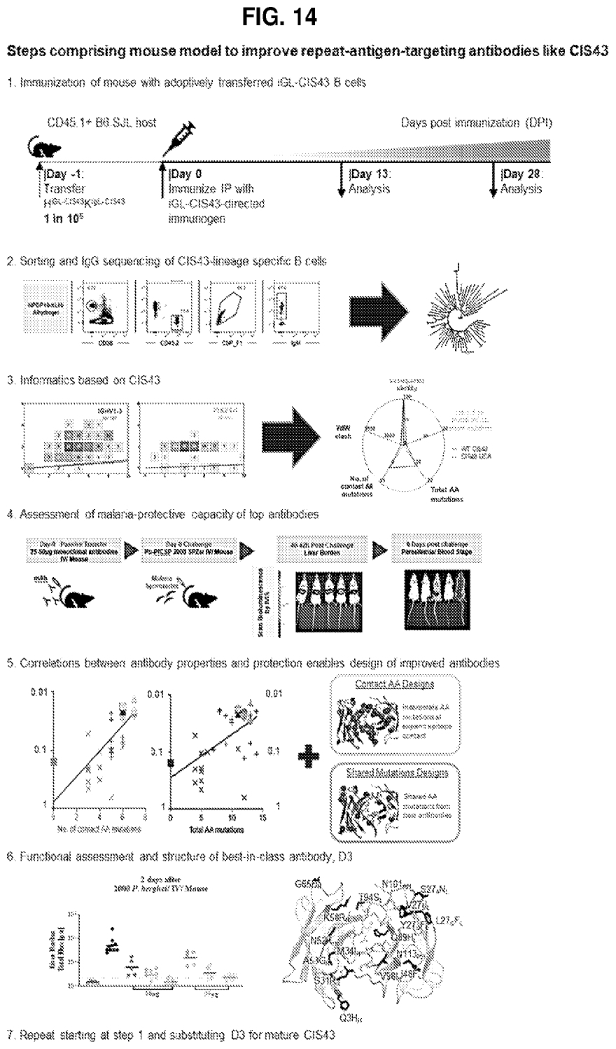

Repeat antigens like PfCSP confound the immune response. While antibodies such as CIS43 and L9, which target junctional or NVDP-minor repeat regions, show remarkable protective capability (Kisalu et al., 2018; Wang et al., 2020a), these antibodies both display low SHM (˜3% for CIS43 and ˜3% for L9)—unlike the highly evolved potent neutralizing antibodies that have been found against other pathogens, such as HIV-1 (Kwong and Mascola, 2018)—and there may be little correlation between SHM and immune protection for PfCSP-specific antibodies (Julien and Wardemann, 2019; Murugan et al., 2018). Moreover, potent antibodies elicited against the NANP-repeat region often exhibit homotypic Fab-Fab interactions (Imkeller et al., 2018; Oyen et al., 2020a; Pholcharee et al., 2021) with alterations in framework regions, which allow them to recognize an unusual, long-range, extended spiral conformation of the repeat region (Oyen et al., 2018). In general, the structural diversity and sequence degeneracy displayed by the repeat region has made it a challenge to improve the rare antibodies that are capable of recognizing PfCSP and imparting protection against malaria. Here, we provide a new mouse model to facility the development of improved repeat antigen-targeting antibodies. This model incorporates vaccination, isolation of elicited B cells, along with their sequencing and informatics to identify correlation between antibody features and improved protection (FIG. 14). The insights provided by the analysis have led not only to an improved CIS43 antibody that is significantly better than the current best-in-class, but to an understanding the mechanistic basis of improved malaria vaccination strategies.

A central finding of our study is the fact that epitope-based immunization induced CIS43 variants with greater potency than mature CIS43. This finding was established by correlating protective function with genetic properties, identifying contact amino acid changes and total SHM as correlating highly with protective function. Mutations defined by these genetic properties were further tested to identify D3, which we demonstrate to be significantly improved versus L9—the current best-in-class (Wang et al., 2020a). Anti-malarial antibodies with improved potency and duration have great promise for the prevention of malaria infection via passive transfer for a variety of clinical use cases which range from travelers to seasonal control and ultimately elimination.

### Tables

Appendix A of U.S. Provisional Application No. 63/275,936, filed Nov. 4, 2021, provides sequences of 161 CIS43 variants, 114 from 13 DPI and 47 from 28 DPI, related to FIG. 5. Appendix A is incorporated by reference herein in its entirety.

TABLE 2
Production yields of 37 ‘top 10’-selected antibodies, related to FIG. 5.

| Antibody | mg/L |
|---|---|
| m30.014 | 50 |
| m31.033 | 25 |
| m31.056 | 80 |
| m32.069 | 50 |
| m32.085 | 35 |
| m33.088 | 50 |
| m33.089 | 30 |
| m33.096 | 40 |
| m33.104 | 55 |
| m33.111 | 25 |
| m33.112 | 40 |
| m42.119 | 90 |
| m42.120 | 20 |
| m42.124 | 55 |
| m42.125 | 25 |
| m42.126 | 105 |
| m42.127 | 105 |
| m42.130 | 135 |
| m42.131 | 50 |
| m42.133 | 40 |
| m42.134 | 60 |
| m43.135 | 45 |
| m43.137 | 25 |
| m43.138 | 55 |
| m43.140 | NE |
| m43.141 | 70 |
| m43.143 | NE |
| m43.144 | 110 |
| m43.145 | 80 |
| m43.146 | NE |
| m43.147 | 25 |
| m43.149 | 50 |
| m43.151 | 100 |
| m43.157 | 125 |
| m43.159 | 40 |
| m43.160 | 40 |
| m43.161 | 55 |

TABLE 3A
Normalize liver burden (relative to max burden) for 50 mg/mouse.
Values from the first and the second experiment are indicated.

| | | | | | | | | | |
|---|---|---|---|---|---|---|---|---|---|
| CIS43 | L9 | m43.149 | m42.126 | m43.138 | m42.127 | m43.151 | m43.159 | m43.160 | Untreated |

First Experiment

| | | | | | | | | | |
|---|---|---|---|---|---|---|---|---|---|
| 0.087 | 0.058 | 0.017 | 0.050 | 0.011 | 0.013 | 0.010 | 0.036 | 0.015 | 1.117 |
| 0.011 | 0.046 | 0.042 | 0.026 | 0.024 | 0.029 | 0.021 | 0.046 | 0.023 | 1.663 |
| 0.108 | 0.064 | 0.049 | 0.012 | 0.023 | 0.050 | 0.024 | 0.021 | 0.061 | 1.102 |
| 0.108 | 0.054 | 0.075 | 0.020 | 0.052 | 0.030 | 0.026 | 0.037 | 0.021 | 0.740 |
| 0.216 | 0.037 | 0.038 | 0.026 | 0.049 | 0.018 | 0.016 | 0.018 | 0.017 | 0.661 |

Second Experiment

| | | | | | | | | | |
|---|---|---|---|---|---|---|---|---|---|
| 0.180 | 0.105 | 0.082 | 0.034 | 0.075 | 0.015 | 0.084 | 0.092 | 0.054 | 0.822 |
| 0.217 | 0.052 | 0.137 | 0.040 | 0.034 | 0.028 | 0.083 | 0.029 | 0.035 | 1.164 |
| 0.326 | 0.058 | 0.037 | 0.051 | 0.081 | 0.060 | 0.023 | 0.031 | 0.066 | 1.500 |
| 0.333 | 0.060 | 0.053 | 0.075 | 0.083 | 0.045 | 0.045 | 0.109 | 0.033 | 1.226 |
| 0.080 | 0.023 | 0.057 | 0.066 | 0.107 | 0.031 | 0.036 | 0.057 | 0.034 | 1.245 |
| | | 0.035 | 0.057 | 0.080 | 0.061 | 0.107 | 0.059 | 0.088 | 0.620 |
| | | 0.067 | 0.101 | 0.121 | 0.060 | 0.049 | 0.068 | 0.064 | 0.732 |
| | | 0.084 | 0.054 | 0.036 | 0.073 | 0.108 | 0.062 | 0.044 | 1.273 |
| | | 0.046 | 0.094 | 0.054 | 0.066 | 0.045 | 0.083 | 0.026 | 0.928 |
| | | 0.105 | 0.086 | 0.132 | 0.055 | 0.045 | 0.058 | 0.087 | 0.852 |

TABLE 3B
P-values between each CIS43 variants and wild-type CIS43 as calculated by
ordinary one-way ANOVA test based on the log of the values in Table S3A.

| | m43.149 | m42.126 | m43.138 | m42.127 | m43.151 | m43.159 | m43.160 |
|---|---|---|---|---|---|---|---|
| P-values | 0.0889 | 0.0312 | 0.0474 | 0.0158 | 0.0159 | 0.073 | 0.0101 |

TABLE 4
BLI-measured affinities of ‘top 10’-selected antibodies, related to FIG. 6.

| Antibody name | PfCSP KD (M) | Error (M) | PfCSP KD2 (M) | Error (M) | NANP KD (M) | Error (M) |
|---|---|---|---|---|---|---|
| m30.014 | 2.22E−07 | 2.64E−09 | 1.61E−06 | 4.22E−08 | 5.46E−07 | 2.40E−08 |
| m31.033 | 1.97E−07 | 2.13E−09 | 1.63E−06 | 4.20E−08 | 4.16E−07 | 1.74E−08 |
| m31.056 | 2.41E−07 | 7.85E−09 | 9.44E−07 | 3.39E−08 | 2.93E−07 | 9.45E−09 |
| m32.069 | 1.80E−06 | 4.36E−08 | 1.95E−07 | 2.60E−09 | 4.69E−07 | 2.12E−08 |
| m32.085 | 5.51E−07 | 9.53E−09 | 2.16E−06 | 4.82E−08 | 1.00E−06 | 5.98E−08 |
| m33.088 | 3.09E−07 | 3.43E−09 | 1.82E−06 | 3.67E−08 | 6.81E−07 | 3.12E−08 |
| m33.089 | 2.56E−07 | 3.34E−09 | 1.34E−06 | 2.99E−08 | 5.88E−07 | 2.74E−08 |
| m33.096 | 1.84E−06 | 3.91E−08 | 2.39E−07 | 2.56E−09 | 5.38E−07 | 2.51E−08 |
| m33.104 | 3.88E−07 | 4.03E−09 | 2.74E−06 | 7.45E−08 | 5.99E−07 | 2.59E−08 |
| m33.111 | 3.08E−07 | 2.88E−09 | 3.86E−06 | 1.45E−07 | 6.45E−07 | 2.59E−08 |
| m33.112 | 3.09E−07 | 3.21E−09 | 1.99E−06 | 4.08E−08 | 6.32E−07 | 2.72E−08 |
| m43.149 | 1.30E−07 | 1.64E−09 | 1.26E−06 | 3.52E−08 | 4.75E−07 | 1.74E−08 |
| m42.119 | 4.69E−07 | 3.87E−09 | 9.86E−07 | 4.42E−08 | 2.91E−07 | 2.09E−08 |
| m42.120 | 1.83E−07 | 1.97E−09 | 8.24E−06 | 1.03E−06 | 4.23E−07 | 1.44E−08 |
| m42.124 | 2.26E−07 | 1.78E−09 | 2.42E−06 | 6.46E−08 | 4.92E−07 | 1.96E−08 |
| m42.125 | 1.17E−07 | 1.56E−09 | 9.91E−06 | 2.48E−06 | 4.36E−07 | 1.26E−08 |
| m42.126 | 1.22E−07 | 2.69E−09 | 5.44E−07 | 2.03E−08 | 4.25E−07 | 1.16E−08 |
| m42.127 | 3.16E−08 | 6.26E−10 | 1.98E−06 | 1.05E−07 | 9.52E−07 | 3.10E−08 |
| m42.130 | 9.63E−07 | 2.97E−09 | 1.40E−06 | 4.58E−08 | 5.90E−07 | 4.06E−08 |
| m42.131 | 1.67E−07 | 1.03E−09 | 3.57E−06 | 1.43E−07 | 4.71E−07 | 1.55E−08 |
| m42.133 | 1.92E−07 | 1.14E−09 | 4.31E−06 | 1.96E−07 | 4.96E−07 | 1.56E−08 |
| m42.134 | 2.33E−07 | 1.78E−09 | 2.15E−06 | 5.76E−08 | 5.01E−07 | 1.79E−08 |
| m43.135 | 1.45E−07 | 1.72E−09 | 1.26E−06 | 3.14E−08 | 3.72E−07 | 1.36E−08 |
| m43.137 | 5.33E−07 | 2.99E−09 | 1.19E−06 | 4.49E−08 | 6.63E−07 | 5.24E−08 |
| m43.138 | 4.90E−08 | 5.47E−10 | 1.70E−06 | 5.10E−08 | 9.88E−07 | 3.30E−08 |
| m43.141 | 6.81E−08 | 1.29E−09 | 6.26E−07 | 1.97E−08 | 9.58E−07 | 3.51E−08 |
| m43.144 | 2.57E−07 | 2.21E−09 | 8.13E−07 | 1.96E−08 | 5.71E−07 | 2.07E−08 |
| m43.145 | 4.29E−08 | 5.82E−10 | 2.54E−06 | 1.06E−07 | 9.67E−07 | 2.36E−08 |
| m43.147 | 3.24E−07 | 3.09E−09 | 1.35E−06 | 2.75E−08 | 7.06E−07 | 3.40E−08 |
| m43.151 | 3.83E−08 | 6.85E−10 | 1.70E−06 | 6.60E−08 | 7.15E−07 | 1.82E−08 |
| m43.157 | 6.88E−07 | 4.07E−09 | 1.15E−06 | 3.54E−08 | 5.79E−07 | 2.76E−08 |
| m43.159 | 4.12E−08 | 6.74E−10 | 1.43E−06 | 4.51E−08 | 8.43E−07 | 2.21E−08 |
| m43.160 | 3.68E−08 | 6.54E−10 | 1.82E−06 | 7.42E−08 | 9.57E−07 | 2.46E−08 |
| m43.161 | 5.05E−08 | 5.71E−10 | 1.48E−06 | 4.19E−08 | 1.13E−06 | 3.58E−08 |
| CIS43 mature | 4.52E−08 | 7.54E−10 | 1.33E−06 | 4.31E−08 | 4.11E−07 | 1.32E−08 |
| CIS43 iGL | 6.79E−07 | 7.30E−08 | 5.05E−06 | 4.72E−07 | 9.65E−06 | 5.14E−06 |

| Antibody name | NPDP19 KD (M) | Error (M) | P21 KD (M) | Error (M) |
|---|---|---|---|---|
| m30.014 | 1.02E−08 | 4.36E−10 | 2.27E−07 | 3.09E−09 |
| m31.033 | 6.62E−08 | 2.18E−09 | 1.90E−07 | 2.18E−09 |
| m31.056 | 2.20E−08 | 1.12E−09 | 2.90E−07 | 1.06E−08 |
| m32.069 | 2.15E−08 | 9.40E−10 | 1.01E−07 | 1.71E−09 |
| m32.085 | 1.09E−07 | 2.76E−09 | 6.23E−07 | 1.19E−08 |
| m33.088 | 3.70E−08 | 9.54E−10 | 3.13E−07 | 5.06E−09 |
| m33.089 | 2.65E−08 | 1.36E−09 | 2.38E−07 | 3.56E−09 |
| m33.096 | 1.51E−08 | 4.08E−10 | 3.01E−07 | 4.00E−09 |
| m33.104 | 1.46E−08 | 3.93E−10 | 5.44E−07 | 1.02E−08 |
| m33.111 | 1.65E−08 | 7.89E−10 | 3.47E−07 | 5.47E−09 |
| m33.112 | 1.83E−08 | 5.06E−10 | 3.08E−07 | 4.47E−09 |
| m43.149 | 1.50E−09 | 1.78E−10 | 4.95E−08 | 8.88E−10 |
| m42.119 | 3.75E−09 | 2.50E−10 | 1.65E−07 | 1.58E−09 |
| m42.120 | 5.10E−09 | 2.11E−10 | 1.22E−07 | 1.55E−09 |
| m42.124 | 4.01E−09 | 2.81E−10 | 1.45E−07 | 1.78E−09 |
| m42.125 | 3.00E−09 | 1.44E−10 | 8.55E−08 | 1.08E−09 |
| m42.126 | 4.13E−09 | 4.59E−11 | 3.73E−08 | 6.41E−10 |
| m42.127 | 2.18E−09 | 1.89E−10 | 3.02E−08 | 1.92E−10 |
| m42.130 | 8.93E−09 | 2.51E−10 | 1.22E−07 | 9.21E−10 |
| m42.131 | 8.46E−09 | 2.69E−10 | 1.90E−07 | 2.88E−09 |
| m42.133 | 2.82E−09 | 1.44E−10 | 1.09E−07 | 1.46E−09 |
| m42.134 | 8.07E−09 | 3.24E−10 | 6.96E−08 | 1.08E−09 |
| m43.135 | 4.50E−09 | 2.08E−10 | 5.26E−08 | 6.09E−10 |
| m43.137 | 2.97E−09 | 1.04E−10 | 6.87E−08 | 8.31E−10 |
| m43.138 | 2.26E−09 | 1.38E−10 | 6.05E−08 | 2.37E−10 |
| m43.141 | 8.06E−09 | 3.07E−10 | 8.15E−08 | 3.47E−10 |
| m43.144 | 6.11E−09 | 2.61E−10 | 5.23E−08 | 8.57E−10 |
| m43.145 | 5.26E−09 | 2.62E−10 | 5.92E−08 | 2.56E−10 |
| m43.147 | 1.11E−08 | 3.73E−10 | 8.74E−08 | 1.20E−09 |
| m43.151 | 2.16E−09 | 2.00E−10 | 2.12E−08 | 1.85E−10 |
| m43.157 | 2.71E−09 | 1.37E−10 | 5.68E−08 | 6.22E−10 |
| m43.159 | 2.01E−09 | 1.83E−10 | 2.93E−08 | 1.93E−10 |
| m43.160 | 1.94E−09 | 1.96E−10 | 3.03E−08 | 2.19E−10 |
| m43.161 | 1.81E−09 | 1.33E−10 | 6.26E−08 | 2.47E−10 |
| CIS43 mature | 8.58E−09 | 2.79E−10 | 6.14E−08 | 2.87E−10 |
| CIS43 iGL | 2.72E−08 | 1.40E−07 | 6.35E−07 | 4.87E−08 |

TABLE 5
Crystallographic data collection and refinement statistics, related to FIGS. 6 and 7.

| | m42.126: P21 | m42.127: P21 | M43.138: P21 | m43.149: P21 |
|---|---|---|---|---|
| PDB accession code | 7RD3 | 7LKB | 7RDA | 7RD4 |
| Data collection | | | | |
| Wavelength (Å) | 1 | 1 | 1 | 1 |
| Resolution range (Å) | 50-1.81 | 50-1.80 | 50-1.92 | 50-1.75 |
| | (1.84-1.81) | (1.83-1.80) | (1.95-1.92) | (1.78-1.75) |
| Space group | P 1 | P 1 | P 2₁2₁2₁ | P 1 |
| Cell dimensions | | | | |
| a, b, c (Å) | 54.9 57.2 74.5 | 54.9 58.1 75.2 | 60.5 79.3 90.9 | 54.8 55.1 74.4 |
| α, β, γ (°) | 87 77 72 | 87, 78, 72 | 90, 90, 90 | 78 79 70 |
| Unique reflections | 66229 (3262) | 70885 (3605) | 33841 (1669) | 67330 (3290) |
| Multiplicity | 3.0 (2.9) | 2.1 (2.1) | 7.0 (6.5) | 1.9 (1.8) |
| Completeness (%) | 86.3 (84.8) | 89.2 (90.3) | 99.4 (99.5) | 84.4 (82.2) |
| I/σI | 18.7 (2.0) | 20.8 (2.1) | 25.4 (2.9) | 13.8 (2.7) |
| Wilson B-factor | 23.84 | 22.59 | 25.87 | 12.74 |
| R_{merge} | 0.068 (0.430) | 0.041 (0.311) | 0.106 (0.564) | 0.062 (0.230) |
| CC_{1/2} | 0.999 (0.876) | 0.998 (0.843) | 0.992 (0.893) | 0.992 (0.862) |
| Refinement | | | | |
| Reflections used in | 66066 (6640) | 70883 (7030) | 33764 (3239) | 67314 (6668) |
| refinement | | | | |
| Reflections used for R-free | 3240 (308) | 3543 (319) | 1666 (171) | 3369 (345) |
| R_{work} | 0.23 (0.29) | 0.18 (0.24) | 0.20 (0.26) | 0.19 (0.23) |
| R_{free} | 0.26 (0.36) | 0.21 (0.28) | 0.23 (0.33) | 0.23 (0.30) |
| Number of non-H atoms | 7403 | 7618 | 3671 | 7739 |
| macromolecules | 6850 | 6952 | 3430 | 6879 |
| ligands | | 5 | | |
| solvent | 553 | 661 | 241 | 860 |
| Protein residues | 898 | 912 | 452 | 902 |
| RMS(bonds) (Å) | 0.004 | 0.008 | 0.02 | 0.007 |
| RMS(angles) (°) | 0.82 | 1.17 | 1.75 | 1.02 |
| Ramachandran favored (%) | 98.3 | 98.78 | 97.53 | 98.2 |
| Ramachandran outliers (%) | 0 | 0 | 0 | 0 |
| Average B-factor (Å²) | 34.41 | 26.75 | 31.47 | 18.2 |
| macromolecules | 34.05 | 26.03 | 31.14 | 17.1 |
| ligands | | 30 | | |
| solvent | 38.83 | 34.29 | 36.24 | 26.99 |
| | | | | iGL-CIS43.D3: |
| | m43.151: P21 | M43.159: P21 | m43.160: P21 | P21 |
| PDB accession code | 7LKG | 7RD9 | 7RCS | 7RAJ |
| Data collection | | | | |
| Wavelength (Å) | 1 | 1 | 1 | 1 |
| Resolution range (Å) | 50-2.05 | 50-1.91 | 50-2.40 | 34-3.0 |
| | (2.09-2.05) | (1.94-1.91) | (2.44-2.40) | (3.10-3.00) |
| Space group | P 1 | C 2 | P 1 | P 2₁ |
| Cell dimensions | | | | |
| a, b, c (Å) | 54.4 54.2 75.7 | 93.9 62.0 75.1 | 54.8 57.5 74.9 | 37.3 89.7 72.0 |
| α, β, γ (°) | 78, 77, 68 | 90, 106 90 | 86 77 73 | 90, 99, 90 |
| Unique reflections | 43488 (2295) | 30463 (1501) | 27527 (1013) | 9207 (859) |
| Multiplicity | 2.5 (2.4) | 4.7 (4.4) | 2.0 (1.7) | 6.8 (6.6) |
| Completeness (%) | 87.8 (90.6) | 94.1 (92.1) | 82.7 (61.5) | 97.2 (88.6) |
| I/σI | 13.1 (2.0) | 17.8 (3.3) | 13.6 (2.0) | 8.3 (1.6) |
| Wilson B-factor | 26.9 | 16.72 | 38.7 | 62.07 |
| R_{merge} | 0.134 (0.480) | 0.177 (0.555) | 0.055 (0.200) | 0.188 (1.181) |
| CC_{1/2} | 0.981 (0.647) | 0.981 (0.783) | 0.995 (0.921) | 0.992 (0.717) |
| Refinement | | | | |
| Reflections used in | 43460 (3978) | 30453 (3078) | 27483 (2120) | 9195 (859) |
| refinement | | | | |
| Reflections used for R-free | 2034 (177) | 1519 (172) | 1351 (93) | 471 (47) |
| R_{work} | 0.21 (0.26) | 0.19 (0.21) | 0.19 (0.28) | 0.25 (0.35) |
| R_{free} | 0.25 (0.32) | 0.23 (0.27) | 0.25 (0.34) | 0.31 (0.39) |
| Number of non-H atoms | 7340 | 3902 | 7019 | 3463 |
| macromolecules | 6938 | 3436 | 6843 | 3447 |
| ligands | | | | 14 |
| solvent | 402 | 466 | 176 | 2 |
| Protein residues | 910 | 452 | 902 | 451 |
| RMS(bonds) (Å) | 0.006 | 0.003 | 0.009 | 0.006 |
| RMS(angles) (°) | 0.99 | 0.72 | 1.04 | 1.07 |
| Ramachandran favored (%) | 97.1 | 98.21 | 96.85 | 95.51 |
| Ramachandran outliers (%) | 0.11 | 0 | 0 | 0 |
| Average B-factor (Å²) | 32.96 | 21.28 | 42.41 | 58.68 |
| macromolecules | 32.78 | 20.05 | 42.46 | 58.68 |
| ligands | | | | 59.91 |
| solvent | 36.08 | 30.31 | 40.25 | 55.22 |

TABLE 6
Sequence feature analysis related to restricted set of 8 SHM, observed in a majority of the improved
antibodies, with respective to peptide 21 and PfCSP binding, and reduction of liver burden, related to FIG. 6.
Peptide 21

| Position | Amino acid | P-value | Odds ratio* | Adjusted P-value |
|---|---|---|---|---|
| HC34 | Ile | 0.0491 | 0.15 | 0.2457 |
| HC52 | Lys | 0.0035 | 0.06 | 0.0247 |
| HC58 | Arg | 1.6e−05 | 0.02 | 0.0001 |
| HC98 | Ile | 1 | 0.78 | 1 |
| LC27a | Asn | 0.0527 | 0 | 0.2457 |
| LC27b | Ile | 0.0219 | 0.09 | 0.1311 |
| LC89 | His | 0.1493 | 0.17 | 0.4478 |
| LC94 | Ser | 1 | 0.80 | 1 |

PfCSP binding

| | | | | |
|---|---|---|---|---|
| HC34 | Ile | 0.2449 | 0.28 | 1 |
| HC52 | Lys | 7.12E−05 | 0.03 | 0.0006 |
| HC58 | Arg | 0.0140 | 0.12 | 0.0837 |
| HC98 | Ile | 1 | 1.28 | 1 |
| LC27a | Asn | 0.0116 | 0 | 0.0811 |
| LC27b | Ile | 0.2853 | 0.43 | 1 |
| LC89 | His | 0.3549 | 0.28 | 1 |
| LC94 | Ser | 1 | 1.26 | 1 |

Reduction of liver burden

| Position | Amino acid | P-value | Odds ratio** | Adjusted P-value |
|---|---|---|---|---|
| HC34 | Ile | 0.2553 | 0.35 | 1 |
| HC52 | Lys | 4.52E−05 | 0 | 0.0004 |
| HC58 | Arg | 0.0014 | 0.07 | 0.0095 |
| HC98 | Ile | 0.3255 | 0.27 | 1 |
| LC27a | Asn | 0.0473 | 0 | 0.2836 |
| LC27b | Ile | 0.2742 | 0.38 | 1 |
| LC89 | His | 0.1672 | 0.19 | 0.8360 |
| LC94 | Ser | 1 | 0.89 | 1 |

*Odds ratio >1 => Associated with lower binding
Odds ratio <1 => Associated with higher binding
**Odds ratio >1 => Associated with higher liver burden
Odds ratio <1 => Associated with lower liver burden

TABLE 7
Resources

| | | |
|---|---|---|
| REAGENT or RESOURCE | SOURCE | IDENTIFIER |

Antibodies

| | | |
|---|---|---|
| Rat monoclonal anti-mouse-CD16/32 purified (clone 2.4G2) | BD Biosciences | Cat#: 553142 |
| Rat monoclonal anti-mouse CD4 APC-eF780 (clone: RM4-5) | Invitrogen | CAT#: 47-0042-80 |
| Rat monoclonal anti-mouse CD8 APC-eF780 (clone: 53-6.7) | Invitrogen | CAT#: 47-0081-80 |
| Rat monoclonal anti-mouse F4/80 APC-eF780 (clone: BM8) | Invitrogen | CAT#: 47-4801-80 |
| Rat monoclonal anti-mouse Ly-6G APC-eF780 (clone: RB6-8C5) | Invitrogen | CAT#: 47-5931-80 |
| Rat monoclonal anti-mouse B220 BV510 (clone: RA3-6B2) | BD Biosciences | CAT#: 563103 |
| Hamster monoclonal anti-mouse CD95 PE-Cy7 (clone: Jo2) | BD Biosciences | CAT#: 557653 |
| Rat monoclonal anti-mouse CD38 A700 or A488 (clone: 90) | Invitrogen, | CAT#: 56-0381-82, 102714 |
| | Biolegend | |
| Mouse monoclonal anti-mouse CD45.2 PE (clone: 104) | Biolegend | CAT#: 109808 |
| Mouse monoclonal anti-mouse CD45.1 PerCP Cy5.5 (clone: | Biolegend | CAT#: 110728 |
| A20) | | |
| Rat monoclonal anti-mouse GL7 A647 (clone: GL7) | Biolegend | CAT#: 144606 |
| Rat monoclonal anti-mouse IgM BUV395 or BV421 (clone: | BD Biosciences | CAT#: 743329, 743323 |
| II/41) | | |
| Rat monoclonal anti-mouse IgD BV786 (clone: 11-26c.2a) | BD Biosciences | CAT#: 563618 |
| Rat monoclonal anti-mouse IgD PE-Cy7 (clone: 11-26c.2a) | Biolegend | CAT#: 405720 |
| Rat monoclonal anti-mouse IgG1 BV421 (clone: A85-1) | BD Biosciences | CAT#: 562580 |
| Rat monoclonal anti-mouse Ig, κ light chain BUV395 (clone: | BD Biosciences | CAT#: 742839 |
| 187.1) | | |
| Goat Anti-Mouse IgG Fcy ALP | Jackson Immuno | CAT#: 115-055-071 |
| | Research | |

Chemicals, Peptides, and Recombinant Proteins

| | | |
|---|---|---|
| Recombinant PfCSP | Produced in house | N/A |
| Biotinylated recombinant PfCSP | Produced in house | N/A |
| Biotinylated junctional peptide NPDP19 | Genscript | Order ID: # U134AFB120 |
| LIVE/DEAD ™ Fixable Blue Dead Cell Stain Kit, for UV | Thermo Fisher | Cat#: L34962 |
| excitation | Scientific | |
| Streptavidin-A488 | Biolegend | CAT#: 405235 |
| Streptavidin-647 | Biolegend | CAT#: 405237 |
| Streptavidin-PE | Biolegend | CAT#: 405204 |
| BD HORIZON BRILLIANT STAIN BUFFER | BD Biosciences | CAT#: 566349 |
| SIGMAFAST ™ p-Nitrophenyl phosphate Tablets | Sigma | CAT#: N2770-50SET |
| SuperScript ™ III Reverse Transcriptase | Thermo Fisher | CAT#: 18080085 |
| | Scientific | |
| RNasin ® Ribonuclease Inhibitors | Promega | CAT#: N2515 |
| dNTP Mix (10 mM each) | Thermo Fisher | CAT#: R0193 |
| | Scientific | |
| HotStarTaq DNA Polymerase | QIAGEN | CAT#: 203209 |

Critical Commercial Assays

| | | |
|---|---|---|
| CountBright ™ Absolute Counting Beads, for flow cytometry | Thermo Fisher | CAT#: C36950 |
| | Scientific | |
| UltraComp eBeads ™ Compensation Beads | Thermo Fisher | CAT#: 01-2222-42 |
| | Scientific | |
| Pan B Cell Isolation Kit II, mouse | Miltenyi Biotec | CAT#: 130-104-443 |

Deposited Data

| | | |
|---|---|---|
| PDB file | This paper. | 7LKB |
| PDB file | This paper. | 7LKG |

Experimental Models: Cell Lines

| | | |
|---|---|---|
| Human: Expi293 cell | Thermo Fisher | Cat#A14527 |
| | Scientific | |

Experimental Models: Organisms/Strains

| | | |
|---|---|---|
| Mouse: B6.SJL-Ptprcapepcb/BoyJ | The Jackson | JAX: 002014 |
| | Laboratory | |
| Mouse: B6(Cg)-Tyrc-2J/J albino | The Jackson | JAX: 000058 |
| | Laboratory | |
| Mouse: C57BL/6J | The Jackson | JAX: 000664 |
| | Laboratory. | |
| Mouse: H^{IGL-CIS43} | This paper | N/A |
| Mouse: K^{IGL-CIS43} | This paper | N/A |
| Mouse: H^{iGL-CIS43}K^{iGL-CIS43} | This paper | N/A |
| Sporozoite: P. berghei sporozoite expressing PfCSP, GFP, and | (Flores-Garcia et | N/A |
| luciferase | al., 2019b) | |

Oligonucleotides and/or other sequence-based reagents

| | | |
|---|---|---|
| sgRNA | (Lin et al., 2018b; | N/A |
| | Wang et al., | |
| | 2020b)(Lin et al., | |
| | 2018b; Wang et | |
| | al., 2020b) | |

Recombinant DNA

| | | |
|---|---|---|
| Plasmodium falciparum circumsporozoite protein (clone 3D7) | PlasmoDB | PF3D7_0304600.1 |
| pVRC8400 hulgG1 | Genscript | N/A |
| pVRC8400 hulgK | Genscript | N/A |

Software and Algorithms

| | | |
|---|---|---|
| FlowJo 10.7.1 | Treestar | flowjo.com/ |
| Prism 9.0.1 | GraphPad | graphpad.com/ |
| Microsoft Office | Microsoft | office.com/ |
| IMGT/V-quest | | imgt.org/IMGTindex/V- |
| | | QUEST.php |
| Geneious 2020.2 | Biomatters | geneious.com |
| The PyMOL Molecular Graphics System | | pymol.org |
| RosettaRemodel | Rosetta | rosettacommons.org/ |
| | | docs/latest/application_ |
| | | documentation/design/ |
| | | Remodel |
| foldx5Linux64 | FoldX | foldxsuite.crg.eu/ |
| YASARA | YASARA | yasara.org/ |
| NAMD_2.14_Linux-x86_64-multicore | NAMD | ks.uiuc.edu/Development/ |
| | | Download/download.cgi |
| | | ?PackageName=NAMD |
| gRINN v1.1.0.hf1 | gRINN | grinn.readthedocs.io/en/ |
| | | latest/index.html |
| Gene-specific substitution profile | | cab- |
| | | rep.c2b2.columbia.edu/ |

Other

| | | |
|---|---|---|
| Armadillo PCR Plate, 96-well, clear, clear wells | Thermo Scientific | CAT#: AB2396 |
| Eppendorf twin.tec PCR Plate 96, semi-skirted, yellow | Genesee/ | CAT#: 951020320 |
| | Eppendorf | |
| Eppendorf twin.tec ® PCR Plate 96, skirted, clear wells, blue | Genesee/ | CAT#: 951020460 |
| | Eppendorf | |
| Adhesive Sealing Sheets | Thermo Scientific | CAT#: AB-0558 |
| Microseal ® ′F′ PCR Plate Seal, foil, pierceable #msf1001 | Bio-Rad | CAT#: MSF1001 |
| E-Gel 96 2% with SYBR Safe | Thermo Fisher | CAT#: G720802 |
| | Scientific | |
| E-Gel 96 Low Range Quantitative DNA Ladder | Thermo Fisher | CAT#: 12373-031 |
| | Scientific | |
| BD FACSymphony ™ | BD Biosciences | N/A |
| BD FACS Aria II cell sorter | BD Biosciences | N/A |
| VP-ITC microcalorimeter | Malvern | N/A |
| | Panalytical | |
| IVIS ® Spectrum In Vivo Imaging System | Perkin Elmer | N/A |
| Eppendorf ® Mastercycler ® | Eppendorf | N/A |

### Experimental Model and Subject Details

Data and Code Availability. Crystal structures have been deposited to PDB with accession codes 7RD3, 7LKB, 7RDA, 7RD4, 7LKG, 7RD9, 7RCS, and 7RAJ.

Mice and immunizations. For experiments male B6.SJL-Ptprc^{a}pepc^{b}/BoyJ mice (CD45.1^{+/+}) between 7-12 weeks were purchased from The Jackson Laboratory (Bar Harbor ME). F0-mice from the inferred germline CIS43 KI mouse (CD45.2+/+) colony were bred at the animal facility of the Gene Modification Facility (Harvard University) and breeding for colony expansion and experimental procedures was subsequently performed at the Ragon Institute of MGH, MIT and Harvard. Ear or tail snips from CIS43-germline KI mice were used for genotyping by TaqMan assay for a fee for service agreement (TransnetYX). TaqMan probes for the genotyping assay were developed by TransnetYX. CD45.2⁺ B cells from iGL-CIS43 donor KI mice were enriched using the Pan B Cell Isolation Kit II (Miltenyi Biotec), enumerated, diluted to desired cell numbers in PBS and adoptively transferred into CD45.1⁺ recipient mice as reported previously (Abbott et al., 2018).

Preparations of immunogens (PfCSP at 5 μg/mouse (or 50 μg/mouse) and/or NPDP19-KLH at 50 μg/mouse) were diluted in PBS at a volume of 100 μl/mouse and mixed at a 1:1 ratio with 100 μl/mouse Alhydrogel 2% (Invivogen) for at least 25 min, and then injected intraperitoneally (i.p.) (total volume of 200 μl/mouse). All experiments were done with approval by the Institutional Animal Care and Use Committee (IACUC) of Harvard University and the Massachusetts General Hospital and conducted in accordance with the regulations of the American Association for the Accreditation of Laboratory Animal Care (AAALAC).

Female 6-8-week-old B6(Cg)-Tyrc-2J/J albino mice were obtained from The Jackson Laboratory. All animals were cared for in accordance with American Association for Accreditation of Laboratory Animal Care standards in accredited facilities. All animal procedures were performed according to protocols approved by the Institutional Animal Care and Use Committees of the National Institute of Allergy and Infectious Diseases, National Institutes of Health, specifically: Animal Study Protocol VRC-17-702.

Generation of CIS43-germline knock-in (KI) mice. Inferred germline CIS43 KI mice were generated following published protocols (Lin et al., 2018b; Wang et al., 2020b). In brief, the targeting vector 4E10 (Ota et al., 2013) was modified by the incorporation of human rearranged CIS43-germline VDJ (heavy chain construct) or VJ (light chain construct) sequences downstream of the promoter region and by elongation of the 5′ and 3′ homology regions utilizing the Gibson assembly method (NEB). The targeting vector DNA was confirmed by Sanger sequencing (Eton Bioscience Inc.). sgRNAs used here were identical to sgRNAs previously validated for BG18^{gH} and PGT121 κ (Lin et al., 2018b).

Next, an injection mix containing both heavy and light chain DNA constructs described above (200 ng/μl), Cas9 protein (50 ng/μl), the corresponding sgRNAs (25 ng/μl) and injection buffer was prepared for microinjecting 200 fertilized oocytes. Following culture, resulting zygotes were implanted into the uteri of pseudopregnant surrogate mothers.

Immunogen and FACS probe production. Full length recombinant Plasmodium falciparum circumsporozoite protein (rPfCSP) was generated as previously described (Kisalu et al., 2018). The peptide-based prototypical immunogen was generated by conjugating NPDP19 (KQPADGNPDPNANPNVDPN, SEQ ID NO: 51) via a maleimide linker to KLH that had been equipped with free —SH groups via Trauts reagents (GenScript). For flow cytometric probe binding rPfCSP was biotinylated by BirA enzymatic reaction (Avidity, Inc) according to the manufacturer's protocol. The junctional peptide (NPDP19) was synthetically made and biotinylated at its N terminus (GenScript). Biotinylated rPfCSP and NPDP19 peptide were pre-reacted in independent tubes for at least 30 min in a 4:1 molar ratio with fluorescently labeled streptavidin (SA-A488 and/or SA-647). Reagents were then combined with fluorescently labeled antibodies for FACS-staining.

ELISA. 96-well plates were coated overnight at 4° C. with one of the following: NPDP19 at 50 ng per well, NANP5 at 125 ng per well or PfCSP 25 ng per well. Plates were washed 5 times with 0.05% Tween 20 in PBS, blocked with 100 μl of 3% BSA in PBS for 1 h at room temperature (RT), and washed again prior to incubation with 1:3 or 1:5 serially diluted mouse serum samples for 1 h at RT. Wells were washed and incubated with Alkaline Phosphatase AffiniPure Goat Anti-Mouse IgG (Jackson Immuno Research) at 1:1,000 in PBS with 0.5% BSA for 1 h at RT. p-Nitrophenyl phosphate dissolved in ddH₂O (50 μl/well, RT, 25 min) was used for detection. A chimeric version of the anti-PfCSP antibody 2A10 (Fisher et al., 2017; Hollingdale et al., 1984) with human Ig heavy and Ig kappa and the fully human mature anti-PfCSP CIS43 antibody were used as standard reference materials. ELISA curves were calculated and analyzed using GraphPad Prism 8.4.3 (GraphPad).

ALPHALISA®. ALPHALISA® (Perkin-Elmer) is a bead-based proximity assay in which singlet oxygen molecules, generated by high energy irradiation of Donor beads, transfer to Acceptor beads, which are within a distance of approximately 200 nm. It is a sensitive high throughput screening assay that does not require washing steps. A cascading series of chemical reactions results in a chemiluminescent signal. Purified antibodies were diluted to 100 nM in ALPHALISA® buffer (PBS+0.05% Tween-20+0.5 mg/mL BSA). Subsequently, 5 μL of the IgGs were transferred to an OptiPlate-384 assay plate (white opaque, PerkinElmer), mixed with 10 μL (10 nM final conc.) of biotinylated peptide probe and 10 uL (10 μg/mL final conc.) of Anti-human IgG (Fc specific; Perkin-Elmer) acceptor beads. After an hour of incubation at RT, non-shaking, 25 uL (40 μg/mL final conc.) of streptavidin donor beads (Perkin-Elmer) were added. The plate was then incubated for 30 min at RT in the dark before the ALPHALISA® signal was detected using a SpectraMax® i3x multi-mode microplate reader (Molecular Devices).

Flow Cytometry. At select time points following immunization, whole spleens were mechanically dissociated using 5 ml syringe plungers to generate single-cell suspensions. ACK lysis buffer was used to remove red blood cells and splenocytes were then resuspended in FACS buffer (2% FBS/PBS), Fc-blocked (clone 2.4G2, BD Biosciences) and stained for viability with Live/Dead Blue (Thermo Fisher Scientific) for 20 min at 4° C. For surface staining tetramer rPfCSP and/or NPDP19 probes (described above), as well as antibodies against CD4-APC-eF780, CD8-APC-eF780, Gr-1-APC-eF780, F4/80-APC-eF780, B220-B510, CD95-PE-Cy7, CD38-A700, CD45.1-PerCP-Cy5.5, CD45.2-PE, IgD-BV786, IgM-BUV395 and IgG1-BV421, were used. Cells were acquired by a BD FACSymphony (BD Biosciences) for flow cytometric analysis and sorted using a BD FACS Aria II instrument (BD Biosciences). Data was analyzed using FlowJo software (Tree Star). The gating strategy is shown in FIG. 11E. CD95⁺CD38⁺CD45.2⁺IgG1⁺ B cells were single-cell dry-sorted into 96-well PCR plates, rapidly frozen on dry ice and stored at −80° C. until processing.

BCR sequencing. Following single-cell sorting of antigen-specific B cells, the genes encoding the variable region of the heavy and light chains of IgG were amplified through RT-PCR. In brief, first strand cDNA synthesis was carried out using SuperScript III Reverse Transcriptase (Invitrogen) according to manufacturer's instructions. Nested PCR reactions consisting of PCR-1 and PCR-2 were performed as 25 μl reactions using HotStarTaq enzyme (QIAGEN), 10 mM dNTPS (Thermo Fisher Scientific) and cocktails of IgG- and IgK-specific primers and thermocycling conditions described previously (von Boehmer et al., 2016). PCR products were run on precast E-Gels 96 2% with SYBR Safe (Thermo Fisher Scientific) and wells with bands of the correct size were submitted to GENEWIZ company for Sanger sequencing. HC products were sequenced using the HC reverse primer from PCR-2 (5′ GCTCAGGGAARTAGCCCTTGAC 3′, SEQ ID NO: 223) and the LC was sequenced using the LC reverse primer (5′ TGGGAAGATGGATACAGTT 3′, SEQ ID NO: 224) from PCR-2.

Reads were quality-checked, trimmed, aligned and analyzed using the Geneious software (Geneious). IMGT/V-QUEST (imgt.org) was used for mouse/Human Ig gene assignments. CIS43-like mutation calculation (FIG. 4D) were done as described previously (Briney et al., 2019; Soto et al., 2019).

Epitope mapping and competition ELISAs. Competitive ELISAs using overlapping linear PfCSP peptides (peptides 16-61; Genscript) that span the R1 and repeat region of PfCSP were performed on the Meso Scale Discovery (MSD) U-Plex Assay platform. Peptides were all 15 amino acids in length, overlapping by 11 residues, and numbered according to their position on the protein. Briefly, streptavidin-coated plates (Meso Scale Discovery, MSD) were blocked with 5% BSA in PBS for 30 min at room temperature (RT), washed five times (wash buffer, 0.05% Tween-20 in PBS), then coated with biotinylated-recombinant PfCSP (0.2 μg/mL, Genscript) in PBS with 1% BSA, and allowed to incubate for 1 h at RT. Either PfCSP specific monoclonal antibodies (all at 10 ng/mL except iGL-CIS43 at 100 ng/mL), or polyclonal mouse sera (pooled per group then diluted 1:250) were preincubated with varying concentrations (0-1,000 mg/mL) of selected PfCSP peptides in PBS with 1% BSA/0.05% Tween-20 for 2 hrs at 37 C, then added onto the rPfCSP-coated plates. Plates were incubated for 1 h at RT, washed five times, then incubated for an additional 1 h at RT with 1 μg/mL of appropriate secondary (either anti-human or anti-mouse) IgG SULFO-TAG (Meso Scale Discovery) in PBS with 1% BSA/0.05% Tween-20. After washing, plates were read using 1×MSD Read T Buffer (Meso Scale Discovery) on an MSD SECTOR © Imager 6000 instrument.

Sporozoites. Transgenic P. berghei (strain ANKA 676m1c11, MRA-868) expressing full-length P. falciparum PfCSP and a green fluorescent protein/luciferase fusion protein (Pb-PfCSP-GFP/Luc-SPZ) were obtained as previously described (Flores-Garcia et al., 2019b).

IV challenge and quantification of protection. IV challenges were performed as previously described (Wang et al., 2020a). Briefly, mAbs were diluted in sterile PBS (pH 7.4) to give a final dose of 50-200 μg, as indicated, in a total volume 200 l/mouse) and were injected IV via the tail vein. Approximately 4 hours later, mice were then intravenously challenged in the tail vein with 2,000 freshly harvested Pb-PfCSP-GFP/Luc-SPZ in Leibovitz's L-15 Medium (Gibco). 40-42 h post-challenge, mice were injected intraperitoneally with 150 μL of D-luciferin (PerkinElmer; 30 mg/mL), anesthetized with isoflurane and imaged with the IVIS® Spectrum in vivo imaging system (PerkinElmer) 10 min after luciferin injection. Liver burden was quantified by analyzing a region of interest (ROI) in the upper abdominal region; the total flux (p/s) was measured using the manufacturer's software (Living Image 4.5, PerkinElmer). To measure parasitemia, luciferin was re-injected 7 days post-challenge and quantification was performed with an ROI encompassing the whole body.

$$For the correlation of mAb sequence characteristics with protection , \\ percent ( \% ) protection for each mouse was calculated \\ based on the liver burden flux ( p / s ) data . \% protection = [ 100 - ( ( antibody - treated mouse flux / mean flux of untreated mice ) * 100 ) ] .$$

### Bioinformatics

Accumulation of CIS43-like mutation. Non-duplicated VH1-3 sequences were obtained from 13 healthy donors (PRJNA511481, (Soto et al., 2019), PRJNA406949, (Briney et al., 2019)), and non-duplicated VK4-1 sequences from 3 healthy donors (PRJNA511481). We calculated per donor mutation profile for VH1-3 and VK4-1 germline. In brief, we aligned the heavy and light chain protein sequences to VH1-3 and VK4-1 germline protein sequence, respectively, and calculated the probability distributions: 1. the number of mutations, 2. per site mutation rate, and 3. per site amino acid frequency. Based on per donor probability distribution, we generate synthetic VH1-3 and VK4-1 antibody sequences. The CIS43-like mutations were defined as all the SHM on CIS43 V germline gene. The mean frequency of randomly having CIS43 mutation in synthetic VH1-3/VK4-1 sequences and the 95% confidence intervals were calculated by using Python script.

Sequence-based sieving. Based on the antibody sequence, we defined five properties, V_{d}W clashes, sequence identity to mature CIS43, SHM on peptide contact residues, total SHM, and total SHM divided by silent mutations. We used RosettaRemodel to predict the structure model of CIS43 like variants (Huang et al., 2011). CIS43 in complexed with peptide2l (PDB ID 6B5M) was used as template for model building of CIS43 like variants and defined peptide contact residues (Kisalu et al., 2018). The value of fa_rep reported by RosettaRemodel used as the VdW clash score. The sequences of CIS43 like variants were aligned with CIS43 iGL or CIS43 mature sequence to obtain CIS43 sequence identity, SHM peptide on contact residues, total SHM and Total A.A. mutations divided by silent mutations.

Gene-specific substitution profile. Per residue mutation profiles of IGHV1-3 and IGKV4-1 germline genes were obtained from cAb-Rep server, and the substitution frequency less than 0.5% was defined as rare mutation (Sheng et al., 2017).

Informatic analysis on Van der Waals binding energy and WT CIS43_L98I and I98L mutations. Van der Waals (VdW) pairwise binding energy between peptide and Fab residues were calculated upon mutation to determine the importance of the L98I mutation. The mature CIS43 antibody (PDB: 6B5M) and iGL-CIS43-HL.K28.m43.151 complex structures were used as templates to model the 34 CIS43-variant antibodies by using the FoldX software (foldxsuite.crg.eu). The variants were then minimized using YASARA (yasara.org). The reference structures were also modeled and minimized by making VH1V and QH1Q identity mapping mutations. A single DCD trajectory frame was generated with VMD's Autopsf software (ks.uiuc.edu/Research/vmd). The residue interaction energies were calculated using gRINN (grinn.readthedocs.io/en/latest/index.html) and NAMD_2.14_Linux-×86_64-multicore with CHARMM36 force field (Best et al., 2012; Guvench et al., 2011) a default NAMD solute dielectric of 1, non-bonded cutoff 12 Å, filtering cutoff of 15. The resulting VdW energy matrix M_{A}b∈ [symbol] between peptide 21 and m heavy/light chain residues within the cutoffs was generated for all 34 variants. The energy differences were calculated by subtracting the variant matrix from the aligned reference matrix. Only mutated entries strictly greater than zero were selected and summed across peptide positions. Finally, ΔE values were grouped by mutation position and type and averaged across variants. The CIS43_L98I and m43.151_I98L variants were also generated and analyzed by following the same procedure.

Production of antibodies and antigen-binding fragments (FABs). Antibody heavy and light chain genes were synthesized (Gene Universal Inc, Newark DE) and subcloned into corresponding pVRC8400 vectors. To express the antibodies, equal amounts of heavy and light chain plasmids were transfected into Expi293F cells (Life Technology) by using Turbo293 transfection reagent (Speed BioSystems). Transfected cells were cultured in shaker incubator at 120 rpm, 37° C., 9% CO₂ for 5 days. Culture supernatants were harvested and purified over Protein A (GE Health Science) resin in columns. Each antibody was eluted with IgG elution buffer (Pierce), immediately neutralized with one tenth volume of 1M Tris-HCl pH 8.0. The antibodies were then buffer exchanged in PBS by dialysis.

Fabs containing a 6×His-tag on heavy chain were expressed as above. On day 6 post transfection, culture supernatants were harvested and incubated with cOmplete His-Tag Purification resin. After washing with PBS containing 20 mM imidazole, Fabs were eluted in 50 mM sodium phosphate pH 8.0, 300 mM NaCl, and 250 mM imidazole. The protein was further purified by size exclusion chromatography (SEC) on a Superose 6 10/300 GL column in PBS.

Affinity measurements by BLI. Antibody Fab binding affinity to various ligands were measured using biolayer interferometry on an Octet Red384 instrument (fortdBio) with streptavidin capture biosensors (fortdBio) in solid black tilt-well 96-well plates (Geiger Bio-One). Assays were performed with agitation at 25° C. Immobilization of biotinylated rPfCSP, peptides 21 or peptide NANP5 was performed for 60 s, followed by a 60 s baseline in buffer (PBS+1% BSA). Association with Fab (serially diluted from 1000 to 62.5 nM) was done for 60 s, followed by a dissociation step in buffer for 120 s. In all Octet measurements, parallel correction to subtract systematic baseline drift was carried out by subtracting the measurements recorded for a loaded sensor incubated in PBS. Data analysis was carried out using Octet software, version 9.0. Experimental data were fitted globally with a 1:1 Langmuir model of binding.

ITC. A stabilized version of PfCSP with increased expression (3D7 clone of the NF54 strain (PlasmoDB ID: PF3D7_0304600.1)) was used for the ITC experiments. This construct, termed PfCSP_SAmut_C5S (Wang et al., 2020a) was modified from rPfCSP (Kisalu et al., 2018) by introducing four amino acid mutations in the N-terminal domain that removed processing sites and prevented dimerization upon solubilization to increase yield and facilitate consistent analyses. The protein was expressed through transient transfection in 293F cells (Thermo Fisher Scientific) and purified from culture supernatants through polyhistidine-tag affinity chromatography followed by size-exclusion chromatography (GE Healthcare). Monomer-containing fractions were pooled, concentrated, snap frozen, and stored at −80° C.

Calorimetric titrations of full-length rPfCSP with selected antibodies, m42.126, m42.127, m43.138, m43.149, m43.151, m43.159, m43.160 and CIS43 mature, were made using a MicroCal VP-ITC from Malvern Panalytical (Northampton, MA, USA). rPfCSP and the antibodies were prepared in PBS, pH 7.4, and all the titrations were performed at 25° C. The concentration of rPfCSP in the calorimetric cell (˜1.4 mL) was 0.20-0.25 μM and antibody solution at a concentration of 23-28 μM antigen binding sites was added in 7-μL aliquots until saturation was reached. The injections were made at 300 s intervals. The exact concentration of the experimental solutions was determined from the absorbance at 280 nm. The heat produced upon each injection was obtained by integration of the calorimetric signal and the heat associated with antibody binding to rPfCSP was obtained after subtraction of the heat of dilution from the heat of reaction. The individual heats of binding were expressed as a function of the molar ratio and the association constant, Kₐ=1/K_{d}, the enthalpy, ΔH, and the stoichiometry, N, were obtained by nonlinear regression of the data to a sequential binding model to two sets of sites with different binding energetics and stoichiometries (Freire et al., 2009).

Crystallization and structural analysis. Antibody Fab and peptide 21 (PfCSP residues 101-115) complexes were prepared by mixing 1:2 molar ration to a concentration of 15 mg/ml. Crystallization conditions were screened in Hampton Research screening kits, Wizard screening kits, Precipitant Synergy screening kits, JCSG1-4 screening kits using a mosquito robot. Crystals initially observed from the wells were manually reproduced. The m42.127: P21 complex crystal grew in 0.1 M Sodium acetate trihydrate pH 4.5 and 30% w/v polyethylene glycol 1,500; the m43.151: P21 complex crystal grew in 0.1 M Citric acid pH 3.5 and 25% w/v polyethylene glycol 3,350; the m43.160:P21 complex crystal grew in 0.1 M BIS-TRIS pH 5.5, 25% w/v Polyethylene glycol 3,350; the m42.126:P21 complex crystal grew in 0.1 M Sodium citrate tribasic dihydrate pH 5.5, 18% w/v Polyethylene glycol 3,350; the m43.149:P21 complex crystal grew in 0.2 M Sodium formate, 20% w/v Polyethylene glycol 3,350; the m43.159:P21 complex crystal grew in 0.1 M Sodium acetate trihydrate pH 4.0, 10% w/v Polyethylene glycol 4,000; the m43.138:P21 complex crystal grew in 0.1 M Sodium citrate tribasic dihydrate pH 5.0, 10% w/v Polyethylene glycol 6,000; the iGL-CIS43.D3 crystals grew in 0.2 M Zinc acetate, 0.1 M MES pH 6.0, and 20% w/v polyethylene glycol 8,000. Crystals were cryoprotected in 25% glycerol and flash-frozen in liquid nitrogen. Data were collected at a temperature of 100 K and a wavelength of 1.00 Å at the SER-CAT beamline ID-22 (Advanced Photon Source, Argonne National Laboratory). Diffraction data were processed with the HKL2000 suite (Otwinowski and Minor, 1997). Structure solution was obtained by molecular replacement with Phaser using CIS43 Fab structures (PDB ID: 6B5L) as a search model. Model building was carried out with Coot (Emsley and Cowtan, 2004). Refinement was carried out with Phenix (Liebschner et al., 2019). Ramachandran statistical analysis indicated that the final structures contained no disallowed residues or no more than 0.11% disallowed residues. Data collection and refinement statistics are shown in Table S-Structure.

Statistics. For immunization studies, statistical analysis was performed in Prism 9.01 (GraphPad) using either two-tailed Mann-Whitney test assuming non-normal distribution, Wilcoxon matched pairs signed rank test or Kruskal-Wallis test with Dunn's correction, as described in the figure legends. One-way ANOVA test with Dunnett's multiple comparisons was used to calculate the statistical differences of log₁₀(normalized liver burden) between CIS43 or LS wildtype and each of the six CIS43 variants in the low-dose protection study. The correlation between the normalized liver burden and various kinetics, sequence, and structural properties were calculated using two-tailed Pearson's correlation method. To compare the liver burden between different studies, the liver burden of each group was normalized based on the geometric mean of the liver burden values from the untreated mice in the same experiment. In terms of Feature-Sequence Associations, an in-house version of SeqFeatR was used to down-select antibody sequence position-amino acid combinations associated with peptide2l and PfCSP binding or liver burden function (Budeus et al., 2016). Further, we used Fisher's exact test to determine the significance of the association between amino acid frequencies and binding or liver burden. The peptide21 binding, PfCSP binding, and liver burden data was divided into two classes by using the geometric mean as split point, hereby generating low and high binding, or low and high liver burden classes. P values less than 0.05 were considered significant (*P<0.05; **P<0.01; ***P<0.001; ****P<0.0001) as indicated in the figure legends.

### References for Example 1

Abbott et al. (2018). Precursor Frequency and Affinity Determine B Cell Competitive Fitness in Germinal Centers, Tested with Germline-Targeting HIV Vaccine Immunogens. Immunity 48, 133-146.e6.
Atcheson et al. (2021). A VLP for validation of the Plasmodium falciparum circumsporozoite protein junctional epitope for vaccine development. Npj Vaccines 6.
Best et al. (2012). Optimization of the Additive CHARMM All-Atom Protein Force Field Targeting Improved Sampling of the Backbone ϕ, ψ and Side-Chain χ1 and χ2 Dihedral Angles. J. Chem. Theory Comput. 8, 3257-3273.
von Boehmer et al. (2016). Sequencing and cloning of antigen-specific antibodies from mouse memory B cells. Nat. Protoc. 11, 1908-1923.
Briney et al. (2019). Commonality despite exceptional diversity in the baseline human antibody repertoire. Nature 566, 393-397.
Budeus, B., Timm, J., and Hoffmann, D. (2016). SeqFeatR for the Discovery of Feature-Sequence Associations. PLoS One 11, e0146409.
Calvo-Calle et al. (2021). Identification of a neutralizing epitope within minor repeat region of Plasmodium falciparum CS protein. Npj Vaccines 6, 1-8.
Cerami et al. (1992). The basolateral domain of the hepatocyte plasma membrane bears receptors for the circumsporozoite protein of Plasmodium falciparum sporozoites. Cell 70, 1021-1033.
Cockburn, I. A., and Seder, R. A. (2018a). To Effective Vaccines and Protective Antibodies. Nat. Immunol. 19.
Cockburn, I. A., and Seder, R. A. (2018b). Malaria prevention: from immunological concepts to effective vaccines and protective antibodies. Nat. Immunol. 19, 1199-1211.
Dosenovic et al. (2018). Anti-HIV-1 B cell responses are dependent on B cell precursor frequency and antigen-binding affinity. PNAS. 115, 4743-4748.
Draper et al. (2018). Malaria Vaccines: Recent Advances and New Horizons. Cell Host Microbe.
Emsley, P., and Cowtan, K. (2004). Coot: model-building tools for molecular graphics. Acta Crystallogr. D. Biol. Crystallogr. 60, 2126-2132.
Fisher et al. (2017). T-dependent B cell responses to Plasmodium induce antibodies that form a high-avidity multivalent complex with the circumsporozoite protein. PLoS Pathog. 13, 1-23.
Flores-Garcia et al. (2019a). Optimization of an in vivo model to study immunity to Plasmodium falciparum pre-erythrocytic stages. Malar. J. 18, 426.
Flores-Garcia et al. (2019b). Optimization of an in vivo model to study immunity to Plasmodium falciparum pre-erythrocytic stages. Malar. J. 18, 426.
Foquet et al. (2014). Vaccine-induced monoclonal antibodies targeting circumsporozoite protein prevent Plasmodium falciparum infection. J. Clin. Invest. 124, 140.
Francica et al. (2021). Design of alphavirus virus-like particles presenting circumsporozoite junctional epitopes that elicit protection against malaria. Vaccines 9.
Freire, E., Schön, A., and Velazquez-Campoy, A. (2009). Chapter 5 Isothermal Titration Calorimetry: General Formalism Using Binding Polynomials. Methods Enzymol. 455, 127-155. Global Malaria Programme, W. G. (2020). World Malaria Report 2020.
Guo et al. (2019). cAb-Rep: A Database of Curated Antibody Repertoires for Exploring Antibody Diversity and Predicting Antibody Prevalence. Front. Immunol. 10, 2365.
Guvench et al. (2011). CHARMM Additive All-Atom Force Field for Carbohydrate Derivatives and Its Utility in Polysaccharide and Carbohydrate-Protein Modeling. J. Chem. Theory Comput. 7, 3162-3180.
Havenar-Daughton et al. (2018). When designing vaccines, consider the starting material: the human B cell repertoire. Curr. Opin. Immunol. 53, 209.
Hollingdale et al. (1984). Inhibition of entry of Plasmodium falciparum and P. vivax sporozoites into cultured cells; an in vitro assay of protective antibodies. J. Immunol. 132, 909-913.
Huang et al. (2011). RosettaRemodel: a generalized framework for flexible backbone protein design. PLoS One 6, e24109.
Imkeller et al. (2018). Anti-idiotope affinity maturation improves human B cell responses against a repetitive parasite antigen. Revis. 5304, 1-10.
Jelinkovi et al. (2021). An epitope-based malaria vaccine targeting the junctional region of circumsporozoite protein. Npj Vaccines 6, 1-10.
Julien, J.-P., and Wardemann, H. (2019). Antibodies against Plasmodium falciparum malaria at the molecular level. Nat. Rev. Immunol. 19.
Kisalu et al. (2018). A human monoclonal antibody prevents malaria infection by targeting a new site of vulnerability on the parasite. Nat. Med. 24, 408-416.
Kisalu et al. (2020). Enhancing durability of CIS43 monoclonal antibody by Fc mutation or AAV delivery for malaria prevention. JCI Insight.
Kwong, P. D., and Mascola, J. R. (2018). HIV-1 Vaccines Based on Antibody Identification, B Cell Ontogeny, and Epitope Structure. Immunity 48, 855-871.
Liebschner et al. (2019). Macromolecular structure determination using X-rays, neutrons and electrons: recent developments in Phenix. Acta Crystallogr. Sect. D, Struct. Biol. 75, 861-877.
Lin et al. (2018a). One-step CRISPR/Cas9 method for the rapid generation of human antibody heavy chain knock-in mice. EMBO J. e99243.
Lin et al. (2018b). One-step CRISPR/Cas9 method for the rapid generation of human antibody heavy chain knock-in mice. EMBO J. e99243.
McNamara et al. (2020). Antibody Feedback Limits the Expansion of B Cell Responses to Malaria Vaccination but Drives Diversification of the Humoral Response. Cell Host Microbe 28, 572-585.e7.
Murugan et al. (2018). Clonal selection drives protective memory B cell responses in controlled human malaria infection. Sci. Immunol. 3.
Murugan et al. (2020). Evolution of protective human antibodies against Plasmodium falciparum circumsporozoite protein repeat motifs. Nat. Med. 1-11.
Nilsson et al. (2015). Targeting Human Transmission Biology for Malaria Elimination. PLoS Pathog. 11.
Ota et al. (2013). B Cells from Knock-in Mice Expressing Broadly Neutralizing HIV Antibody b12 Carry an Innocuous B Cell Receptor Responsive to HIV Vaccine Candidates. J. Immunol. 191, 3179-3185.
Otwinowski, Z., and Minor, W. (1997). Processing of X-ray diffraction data collected in oscillation mode. Methods Enzymol. 276, 307-326.
Oyen et al. (2017). Structural basis for antibody recognition of the NANP repeats in Plasmodium falciparum circumsporozoite protein. Proc. Natl. Acad. Sci. U.S.A 114, E10438-E10445.
Oyen et al. (2018). Cryo-EM structure of P. falciparum circumsporozoite protein with a vaccine-elicited antibody is stabilized by somatically mutated inter-Fab contacts. Sci. Adv. 4, eaau8529.
Oyen et al. (2020a). Structure and mechanism of monoclonal antibody binding to the junctional epitope of Plasmodium falciparum circumsporozoite protein. PLoS Pathog. 16, 1-22.
Oyen et al. (2020b). Structure and mechanism of monoclonal antibody binding to the junctional epitope of Plasmodium falciparum circumsporozoite protein.
Partnership, S. C. T. (2012). A Phase 3 Trial of RTS,S/AS01 Malaria Vaccine in African Infants. N.
Engl. J. Med. 367, 2284-2295.
Phillips et al. (2017). Malaria. Nat. Rev. Dis. Prim. 3, 17050.
Pholcharee et al. (2021). Structural and biophysical correlation of anti-NANP antibodies with in vivo protection against P. falciparum. Nat. Commun. 12, 1063.
Raghunandan et al. (2020). Characterization of two in vivo challenge models to measure functional activity of monoclonal antibodies to Plasmodium falciparum circumsporozoite protein. Malar. J. 19, 113.
RTS, S. C. T. P. (2015). Efficacy and safety of RTS,S/AS01 malaria vaccine with or without a booster dose in infants and children in Africa: Final results of a phase 3, individually randomised, controlled trial. Lancet 386, 31-45.
Seder et al. (2013). Protection against malaria by intravenous immunization with a nonreplicating sporozoite vaccine. Science 341, 1359-1365.
Sheng et al. (2017). Gene-Specific Substitution Profiles Describe the Types and Frequencies of Amino Acid Changes during Antibody Somatic Hypermutation. Front. Immunol. 8, 537.
Soto et al. (2019). High frequency of shared clonotypes in human B cell receptor repertoires. Nature 566, 398-402.
Tan et al. (2018). A public antibody lineage that potently inhibits malaria infection through dual binding to the circumsporozoite protein. Nat. Med. 24, 401-407.
Tan, J., Piccoli, L., and Lanzavecchia, A. (2019). The Antibody Response to Plasmodium falciparum: Cues for Vaccine Design and the Discovery of Receptor-Based Antibodies. Annu. Rev. Immunol 37, 225-246.
Tewari et al. (2002). Function of Region I and II Adhesive Motifs of Plasmodium falciparum Circumsporozoite Protein in Sporozoite Motility and Infectivity. J. Biol. Chem. 277, 47613-47618.
Triller et al. (2017). Natural Parasite Exposure Induces Protective Human Anti-Malarial Antibodies Article Natural Parasite Exposure Induces Protective Human Anti-Malarial Antibodies. Immunity 47.
Wang et al. (2020a). A Potent Anti-Malarial Human Monoclonal Antibody Targets Circumsporozoite Protein Minor Repeats and Neutralizes Sporozoites in the Liver. Immunity 53, 733-744.e8.
Wang et al. (2020b). Multiplexed CRISPR/CAS9-mediated engineering of pre-clinical mouse models bearing native human B cell receptors. EMBO J.
Wardemann, H., and Murugan, R. (2018). From human antibody structure and function towards the design of a novel Plasmodium falciparum circumsporozoite protein malaria vaccine. Curr. Opin. Immunol. 53, 119-123.
White et al. (2014). A combined analysis of immunogenicity, antibody kinetics and vaccine efficacy from phase 2 trials of the RTS,S malaria vaccine.
Xu et al. (2018). Epitope-based vaccine design yields fusion peptide-directed antibodies that neutralize diverse strains of HIV-1. Nat. Med. 24, 857-867.
Zavala et al. (1983). Circumsporozoite proteins of malaria parasites contain a single immunodominant region with two or more identical epitopes. J. Exp. Med. 157, 1947-1957.

### Example 2

### Variant CIS43 Antibodies

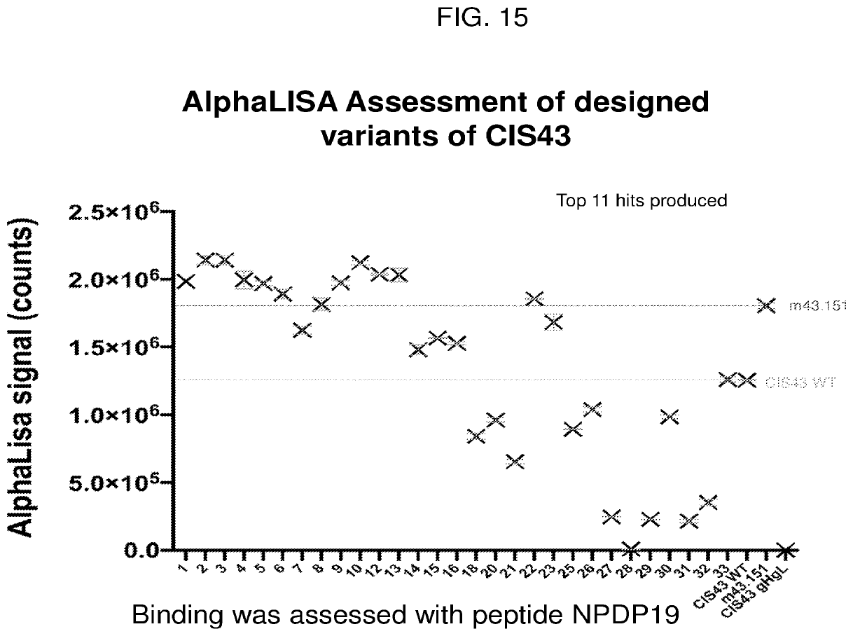

This example illustrates the design and assessment of additional variant CIS43 antibodies. Variant CIS43 antibodies were designed based on information gleaned from the study of CIS43 and variant CIS43 antibodies presented in Example 1. In addition to the D1-D11 antibodies discussed in Example 1, 20 new antibodies were designed for improved binding to NPDP19 peptide. The antibody names are listed in the following table, and VH and VL antibody sequences are provided below (except D1-D11, which are provided in Example 1). The VH and/or VL sequences include one or more amino acid mutations designed to increase binding to the NPDP19 peptide. The designed VH and VL sequences were cloned into IgG expression plasmid and assessed for NPDP19 binding by ALPHALISA® as described in Example 1, the results are shown in FIG. 15, with the antibody ID in the figure according to the following table. For this assay, antibody IgG present in supernatant from antibody expressing cells (0.6 nM antibody) was assessed for NPDP19 binding.

| | | ID in FIG. | |
|---|---|---|---|
| ID in FIG. 15 | Antibody name | 15 | Antibody name |
| 1 | D1 | 18 | m43_HH28K.17_RR_MR_AH33W |
| 2 | D2 | 20 | m43_HH28K.17_RR_MR_VH101W |
| 3 | D3 | 21 | m43_HH28K.17_RR_MR_AH33W_KH52D |
| 4 | D4 | 22 | D22 |
| 5 | D5 | 23 | m43_HH28K.17_RR_MR_AH33W_NL34Y_ |
| | | | VH101W |
| 6 | D6 | 25 | m43_HH28K.17_RR_MR_AH33W_NL34Y |
| 7 | m43-HH28K.17_m43-HH28-K.15 | 26 | CIS43_RR_MR_SL99R |
| 8 | D7 | 27 | CIS43_RR_MR_AH32W |
| 9 | D9 | 28 | CIS43_RR_MR_YL31W |
| 10 | D10 | 29 | m42_HH28K.13_RR_MR_AH33W |
| 12 | D12 | 30 | m42_HH28K.13_RR_MR_TH103R |
| 13 | D13 | 31 | m42_HH28K.13_RR_MR_TH103R_AH33W |
| 14 | m43-HHK28.15_m43-HH28K.04 | 32 | CIS43_RR_MR_SL99N |
| 15 | m43-HHK28.15_m42-HH28K.13 | 33 | CIS43_RR_MR_LH98F |
| 16 | m43-HHK28.15_m43-HH28K.25 | 34 | CIS43 WT |
| 18 | m43_HH28K.17_RR_MR_AH33W | 35 | m43.151 |
| 20 | m43_HH28K.17_RR_MR_VH101W | 36 | CIS43 gHgL |

In FIG. 15, the m43.151 and CIS43 WT antibodies are provided as controls, with the ALPHALISA® threshold for each antibody indicated. Several of the variant antibodies exhibited increased binding to the NPDP19 peptide compared to m43.151 and CIS43 WT.

>m43-HH28K.17_m43-HH28-K. 15
(SEQ ID NO: 57)
VH: m43-HH28K.17, QVHLVQSGAEVKKPGASVKVSCKASGYTFTRYAIHWVRQAPGQRLEWMGWIKGGNGNTR
YSQKFQDRVTITRDTSASTAYMELSSLRSEDTAVYYCALLTVITPDDAFDIWGQGTMVTVSS
(SEQ ID NO: 58)
VL: m43-HH28-K.15, DIVMTQSPDSLAVSLGERATINCKSSONIFFSSNNKNYLAWYQQIPGQPPKLLFYWA
STRESGVPDRFSGSGSGTDFTLTISSLQAEDVAVYYCHQYYSSPLTFGGGTKVEIQ
>D12
(SEQ ID NO: 59)
VH: m43-HH28K.15_Y27dF, QVQLVQSGAEVKKPGASVKVSCKASGYTFTRYAIHWVRQAPGQRLEWMGWIKA
GNGNTRYSQKFQDRVTITRDTSASTAYMELSSLRSEDTAVYYCALLTVITPDDAFDIWGQGTMVTVSS
(SEQ ID NO: 60)
VL: m43-HH28K.15_Y27dF, DIVMTQSPDSLAVSLGERATINCKSSQNIFFSSNNKNYLAWYQQIPGQPPKLL
IYWASTRESGVPDRF SGSGSGTDFTLTISSLQAEDVAVYYCHQYYSSPLTFGGGTKVEIQ
>D13
(SEQ ID NO: 61)
VH: m43-HH28K.15_K_N28K, QVQLVQSGAEVKKPGASVKVSCKASGYTFTRYAIHWVRQAPGQRLEWMGWIK
AGNGNTRYSQKFQDRVTITRDTSASTAYMELSSLRSEDTAVYYCALLTVITPDDAFDIWGQGTMVTVSS
(SEQ ID NO: 62)
VL: m43-HH28K.15_K_N28K, DIVMTQSPDSLAVSLGERATINCKSSQNIFFSSKNKNYLAWYQQIPGQPPKL
LIYWASTRESGVPDRFSGSGSGTDFTLTISSLQAEDVAVYYCHQYYSSPLTFGGGTKVEIQ
>m43-HHK28.15_m43-HH28K. 04
(SEQ ID NO: 63)
VH: m43-HHK28.15_m43-HH28K.04, QVQLVQSGAEVKKPGASVKVSCKASGYTFTTYAIHWVRQAPGQRLE
WMGWIKVGDGNTRYSPKFQDRVTITRDTSASTAYMELSSLRSEDTAVYFCALLTVITPDDAFDIWGQGTMVTVSS
(SEQ ID NO: 64)
VL: m43-HHK28.15_m43-HH28K.04, DIVMTQSPDSLAVSLGERATINCKSSQNIFFSSKNKNYLAWYQQIP
GQSPKLLIYWASTRESGVPDRFSGSGSGTDFTLTISSLQAEDVAVYYCHQYYSSPLITFGGGTKVEIQ
>m43-HHK28.15_m42-HH28K.13
(SEQ ID NO: 65)
VH: m43-HHK28.15_m42-HH28K.13, QVQLVQSGAEVKKPGASVKVSCKASGYTFTRYAIHWVRQAPGQRLE
WMGWI KAGNGNTRYSQKFQDRVTITRDTSASTAYMELSSLRSEDTAVYYCALLTVITPDDTEDIWGQGTMVTVSS
(SEQ ID NO: 66)
VL: m43-HHK28.15_m42-HH28K.13, DIVMTQSPDSLAVSLGERATINCKSSQNIFFSSNNKNYLAWYQQIP
GQAPKLLIYWASTRESGVPDRESGSGSGTDFTLTISSLQAEDVAVYYCHQYYSSPLTFGGGTKVEIQ
>m43-HHK28.15_m43-HH28K.25
(SEQ ID NO: 67)
VH: m43-HHK28.15, QVQLVQSGAELKKPGASVKVSCKASGYTFTRYAIHWVRQAPGQRLEWMGWIKAGNGNTR
YSQKFQDRVTITRDTSASTAYMELNSLRSEDTAVYYCALLTVITPDDAFDIWGQGTMVTVSS
(SEQ ID NO: 68)
VL: m43-HH28K.25, DIVMTQSPDSLAVSLGERATINCKSSQNIFFSSNNKNYLAWYQQIPGQPPOLLIYWAST
RESGVPDRFSGSGSGTDFTLTISSLQAEDVAVYYCHQYYSSPLTFGGGTKVEIQ
>m43_HH28K.17_RR_MR_AH33W
(SEQ ID NO: 69)
VH: m43_HH28K.17_AH33W, QVHLVQSGAEVKKPGASVKVSCKASGYTFTSYWIHWVRQAPGQRLEWMGWIKG
GNGNTRYSQKFQDRVTI TRDTSASTAYMELSSLRSEDTAVYYCALLTVITPDDAFDIWGQGTMVTVSS
(SEQ ID NO: 70)
VL: m43_HH28K.17_WT, DIVMTQSPDSLAVSLGERATINCKSSQNILYSSNNKNYLAWYQQKPGQPPKLLFYW
ASTRESGVPDRFSGSGSGTDFTLTISSLQAEDVAVYYCHQYYSSPLITFGGGTKVEIK
>m43_HH28K.17_RR_MR_VH101W
(SEQ ID NO: 71)
VH: m43_HH28K.17_VH101W, QVHLVQSGAEVKKPGASVKVSCKASGYTFTSYAIHWVRQAPGQRLEWMGWIK
GGNGNTRYSQKFQDRVTITRDTSASTAYMELSSLRSEDTAVYYCALLTWITPDDAFDIWGQGTMVTVSS
(SEQ ID NO: 72)
VL: m43_HH28K.17_WT, DIVMTQSPDSLAVSLGERATINCKSSQNILYSSNNKNYLAWYQQKPGQPPKLLFYW
ASTRESGVPDRFSGSGSGTDFTLTISSLQAEDVAVYYCHQYYSSPLTFGGGTKVEIK
>m43_HH28K.17_RR_MR_AH33W_KH52D
(SEQ ID NO: 73)
VH: m43_HH28K.17_AH33W_KH52D, QVHLVQSGAEVKKPGASVKVSCKASGYTFTSYWIHWVRQAPGQRLEW
MGWIDGGNGNTRYSQKFQDRVTITRDTSASTAYMELSSLRSEDTAVYYCALLTVITPDDAFDIWGQGTMVTVSS
(SEQ ID NO: 74)
VL: m43_HH28K.17_WT, DIVMTQSPDSLAVSLGERATINCKSSQNILYSSNNKNYLAWYQQKPGQPPKLLFYW
ASTRESGVPDRFSGSGSGTDFTLTISSLQAEDVAVYYCHQYYSSPLTFGGGTKVEIK
>D22
(SEQ ID NO: 75)
VH: m43_HH28K.17_AH33W_KH52D_NH56K, QVHLVQSGAEVKKPGASVKVSCKASGYTFTSYWIHWVRQAP
GQRLEWMGWIDGGKGNTRYSQKFQDRVTITRDTSASTAYMELSSLRSEDTAVYYCALLTVITPDDAFDIWGQGT
MVTVSS
(SEQ ID NO: 76)
VL: m43_HH28K.17_WT, DIVMTQSPDSLAVSLGERATINCKSSQNILYSSNNKNYLAWYQQKPGQPPKLLFYW
ASTRESGVPDRFSGSGSGTDFTLTISSLQAEDVAVYYCHQYYSSPLTFGGGTKVEIK
>m43_HH28K.17_RR_MR_AH33W_NL34Y_VH101W
(SEQ ID NO: 77)
VH: m43_HH28K.17_AH33W_VH101W, QVHLVQSGAEVKKPGASVKVSCKASGYTFTSYWIHWVRQAPGQRLE
WMGWIKGGNGNTRYSQKFQDRVTITRDTSASTAYMELSSLRSEDTAVYYCALLTWITPDDAFDIWGQGTMVTVSS
(SEQ ID NO: 78)
VL: m43_HH28K.17_NL34Y, DIVMTQSPDSLAVSLGERATINCKSSONILYSSYNKNYLAWYQQKPGQPPKLL
FYWASTRESGVPDRFSGSGSGTDFTLTISSLQAEDVAVYYCHQYYSSPLTFGGGTKVEIK
>m43_HH28K.17_RR_MR_AH33W_NL34Y
VH: m43_HH28K.17_AH33W
VL: m43_HH28K.17_NL34Y
>CIS43_RR_MR_SL99R
(SEQ ID NO: 79)
VH: CIS43_WT, QVQLVQSGAEVKKPGASVKVSCKASGYTFTSYAIHWVRQAPGQRLEWMGWIKAGNGNTRYSQK
FQDRVTITRDTSTTTAYMELSSLRSEDTAVYYCALLTVLTPDDAFDIWGQGTMVTVSS
(SEQ ID NO: 80)
VL: CIS43_SL99R, DIVMTQSPDSLAVSLGERATINCKSSQSVLYSSNNKNYLAWYQQKPGQPPNLLIYWASTR
QSGVPDRFSGSGSGTDFTLTISSLQAEDVAVYYCHQYYRSPLTFGGGTKVEIK
>CIS43_RR_MR_AH32W
(SEQ ID NO: 81)
VH: CIS43_AH32W, QVQLVQSGAEVKKPGASVKVSCKASGYTFTSYWIHWVRQAPGQRLEWMGWIKAGNGNTRY
SQKFQDRVTITRDTSTTTAYMELSSLRSEDTAVYYCALLTVLTPDDAFDIWGQGTMVTVSS
(SEQ ID NO: 82)
VL: CIS43_WT, DIVMTQSPDSLAVSLGERATINCKSSQSVLYSSNNKNYLAWYQQKPGQPPNLLIYWASTRQSG
VPDRFSGSGSGTDFTLTISSLQAEDVAVYYCHQYYSSPLTFGGGTKVEIK
>CIS43_RR_MR_YL31W
VH: CIS43_WT
(SEQ ID NO: 83)
VL: CIS43_YL31W, DIVMTQSPDSLAVSLGERATINCKSSQSVLWSSNNKNYLAWYQQKPGQPPNLLIYWASTR
QSGVPDRFSGSGSGTDFTLTISSLQAEDVAVYYCHQYYSSPLITFGGGTKVEIK
>m42_HH28K.13_RR_MR_AH33W
(SEQ ID NO: 84)
VH: m42_HH28K.13_AH33W, QVQLVQSGAEVKKPGASVKVSCKASGYTFTNYWIHWVRQAPGQRLEWMGWINA
VIGNTRYSQKFQGRVTITRDTSASTAYMELSSLRSEDTAVYYCALLTVITPDDAFDVWGQGTMVTVSS
(SEQ ID NO: 85)
VL: m42_HH28K.13_WT, DIVMTQSPDSLAVSLGERATINCKSSQSILYTSNNKKYLAWYQQKPGQPPKLLIYW
ASTRISGVPDRFSGSGSGTDFTLTISSLQAEDVAVYFCHQYYSSPLTFGGGTKVEIK
>m42_HH28K.13_RR_MR_TH103R
(SEQ ID NO: 86)
VH: m42_HH28K.13_TH103R, QVQLVQSGAEVKKPGASVKVSCKASGYTFTNYAIHWVRQAPGQRLEWMGWIN
AVIGNTRYSQKFQGRVTITRDTSASTAYMELSSLRSEDTAVYYCALLTVIRPDDAFDVWGQGTMVTVSS
(SEQ ID NO: 87)
VL: m42_HH28K.13_WT, DIVMTQSPDSLAVSLGERATINCKSSQSILYTSNNKKYLAWYQQKPGQPPKLLIYW
ASTRISGVPDRFSGSGSGTDFTLTISSLQAEDVAVYFCHQYYSSPLITFGGGTKVEIK
>m42_HH28K.13_RR_MR_TH103R_AH33W
(SEQ ID NO: 88)
VH: m42_HH28K.13_TH103R_AH33W, QVQLVQSGAEVKKPGASVKVSCKASGYTFTNYWIHWVRQAPGQRLE
WMGWINAVIGNTRYSQKFQGRVTITRDTSASTAYMELSSLRSEDTAVYYCALLTVIRPDDAFDVWGQGTMVTVSS
(SEQ ID NO: 89)
VL: m42_HH28K.13_WT, DIVMTQSPDSLAVSLGERATINCKSSQSILYTSNNKKYLAWYQQKPGQPPKLLIYW
ASTRISGVPDRFSGSGSGTDFTLTISSLQAEDVAVYFCHQYYSSPLTFGGGTKVEIK
>CIS43_RR_MR_SL99N
VH: CIS43_RR_MR_LH95F
(SEQ ID NO: 90)
VL: CIS43_WT, DIVMTQSPDSLAVSLGERATINCKSSQSVLYSSNNKNYLAWYQQKPGQPPNLLIYWASTRQSG
VPDRFSGSGSGTDFTLTISSLQAEDVAVYYCHQYYNSPLTFGGGTKVEIK
>CIS43_RR_MR_LH98F
(SEQ ID NO: 91)
VH: CIS43_RR_MR_NH56Y, QVQLVQSGAEVKKPGASVKVSCKASGYTFTSYAIHWVRQAPGQRLEWMGWIKAG
NGNTRYSQKFQDRVTITRDTSTTTAYMELSSLRSEDTAVYYCALFTVLTPDDAFDIWGQGTMVTVSS
VL: CIS43_WT

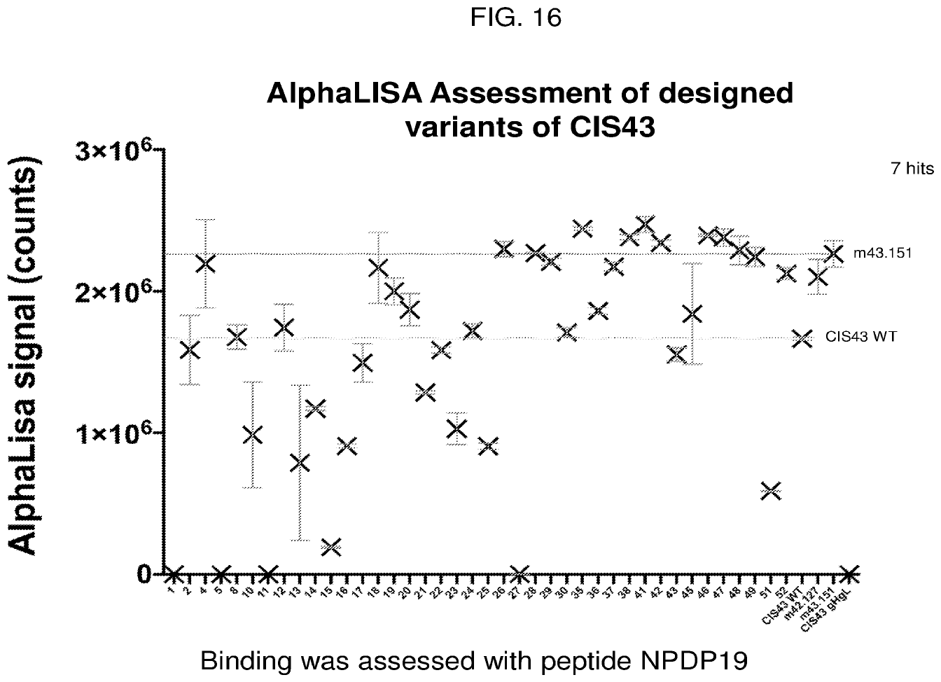

A further series of 39 new variant CIS43 antibodies were designed for improved binding to NPDP19 peptide. The antibody names with mutations indicated are listed in the following table, and VH and VL antibody sequences are provided in Appendix C of U.S. Provisional Application No. 63/275,936, filed Nov. 4, 2021; Appendix C is incorporated by reference herein in its entirety. The VH and/or VL sequences include one or more amino acid mutations designed to increase binding to the NPDP19 peptide. The designed VH and VL sequences were cloned into IgG expression plasmid and assessed for NPDP19 binding by ALPHALISA® as described in Example 1, the results are shown in FIG. 16, with the antibody ID in the figure according to the following table. For this assay, antibody IgG present in supernatant from antibody expressing cells (0.6 nM antibody) was assessed for NPDP19 binding.

| ID in FIG. 16 | Antibody name | ID in FIG. 16 | Antibody name |
|---|---|---|---|
| 1 | MP_1_m43.151HC_H35R | 24 | m43.151-HC_W47R |
| 2 | MP_2_m43.151HC_V97I | 25 | m43.151-HC_W47K |
| 4 | MP_4_m43.151HC_P100W | 26 | m43.151-HC_W50Y |
| 5 | MP_5_CIS43HC_H35R | 27 | m43.151-HC_W50K |
| 8 | MP_8_CIS43HC_P100F | 28 | m43.151-HC_K52R |
| 10 | MP_9_m42.127HC_A33F | 29 | m43.151-HC_K52L |
| 11 | MP_10_m42.127HC_H35R | 30 | m43.151-HC_K52F |
| 12 | MP_11_m42.127HC_V97I | 35 | m43.151-LC_Y27dW |
| 13 | MP_12_m42.127HC_I98P | 36 | m43.151-LC_Y27dE |
| 14 | MP_13_CIS43LC_Y27dW-Y32W-Y92W | 37 | m43.151-LC_Y27dD |
| 15 | MP_14_m42.127LC_Y27dW-Y32W-Y92W | 38 | m43.151-LC_S27fR |
| 16 | MP_15_m42.127LC_S94L | 41 | m43.151-LC_N28Q |
| 17 | m43.151-HC_Y32Q | 42 | m43.151-LC_K30R |
| 18 | m43.151-HC_Y32L | 43 | m43.151-LC_Y32W |
| 19 | m43.151-HC_A33S | 45 | m43.151-LC_Y92F |
| 20 | m43.151-HC_A33T | 46 | m43.151-LC_S93R |
| 21 | m43.151-HC_H35Q | 47 | m43.151-LC_S93K |
| 22 | m43.151-HC_H35F | 48 | m43.151-LC_S93L |
| 23 | m43.151-HC_H35A | 49 | m43.151-LC_S94H |
| 24 | m43.151-HC_W47R | 51 | m43.151-LC_L96N |
| 25 | m43.151-HC_W47K | | |

In FIG. 16, the m43.151 and CIS43 WT antibodies are provided as controls, with the ALPHALISA® threshold for each antibody indicated. Several of the variant antibodies exhibited increased binding to the NPDP19 peptide compared to m43.151 and CIS43 WT.

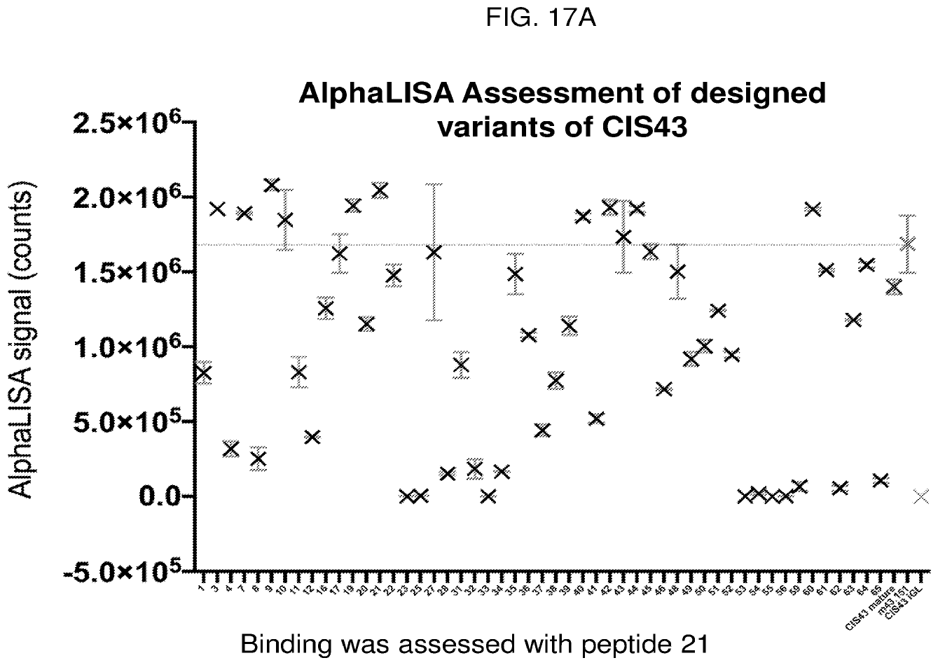

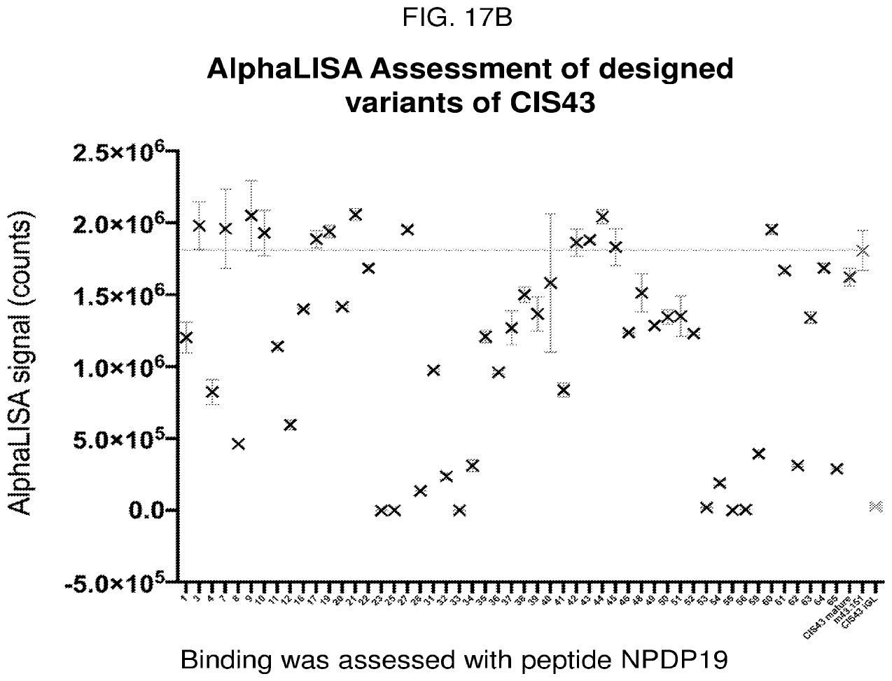

A further series of 65 new variant CIS43 antibodies were designed for improved binding to NPDP19 peptide. The antibody names with mutations indicated are listed in the following table, and VH and VL antibody sequences are provided below. The VH and/or VL sequences include one or more amino acid mutations designed to increase binding to the NPDP19 peptide. The designed VH and VL sequences were cloned into IgG expression plasmid and assessed for peptide 21 binding (FIG. 17A) or NPDP19 binding (FIG. 17B) by ALPHALISA® as described in Example 1, with the antibody ID in the figure according to the following table. For this assay, antibody IgG present in supernatant from antibody expressing cells (0.6 nM antibody) was assessed for NPDP19 binding.

| ID in | |
|---|---|
| FIG. 17 | Antibody Name |
| 1 | m42_HH28K_13_TH103R |
| 2 | m42_HH28K_13_AH33W |
| 3 | m42_HH28K_13_TH103K |
| 4 | m42_HH28K_13_VH101R |
| 5 | m42_HH28K_13_NL34W |
| 6 | m42_HH28K_13_SL99H |
| 7 | m42_HH28K_13_TH100M |
| 8 | m42_HH28K_13_VH101K |
| 9 | m42_HH28K_13_PH104M |
| 10 | m42_HH28K_13_PH104Q |
| 11 | m42_HH28K_13_VH101W |
| 12 | m42_HH28K_13_AH97E |
| 13 | m42_HH28K_13_NL34R |
| 14 | m42_HH28K_13_SL33W |
| 15 | m42_HH28K_13_SL99F |
| 16 | m42_HH28K_13_TH100C |
| 17 | m42_HH28K_13_YH32K |
| 18 | m42_HH28K_13_YL38W |
| 19 | m42_HH28K_13_TH103R_PH104R |
| 20 | m42_HH28K_13_TH103R_NL34W |
| 21 | m42_HH28K_13_TH103R_PH104Q |
| 22 | m42_HH28K_13_TH103R_SL33W |
| 23 | m42_HH28K_13_AH33W_IL29H_NL34 |
| | W_SL99F_TH103R |
| 24 | m42_HH28K_13_AH33W_IL29H_PH10 |
| | 4K_SL99Y_TH103R |
| 25 | m42_HH28K_13_AH33W_NL34W_PH1 |
| | 04M_PL101M_TH103R |
| 26 | m42_HH28K_13_AH53R_HH35E_PH10 |
| | 4R_SL99F_YH32R_YL38W |
| 27 | m42_HH28K_13_PH104R_SH31R_SL3 |
| | 3I_TH103R |
| 28 | m42_HH28K_13_AH33W_NL34W_SH3 |
| | 1R_SL99F_TH103R_VH101Q |
| 29 | m42_HH28K_13_AH53R_IL29H_PH10 |
| | 4M_SL99F_YH32R_YL38W |
| 30 | m42_HH28K_13_AH53R_NL34W_PH1 |
| | 04M_SL99H_YL38W |
| 31 | m42_HH28K_13_AH53R_NL34W_PH1 |
| | 04R_SL99H_YH32R |
| 32 | m42_HH28K_13_AH53R_PH104R_SL3 |
| | 2T_SL33M_TH103K_TL59Y_YH32R |
| 33 | m42_HH28K_13_AH53R_PL101M_SL3 |
| | 3I_TH103R_YH32R |
| 34 | m42_HH28K_13_IL29H_PH104R_SL99F_TH1 |
| | 07V_TL59H_YH32R_YL38W |
| 35 | m42_HH28K_13_PH104M_SH31R_SL99F_TH |
| | 103R_YL38W |
| 36 | m42_HH28K_13_PH104R_SL32T_TH103R_YL |
| | 97W |
| 37 | m43_HH28K_17_VH101R |
| 38 | m43_HH28K_17_VH101W |
| 39 | m43_HH28K_17_TH100I |
| 40 | m43_HH28K_17_TH103N |
| 41 | m43_HH28K_17_YL38R |
| 42 | m43_HH28K_17_AH107R |
| 43 | m43_HH28K_17_PH104K |
| 44 | m43_HH28K_17_PH104R |
| 45 | m43_HH28K_17_TH100M |
| 46 | m43_HH28K_17_TH100Q |
| 47 | m43_HH28K_17_YH32R |
| 48 | m43_HH28K_17_VH101R_PH104R |
| 49 | m43_HH28K_17_TH100I_YH32R |
| 50 | m43_HH28K_17_TH100I_YH32R |
| 51 | m43_HH28K_17_TH100I_YH32R |
| 52 | m43_HH28K_17_TH100I_YH32R |
| 53 | m43_HH28K_17_AH33W_SL99E_VH101R |
| 54 | m43_HH28K_17_AH33W_VH101R |
| 55 | m43_HH28K_17_LL102Q_NL34M_VH101R |
| 56 | m43_HH28K_17_AH33W_SL32F_TH103Q_ |
| | YL38R |
| 57 | m43_HH28K_17_NL34M_VH101R |
| 58 | m43_HH28K_17_AH24R_LL52Y_PH104R_ |
| | VH101K |
| 59 | m43_HH28K_17_AH33W_LH99F |
| 60 | m43_HH28K_17_PH104R_SH31T_YH32R |
| 61 | m43_HH28K_17_IH34W_PH104H_SL32D_ |
| | VH101R |
| 62 | m43_HH28K_17_PL101Y_SH31R_VH101R |
| 63 | m43_HH28K_17_SL99H_TH100I |
| 64 | m43_HH28K_17_TH100M_YH32R |
| 65 | m43_HH28K_17_VH101R_YH32R |

In FIGS. 17A and 17B, the m43.151 and CIS43 WT antibodies are provided as controls, with the ALPHALISA® threshold for the m43. 151 antibody indicated. Several of the variant antibodies exhibited increased binding to the NPDP19 peptide compared to m43.151 and CIS43 WT.

>m42_HH28K_13_TH103R
(SEQ ID NO: 92)
VH: m42.127_HC_TH103R, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTVIRPDDTFDIWGQGTMVTVSS
(SEQ ID NO: 92)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTI SSLQAEDVAVYYCHQYYSSPLTFGGGTKVEIK
>m42_HH28K_13_AH33W
(SEQ ID NO: 94)
VH: m42.127_HC_AH33W, QVQLVQSGAEVKKPGASVKVSCKASGYTFTSYW IHWVRQAPGQRLEWMGWIKAGN
GNTRYSQKFQDRVTITRDTSASTAYMELSSLRSEDTAVYYCALLTVITPDDTFDIWGQGTMVTVSS
(SEQ ID NO: 95)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_TH103K
(SEQ ID NO: 96)
VH: m42.127_HC_TH103K, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTVIKPDDTFDIWGQGTMVTVSS
(SEQ ID NO: 97)
VL: m42.127_LC_WT, DIVMTQSPDSLAVSLGERATINCKSSQNILYSSNNKNYLAWYQQKPGQAPKLLIYWAS
TRESGVPDRESGSGSGTDFTLTISSLQAEDVAVYYCHQYYSSPLTFGGGTKVEIK
>m42_HH28K_13_VH101R
(SEQ ID NO: 98)
VH: m42.127_HC_VH101R, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTRITPDDTFDIWGQGTMVTVSS
(SEQ ID NO: 99)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLIFGGGTKVEIK
>m42_HH28K_13_NL34W
(SEQ ID NO: 100)
VH: m42.127_HC_WT, QVQLVQSGAEVKKPGASVKVSCKASGYTFTSYAIHWVRQAPGQRLEWMGWIKAGNGNT
RYSQKFQDRVTITRDTSASTAYMELSSLRSEDTAVYYCALLTVITPDDTFDIWGQGTMVTVSS
(SEQ ID NO: 101)
VL: m42.127_LC_NL34W, DIVMTQSPDSLAVSLGERATINCKSSQNILYSSWNKNYLAWYQQKPGQAPKLLIY
WASTRESGVPDRFSGSGSGTDFTLTISSLQAEDVAVYYCHQYYSSPLTFGGGTKVEIK
>m42_HH28K_13_SL99H
(SEQ ID NO: 102)
VH: m42.127_HC_WT, QVQLVQSGAEVKKPGASVKVSCKASGYTFTSYAIHWVRQAPGQRLEWMGWIKAGNGNT
RYSQKFQDRVTITRDTSASTAYMELSSLRSEDTAVYYCALLTVITPDDTFDIWGQGTMVTVSS
(SEQ ID NO: 103)
VL: m42.127_LC_SL99H, DIVMTQSPDSLAVSLGERATINCKSSQNILYSSNNKNYLAWYQQKPGQAPKLLIY
WASTRESGVPDRFSGSGSGTDFTLTISSLQAEDVAVYYCHQYYhSPLTFGGGTKVEIK
>m42_HH28K_13_TH100M
(SEQ ID NO: 104)
VH: m42.127_HC_TH100M, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLMVITPDDTFDIWGQGTMVTVSS
(SEQ ID NO: 105)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_VH101K
(SEQ ID NO: 106)
VH: m42.127_HC_VH101K, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTKITPDDTFDIWGQGTMVTVSS
(SEQ ID NO: 107)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_PH104M
(SEQ ID NO: 108)
VH: m42.127_HC_PH104M, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTVITMDDTFDIWGQGTMVTVSS
(SEQ ID NO: 109)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_PH104Q
(SEQ ID NO: 110)
VH: m42.127_HC_PH104Q, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTVITQDDTFDIWGQGTMVTVSS
(SEQ ID NO: 111)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_VH101W
(SEQ ID NO: 112)
VH: m42.127_HC_VH101W, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTWITPDDTFDIWGQGTMVTVSS
(SEQ ID NO: 113)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_AH97E
(SEQ ID NO: 114)
VH: m42.127_HC_AH97E, QVQLVQSGAEVKKPGASVKVSCKASGYTFTSYAIHWVRQAPGQRLEWMGWIKAGN
GNTRYSQKFQDRVTITRDTSASTAYMELSSLRSEDTAVYYCELLTVITPDDTFDIWGQGTMVTVSS
(SEQ ID NO: 115)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_NL34R
(SEQ ID NO: 116)
VH: m42.127_HC_WT, QVQLVQSGAEVKKPGASVKVSCKASGYTFTSYAIHWVRQAPGQRLEWMGWIKAGNGNT
RYSQKFQDRVTITRDTSASTAYMELSSLRSEDTAVYYCALLTVITPDDTFDIWGQGTMVTVSS
(SEQ ID NO: 117)
VL: m42.127_LC_NL34R, DIVMTQSPDSLAVSLGERATINCKSSQNILYSSrNKNYLAWYQQKPGQAPKLLIY
WASTRESGVPDRF SGSGSGTDFTLTISSLQAEDVAVYYCHQYYSSPLTFGGGTKVEIK
>m42_HH28K_13_SL33W
(SEQ ID NO: 118)
VH: m42.127_HC_WT, QVQLVQSGAEVKKPGASVKVSCKASGYTFTSYAIHWVRQAPGQRLEWMGWIKAGNGNT
RYSQKFQDRVTITRDTSASTAYMELSSLRSEDTAVYYCALLTVITPDDTFDIWGQGTMVTVSS
(SEQ ID NO: 119)
VL: m42.127_LC_SL33W, DIVMTQSPDSLAVSLGERATINCKSSONILYSWNNKNYLAWYQQKPGQAPKLLIY
WASTRESGVPDRFSGSGSGTDFTLTISSLQAEDVAVYYCHQYYSSPLTFGGGTKVEIK
>m42_HH28K_13_SL99F
(SEQ ID NO: 120)
VH: m42.127_HC_WT, QVQLVQSGAEVKKPGASVKVSCKASGYTFTSYAIHWVRQAPGQRLEWMGWIKAGNGNT
RYSQKFQDRVTITRDTSASTAYMELSSLRSEDTAVYYCALLTVITPDDTFDIWGQGTMVTVSS
(SEQ ID NO: 121)
VL: m42.127_LC_SL99F, DIVMTQSPDSLAVSLGERATINCKSSQNILYSSNNKNYLAWYQQKPGQAPKLLIY
WASTRESGVPDRFSGSGSGTDFTLTISSLQAEDVAVYYCHQYYfSPLTFGGGTKVEIK
>m42_HH28K_13_TH100C
(SEQ ID NO: 122)
VH: m42.127_HC_TH100C, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLCVITPDDTFDIWGQGTMVTVSS
(SEQ ID NO: 123)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_YH32K
(SEQ ID NO: 124)
VH: m42.127_HC_YH32K, QVQLVQSGAEVKKPGASVKVSCKASGYTFTSKAIHWVRQAPGQRLEWMGWIKAGN
GNTRYSQKFQDRVTITRDTSASTAYMELSSLRSEDTAVYYCALLTVITPDDTFDIWGQGTMVTVSS
(SEQ ID NO: 125)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_YL38W
(SEQ ID NO: 126)
VH: m42.127_HC_WT, QVQLVQSGAEVKKPGASVKVSCKASGYTFTSYAIHWVRQAPGQRLEWMGWIKAGNGNT
RYSQKFQDRVTITRDTSASTAYMELSSLRSEDTAVYYCALLTVITPDDTFDIWGQGTMVTVSS
(SEQ ID NO: 127)
VL: m42.127_LC_YL38W, DIVMTQSPDSLAVSLGERATINCKSSQNILYSSNNKNWLAWYQQKPGQAPKLLIY
WASTRESGVPDRFSGSGSGTDFTLTISSLQAEDVAVYYCHQYYSSPLITFGGGTKVEIK
>m42_HH28K_13_TH103R_PH104R
(SEQ ID NO: 128)
VH: m42.127_HC_TH103R_PH104R, QVQLVQSGAEVKKPGASVKVSCKASGYTFTSYAIHWVRQAPGQRLEW
MGWIKAGNGNTRYSQKFQDRVTITRDTSASTAYMELSSLRSEDTAVYYCALLTVIRRDDTFDIWGQGTMVTVSS
(SEQ ID NO: 129)
VL: m42.127_LC_WT, DIVMTQSPDSLAVSLGERATINCKSSQNILYSSNNKNYLAWYQQKPGQAPKLLIYWAS
TRESGVPDRESGSGSGTDFTLTISSLQAEDVAVYYCHQYYSSPLTFGGGTKVEIK
>m42_HH28K_13_TH103R_NL34W
(SEQ ID NO: 130)
VH: m42.127_HC_TH103R, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTVIRPDDTFDIWGQGTMVTVSS
VL: m42.127_LC_NL34W
>m42_HH28K_13_TH103R_PH104Q
(SEQ ID NO: 131)
VH: m42.127_HC_TH103R_PH1040, QVQLVQSGAEVKKPGASVKVSCKASGYTFTSYAIHWVRQAPGQRLEW
MGWIKAGNGNTRYSQKFQDRVTITRDTSASTAYMELSSLRSEDTAVYYCALLTVIRQDDTFDIWGQGTMVTVSS
(SEQ ID NO: 132)
VL: m42.127_LC_WT, DIVMTQSPDSLAVSLGERATINCKSSQNILYSSNNKNYLAWYQQKPGQAPKLLIYWAS
TRESGVPDRFSGSGSGTDFTLTISSLQAEDVAVYYCHQYYSSPLTFGGGTKVEIK
>m42_HH28K_13_TH103R_SL33W
(SEQ ID NO: 133)
VH: m42.127_HC_TH103R, QVQLVQSGAEVKKPGASVKVSCKASGYTFTSYAIHWVRQAPGQRLEWMGWIKAG
NGNTRYSQKFQDRVTITRDTSASTAYMELSSLRSEDTAVYYCALLTVIRPDDTFDIWGQGTMVTVSS
(SEQ ID NO: 134)
VL: m42.127_LC_SL33W, DIVMTQSPDSLAVSLGERATINCKSSQNILYSWNNKNYLAWYQQKPGQAPKLLIY
WASTRESGVPDRFSGSGSGTDFTLTISSLQAEDVAVYYCHQYYSSPLTFGGGTKVEIK
>m42_HH28K_13_AH33W_IL29H_NL34W_SL99F_TH103R
(SEQ ID NO: 135)
VH: m42.127_HC_AH33W_TH103R, QVQLVQSGAEVKKPGASVKVSCKASGYTFTSYWIHWVRQAPGQRLEWM
GWIKAGNGNTRYSQKFQDRVTITRDTSASTAYMELSSLRSEDTAVYYCALLTVIRPDDTFDIWGQGTMVTVSS
(SEQ ID NO: 136)
VL: m42.127_LC_IL29H_NL34W_SL99F, DIVMTQSPDSLAVSLGERATINCKSSONHLYSSWNKNYLAWYQ
QKPGQAPKLLIYWASTRESGVPDRFSGSGSGTDFTLTISSLQAEDVAVYYCHQYYFSPLTFGGGTKVEIK
>m42_HH28K_13_AH33W_IL29H_PH104K_SL99Y_TH103R
(SEQ ID NO: 137)
VH: m42.127_HC_AH33W_PH104K_TH103R, QVQLVQSGAEVKKPGASVKVSCKASGYTFTSYWIHWVRQAP
GQRLEWMGWIKAGNGNTRYSQKFQDRVTITRDTSASTAYMELSSLRSEDTAVYYCALLTVIrkDDTFDIWGQGT
MVTVSS
(SEQ ID NO: 138)
VL: m42.127_LC_IL29H_SL99Y, DIVMTQSPDSLAVSLGERATINCKSSQNhLYSSNNKNYLAWYQQKPGQA
PKLLIYWASTRESGVPDRFSGSGSGTDFTLTISSLQAEDVAVYYCHQYYYSPLTFGGGTKVEIK
>m42_HH28K_13_AH33W_NL34W_PH104M_PL101M_TH103R
(SEQ ID NO: 139)
VH: m42.127_HC_AH33W_PH104M_TH103R, QVQLVQSGAEVKKPGASVKVSCKASGYTFTSYWIHWVRQAP
GQRLEWMGWIKAGNGNTRYSQKFQDRVTITRDTSASTAYMELSSLRSEDTAVYYCALLTVIRMDDTFDIWGQGT
MVTVSS
(SEQ ID NO: 140)
VL: m42.127_LC_NL34W_PL101M, DIVMTQSPDSLAVSLGERATINCKSSQNILYSSWNKNYLAWYQQKPGQ
APKLLIYWASTRESGVPDRFSGSGSGTDFTLTISSLQAEDVAVYYCHQYYSSMLTFGGGTKVEIK
>m42_HH28K_13_AH53R_HH35E_PH104R_SL99F_YH32R_YL38W
(SEQ ID NO: 141)
VH: m42.127_HC_AH53R_HH35E_PH104R_YH32R, QVQLVQSGAEVKKPGASVKVSCKASGYTFTSrAIeW
VRQAPGQRLEWMGWIKrGNGNTRYSQKFQDRVTITRDTSASTAYMELSSLRSEDTAVYYCALLTVITrDDTEDI
WGQGTMVTVSS
(SEQ ID NO: 142)
VL: m42.127_LC_SL99F_YL38W, DIVMTQSPDSLAVSLGERATINCKSSQNILYSSNNKNWLAWYQQKPGQA
PKLLIYWASTRESGVPDRFSGSGSGTDFTLTISSLQAEDVAVYYCHQYYfSPLTFGGGTKVEIK
>m42_HH28K_13_PH104R_SH31R_SL33I_TH103R
(SEQ ID NO: 143)
VH: m42.127_HC_PH104R_SH31R_TH103R, QVQLVQSGAEVKKPGASVKVSCKASGYTFTRYAIHWVRQAP
GQRLEWMGWIKAGNGNTRYSQKFQDRVTITRDTSASTAYMELSSLRSEDTAVYYCALLTVIRRDDTFDIWGQGT
MVTVSS
(SEQ ID NO: 144)
VL: m42.127_LC_SL33I, DIVMTQSPDSLAVSLGERATINCKSSQNILYSINNKNYLAWYQQKPGQAPKLLIY
WASTRESGVPDRFSGSGSGTDFTLTISSLQAEDVAVYYCHQYYSSPLTFGGGTKVEIK
>m42_HH28K_13_AH33W_NL34W_SH31R_SL99F_TH103R_VH101Q
(SEQ ID NO: 145)
VH: m42.127_HC_AH33W_SH31R_TH103R_VH101Q, QVQLVQSGAEVKKPGASVKVSCKASGYTFTRYWIH
WVRQAPGQRLEWMGWIKAGNGNTRYSQKFQDRVTITRDTSASTAYMELSSLRSEDTAVYYCALLTQIRPDDTED
IWGQGTMVTVSS
(SEQ ID NO: 146)
VL: m42.127_LC_NL34W_SL99F, DIVMTQSPDSLAVSLGERATINCKSSONILYSSWNKNYLAWYQQKPGQA
PKLLIYWASTRESGVPDRFSGSGSGTDFTLTISSLQAEDVAVYYCHQYYFSPLTFGGGTKVEIK
>m42_HH28K_13_AH53R_IL29H_PH104M_SL99F_YH32R_YL38W
(SEQ ID NO: 147)
VH: m42.127_HC_AH53R_PH104M_YH32R, QVQLVQSGAEVKKPGASVKVSCKASGYTFTSrAIHWVRQAPG
QRLEWMGWIKrGNGNTRYSQKFQDRVTITRDTSASTAYMELSSLRSEDTAVYYCALLTVITmDDTFDIWGQGTM
VTVSS
(SEQ ID NO: 148)
VL: m42.127_LC_IL29H_SL99F_YL38W, DIVMTQSPDSLAVSLGERATINCKSSQNhLYSSNNKNwLAWYQ
QKPGQAPKLLIYWASTRESGVPDRFSGSGSGTDFTLTISSLQAEDVAVYYCHQYYfSPLTFGGGTKVEIK
>m42_HH28K_13_AH53R_NL34W_PH104M_SL99H_YL38W
(SEQ ID NO: 149)
VH: m42.127_HC_AH53R_PH104M, QVQLVQSGAEVKKPGASVKVSCKASGYTFTSYAIHWVRQAPGQRLEWM
GWIKrGNGNTRYSQKFQDRVTITRDTSASTAYMELSSLRSEDTAVYYCALLTVITmDDTFDIWGQGTMVTVSS
(SEQ ID NO: 150)
VL: m42.127_LC_NL34W_SL99H_YL38W, DIVMTQSPDSLAVSLGERATINCKSSQNILYSSWNKNWLAWYQ
QKPGQAPKLLIYWASTRESGVPDRFSGSGSGTDFTLTISSLQAEDVAVYYCHQYYhSPLTFGGGTKVEIK
>m42_HH28K_13_AH53R_NL34W_PH104R_SL99H_YH32R
(SEQ ID NO: 151)
VH: m42.127_HC_AH53R_PH104R_YH32R, QVQLVQSGAEVKKPGASVKVSCKASGYTFTSRAIHWVRQAPG
QRLEWMGWIKRGNGNTRYSQKFQDRVTITRDTSASTAYMELSSLRSEDTAVYYCALLTVITRDDTFDIWGQGTM
VTVSS
(SEQ ID NO: 152)
VL: m42.127_LC_NL34W_SL99H, DIVMTQSPDSLAVSLGERATINCKSSQNILYSSWNKNYLAWYQQKPGQA
PKLLIYWASTRESGVPDRFSGSGSGTDFTLTISSLQAEDVAVYYCHQYYHSPLTFGGGTKVEIK
>m42_HH28K_13_AH53R_PH104R_SL32T_SL33M_TH103K_TL59Y_YH32R
(SEQ ID NO: 153)
VH: m42.127_HC_AH53R_PH104R_TH103K_YH32R, QVQLVQSGAEVKKPGASVKVSCKASGYTFTSRAIH
WVRQAPGQRLEWMGWIKRGNGNTRYSQKFQDRVTITRDTSASTAYMELSSLRSEDTAVYYCALLTVIKRDDTED
IWGQGTMVTVSS
(SEQ ID NO: 154)
VL: m42.127_LC_SL32T_SL33M_TL59Y, DIVMTQSPDSLAVSLGERATINCKSSQNILYTMNNKNYLAWYQ
QKPGQAPKLLIYWASYRESGVPDRFSGSGSGTDFTLTISSLQAEDVAVYYCHQYYSSPLTFGGGTKVEIK
>m42_HH28K_13_AH53R_PL101M_SL33I_TH103R_YH32R
(SEQ ID NO: 155)
VH: m42.127_HC_AH53R_TH103R_YH32R, QVQLVQSGAEVKKPGASVKVSCKASGYTFTSRAIHWVRQAPG
QRLEWMGWIKRGNGNTRYSQKFQDRVTITRDTSASTAYMELSSLRSEDTAVYYCALLTVIRPDDTEDIWGQGTM
VTVSS
(SEQ ID NO: 156)
VL: m42.127_LC_PL101M_SL33I, DIVMTQSPDSLAVSLGERATINCKSSQNILYSINNKNYLAWYQQKPGQ
APKLLIYWASTRESGVPDRFSGSGSGTDFTLTISSLQAEDVAVYYCHQYYSSMLTFGGGTKVEIK
>m42_HH28K_13_IL29H_PH104R_SL99F_TH107V_TL59H_YH32R_YL38W
(SEQ ID NO: 157)
VH: m42.127_HC_PH104R_TH107V_YH32R, QVQLVQSGAEVKKPGASVKVSCKASGYTFTSRAIHWVRQAP
GQRLEWMGWIKAGNGNTRYSQKFQDRVTITRDTSASTAYMELSSLRSEDTAVYYCALLTVITRDDVEDIWGQGT
MVTVSS
(SEQ ID NO: 158)
VL: m42.127_LC_IL29H_SL99F_TL59H_YL38W, DIVMTQSPDSLAVSLGERATINCKSSQNHLYSSNNKN
WLAWYQQKPGQAPKLLIYWASHRESGVPDRFSGSGSGTDFTLTISSLQAEDVAVYYCHQYYF SPL TFGGGTKVE
IK
>m42_HH28K_13_PH104M_SH31R_SL99F_TH103R_YL38W
(SEQ ID NO: 159)
VH: m42.127_HC_PH104M_SH31R_TH103R, QVQLVQSGAEVKKPGASVKVSCKASGYTFTRYAIHWVRQAP
GQRLEWMGWIKAGNGNTRYSQKFQDRVTITRDTSASTAYMELSSLRSEDTAVYYCALLTVIRMDDTEDIWGQGT
MVTVSS
(SEQ ID NO: 160)
VL: m42.127_LC_SL99F_YL38W, DIVMTQSPDSLAVSLGERATINCKSSONILYSSNNKNWLAWYQQKPGQA
PKLLIYWASTRESGVPDRFSGSGSGTDFTLTISSLQAEDVAVYYCHQYYfSPLTFGGGTKVEIK
>m42_HH28K_13_PH104R_SL32T_TH103R_YL97W
(SEQ ID NO: 161)
VH: m42.127_HC_TH103R_PH104R, QVQLVQSGAEVKKPGASVKVSCKASGYTFTSYAIHWVRQAPGQRLEW
MGWIKAGNGNTRYSQKFQDRVTITRDTSASTAYMELSSLRSEDTAVYYCALLTVIRRDDTFDIWGQGTMVTVSS
(SEQ ID NO: 162)
VL: m42.127_LC_SL32T_YL97W, DIVMTQSPDSLAVSLGERATINCKSSQNILYTSNNKNYLAWYQQKPGQA
PKLLIYWASTRESGVPDRFSGSGSGTDFTLTISSLQAEDVAVYYCHQWYSSPLTFGGGTKVEIK
>m43_HH28K_17_VH101R
(SEQ ID NO: 163)
VH: m43.151_HC_VH101R, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLTRITPDDAFDIWGQGTMVTVSS
(SEQ ID NO: 164)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_VH101W
(SEQ ID NO: 165)
VH: m43.151_HC_VH101W, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLTWITPDDAFDIWGQGTMVTVSS
(SEQ ID NO: 166)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_TH100I
(SEQ ID NO: 167)
VH: m43.151_HC_TH100I, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLIVITPDDAFDIWGQGTMVTVSS
(SEQ ID NO: 168)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_TH103N
(SEQ ID NO: 169)
VH: m43.151_HC_TH103N, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLTVINPDDAFDIWGQGTMVTVSS
(SEQ ID NO: 170)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_YL38R
(SEQ ID NO: 171)
VH: m43.151_HC_WT, QVHLVQSGAEVKKPGASVKVSCKASGYTFTSYAIHWVRQAPGQRLEWMGWIKGGNGNT
RYSQKFQDRVTITRDTSASTAYMELSSLRSEDTAVYYCALLTVITPDDAFDIWGQGTMVTVSS
(SEQ ID NO: 172)
VL: m43.151_LC_YL38R, DIVMTQSPDSLAVSLGERATINCKSSQNILYSSNNKNRLAWYQQKPGQPPKLLFY
WASTRESGVPDRFSGSGSGTDFTLTISSLQAEDVAVYYCHQYYSSPLTFGGGTKVEIK
>m43_HH28K_17_AH107R
(SEQ ID NO: 173)
VH: m43.151_HC_AH107R, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLTVITPDDRFDIWGQGTMVTVSS
(SEQ ID NO: 174)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLITFGGGTKVEIK
>m43_HH28K_17_PH104K
(SEQ ID NO: 175)
VH: m43.151_HC_PH104K, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLTVITKDDAFDIWGQGTMVTVSS
(SEQ ID NO: 176)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_PH104R
(SEQ ID NO: 177)
VH: m43.151_HC_PH104R, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLTVITRDDAFDIWGQGTMVTVSS
(SEQ ID NO: 178)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_TH100M
(SEQ ID NO: 179)
VH: m43.151_HC_TH100M, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLMVITPDDAFDIWGQGTMVTVSS
(SEQ ID NO: 180)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_TH100Q
(SEQ ID NO: 181)
VH: m43.151_HC_TH100Q, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLQVITPDDAFDIWGQGTMVTVSS
(SEQ ID NO: 182)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_YH32R
(SEQ ID NO: 183)
VH: m43.151_HC_YH32R, QVHLVQSGAEVKKPGASVKVSCKASGYTFTSrAIHWVRQAPGQRLEWMGWIKGGN
GNTRYSQKFQDRVTITRDTSASTAYMELSSLRSEDTAVYYCALLTVITPDDAFDIWGQGTMVTVSS
(SEQ ID NO: 184)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_VH101R_PH104R
(SEQ ID NO: 185)
VH: m43.151_HC_VH101R_PH104R, QVHLVQSGAEVKKPGASVKVSCKASGYTFTSYAIHWVRQAPGQRLEW
MGWIKGGNGNTRYSQKFQDRVTI TRDTSASTAYMELSSLRSEDTAVYYCALLTRI TRDDAFDIWGQGTMVTVSS
(SEQ ID NO: 186)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_TH100I_YH32R
(SEQ ID NO: 187)
VH: m43.151_HC_TH100I_YH32R, QVHLVQSGAEVKKPGASVKVSCKASGYTFTSRAIHWVRQAPGQRLEWM
GWIKGGNGNTRYSQKFQDRVTITRDTSASTAYMELSSLRSEDTAVYYCALLIVITPDDAFDIWGQGTMVTVSS
(SEQ ID NO: 188)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_AH33W_SL99E_VH101R
(SEQ ID NO: 189)
VH: m43.151_HC_AH33W_VH101R, QVHLVQSGAEVKKPGASVKVSCKASGYTFTSYWIHWVRQAPGQRLEWM
GWIKGGNGNTRYSQKFQDRVTITRDTSASTAYMELSSLRSEDTAVYYCALLTRITPDDAFDIWGQGTMVTVSS
(SEQ ID NO: 190)
VL: m43.151_LC_SL99E, DIVMTQSPDSLAVSLGERATINCKSSQNILYSSNNKNYLAWYQQKPGQPPKLLFY
WASTRESGVPDRFSGSGSGTDFTLTISSLQAEDVAVYYCHQYYESPLTFGGGTKVEIK
>m43_HH28K_17_AH33W_VH101R
(SEQ ID NO: 191)
VH: m43.151_HC_AH33W_VH101R, QVHLVQSGAEVKKPGASVKVSCKASGYTFTSYWIHWVRQAPGQRLEWM
GWIKGGNGNTRYSQKFQDRVTITRDTSASTAYMELSSLRSEDTAVYYCALLTRITPDDAFDIWGQGTMVTVSS
(SEQ ID NO: 192)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLITFGGGTKVEIK
>m43_HH28K_17_LL102Q_NL34M_VH101R
(SEQ ID NO: 193)
VH: m43.151_HC_VH101R, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLTRITPDDAFDIWGQGTMVTVSS
(SEQ ID NO: 194)
VL: m43.151_LC_LL102Q_NL34M, DIVMTQSPDSLAVSLGERATINCKSSQNILYSSMNKNYLAWYQQKPGQ
PPKLLFYWASTRESGVPDRFSGSGSGTDFTLTISSLQAEDVAVYYCHQYYSSPQTFGGGTKVEIK
>m43_HH28K_17_AH33W_SL32F_TH103Q_YL38R
(SEQ ID NO: 195)
VH: m43.151_HC_AH33W_TH103Q, QVHLVQSGAEVKKPGASVKVSCKASGYTFTSYWI HWVRQAPGQRLEWM
GWIKGGNGNTRYSQKFQDRVTITRDTSASTAYMELSSLRSEDTAVYYCALLTVIQPDDAFDIWGQGTMVTVSS
(SEQ ID NO: 196)
VL: m43.151_LC_SL32F_YL38R, DIVMTQSPDSLAVSLGERATINCKSSQNILYFSNNKNRLAWYQQKPGQP
PKLLFYWASTRESGVPDRFSGSGSGTDFTLTISSLQAEDVAVYYCHQYYSSPLTFGGGTKVEIK
>m43_HH28K_17_NL34M_VH101R
(SEQ ID NO: 197)
VH: m43.151_HC_VH101R, QVHLVQSGAEVKKPGASVKVSCKASGYTFTSYAIHWVRQAPGQRLEWMGWIKGG
NGNTRYSQKFQDRVTITRDTSASTAYMELSSLRSEDTAVYYCALLTRITPDDAFDIWGQGTMVTVSS
(SEQ ID NO: 198)
VL: m43.151_LC_NL34M, DIVMTQSPDSLAVSLGERATINCKSSQNILYSSMNKNYLAWYQQKPGQPPKLLFY
WASTRESGVPDRFSGSGSGTDFTLTISSLQAEDVAVYYCHQYYSSPLTFGGGTKVEIK
>m43_HH28K_17_AH24R_LL52Y_PH104R_VH101K
(SEQ ID NO: 199)
VH: m43.151_HC_AH24R_PH104R_VH101K, QVHLVQSGAEVKKPGASVKVSCKrSGYTFTSYAIHWVRQAP
GQRLEWMGWIKGGNGNTRYSQKFQDRVTITRDTSASTAYMELSSLRSEDTAVYYCALLTKITr DDAFDIWGQGT
MVTVSS
(SEQ ID NO: 200)
VL: m43.151_LC_LL52Y, DIVMTQSPDSLAVSLGERATINCKSSQNILYSSNNKNYLAWYQQKPGQPPKYLFY
WASTRESGVPDRFSGSGSGTDFTLTISSLQAEDVAVYYCHQYYSSPLTFGGGTKVEIK
>m43_HH28K_17_AH33W_LH99F
(SEQ ID NO: 201)
VH: m43.151_HC_AH33W_LH99F, QVHLVQSGAEVKKPGASVKVSCKASGYTFTSYWIHWVRQAPGQRLEWMG
WIKGGNGNTRYSQKFQDRVTI TRDTSASTAYMELSSLRSEDTAVYYCALFTVITPDDAFDIWGQGTMVTVSS
(SEQ ID NO: 202)
VL: m43.151_LC_WT, DIVMTQSPDSLAVSLGERATINCKSSONILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_PH104R_SH31T_YH32R
(SEQ ID NO: 203)
VH: m43.151_HC_PH104R_SH31T_YH32R, QVHLVQSGAEVKKPGASVKVSCKASGYTFTTRAIHWVRQAPG
QRLEWMGWIKGGNGNTRYSQKFQDRVTITRDTSASTAYMELSSLRSEDTAVYYCALLTVITRDDAFDIWGQGTM
VTVSS
(SEQ ID NO: 204)
VL: m43.151_LC_WT, DIVMTQSPDSLAVSLGERATINCKSSONILYSSNNKNYLAWYQQKPGQPPKLLFYWAS
TRESGVPDRFSGSGSGTDFTLTISSLQAEDVAVYYCHQYYSSPLITFGGGTKVEIK
>m43_HH28K_17_IH34W_PH104H_SL32D_VH101R
(SEQ ID NO: 205)
VH: m43.151_HC_IH34W_PH104H_VH101R, QVHLVQSGAEVKKPGASVKVSCKASGYTFTSYAWHWVRQAP
GQRLEWMGWIKGGNGNTRYSQKFQDRVTITRDTSASTAYMELSSLRSEDTAVYYCALLTRITHDDAFDIWGQGT
MVTVSS
(SEQ ID NO: 206)
VL: m43.151_LC_SL32D, DIVMTQSPDSLAVSLGERATINCKSSQNILYDSNNKNYLAWYQQKPGQPPKLLFY
WASTRESGVPDRFSGSGSGTDFTLTISSLQAEDVAVYYCHQYYSSPLTFGGGTKVEIK
>m43_HH28K_17_PL101Y_SH31R_VH101R
(SEQ ID NO: 207)
VH: m43.151_HC_SH31R_VH101R, QVHLVQSGAEVKKPGASVKVSCKASGYTFTRYAIHWVRQAPGQRLEWM
GWIKGGNGNTRYSQKFQDRVTITRDTSASTAYMELSSLRSEDTAVYYCALLTRITPDDAFDIWGQGTMVTVSS
(SEQ ID NO: 208)
VL: m43.151_LC_PL101Y, DIVMTQSPDSLAVSLGERATINCKSSONILYSSNNKNYLAWYQQKPGQPPKLLF
YWASTRESGVPDRFSGSGSGTDFTLTISSLQAEDVAVYYCHQYYSSYLTFGGGTKVEIK
>m43_HH28K_17_SL99H_TH100I
(SEQ ID NO: 209)
VH: m43.151_HC_TH100I, QVHLVQSGAEVKKPGASVKVSCKASGYTFTSYAIHWVRQAPGQRLEWMGWIKG
GNGNTRYSQKFQDRVTITRDTSASTAYMELSSLRSEDTAVYYCALLIVITPDDAFDIWGQGTMVTVSS
(SEQ ID NO: 210)
VL: m43.151_LC_SL99H, DIVMTQSPDSLAVSLGERATINCKSSQNILYSSNNKNYLAWYQQKPGQPPKLLFY
WASTRESGVPDRFSGSGSGTDFTLTISSLQAEDVAVYYCHQYYHSPLTFGGGTKVEIK
>m43_HH28K_17_TH100M_YH32R
(SEQ ID NO: 211)
VH: m43.151_HC_TH100M_YH32R, QVHLVQSGAEVKKPGASVKVSCKASGYTFTSRAIHWVRQAPGQRLEWM
GWIKGGNGNTRYSQKFQDRVTITRDTSASTAYMELSSLRSEDTAVYYCALLMVITPDDAFDIWGQGTMVTVSS
(SEQ ID NO: 212)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK
>m43_HH28K_17_VH101R_YH32R
(SEQ ID NO: 213)
VH: m43.151_HC_VH101R_YH32R, QVHLVQSGAEVKKPGASVKVSCKASGYTFTSRAIHWVRQAPGQRLEWM
GWIKGGNGNTRYSQKFQDRVTITRDTSASTAYMELSSLRSEDTAVYYCALLTRITPDDAFDIWGQGTMVTVSS
(SEQ ID NO: 214)
VL: m43.151_LC_WT, DIVMTQSPDSLAVSLGERATINCKSSQNILYSSNNKNYLAWYQQKPGQPPKLLFYWAS
TRESGVPDRFSGSGSGTDFTLTISSLQAEDVAVYYCHQYYSSPLTFGGGTKVEIK

### Example 3

### Variant CIS43 Antibodies

This example illustrates the isolation and assessment of additional variant CIS43 antibodies using the iGL-CIS43 mouse model described in Example 1.

H^{iGL-CIS43}κ^{iGL-CIS43} B cells were adoptively transferred into congenic mice that were then immunized with NPDP19-KLH/Alhydrogel, as discussed in Example 1. 42 days post-immunization, splenocytes were isolated from immunized mice and FACS-sorted for binding to NPDP19-KLH as described above. Isolated B-cells positive for binding to VLP-4 or NPDP19-KLH were single cell sorted and sequenced (50 antibodies from each assay, for a total of 100 sequenced antibodies). The antibody sequences are provided as Appendix F of U.S. Provisional Application No. 63/275,936, filed Nov. 4, 2021; Appendices F is incorporated by reference herein in its entirety.

The NPDP19-KLH- selected antibody VH and VL sequences were cloned into IgG expression plasmid and assessed for NPDP19 binding by ALPHALISA® as described in Example 1, with the antibody ID according to the following table. For these assays, antibody IgG present in supernatant from antibody expressing cells was assessed for NPDP19 binding. Several antibodies were assessed for further assessment, including Antibody ID No. 39 (P4-39 or iGL-CIS43-KLH-D42.39) which was selected for assessment via mouse challenge study.

| ID | Antibody |
|---|---|
| 1 | iGL-CIS43-KLH-D42.1 |
| 2 | iGL-CIS43-KLH-D42.2 |
| 3 | iGL-CIS43-KLH-D42.3 |
| 4 | iGL-CIS43-KLH-D42.4 |
| 5 | iGL-CIS43-KLH-D42.5 |
| 6 | iGL-CIS43-KLH-D42.6 |
| 7 | iGL-CIS43-KLH-D42.7 |
| 8 | iGL-CIS43-KLH-D42.8 |
| 9 | iGL-CIS43-KLH-D42.9 |
| 10 | iGL-CIS43-KLH-D42.10 |
| 11 | iGL-CIS43-KLH-D42.11 |
| 12 | iGL-CIS43-KLH-D42.12 |
| 13 | iGL-CIS43-KLH-D42.13 |
| 14 | iGL-CIS43-KLH-D42.14 |
| 15 | iGL-CIS43-KLH-D42.15 |
| 16 | iGL-CIS43-KLH-D42.16 |
| 17 | iGL-CIS43-KLH-D42.17 |
| 18 | iGL-CIS43-KLH-D42.18 |
| 19 | iGL-CIS43-KLH-D42.19 |
| 20 | iGL-CIS43-KLH-D42.20 |
| 21 | iGL-CIS43-KLH-D42.21 |
| 22 | iGL-CIS43-KLH-D42.22 |
| 23 | iGL-CIS43-KLH-D42.23 |
| 24 | iGL-CIS43-KLH-D42.24 |
| 25 | iGL-CIS43-KLH-D42.25 |
| 26 | iGL-CIS43-KLH-D42.26 |
| 27 | iGL-CIS43-KLH-D42.27 |
| 28 | iGL-CIS43-KLH-D42.28 |
| 29 | iGL-CIS43-KLH-D42.29 |
| 30 | iGL-CIS43-KLH-D42.30 |
| 31 | iGL-CIS43-KLH-D42.31 |
| 32 | iGL-CIS43-KLH-D42.32 |
| 33 | iGL-CIS43-KLH-D42.33 |
| 34 | iGL-CIS43-KLH-D42.34 |
| 35 | iGL-CIS43-KLH-D42.35 |
| 36 | iGL-CIS43-KLH-D42.36 |
| 37 | iGL-CIS43-KLH-D42.37 |
| 38 | iGL-CIS43-KLH-D42.38 |
| 39 | iGL-CIS43-KLH-D42.39 |
| 40 | iGL-CIS43-KLH-D42.40 |
| 41 | iGL-CIS43-KLH-D42.41 |
| 42 | iGL-CIS43-KLH-D42.42 |
| 43 | iGL-CIS43-KLH-D42.43 |
| 44 | iGL-CIS43-KLH-D42.44 |
| 45 | iGL-CIS43-KLH-D42.45 |
| 46 | iGL-CIS43-KLH-D42.46 |
| 47 | iGL-CIS43-KLH-D42.47 |
| 48 | iGL-CIS43-KLH-D42.48 |
| 49 | iGL-CIS43-KLH-D42.49 |
| 50 | iGL-CIS43-KLH-D42.50 |

### Example 4

### Assessment of Variant CIS43 Antibodies

This example describes assessment of purified variant CIS43 antibodies binding to NPDP peptide. The VH and VL of selected CIS43 variants were expressed as IgG as discussed above, purified, and assessed for binding to NPDP19 peptide by ALPHALISA®.

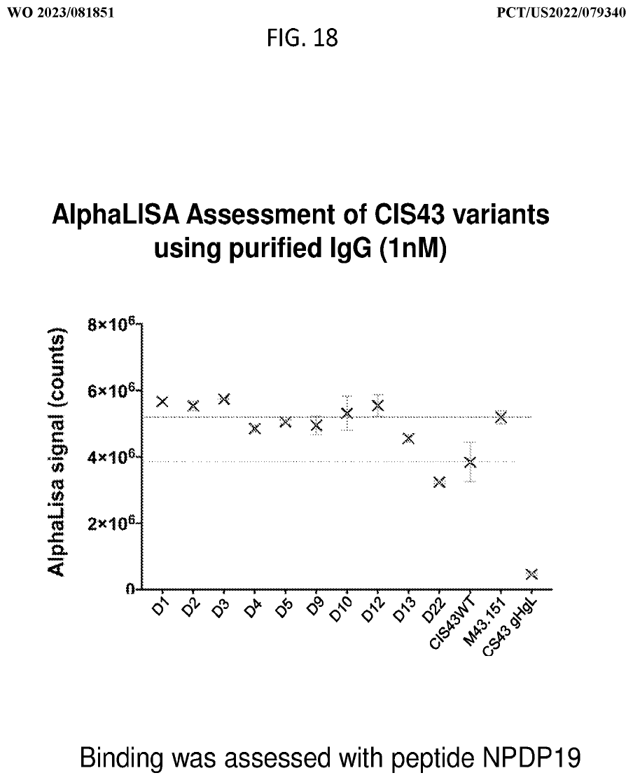

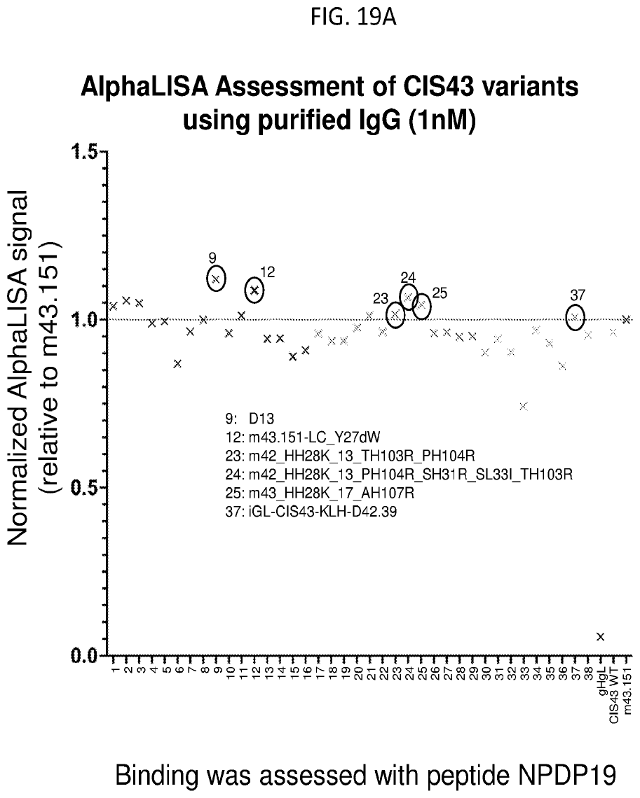

FIG. 18 shows binding of purified D1, D2, D3, D5, D9, D10, D12, D13, and D22 antibodies to NPDP19 peptide. The m43.151, CIS43 WT, and CIS43 gHgL antibodies are provided as controls, with the ALPHALISA® threshold m43.151 antibody indicated. Several of the variant antibodies exhibited increased binding to the NPDP19 peptide compared to m43.151 and CIS43 WT.

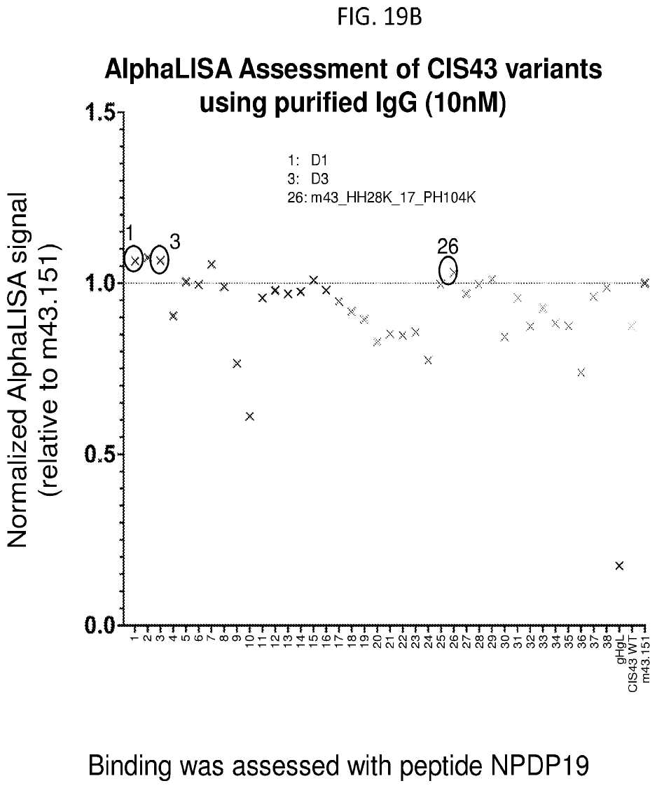

FIGS. 19A and 19B show binding of a series of variant CIS43 antibodies selected from those identified in the prior examples to NPDP19 peptide. Antibody concentrations of 1 nM (FIG. 19A) and 10 nM (FIG. 19B) were assessed. The antibody ID in the figure is according to the following table.

| ID in FIG. | |
|---|---|
| 19 | Antibody |
| 1 | D1 |
| 2 | D2 |
| 3 | D3 |
| 4 | D4 |
| 5 | D5 |
| 6 | D9 |
| 7 | D10 |
| 8 | D12 |
| 9 | D13 |
| 10 | D22 |
| 11 | m43.151-HC_W50Y |
| 12 | m43.151-LC_Y27dW |
| 13 | m43.151-LC_N28Q |
| 14 | m43.151-LC_S93R |
| 15 | m43.151-LC_S93K |
| 16 | m43.151-LC_S93L |
| 17 | m42_HH28K_13_TH103K |
| 18 | m42_HH28K_13_TH100M |
| 19 | m42_HH28K_13_PH104M |
| 20 | m42_HH28K_13_PH104Q |
| 21 | m42_HH28K_13_YH32K |
| 22 | m42_HH28K_13_TH103R_PH104R |
| 23 | m42_HH28K_13_TH103R_PH104Q |
| 24 | m42_HH28K_13_PH104R_SH31R_SL33I_TH103R |
| 25 | m43_HH28K_17_AH107R |
| 26 | m43_HH28K_17_PH104K |
| 27 | m43_HH28K_17_PH104R |
| 28 | m43_HH28K_17_TH100M |
| 29 | m43_HH28K_17_PH104R_SH31T_YH32R |
| 30 | iGL-CIS43-KLH-D42.2 |
| 31 | iGL-CIS43-KLH-D42.19 |
| 32 | iGL-CIS43-KLH-D42.24 |
| 33 | iGL-CIS43-KLH-D42.26 |
| 34 | iGL-CIS43-KLH-D42.34 |
| 35 | iGL-CIS43-KLH-D42.36 |
| 36 | iGL-CIS43-KLH-D42.37 |
| 37 | iGL-CIS43-KLH-D42.39 |
| 38 | iGL-CIS43-KLH-D42.45 |
| 39 | CIS43 gHgL |
| 40 | CIS43 WT |
| 41 | M43.151 |

In FIGS. 19A and 19B, the m43.151 and CIS43 WT antibodies are provided as controls, with the ALPHALISA® threshold for the m43.151 antibody indicated. Several of the variant antibodies exhibited increased binding to the NPDP19 peptide compared to m43.151 and CIS43 WT.

### Example 5

### Assessment of Variant CIS43 Antibodies in Animals

This example illustrates use of the variant CIS43 antibodies described herein to inhibit liver invasion of P. berghei in a mouse model.

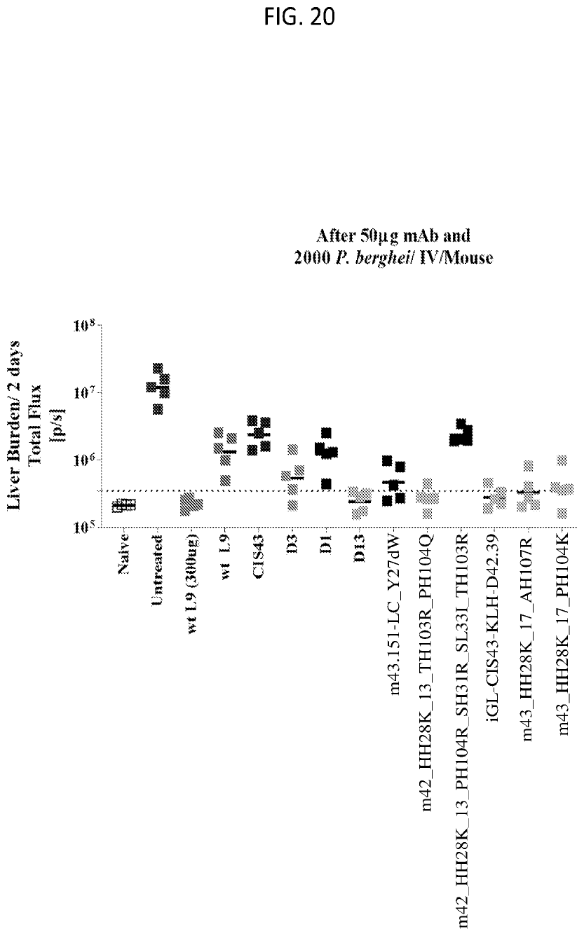

Mice were infected with P. berghei as described in Example 1 and treated with a panel of variant CIS43 antibodies identified herein. FIG. 20 presents results from assessment of the D1, D3, D13, m43.151-LC_Y27dW, m42_HH28K_13_TH103R_PH104Q, m42_HH28K_13_PH104R_SH31R_SL33I_TH103R, iGL-CIS43-KLH-D42.39, m43_HH28K_17_AH107R, and m43_HH28K_17_PH104K antibodies. The assay was performed with 50 μg antibody and 2000 P. berghei administered IV per mouse. Controls included naïve and untreated antibodies, as well as animals treated with L9 at 300 and 50 μg, and CIS43. All the assessed CIS43 variants were more protective than CIS43, and most were more protective than L9 at comparable antibody levels.

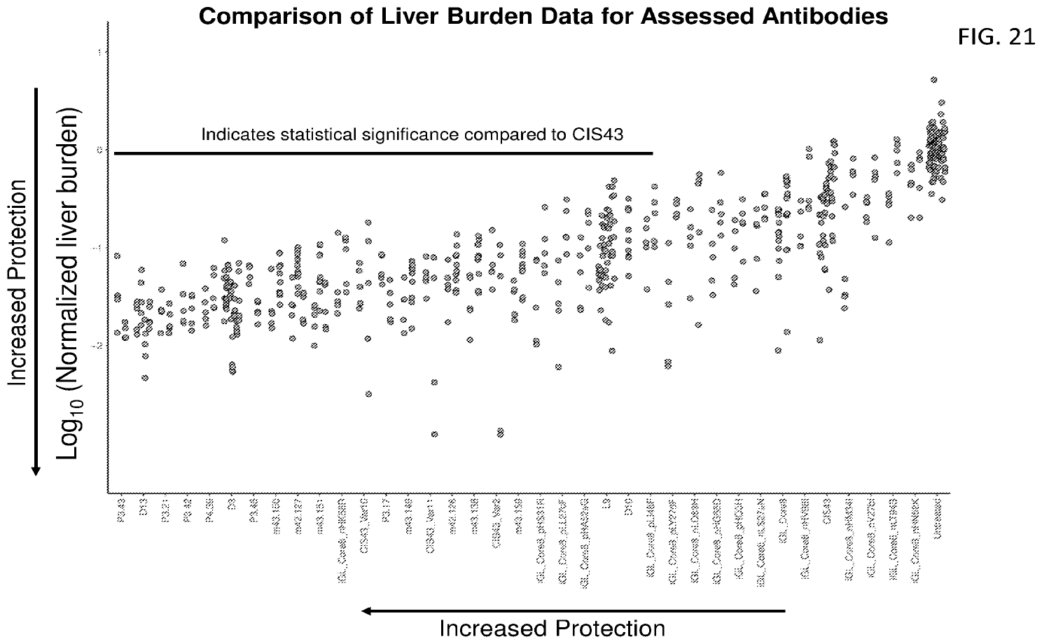

FIG. 21 provides a summary of liver burden data for variant CIS43 antibodies assessed using the P. berghei liver invasion mouse model collected across 16 different experiments. For comparison purposes, the results are normalized with the CIS43 condition. The antibodies are organized left to right from greatest to least median level of protection (normalized to CIS43) across assays, with the P3-43 antibody having the greatest level of protection/lowest liver burden and the untreated condition have the least level of protection/highest liver burder. Several of the variant antibodies provided a statistically significant improvement in liver burden compared to CIS43 (indicated).

### Example 6

### CIS43 Variants with Improved Half-Life

This example illustrates variant CIS43 antibodies with improved serum half-life that maintain high potency and breadth.

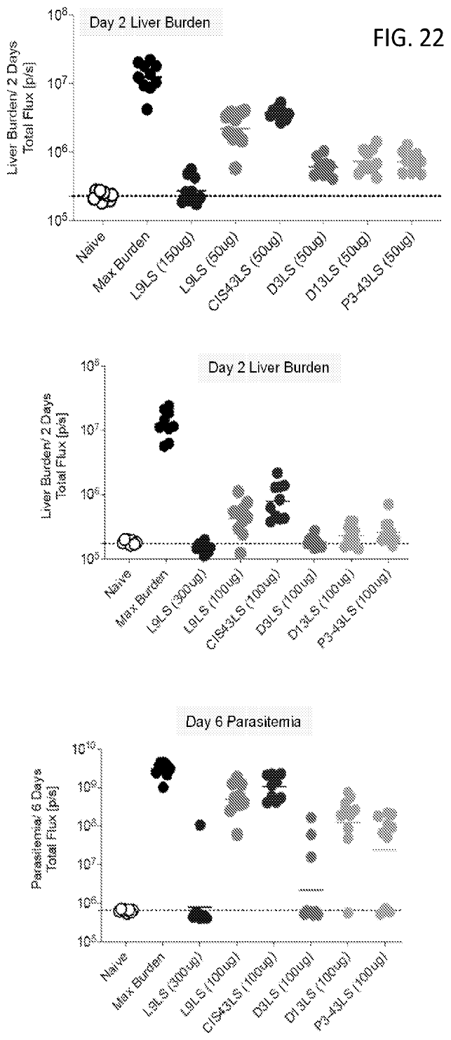

First, several of the variant CIS43 antibodies provided herein were modified with the “LS” mutation in the constant domain to increase binding to the neonatal Fc receptor. These antibodies were assessed functionally using the P. berghei liver invasion in a mouse model. Mice were infected with P. berghei as described in Example 1 and treated with the panel of variant CIS43 antibodies shown in FIG. 22. As indicated, the D3, D13, and P3-24 antibodies with the “LS” mutation all retained functional activity to block P. berghei liver invasion.

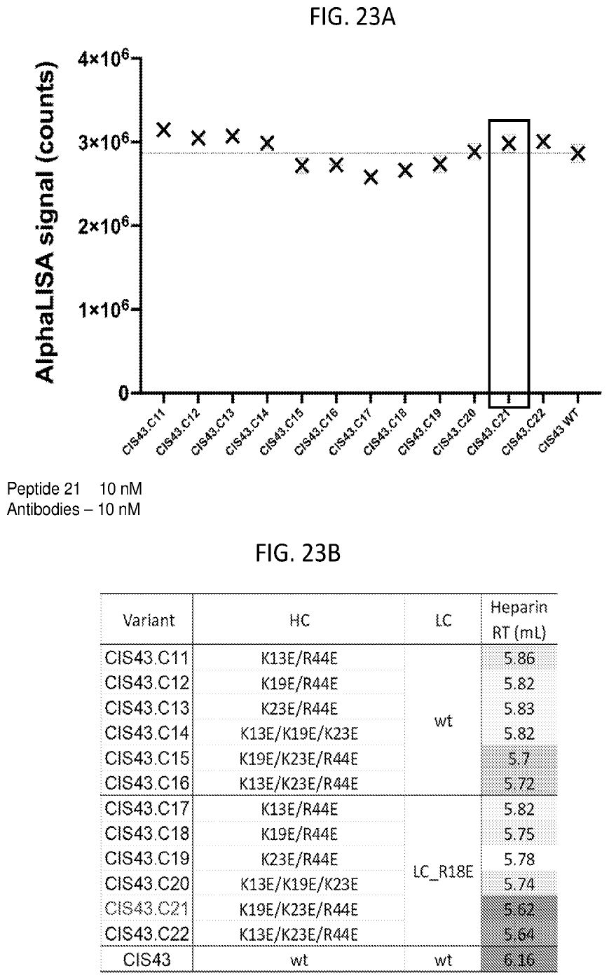

Additionally, a panel of CIS43LS variants (CIS43.C11-C12), incorporating select Arg or Lys residues in the variable domain substituted with Glu or Gln mutations, was assessed. The antibodies contain variable region substitutions to reduce off-target interactions mediated by charge-charge interaction while maintaining high potency and breadth. Combinations of the following substitutions were tested: K13E, K19E, K23E, R44E in the heavy chain and R18E in the light chain (see FIG. 23B).

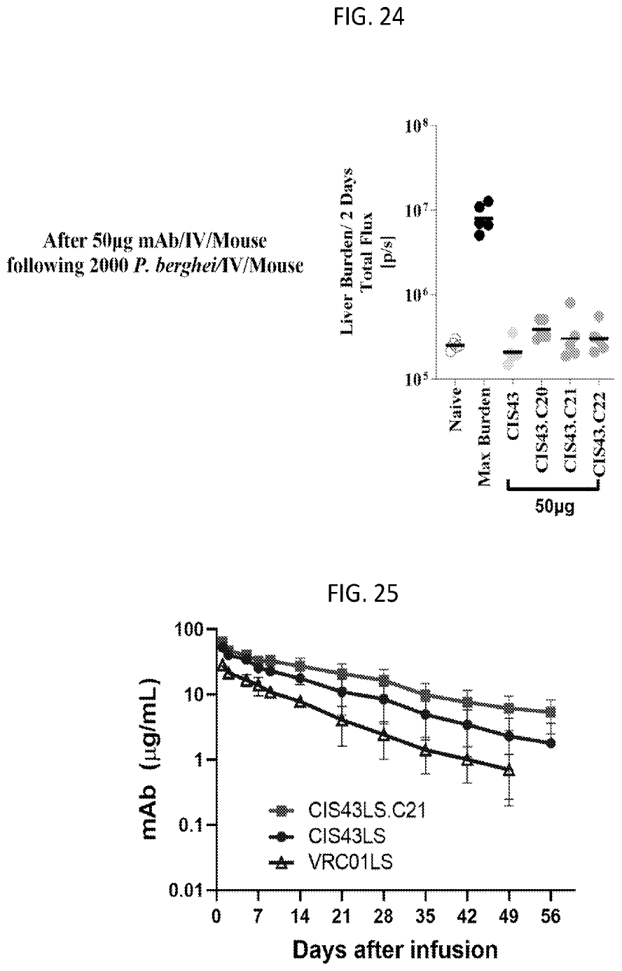

Assessment was done by affinity to CIS43 peptide (Peptide 21) by ALPHALISA® (FIG. 24A), Heparin chromatography (FIG. 23B), functional reduction in P. berghei infection (FIG. 24), and PK assessment in the human FcRn knock-in mice (FIG. 25).

FIG. 23A shows data from the testing of the CIS43 variants C11-C22 for binding to peptide 21 by ALPHALISA®. 10 nM of peptide 21 and 10 nM of CIS43 variants were used. The ALPHALISA® signals above the dotted horizontal line represent the affinity maintained or better than WT.

FIG. 23B shows mutational information for the CIS43 variants and their affinity to heparin by chromatography. The Heparin chromatography was run at a flowrate of 1 ml/min with running buffer A containing 10 mM Sodium phosphate at pH 7.4, Buffer B containing 10 mM Sodium phosphate, pH 7.4, 1M NaCl. The net positivity of the variants, i.e. affinity to Heparin, was measured by their “Retention Time (RT) or Retention Volume (RV).” The higher the net positivity the longer the RT or RV. The CIS43.C21 variant exhibited the greatest reduction in positivity while also retaining affinity to peptide 21.

CIS43 variants with the C20, C21, and C22 sets of mutations were assessed functionally using the P. berghei liver invasion in a mouse model (FIG. 24). Mice were infected with P. berghei as described in Example 1 and treated with the variant CIS43 antibodies shown in FIG. 24. The C21 set of mutations did not significantly change the CIS43 functional activity to block P. berghei liver invasion.

The pharmokinetic parameters of CIS43 with the “C21” set of mutations and with or without the “LS” mutation were assessed in a human FcRn knock-in mouse model. Results are shown in FIG. 25. CIS43LS.C21 showed 26%, 73%, and 34% improved half-life, AUC, and clearance rate, respectively, over CIS43LS.

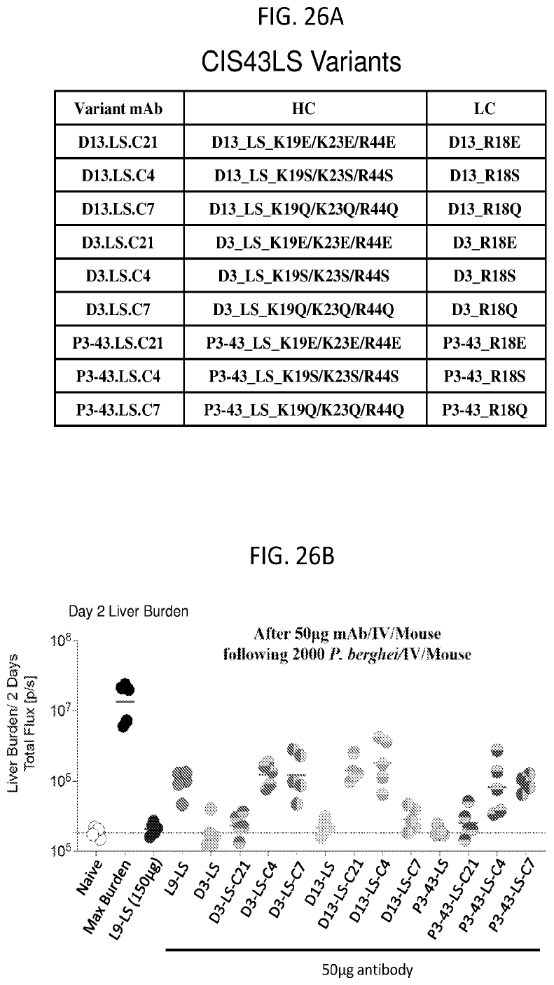

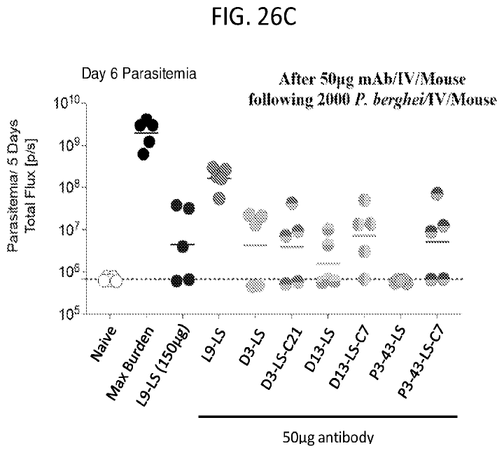

An additional set of CIS43 variants was created, based on the D13, D3, and P3-43 antibodies, with the “LS” substitution, and glutamate, serine, or glutamine substitutions at K19, K23 and R44 of the heavy chain and R18 of the light chain (FIG. 26A). Sequences are provided below.

>CIS43LS.C21 HC
(SEQ ID NO: 215)
QVQLVQSGAEVKKPGASVEVSCEASGYTFTSYAIHWVRQAPGQELEWMGW
IKAGNGNTRYSQKFQDRVTITRDTSTTTAYMELSSLRSEDTAVYYCALLT
VLTPDDAFDIWGQGTMVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKENWYVDGVEVHNAKTKPREEQY
NSTYRVVSVLIVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVLHEALHSHYTQKSLSLSPG
K
>CIS43LS.C21 LC
(SEQ ID NO: 216)
DIVMTQSPDSLAVSLGEEATINCKSSQSVLYSSNNKNYLAWYQQKPGQPP
NLLIYWASTRQSGVPDRFSGSGSGTDFTLTISSLQAEDVAVYYCHQYYSS
PLTFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSENRGEC
>CIS43_D3.C21 VH
(SEQ ID NO: 217)
QVHLVQSGAEVKKPGASVEVSCEASGYTFTRYAIHWVRQAPGQELEWMGW
IKGGNGNTRYSQKFQDRVTITRDTSASTAYMELSSLRSEDTAVYYCALLT
VITPDDAFDIWGQGTMVTVSS
>CIS43_D3.C21 VL
(SEQ ID NO: 218)
DIVMTQSPDSLAVSLGEEATINCKSSQNIFFSSNNKNYLAWYQQKPGQPP
KLLFYWASTRESGVPDRFSGSGSGTDFTLTISSLQAEDVAVYYCHQYYSS
PLTFGGGTKVEIK
>CIS43_D13.C7 VH
(SEQ ID NO: 219)
QVQLVQSGAEVKKPGASVQVSCQASGYTFTRYAIHWVRQAPGQQLEWMGW
IKAGNGNTRYSQKFQDRVTITRDTSASTAYMELSSLRSEDTAVYYCALLT
VITPDDAFDIWGQGTMVTVSS
>CIS43_D13.C7 VL
(SEQ ID NO: 220)
DIVMTQSPDSLAVSLGEEATINCKSSQNIFFSSKNKNYLAWYQQIPGQPP
KLLIYWASTRESGVPDRFSGSGSGTDFTLTISSLQAEDVAVYYCHQYYSS
PLTFGGGTKVEIQ
>CIS43_P3-43.C21 VH
(SEQ ID NO: 221)
QVHLVQSGAEVKKPGASVEVSCEASGYTFTSYAIHWVRQAPGQELEWMGW
IKGGNGNTRYSQKFQDRVTITRDTSASTAYMELSSLRSEDTAVYYCALLT
VITKDDAFDIWGQGTMVTVSS
>CIS43_P3-43.C21 VL
(SEQ ID NO: 222)
DIVMTQSPDSLAVSLGEEATINCKSSQNILYSSNNKNYLAWYQQKPGQPP
KLLFYWASTRESGVPDRFSGSGSGTDFTLTISSLQAEDVAVYYCHQYYSS
PLTFGGGTKVEIK

These antibodies were assessed functionally using the P. berghei liver invasion in a mouse model as discussed above. Mice (n=5/group) were passively infused with 50 μg of mAbs shown. Mice were then challenged with Pb-PfCSP sporozoites, and bioluminescence or total flux (photons/second) was quantified at day 2 as a measure of liver stage infection, and again at day 6 to determine parasitemia (blood stage infection). Results are shown in FIG. 26B (day 2 liver burden) and FIG. 26C (day 6 parasitemia).

A linear regression model and a population pharmacokinetic model were used to further interrogate the serum half-life of the variant CIS43 antibodies.

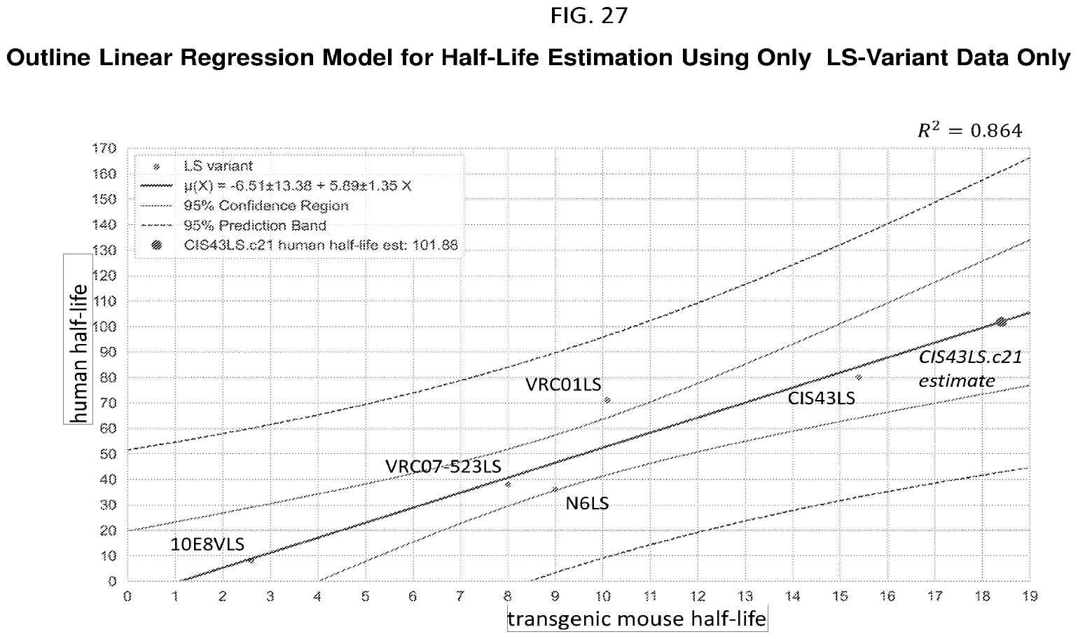

Based on the linear regression model (see FIG. 27), an estimate of the serum half-life in humans for the CIS43-LS.C21 antibody is 101.88±59.1 days. The linear model parameters were aslope=5.891±1.350 and bintercept=−6.517±13.376. The data used for the model is shown in the following table.

| antibody | human | hFcRn | ref |
|---|---|---|---|
| 1 10E8VLS | 8.1 | 2.6 | LS variant |
| 2 VRC07-523LS | 38 | 8 | LS variant |
| 3 N6LS | 36 | 9 | LS variant |
| 4 VRC01LS | 71 | 10.1 | LS variant |
| 5 CIS43LS | 80 | 15.4 | LS variant |

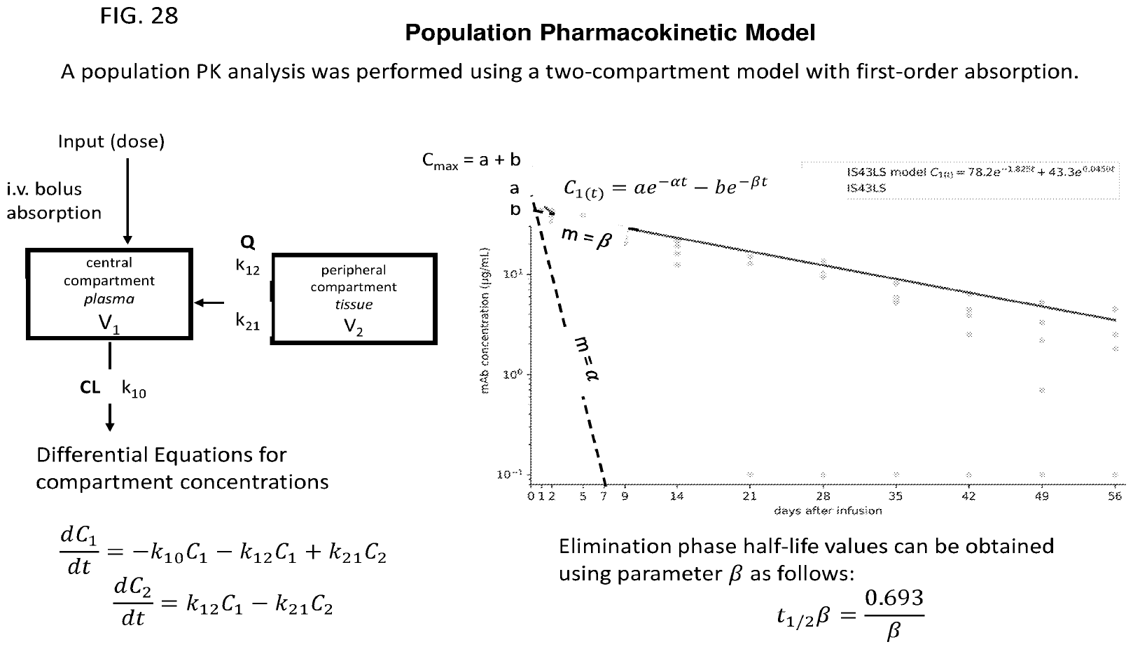

Additionally, a population pharmacokinetic model was used to interrogate the serum half-life of the variant CIS43 antibodies (FIG. 28.

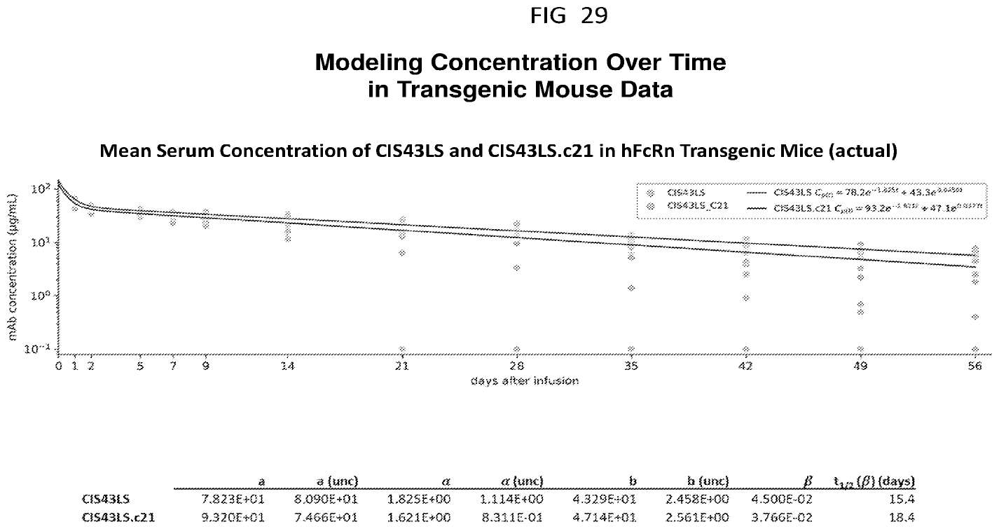

The concentration of CIS43LS.C21 over time in transgenic mouse was modeled (FIG. 29). Mouse data was fit to the following two-compartment PK model:

$${C}_{p ( t )} = a {e}^{- α t} - b {e}^{- β t}$$

Cₚ₍ₜ₎: plasma concentration, a: absorbance phase intercept, a: absorbance phase slope, b: elimination phase intercept, β: elimination phase slope.

The parameters a, α and β were fit using nonlinear least squares. Relevant data is shown in the figure. The differential equations for the two compartments were used to calculate uncertainty estimates from error propagation theory. Parameter β was obtained from measured elimination phase half-life (t_{1/2}β) values for CIS43LS (15.4 days) and CIS43LS.c21 (18.4 days).

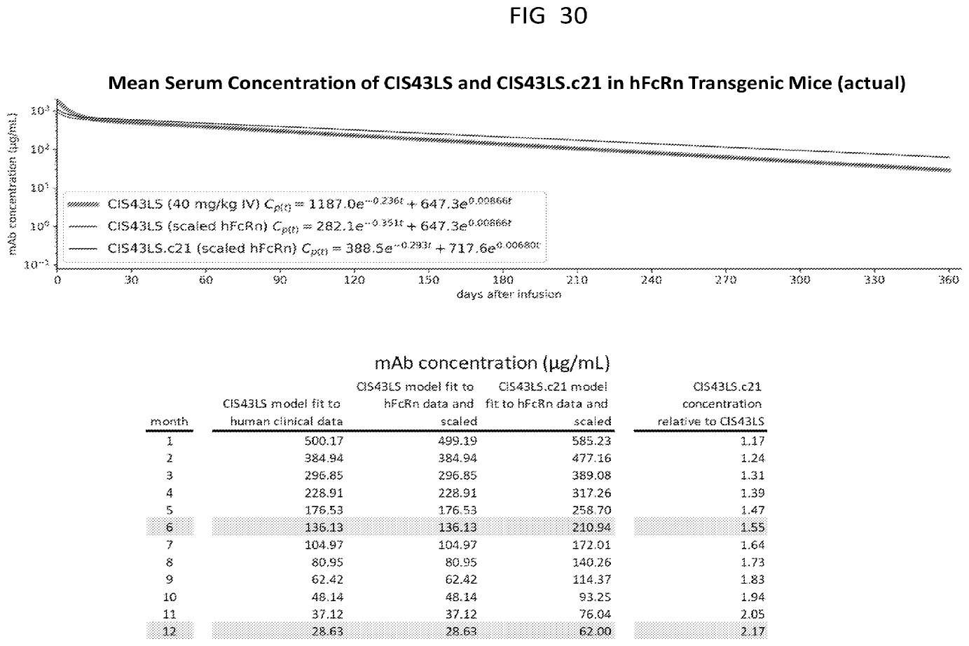

The concentration of CIS43LS.C21 over time in humans was also modeled (FIG. 30). An allometric scaling approach was used to scale time and concentration values for the transgenic mouse data. An initial two-compartment model was fit to human clinical data for CIS43LS:

$${C}_{p ( t )} = a {e}^{- α t} - b {e}^{- β t} = 1190.0 {e}^{- 0.236 t} - 647.3 {e}^{- 0.866 t} [ two - compartment model for CIS 43 LS ( 40 mg / kg IV ) ]$$

Parameter b in the two-compartment models of the transgenic mice and human clinical data for CIS43LS (40 mg/kg IV), and the elimination-phase phase half-life values were used as allometric scaling factors. CIS43LS.c21 half-life value in humans was estimated from the linear regression model:

$$scaling {factor}_{concentration} = \frac{{b}_{CIS 43 LS , trαngenic mouse data}}{{b}_{CIS 43 LS , clinical data}} = \frac{4.329 E + 01}{6.473 E + 02} = 15 \text{.739}$$

$$CIS 43 LS scaling {factor}_{time} = \frac{{t}_{\frac{1}{2} β transgenic mouse}}{{t}_{\frac{1}{2} β human}} = \frac{80}{15.4} = 5.1 \; 95$$

$$CIS 43 LS . c 21 scaling {factor}_{time} = \frac{{t}_{\frac{1}{2} β transgenic mouse}}{{r}_{\frac{1}{2} β human ( estimate )}} = \frac{101.88}{18.4} = 5.537$$

Based on this modeling, the estimated serum concentration of CIS43LS.c21 at 6 months is 210.94 μg/mL and at 12 months is 62.00 μg/mL. Based on this modeling, the estimated serum concentration of CIS43LS1 at 6 months is 136.13 μg/mL and at 12 months is 28.63 μg/mL. Thus, at 6 months, CIS43LS.c21 is estimated to have 1.55× higher concentration relative to CIS43LS, and at 12 months, CIS43LS.c21 is estimated to have 2.17× higher concentration relative to CIS43LS.

It will be apparent that the precise details of the aspects described may be varied or modified without departing from the spirit of the described aspects. We claim all such modifications and variations that fall within the scope and spirit of the claims below.

1. A monoclonal antibody, comprising a heavy chain variable region (V_{H}) and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as:
a) SEQ ID NOs: 1 and 2, respectively (m43_HH28K_17_PH104K; P3-43);
b) SEQ ID NOs: 3 and 4, respectively (D13);
c) SEQ ID NOs: 5 and 6, respectively (m42_HH28K_13_TH103R_PH104Q; P3-21);
d) SEQ ID NOs: 7 and 8, respectively (m43_HH28K_17_AH107R; P3-42);
e) SEQ ID NOs: 9 and 10, respectively (iGL-CIS43-KLH-D42.39, P4-39);
f) SEQ ID NOs: 11 and 12, respectively (D3);
g) SEQ ID NOs: 13 and 14, respectively (m43_HH28K_17_TH100M; P3-45);
h) SEQ ID NOs: 15 and 16, respectively (m43.160);
i) SEQ ID NOs: 17 and 18, respectively (m42.127);
j) SEQ ID NOs: 19 and 20, respectively (m43.151); or
k) SEQ ID NOs: 21 and 22, respectively (Core8_H-K58R); and
wherein the monoclonal antibody specifically binds to P. falciparum circumsporozoite protein (PfCSP) and neutralizes P. falciparum; and
optionally wherein the V_{H} and the V_{L} further comprise glutamate or glutamine substitutions at one or more of K13, K19, K23, or R44 in the V_{H} and R18 in the V_{L}.

2. The monoclonal antibody of claim 1, wherein the HCDR1, the HCDR2, the HCDR3, the LCDR1, the LCDR2, and the LCDR3 are set forth as:
a) SEQ ID NOs: 23, 24, 25, 26, 27, and 28, respectively;
b) SEQ ID NOs: 29, 30, 31, 32, 27, and 28, respectively;
c) SEQ ID NOs: 23, 30, 33, 26, 27, and 28, respectively;
d) SEQ ID NOs: 23, 24, 34, 26, 27, and 28, respectively;
e) SEQ ID NOs: 35, 36, 31, 37, 27, and 28, respectively;
f) SEQ ID NOs: 29, 24, 31, 38, 27, and 28, respectively;
g) SEQ ID NOs: 23, 24, 39, 26, 27, and 28, respectively;
h) SEQ ID NOs: 23, 40, 31, 41, 27, and 28, respectively;
i) SEQ ID NOs: 23, 30, 42, 26, 27, and 28, respectively;
j) SEQ ID NOs: 23, 24, 31, 26, 27, and 28, respectively; or
k) SEQ ID NOs: 23, 43, 31, 26, 27, and 28, respectively.

3. The antibody of claim 1, wherein the V_{H} and the V_{L} comprise amino acid sequences at least 90% identical to:
a) SEQ ID NOs: 1 and 2, respectively, or SEQ ID NOs: 221 and 222, respectively;
b) SEQ ID NOs: 3 and 4, respectively, or SEQ ID NOs: 219 and 220, respectively;
c) SEQ ID NOs: 5 and 6, respectively;
d) SEQ ID NOs: 7 and 8, respectively;
e) SEQ ID NOs: 9 and 10, respectively;
f) SEQ ID NOs: 11 and 12, respectively, or SEQ ID NOs: 217 and 218, respectively;
g) SEQ ID NOs: 13 and 14, respectively;
h) SEQ ID NOs: 15 and 16, respectively;
i) SEQ ID NOs: 17 and 18, respectively;
j) SEQ ID NOs: 19 and 20, respectively; or
k) SEQ ID NOs: 21 and 22, respectively.

4. The antibody of claim 1, wherein the V_{H}; and the V_{L} comprise amino acid sequences set forth as:
a) SEQ ID NOs: 1 and 2, respectively, or SEQ ID NOs: 221 and 222, respectively;
b) SEQ ID NOs: 3 and 4, respectively, or SEQ ID NOs: 219 and 220, respectively;
c) SEQ ID NOs: 5 and 6, respectively;
d) SEQ ID NOs: 7 and 8, respectively;
e) SEQ ID NOs: 9 and 10, respectively;
f) SEQ ID NOs: 11 and 12, respectively, or SEQ ID NOs: 218 and 219, respectively;
g) SEQ ID NOs: 13 and 14, respectively;
h) SEQ ID NOs: 15 and 16, respectively;
i) SEQ ID NOs: 17 and 18, respectively;
j) SEQ ID NOs: 19 and 20, respectively; or
k) SEQ ID NOs: 21 and 22, respectively.

5. The antibody of claim 1, wherein the V_{H} and the V_{L} optionally comprise one or more of K13E, K19E, K23E, or R44E substitutions in the V_{H} and a R18E substitution in the V_{L}.

6. The antibody of claim 5, wherein the V_{H} and the V_{L} further comprise the one or more of K13E, K19E, K23E, or R44E substitutions in the V_{H} and a R18E substitution in the V_{L}.

7. The antibody of claim 1, wherein the V_{H} further comprises K19E, K23E, and R44E substitutions, and the V_{L} further comprises a R18E substitution.

8. The antibody of claim 1, wherein the V_{H} and the V_{L} optionally comprise one or more of K13Q, K19Q, K23Q, or R44Q substitutions in the V_{H} and a R18Q substitution in the V_{L}.

9. The antibody of claim 8, wherein the V_{H} and the V_{L} further comprise the one or more of K13Q, K19Q, K23Q, or R44Q substitutions in the V_{H} and a R18Q substitution in the V_{L}.

10. The antibody of claim 1, wherein the V_{H} further comprises K19Q, K23Q, and R44Q substitutions, and the V_{L} further comprises a R18Q substitution.

11. The antibody of claim 1, wherein the antibody comprises a human constant domain.

12. The antibody of claim 1, wherein the antibody is a human antibody.

13. The antibody of claim 1, wherein the antibody is an IgG.

14. The antibody of claim 1, comprising a recombinant constant domain comprising a modification that increases the half-life of the antibody.

15. The antibody of claim 14, wherein the modification increases binding to the neonatal Fc receptor.

16. The isolated monoclonal antibody of claim 15, wherein the recombinant constant domain is an IgG1 constant domain comprising M428L and N434S mutations.

17. An isolated antigen binding fragment of the antibody of claim 1, wherein the antigen binding fragment comprises the V_{H} and the V_{L} of the antibody, specifically binds to PfCSP, and neutralizes P. falciparum.

18. The antigen binding fragment of claim 17, wherein the antigen binding fragment is a Fv, Fab, F(ab′)₂, scFV or a scFV₂ fragment.

19. The antibody of claim 1 or an antigen binding fragment thereof comprising the V_{H} and the V_{L} of the antibody, conjugated to an effector molecule or a detectable marker.

20. The antibody of claim 1 or an antigen binding fragment thereof comprising the V_{H} and the V_{L} of the antibody, wherein the antibody or antigen binding fragment inhibits P. falciparum sporozoite entry into the blood from the skin of the subject and/or inhibits P. falciparum sporozoite entry into hepatocytes in the liver of the subject.

21. A bispecific antibody comprising the antibody of claim 1 or an antigen binding fragment thereof comprising the V_{H} and the V_{L} of the antibody.

22. A nucleic acid molecule encoding the antibody of claim 1 or an antigen binding fragment thereof comprising the V_{H} and the V_{L} of the antibody.

23. The nucleic acid molecule of claim 22, operably linked to a promoter.

24. A vector comprising the nucleic acid molecule of claim 22.

25. A host cell comprising the nucleic acid molecule or vector of claim 22.

26. A composition for use in inhibiting P. falciparum infection, comprising an effective amount of the antibody of claim 1 or an antigen binding fragment thereof comprising the V_{H} and the V_{L} of the antibody, or a nucleic acid molecule encoding the antibody or antigen binding fragment, or a vector comprising the nucleic acid molecule; and
a pharmaceutically acceptable carrier.

27. A method of producing an antibody or antigen binding fragment that specifically binds to PfCSP, comprising:
expressing one or more nucleic acid molecules encoding the antibody claim 1 or an antigen binding fragment thereof comprising the V_{H} and the V_{L} of the antibody in a host cell; and
purifying the antibody or antigen binding fragment.

28. A method of detecting the presence of P. falciparum in a biological sample from a human subject, comprising:
contacting the biological sample with an effective amount of the antibody of claim 1 or an antigen binding fragment thereof comprising the V_{H} and the V_{L} of the antibody under conditions sufficient to form an immune complex; and
detecting the presence of the immune complex in the biological sample, wherein the presence of the immune complex in the biological sample indicates the presence of the P. falciparum in the sample.

29. The method of claim 28, wherein detecting the detecting the presence of the immune complex in the biological sample indicates that the subject has a P. falciparum infection.

30. A method of inhibiting a P. falciparum infection in a subject, comprising administering an effective amount of the, composition of claim 26 to the subject, wherein the subject has or is at risk of a P. falciparum infection.

31. The method of claim 30, wherein the subject is at risk of a P. falciparum infection

32. The method of claim 30, wherein the method inhibits P. falciparum sporozoite entry into the blood from the skin of the subject and/or inhibits P. falciparum sporozoite entry into hepatocytes in the liver of the subject

33. (canceled)
